SEQUENCE LISTING (85 sequences)

SEQ ID NO: 1 - DNA, 312 bp, Homo sapiens
  source 1..312: mol_type = genomic DNA
tttttcttctctcttttttttttattttttgagatggagtttcactctgttgcccaggctggagtgcaatggtgcagtctcagctcactgcaacctccacctcccgggtttgagcagtgattctgcctcagcctcccaagtagctgggattacaggcacccaccaccatgcctggctaatttttgtatttttagtagagatggggtttcaccatgttggccaggctggtctcgaactcctgacctcaggtgatccacctgcctcggcctcccaaagtgctgaaattacaggcatgagccaccgcacctggcc

SEQ ID NO: 2 - DNA, 930 bp, Homo sapiens
  source 1..930: mol_type = genomic DNA
  misc_difference 506: note = a, c, t, g, unknown or other
  misc_difference 513: note = a, c, t, g, unknown or other
  misc_difference 867: note = a, c, t, g, unknown or other
tttttgtatttttagtagagatggggtttcaccatgttggccaggctggtcgaactcctgacctcaggtgatccacctgcctcggcctcccaaagtgctgaaattacaggcatgagccacccccagaactcttctctttcccaaaactaaaattctgcacccattgaacagtaactccccatttctcctttcccctaactcctggcatccaccattctgttttctgtctttacgaattttactacccttggtacttcctttaagtggaatcatacaatatttttctttttctgactggcttatttcactaagcataatatttttaaggttcaagcattttgtagcatatgtcagaatttcctttctttttaaagctcaataatattccattgtatgtatataccacattttacttaaccattcatccgtcagtggacacttgggttacttccaacttttagctattgtgaataatgctgctatgaacatgaatgtacaaatatctnttcgagnccctgctttcaattcttttgggtatatacccagaagtggaattgctggatcatatggtaattctatttttaattttttgaggaaccgccatactgttttccacagcggctgcaccattttacattcccaccagcagtgcacaagggttccaatttctccacatcctcgccaacacttgttattttctgtttttttgatagtagccatcctaatgggtgtgaggtggtatctcattgtggttttgatttgcatttccctaatgattagtgatgttgagcatcttttcatgtgcttattggccatttgtatatcttctttggagaaatgtctattcaagtcctttgcccatttttnaatcgggttgtttgtttttttgttgttgagttgtaggagttctttatatattctggatattaa

SEQ ID NO: 3 - DNA, 848 bp, Homo sapiens
  source 1..848: mol_type = genomic DNA
tctcaccacctctactactgcctatctgcatctgggtgcacgtcctctgctattccttctgtttctacagaagctgtccatatttctaaggctaatcccttgcattttggtaccagacttccctgacctacacaaagatgatcaccccccaaaattgcctttctttcctgcatcaagttttttcttctctactgtattattcctattgcacattagcccaaaaaatgccgttgtttttccatcggtaaaaaaaaaaaatagagagaaaaagaaaaataatccattgccgccaatctatcttctagtcatttttccatttctctgttctccattctgcataaaccatttttacttgaattgtttatacttgctgtttacaacaaacctcctctttctctccaccccaatcagattttggtccacattcctcatgcagtggtcagtccttggtcctcaacctagtctagcctgcttgctggtgggtgtgctctcggggcatttgcttgtgtttgcacgacagctcagtccctcacttctctcaagtctttgctaataacactcttgcatccaagtcttccctgacctgctacttaaaaacaactcacccccacccctagaaatccctatcctcttccctacgctttatttttctccataatacttatcaccatgtagctgaacatgtattttactcacttttttttatattgtccatcttccctcagtgaattgaaagctccatgagggcaggaggttttgtttattgtgtttactgctttatccctagcacccagcacagcatatgtgatatagtagacactcaatacaaatctgatgtgtgtgtgtct

SEQ ID NO: 4 - DNA, 375 bp, Homo sapiens
  source 1..375: mol_type = genomic DNA
  misc_difference 287: note = a, c, t, g, unknown or other
tgatatggtttggctgtgtccccacctaaatctcatcttgaattgtagttcccataatccccatgtgtcatgggagggacccagtgggaggtaatttaatcctgggggcagttaccctcctgctatgtgtgtgtgtgtgtgtgtgttctcacgagatctgatggttttataaggggcttttcccccttcgctcggcacttctccttcctgccgccatgtgaagaaggacgtgtttgcttccccttccgccatgattgtaagtttcctgaggcctccccagccatgcngaactgtgagtcaattaaacctctttcctttataaattacccagtctcgggtgtgtctttattagcagcgtgagaacggactaataca

SEQ ID NO: 5 - DNA, 311 bp, Homo sapiens
  source 1..311: mol_type = genomic DNA
ttttgctcttctttttttttttttttttgagagacggagtctcgctctgtctcccaggctggagtgcagtggcgcgatctcggctcactgcaagctccgcctcccgggttcacgccattctcctgcctcagcctcccgagtagctgggactacaggcgcccgccaccacgcccggctaattttttgtatttttagtagagacggggtttcaccgtgttagccaggatggtctcgatctcctgacctcgtgatccgcccgcctcggcctcccaaagtgctgggattacaggcgtgagccaccgcgcccggcc

SEQ ID NO: 6 - DNA, 892 bp, Homo sapiens
  source 1..892: mol_type = genomic DNA
tatttacattattttaataggtaatgcaattacatcttctaaaattcagaaagcataaaagtacataagagaaaaactcatcatccagcccttgcccttgaggacgctagtttctacccacacagctaaaccttgttacaaattacttgggtgtattaccagtggcattgccaggtttaacaaataaaaacataggattcctgattaagtttaattttcagataaataattacatgctttttaagtagaagtatttcccaaatattacaagagacaatatatttaaaaaattgtttgatatccaaatttaactatgcgtcctatattttgtctggcaaccctaatccttcattgtatggatgtaccccaggttcttatgcaaatagagaaaatatgcactcttttattatcctccgtccctttcttttacagacaatgatagaatgctttatttagtacatacgtcattctgcacctgtgcgagtattttagttaaccacacttttggggattgttctgctatcataaaagatatttctcttttttaccataatgtatttcattaaaccctctttcacaggttaggtcccatatttttctttttttttttttttttgagagacggagtctcgctctgtctcccaggctggagtgcagtggcgcgatctcggctcactgcaagcctgataggtgaaaaatggtatctcagtgtagttttaatttgcatttctcttattatgagtgaggttgagcatcttttcatatgtttaagagccatttgtatttccttttctgtgaactgtctgtccgcctcccgggttcacgccattcttgggttgttggtctttttcttattgacctgcctcagcctcccgagtagctgggaaatcta

SEQ ID NO: 7 - DNA, 312 bp, Homo sapiens
  source 1..312: mol_type = genomic DNA
ggtcagtcgcggtggctcatacttgtaatctcagcactttgggaggccaaggcgggtggatcacctcaggtcaggagttcaagaccagcctgaccaatgtggtaaaaccctgtctctactaaaaatacaaaaattagctgggcgtggtggcacatgcctgtaatcccagctactcaggaggctgaggcaggagaatcgcttgaacccgggaggcagaggttgcagtgagccaagataacaccactgcactccagcctgggtgacagagcaagactccgtctcaaaaaaaaaaaaaaaagatccagtgatagg

SEQ ID NO: 8 - DNA, 311 bp, Homo sapiens
  source 1..311: mol_type = genomic DNA
tatctattcacatttatatgctcttagtcagaaaataaaatacctctcttgaaatgggcctgactccggagagggagaccagattcactgcaagctccgcctcccgggttcccgccattctcctgcctcagcctccccagtagctctgactacaggcgcccgccaccacgcccggctaatttttttattttttagtagagacggggtttcaccgtgttagccaggatggtctcgatctcccaacctcgtgatccgcccgcctcggcctcccaaagtgctgggattacaggcatgagccaccgcgcccggcc

SEQ ID NO: 9 - DNA, 311 bp, Homo sapiens
  source 1..311: mol_type = genomic DNA
tttttttttctttttttttttttttcttttgagacggagtcttgctctgtcgcccaggctggagtgcagtggcgcgatctcggctcactgcaagctccgcctcccaggttcatgcaattctcctgcctcagcctcccgagtagctgggactacaggcgcccgccaccacgcccggctaattttttgtatttttagtagagacggggtttcaccgtgttagccaggatggtctcgatctcctgacctcgtgatccgcccgcctcggcctcccaaagtgctgggattacaggcgtgagccaccgcgcccggcc

SEQ ID NO: 10 - DNA, 311 bp, Homo sapiens
  source 1..311: mol_type = genomic DNA
tgctgggcatggtggctcacgcctgtaatcccagcactttgggaggccaaggcgggcagatcacgaggtcaggagttcgagaccatcctggctaacacagtgaaaccccgtctctactaaaaatacaaaaaattagccgggcgtggtggcgggtgcctgtagtcccagctactcgggaggctgaggcaggagaatggcgtgaacccaggaggcggagcttgcagtgagctgagattgcgccactgcactccagcctgggtgacagagcgagactcctcttcaaaaaaaaaaaaaaaaaaaaaaagactcat

SEQ ID NO: 11 - DNA, 312 bp, Homo sapiens
  source 1..312: mol_type = genomic DNA
  misc_difference 57: note = a, c, t, g, unknown or other
ggccgggcgcggtggctcacgcctgtaatcccagcactttgggaggccgaggcgggnggatcgcttgagcccaggagttcgagaccagcctgggcaacatggcgaaaccccgtctctacaaaaaatacaaaaattagccgggcgtggtggcgcgcgcctgtagtcccagctactcgggaggctgaggcgggaggatcgcttgagcccgggaggtcgaggcttcagtgagctgagatcacgccactgcactccagcctgggtgacagagcgagactctgtctcaaaaaaaataaataaaaaataaaaataaaa

SEQ ID NO: 12 - DNA, 312 bp, Homo sapiens
  source 1..312: mol_type = genomic DNA
ggccgggcgcggtggctcacgcctgtaatcccagcactttgggaggccgaggcgggcggatcacctgaggtcaggagttcgagaccagcctggccaacatggtgaaaccccgtctctactaaaaatacaaaaattagccgggcgtggtggcgcgcgcctgtaatcccagctactcgggaggctgaggcaggagaatcgcttgaacccgggaggcggaggtttcagtgagctgagatcacgccactgcactccagcctgggtgacagagcgagactctgtctcaaaaataaataaataaataaaaaataaaaa

SEQ ID NO: 13 - DNA, 1039 bp, Homo sapiens
  source 1..1039: mol_type = genomic DNA
tatcgaaaattcacccccgatattcacgtaggttcttttctattttccctaagcgtcggcaggtttgagaaataaagggacagagtacaaaagagagaaattttaaagctgggcgtccgggggagacatcacatgttggtaggttccatgatgccccacaagccgtaaaaaccagcaagtttttattagtgattttcaaaaggggagggagtgtacgaataggtgtgggtcacagagatcacgtgcttcacaaggtaatagaatatcacaaggcaaatggaggcagggcgagatcacaggaccacaggaccggggtgaaattaaaattgctaatgaagttttgggcaccattgtcattgataacatcttatcaggagacagggtttgagagacaaccggtctgaccaaaatttattaggtgggaatttcctcgtcctaataagcctgggagtgctatgggagactggggcttatttcatcccgtagaggcaaccataaaagacaggcccaagcggccatttagagcctcccaggacattctctttctcagggtgttccttgctgagaaaaagaattcagatatttctccatttgcttttgaaagaagagaaatatggctctgttcccggctcacggcgcagatttaaggttatctccttttccctgaacaattgctgttatcctgttcttttttcaaggtgcccagatttcatattgttcaaacacacatgcttacaatttgtgcagttaagcaatatcacagggtcctgagggacatacatcctcctcagctgacaggattaagagattaaagtaaagacaggcataggaaatcacaagggtattgattggggaagtgataagtgtccatgaaatcttcacaatttatgtttagagattgcagtaaagacaggcataagaaattataaaagtattaatttggggaactaataaatgtccatgaaatcttcacaatccacgttcttctgccatggcttcagccggtccctccgttcggggtccctgacttcccgcaaca

SEQ ID NO: 14 - DNA, 355 bp, Homo sapiens
  source 1..355: mol_type = genomic DNA
tgatatggcttggctgtgtccccacccaaatctcatcttgaattgtacgttccataattcccatgtgttgtgggagggacccagtgggagataattgaatcatggggagcagtttcccccatactgttctcatggtagtgaataagtctcatgagatctgatggttttatcaggggtttctacttttgcatcttcctcattttcctcttgccaccaccatgtaagaagtgcctttcacctcccaccatgattctgaggcctccccagccatgtggaactgtaagtccaattaaacttctttttcttcccagtcttgggtatatctttatcagcagcatgaaaatggactaataca

SEQ ID NO: 15 - DNA, 311 bp, Homo sapiens
  source 1..311: mol_type = genomic DNA
ttttttttttttttttttttttttttttttgagacggagtctcgctctgtcgcccaggctggagtgcagtggcgcgatctcggctcactgcaagctccgcctcccgggttcacgccattctcctgcctcagcctcccgagtagctgggactacaggcgcccgccaccacgcccggctaattttttgtatttttagtagagacggggtttcaccgtgttagccaggatggtctcgatctcctgacctcgtgatccgcccacctcggcctcccaaagtgctgggattacaggcgtgagccaccgcacccagcc

SEQ ID NO: 16 - DNA, 311 bp, Homo sapiens
  source 1..311: mol_type = genomic DNA
tttctttacttcaaccttctttttttttttgagacagagtctgctctcgttgcccaggctggagtgcaatggcgcaacctcagctcactgcaacctctgcctcctgggttcaagcaattctcctgcctcagcctcccaagtagctgggattacaggcatgcgccaccacgcccagctaattttgtatttttagtagagacggggtttcaccatgttggccaggctggtcttgaactcctgacctcaggtgatccacccgcctcagcctctcaaagtgctgggattacaggcgtcagccaccgcactcagcc

SEQ ID NO: 17 - DNA, 311 bp, Homo sapiens
  source 1..311: mol_type = genomic DNA
ggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcatggtggcaggcacctgtagtcccagctactcaggaggctgaggcaggagaatggcgtgaacccaggaggcggagcttgcagtgagccgagatcatgccactgcactccagcctgggtgacagagtgagactccatctcaaaaaaatgacacaaaataaaataaaataa

SEQ ID NO: 18 - DNA, 312 bp, Homo sapiens
  source 1..312: mol_type = genomic DNA
ggccgggcgtggtggctcatgcctgtaatcctagcactttgggaggccgaggctagcggatcacctgaggtcaggagtttgagaccagcctagccaacatggcgaaaccctgtctctactaaaaatacaaaaaatggctgggcatggtggcagacatctgtaatcccagctactcaggaggttgagacaggagaattgcttgaactcaggaagcggaggttgcagtgagctgagattgcgccactgcactccagtctgggtgacagagcaagactctgtctcaaaaaaataaaataaaataaaaataaaata

SEQ ID NO: 19 - DNA, 311 bp, Homo sapiens
  source 1..311: mol_type = genomic DNA
ggccgggcgtggtggctcatgcctgtaatcctagcactttgggaggccgaggctagcggatcacgaggtcaggagtttgagaccagcctagccaacatggcgaaaccctgtctctactaaaaataccaaaaaatggctgggcatggtggcagacatctgtaatcccagctactcaggaggttgagacaggagaattgcttgaactcaggaagcggaggttgcagtgagctgagattgcgccactgcactccagtctgggtgacagagcaagactctgtctcaaaaaaataaaataaaataaaaataaaata

SEQ ID NO: 20 - DNA, 634 bp, Homo sapiens
  source 1..634: mol_type = genomic DNA
ttcttctcacggagtgaaacttttcctttgattgagcagtttcgaaactctctttttgtaaaatctgcaagtgaacatttggagtgctttgtggcctatgatagaaaaggaaatattttcacttaaaatctagacagaagcaatctgagaaacttctttgtgatgtttgtattcatcttacatagttaaacctttcttttgattgagcagttttgaaactctctttttgtcgaatctgtaagaggacatttggagtgctttcaggcctatggtgaaaaaggaagtatcttctcatgaaaactagacagaagaattcagacaaacttctttgtgatgtgtgcatttatctcacagacttgaacctttcttttgattgagcagtttgaaacactctttttgtagaatctgcaagtggacatttggagcacgtggaggcctatagtggaaatggaaatgtcttcacactaaaaactagacagaagaattctgagaaacttctttgtgatctttgcattcatctcacagagttgaacctttcttttcattgagcagtttgaaaacactctttttgtagaatcggcaagtggacatttggaacgcattgcagactatgatggaaaaggaaatatctcca

SEQ ID NO: 21 - DNA, 135 bp, Homo sapiens
  source 1..135: mol_type = genomic DNA
cagcgcaagtaatatatcctggaattggaagcagaatgggaactatggactcagtgaagattgttctggacattatttgattcttctttctcgctgccaattggatactttttttttttttttgagacagagtct

SEQ ID NO: 22 - DNA, 134 bp, Homo sapiens
  source 1..134: mol_type = genomic DNA
cagcgcaagtaatatatcctggaattggaagcagaatgggaactatggactcagtgaagattgttctggacattatttgattcttctttctcgctgccaattggatacttttttttttttttgagacagagtct

SEQ ID NO: 23 - DNA, 773 bp, Homo sapiens
  source 1..773: mol_type = genomic DNA
ttcatcttacagagataaacatttatcttgattcagcagtttggaaacactgtttttatccattctgtgaatggacatttgggagttcattaaggcacacatgaaaaggtgtatatcccagggtaaaaactagaaggaagctatctgagaaaccactctgtggtgtgtgcattgatctcgctgatttaaagctttcttttgattcagcagtttggaatcactgtttgtgctcattctgtaaatggatatttgggagctcattgaggtcaatggcaaaaaagtgaataactgaggataaaaactagaaataagctatctgagaaacggatttatgatgtgtgcattcatctcgcaaaattaaacatttcttttccttcaacagtttggaaacacggtttttataggatctgcaaagggatattttggagcacattgaggtctatggtgtaaaagaaaatatcttcagataaaacctagaaagaagctttctgagaaattgcattttgatgtgtgcattcatctcacagagttaatacttttttttgattcaccagtgtggatacactgtttttgtccattctgctaatggacctttgggggctcatttaggccaatggcaaaaaagtgaatatcccaggataaaaactagaaggaaggtatctgagaaactgctatgtgatgtgtgccatcatctcacagtcttaaaagtttcttttcattcaccagtttggaaacagtgtttttgtagaatctgcaaagtcatatttttgagt

SEQ ID NO: 24 - DNA, 927 bp, Homo sapiens
  source 1..927: mol_type = genomic DNA
gatgaatgcacatatcacaaaaagtttctcagattgcttctgtctagattttatgtgaagttatttccttttctaccatagaacccaaagcacttataatgtccccttgcagattctacaaaaagaatgtttcaaaactgctcaatcaaaagaaaggttcaaatttgtgagatgaatgctcgcatcaccaagaagtttctcaaaatacttctgtctagtttttatgtgaagacatttctttttccgtgataggtctcaaaatgatcaaaatttccacttgcaaattctacaaaaagagagtttcaaagctgctcaatcaaaagaaaggtttatatctgtgagatgaacacacacttcacaaagcagtttctaagatttcttctgcctagattttatgtaatgatatttccttttctaccatactttgcaaactgctccaaatgtccacctgtagattctacaaaaacagtgtttccaaactgctcaatcaaaagaaaggttaactctgtgagatgaactcacacatcacaaagaagtttctcagaattcttctgtctagttctaatgtgaagatatttccttttggaccagaggccccaaggctatcgaaatgtccccttgtagattctacaaaaagagtatttcaaaactggtccatcaagagaaaggctcaactctgggagatgaatgcacacatcacaaagaagtttctcagaatgcttctatctagtttttacatgaagctatttccttttccaccataagcctcaaagcgctccaaatgtccacttgcaggttctgcaaaaagagagtttccaaagttctcaatcaaaagaaacggttaactctgtgagataaatgcacacatcacaaagaagtttctcagaatttttctgtctagtttttgtgtgaagatatttcattttc

SEQ ID NO: 25 - DNA, 312 bp, Homo sapiens
  source 1..312: mol_type = genomic DNA
agccaggtgcagtggctcacacctgtaatcccagcactttgggaagctgaggcgggtggatcacttgaggtcaggagttcgagaccagcctggccaacatggtgaaaccccgtctctactaaaaaagtaaaaattagccaggtgtggtggcgggcacctgtaatcccagctactcaggaggctgaggcaggagaattgcttgaacccgggaggcggaggttgcagtgagctgagattacaccactgcactccagcctgggcaagagagcgagaatccgtctcaaaaaaaaaaaaacaaaaaaaacaaaaaac

SEQ ID NO: 26 - DNA, 268 bp, Homo sapiens
  source 1..268: mol_type = genomic DNA
aattacaacaacccttaatatttgtaaagcgcttgataattcagggagggcttttaaaaacttctatccatctcttccccccagcaatcctatgaggtaggcagggctggaatcatttgctgcattttatcgaataggaagccatgactcgtagaggtctggtgacttaactgaggtcacccactgaggacgtggcagagctgggtcctgaatctgtggccctcaggctctttgcctggccctgcctcacatagactacgctgcctct

SEQ ID NO: 27 - DNA, 312 bp, Homo sapiens
  source 1..312: mol_type = genomic DNA
tttcttactttttttatttatttttttcttgagacagggtctcacttggtcacccacgctgaagtgcagtggcgtgatcttagcccactgcaacctctacctcccgggctcaagcgatcctccctcctcagcctcctgagtagctgggaccacaggcatctgccaccacacccagctaatttttgtatttttagtagagatggggtttcaccatgttggtcaggctggtctcaaactcctggcttcaagtgatctgcccacctcagcctcccaaagtgctgggattacagatgtgagccactgcgcctggcc

SEQ ID NO: 28 - DNA, 312 bp, Homo sapiens
  source 1..312: mol_type = genomic DNA
ttgatttttttttcttttatctttttttttgagacagagttttgctctgttgcccaggctggagtgcagtggcgtgatcttggctcactgcagtgtccgcctcctgggttcaagcgattctcctgcctcagcctcctgagtagctgggattacaggcatgcaccaccacgcccggcttcattttttatttttagtagagacagggtttcaccatgttggtcaggctggtctcgaactcctgacctcaggtgatccacccgccttggcctcccaaagtgctgggattacaggcgtgagccaccgtgcccggcc

SEQ ID NO: 29 - DNA, 312 bp, Homo sapiens
  source 1..312: mol_type = genomic DNA
ggctgggcatggtggttcacgcctataatcccagcactttgggaggccgaggcaggcagatcacctgaggtcgggagttcgagaccagcctgaccaatatggtgaaaccccgtctctactaaaaatacaaaaattagccgggcgtggtggtgcatgcctgtaatcccagctacttgggaggctgaggcaggagaatcgcttgaacccaggaggcggaggttgcagtgagccgagattgcgccactgcactccagcctgggtaacagagcgaaactccgtctcagaaaaaaaaaaaggcagatatgaaaaagg

SEQ ID NO: 30 - DNA, 520 bp, Homo sapiens
  source 1..520: mol_type = genomic DNA
aatattaatttgggctctccagagaaacagaaccaatagattacacatagatatatagaaagacagttattacaaggaattggctcacaggattatgtaggctgagaagtcccatgacctgccgtctgcaagctggagacccaggaaagccggtggtgtagttccagtccgagttgggaggtctgagaatcgggagagctgacagagtaagctctagttgcagcctggagtccagagggcaaagaacacctatgtcccatctcaagcacggtcaggcgtcccagctcaagcagagagaaagaagactttcctgctcagcctccttgctctaagcagcctcccgacggattggatgaagctcacccacgctggggagggcagccaccttcactcagtccaccaattcaaatgctaatctcatccagaaacaccctcacaggcacacccagaaataatgtttagccacatatctgggcatcctgaggaccagtcaaattgactcataagtctcaccatcata

SEQ ID NO: 31 - DNA, 312 bp, Homo sapiens
  source 1..312: mol_type = genomic DNA
ttatgactattatttcttttttttttttttgagatggagtctcgctctattgcccaggctggagtgcagtggcatgatctcggctcactgcaacctctgcctcccgggttcaagcgattctcctgcctcagcctcctgagtagctgggattacaggtgcctgccaccacacccggctaatttttgtatttttagtagagatggggtttcactgtgttggccaggatggtcttaaactcctgacctcaagtgatccacccacctcggcctcccaaagtgttgggattacaggcgtgagccaccatgcccggcc

SEQ ID NO: 32 - DNA, 517 bp, Homo sapiens
  source 1..517: mol_type = genomic DNA
  misc_difference 279: note = a, c, t, g, unknown or other
  misc_difference 286: note = a, c, t, g, unknown or other
ctccatgttctgacctcaggtgatctgcccgcctccgcctcccataagttccttctgcacctattccattccatctgcaatgtcatcctggccaacctgtgaatgggtatagcaggatagtaagttagtcccagtgtttaggcagactccatagaccagatttaacgtgtttgtataaaccgcatggctcgctcttactcagccttcgggtcggccttaagatacacagttttaagaaaagcattgtcttcactcttagtgtagatcaggtcctcctcncccccancacgttatgtccctgtcattgaacattatacttttatgaagaattgtgagctgtctgagattttaccttacttgcaaactaacaagtgggcctgccatagtttcatgtctggcagaagactccaacaggtcagagattttcatctgtcttgttcactattgtctacatagtgcctggaaaaatgcctggcacacagtaaaccctcaatcagtattttatgaatgagtgaac

SEQ ID NO: 33 - DNA, 448 bp, Homo sapiens
  source 1..448: mol_type = genomic DNA
tgtgttagtcaggataggctaacctctgtaacacacaaccccaatttttcagtggctcagaacaataggagtttctttcttactcatgtaaagtccaggtgtggcagaaacttgcgggggagaaagtctactccacagcatcactcagggacccaggctcctgccttacggtggttccgccattttccagggcctcggagtcctccatcccatctcctccattcagctggtggatgtgggaggagagcgtgtatgaaagaccacacagaagctttttttgggccaggctttgagacggcgcacatcagtttcgctcttgttgcccaggctggagtgcaatggcgcaatctcggctcaccgcaaaagacactgggaaatttggtctggccatgtgtctaggaagcagaggagatggggtttcataaactcctgaactctctctaccacg

SEQ ID NO: 34 - DNA, 312 bp, Homo sapiens
  source 1..312: mol_type = genomic DNA
ggccgggtgcagtggctcatgcctgtaatcccagcactttgggaggccgaggcgggtggatcacctgaggtcaggagatcaagaccatcctggctaacacggtgaaaccccatctctactaaaaatacaaaaattagctgggcgtggtggcaggcgcctgtagtcccagctactcgggaggctgaggcaggagaatggcgtgaacccaggaggcagagcttgcagtgagccgagattgagccactgcactccagcctgggggacagagcaagactctgcctcaaaaaaaaaaaaaaatggaaaaaaaaaaaa

SEQ ID NO: 35 - DNA, 268 bp, Homo sapiens
  source 1..268: mol_type = genomic DNA
aagatggggaggatggtggtggagcaggctggggagatgaggagcttgaggggcctaggagctaaccacccattcactgtttatgggtggggtggctccgggcaggtggcctgacccctctaaacctcagtttccccatctgtaaaatgggagtaattgtacctgcctcacgggcatgtagcaggtgggagagagagtaatacgggcaagacgtctagtgcagtggctggaacataacaagcacccagcaagtggtggccccattgct

SEQ ID NO: 36 - DNA, 312 bp, Homo sapiens
  source 1..312: mol_type = genomic DNA
ggccgggcacggtggctcaaacctataatcccagcactttagaaggccaaggcgggcagatcacctgaggtcaggagttcaagaccagcctggccaacatggtgaaaccccgtctctactaaaaatacaaaaattagccaggtgtggtggcgggcacctgtaatcccagctactcgggaggctgaggcaggagaatcacttgaacccaggaggcggaggttgcagtgagccgagatcatgccactgcactccagcctgggcaacagagtgaaactctgtctcaaaaaaaaaaaaaaaaaaaaaaaaaaaaaa

SEQ ID NO: 37 - DNA, 3809 bp, Homo sapiens
  source 1..3809: mol_type = genomic DNA
  misc_difference 2867: note = a, c, t, g, unknown or other
aaatcgaattctctgtctccaattgcttcttgctttcaagggacccaagcaggaggctgctccgccacatataaatgtaggacctgaagaagcagtctgcagctcaaggctggaattggcctagatcctccctggaggcccagattcacagtgtaatcgttccaagccattaatcacaggcacccggcgggactgtgtctcgtcccaaagggcatctcaagaggttttgcagagtttcatgtaaatgatcattacactactaataaatatggttagtgactttgagccacatcaggtgccttgggccaaggagctggcggaaggagtatctaggcttccttaagtgaaggcactgaacagccagcagaattaatccatcaactgctctcctttcttccccacttctcatacacacacacacgcacacgcgcgcatgtacacacacgctggcgagagttttagaccagagcaccaagccctccaatgcccatgttaagaaaatatgaagttaatatgctcggcttcctgttgaagtccgcctcattcccactaactgatcaggatacactcacagttcacccgcaagagcagacaacaaatgaggagagatagggcaacggcacctctgtctgcagcatttttaaaaatatagatacctactctttcaaacagatttgaagtgagaatatggtttctatgataatacccaaaaatgaactgcataaaccattgcctaatgctgaacagcactcctcgattcttcctaaatctatactcgcagttcttatgcaagaaaaaggtaacaacatccctgttctccatgcttccattttacttcacatctggactgtcactgcaaacagctgaatctgattttaaaaaggtacacatataccactttcattatcaatgtcttatacaaattacctgatacatgtagttacttagaaattatttcccattgcttcaatatctatgaacaaaaagtaaacactacataagataaaataaaccaaaatattaatgaggctcacagaactactgatgaataagctcagaaaactgttagtattttttatactatgtgaaaccaaagaccgatcaagaaaatgtatttactaactgtatttaataaccactgcaataagactgaatgacttaagcaaagataaattgataaaagggccgggcacggtggctcaaacctataatcccagcactttagaatggccaaggcgggcagatcacctgaggtcaggagttcaagaccagcctggccaacatggtgaaaccccgtctctactaaaaatacaaaaaaaattagccaggtgtggtggcggtgcacctgtaatcccagctacttcgggaggctgatcaagtggcaggagaatcacttgaacccaggaggcggaggttgcagtgagccgagaactgcactccagcctgggcaacaagagtgaaactctgtctcaaaaaaaaaaaaaaaaaaaaaaaactgaagcaatcaaagcggctagtaaaagacttacagaaaaagttgatttgtaatagagggtcaatctggcacaactgtagattacctataaatgtttagaattatcctctcttctaaaatgcaaacagaatcagcatgcaaaataactttggcttacatgattcaattctagtcatctaacaaaagaatttactccaaatccgggaagttatggccagcctggagggctaagaagaatctgaaatagaaagaaatttttattatttaaatacttcccctctcttctgctaaacaatctctttaggcttgatgggcaaaaaggatgtcgcaagtttctacattcttctaaaaaactcagtactttaaaagctgatagagaaattcagtcacccaaaggagtcagctgtcacagaaaatgttatctaatagtggaaagattacataatgccgatctgcactcgtatctaatagagattttgaagaactaaggaattaggcctcctaagataaaatctttgcagcatgcaatgagcaagtataaagatctcattctcttcccaggtgactggacaagggaaagattcaagcaagcctctgggaagcggggaatatttcaaggagacttacttatttcaacaaacatttacttgggactggaccatctatgctgggcctaggactgtgctaggagctgatgatacatggatgaacaggattaacatcatataagaaatgatttgttctacgtggacgtactgaatctctaagccaacactaaaatcaaatttcatatactgccaaaaatcaccaagaactttagtgatgatatacaaatggagtttgatatggaaaactggaggccaataaaaatgaggaaagaacaacttcctataacagattagttccctagtaggaaaaaccatgttattaaagctctaagttagggtaacatttaggagtatctaggcttccttgagccaaaacaggttaatctcatcagttttagatcataacttaagaaaatatgaagttaatatgcttagaaagtactaaaaccacacattaaaagaggcaataagaccaaggcaataaatttacttgtagtaccggttccgagacacttatttggtattagaaagccatctaataaaggttaaaatattagagaacataaactataaatgtcaatgacacaaaatctaatctatcatcttaaagctgcaatcgagaggatcactcttacaaatgatagaattaaaaagagggtaattagcatcaaaaattcattttttttcctgaatttaaaactatagggctatttttacagcaaatcaaatgtagagcggtgaaaaataggccccaaaaccgatcncagtgttatgccagcagaacctgaagaaacacatcatggtcctctggtaagtaagctgatgacctaacagcaaattcagacacagaaacaaaaggaactttcaggtgtctacacacatatcattaagaaagattatgtggcagatgctacatcacatttacgtctcaggaaagaccatgtggcccagagatgagggatggtttttatgtcaattcaagtaaaagtggttgcccctaaaaataaccacatctgtatacataaagaaagcttgtctgatgatacatgcagaagatgagaaaaggcttaagtggaaaatgaatataaaattggataatgtgtcaccttggctgacatggattacaccaacaagcatagtaattctgctaagattacataccaataattagctttccagtgtgggactgataatggattaacatagttccattaacgttaactcaagattattttagaaaataactgtaaacgcctgctcagattcagccatataacaggatactagcaagttcttactaacagaccagacattctgctgcttgattcatcttttaatttggtagaacttactaaaataaccttttctcttaaatggcacatccaaaaaccctcacagaaaaaaatatttaaaagtatgtaatccccagtgagattcaaaatattgtggagataataaccaaacagaacaaatgcaccaaccatctcggcgatcacaataaacacaagattaaaaatcaaaaacataactttgcagtaaccttgtgactagctggtatcagagcagcacgcaaatctgacagaaaacagcaattaaacaaaggcatttctgtcttgacaacagacttctataaaacaaggaactaagggccgttttggtaagaaatagaaaggccagattaatcagaggaatgaaattaatattta

SEQ ID NO: 38 - DNA, 291 bp, Homo sapiens
  source 1..291: mol_type = genomic DNA
acagtggcacacgcctgtagtcccagcacctttggaggctgaggtgggtggatcacctgagtccaggagttcgagaccagcctgggcaacatggcatgaccctgtctctgcaaaaaatatgaaaattagccaggcatggtggtgcgtgcctgtagtcctagctacttgggaggctgaaataggaggatcgcttgagcccaagagttgaaggttgctgtgagccaagatcgcaccactacaatccagcttgggtgtcagagtaagaccctgtctcaaaacaaaaagataaat

SEQ ID NO: 39 - DNA, 261 bp, Homo sapiens
  source 1..261: mol_type = genomic DNA
gtatttagattcccagactttggagccagatcggctgagttcaaaccctgtctgtgcaactatcagctgttcagctgtgtgaccttgggcaagttacttaacctctctgaaccctgattcctcctgcaaacggtgggaaaaataatagaaccaatctctctgggttgctgtaaggattaaattaatttacagtgcatgtaacatgcttacagcaatgtctggcacacagtaagcactaagtatttgttgttatttgtcatc

SEQ ID NO: 40 - DNA, 4705 bp, Homo sapiens
  source 1..4705: mol_type = genomic DNA
aggattcatcaccttctgacttaggagctgctggtctctgaataacctacagtggtaccccaataacacatcatgaacattggactccgagatgtgctggcttcaggtgtgacccagaacatttcccactgtggtgggtatagtgaaagacaccttctgtttgagcaaagcagaggaaaaagtaaatgggattctgtcttgcacctgaggtaccagcttggccacagtggggtagagcaccaagcagactcttagggtccctgagtcagcatttctggacctgccctgcaccaaagagtaagtcccagttgtggaagcagtcatcataagctgactgaagagtgcttgggtgttgagtgaacatcagtgatagcccagtggaaacccctgtgggctggtggtggtggcagacacattgtgaggctcctctgcctgcagaaaggggagggaggagtggtaaggatgttgtcttgtaatttgaatgccagcttagctgcagtagaatagaacacgagataaataaggcttctgacttcaatacctggctcccacacaatatctctgaacctacctggggccaaagggaactcactgccctgaggagacacaaacctggctggttaccacctgctgattgtagaggcctaaggtcttgagtaaacttaggtggtagccaggcagtaattacactgggccttgggtgacacccagtgctgtgctcacttcaagtctaaattagcacagtcccagtggtgggggcaacagggaagcttatgtcatcccaccccagctccaggtagctcagcatagagaaagaaattccattcgtctggaagaaactaagggaagagaacaagagtctctgcttggtaatccagataattcatcgattttaaccaagaccaaccacatggtacctctatgagtctgcaagaacaacgttattgagtttgagatccaagtaccttcaaattctggaaagccttcccaacaagggtggacacaaagcccagactgcgaaggctacaataaatgcctaactcttcaatgccaagacacagatgaacatctacacattaagaccatccagtaaaatatggcctcacctaacaaactgaataaggcataagaggccaatcctggagaaacaaaataagtaacttttcagacagatgattcaacatagttgtgttgaggaaacaaattcaagataaaatagagaaggaattcagattctatcagataaatcaaacaaagaggttgaaataattaaaaggaatcaagcagaaattgaagttaaaaaagcaattggcatactgaagaatgcatcagtctcttaatagcaaaactgatcaggcaaaagaaaaaattattgagcttgaacataggcaatttgaaaatatgtagagaagacaaaaaagaatgaagtatgcctacaagatttagaaaacaccctgaaaagggcaaatctaagagttgttagccttaaagagagaaagagaaaggggtagaaagtttcaagaatacgatcagagaacttcccaaatctataaagatatcaatatccaagtacaaccaggttacagaacacaaagcagattgaacccaaagacgactacctcaagacatttaataatcaaactcccaaaaattaagaataaaggatcctaaaagcagcaagaaaaaagaaacagcatacaatgaaactccagtacgtctggcagcagattttcagtggaaatcttacaggccaggagagaatagcatgacatatttaaagtgctgaagtagcaaagcttttacactagaatagtctatcccgtgaaaatattccatagccatgaaggagaaaaaaatgaccttcccagacaaacactgagagatttcattaaaaccagacctgtcctacaagtaatactaaggggcattctcttccatctgtaagaaaagaatgttaatgaacaataagaagtcatctgaaggtacagaactcattggtaatagcacagagaaaaactgagtattataatatggtaactgtggtgtgtaaaatactcaagtagaaatactcaatgatgactcaatacaactacaacaatgtttcaagacaaagacagtacaataagacataaagagaaccaacaaaaagttgaaaaatggggaagcaaagttaaaggtagagtttttactagttttcttattgcttatttgttcacttaggcaatcagtgtgaagttgtcagcagttaaaacaataggttataagatagtatttgtaagtttcatggtaacctcaaataaaaaaacatacaacagacacagaaaccaaaaagcaagaaattaaatcataccaccaagaaaaatcatgttcactaaaatgaagacagaaaggaaggacaaaagaaagagaagatgcaaaaacaaataacaaaatggcaggagtgaacacatacttatcaataataatgctgaatggaaatgcactaaacactccaattagatgacatagaatgggtgaaaaaaagaaaaaggaagacccaattatctgctgcccacaagaaatacacttcatctataaagatacacatagactgaaaaataaaagaatggaaaaatgttccatgccaatgaaaacaaaaaaaaagaaccaaagtagctatacttacatcagataaaatatatttcaagataaaaactgtgaagacagaacaaatgtcattttatgataaagggatcaattcagtcagagaatataacaattttaaatatccatgcaccctacactaaagcactgagacatagagtattagagctaaagagagaggccccaatacaataataattgagacttcaacacaccactttcagcattgtacagatcttacagatagaaaatcatgaaagaaacatcagacttaatctgtgctataaaccaaatatacctaataaatatttgcagaacattttatctaatggctacagaacacacattcttctcagcacatagataattttcagatacacaacattttaggttacaaaacaagtcttaaaacattcagaaaagtgaaataatatcaaacatctctgcccacaatggaatataactagaaatcagtaacaagaattttggaaactatacaaacaaatgtaaattaaacaatacgctcctcaatgaaaattggatcagtaaagaaattaagaacgaaattgaaaaatttcttcaaacaaataatgaaaaaacacataccaaaacctatgggatacagcaaaagcagtactgagaaatgtatagatatatgtgcttacatcaaaaaagaaaaaaatcatataacctaatgttgcatcttaaagaaaaaacagcaaaacaaaaattataataaacatcacagcagaaataaaattcaaataaaaaatacaaaaaaaatcaatgaaatgaaaagttgatttttttaagcagataaataaaatttacaaacctttagcttgactaagaaaaaaagaagacccacataaaaccagagatgaaaaaggagacattataactgacactgcagaaattcaaaagattattagtggctattattaacaactgtatgccaactaattggaaaatctaacaaaaatgggtaagttcccagatatatacaacttaccaagattaaacaacgaagaaatccaaaacatgaacagaccaataagaagtaacaagttcaaagccataataaaaagtctcccagtaaagaaaagcccaggacctgatggcttcaccactaaattctacccaacatttaaagatctaataccagctctactcaaaaacttccaaaacacaggaggaaggaatacttcccaactcattctttgaggccagtattaccataatacccaaatcagaaaaagatacatcaaaagatgaaaactgtacgccaatatctataataaatatttatgtaaaaataacaaagtgttagcaaacagaattcaacaacacattaaaagattgttcattatgacaaagtgggatttatcccagggatgcaagaatggttcatcatatgcaatcaatcagtatgatacatcaacagaatgaaggacaaaatcatatgatcatttcaactgctgctgaaaaaaggatttaataaaattcaacatcctttgataataaaaactctcaaaaagctgggtatagaaagaacatacctcaaaataataaaaaccatatatgacagatccacagctagtatcatcctgaatggaggaaaaaaatctgaaagtcttttctctaagatctggaacataacaaggatgtccactttcaacactgttattcaacatagtactggaagtgctagctacagcagcagtaaaacgagaaaaaactaaatggcatccaaattggaaaggaagtcgtcaaattattcttgtttgcagatggtacagtcttatattaagaaaaatctaaagactccaccaaaaaacagtaagaactgataaagttgcaggatataaaatgaacatacaaacatcaatagcatttctatatgccaacagagaatctgaaaaaagagattagaaagtaattccatttacagtagccacaaataaaattaaatatctagacattaacttaaccaaagaagtgaaagatctgtacaatgaaaactataaaacatgaatgaaagacattgaagaggatggctggg

SEQ ID NO: 41 - DNA, 691 bp, Homo sapiens
  source 1..691: mol_type = genomic DNA
cccttgaacttattaaaatcttttataatgtagtacatttactgactgctttcaggcaggcatgaggacctttcactccatttgctccattgtcccttttgaaggattcttgtcatatatataaaacctcttaagacataatgttattgttttatatactctttaatcacctacatctttgcactttaatatttcctaaatttgcattcttctctctgggactagaagacctcagcttagcggttcttttcatgtgggcctgagaaattttctcactttttgtttcactttcattcttgaaggaacgtttcactggttataggatgcaaggttagcggttattctcttctgcatcttcccactgcattgtcttctgaatcttataatttatgtaaaaacattagttgtctgtcttcccattgcttctgtgaaaataatgcatgttttcctctggttgtttttgacattttctttgtgtctgtttctcagcaatttttccatgatatatgctcggagcgggcttacttatcctacaccctgaatctgtagcttgacgtctttttgtcaactttggaaagtttttggcattatctcatcaaatatttcccctgtcctgttcttgctcttctcttgctggagtgcagtcgttcctgtattagacctttgcatcatgttctaggtatttttatct

SEQ ID NO: 42 - DNA, 301 bp, Homo sapiens
  source 1..301: mol_type = genomic DNA
gcggtggctcacgcctgtaatcccagcactttgggaggctgaggcgggtggatcacttgaggtcaggagttcgagaccagcctggccaacatggagaaaccccatctctactaaaaatacaaaaattagctgggcgtggtggctcatgcctgtaatcccagctacttgggaggctgagacaggagaattgcttgaacccaggaggtggaggttgcagtgagccaagaacacactattgcactgcagcctgggtgacagagccagactctgcctgaaaagaaaagaaaagaaaaagaatatg

SEQ ID NO: 43 - DNA, 299 bp, Homo sapiens
  source 1..299: mol_type = genomic DNA
tttttctttgaggtagagtctcactctgtcacctaggctggagtacagtggtgcaatctcagctcactgcaacctctacttcccaggttcaagtgattttcgtgcctcagccacccaagtagctgggattatagccatgcgccaccacacacagctaatttttttgtattttttagtagagatggggttcgccatgttggctaggctggtctcaaactcctggccttaaatgatccacccacctcagcctcccaaattgctgggattgcaagcatgagccatcgcacctggcgcagaac

SEQ ID NO: 44 - DNA, 2458 bp, Homo sapiens
  source 1..2458: mol_type = genomic DNA
tcaactcacagagttgaacaatccttttcatacagcagttttgaatctctctttttgtagaatctccaatggacatttggagcactttggggccttcgctcgaaacgggaatatcttcccataaaaactggacagaagctttatcagaaactactttgggatatgtgctttcaactcacatagttgaacccttcttttgatagagcagttttgaaacactctcattgtagaatctgcaagtggacatctggtgtgctttgaggcctatggagaaaaaggaaatatcttcacataaaaactagacagaagcattctcagaaacttatttttgatgtgtgtactcaactgactcagttacacctttcttttgatagagcagttttgaaacactcttcttgcagaatttataagtggatattaggaaagctttgaggcttttgttggaaacgggaatgtcttcacataaaaaccagacagaagcattctcaggaacatctttgttttacgcgcattcatctcacagacttgaaactttcttttgatagagcagctttgaaacaaactttctgtaaaatttgcaagtggatatttggacagctttgaagcctttgctggaaacgggagtatcttcacataaaaactagatggaaggattctcagacacttccttgggaggtttgcattcaactcaaagagttgaacattccttttcatagagctgttttgaaacactctttttgtagaatctgctagtggacatttggagcaacttgaggcctgtggtgaaaaatgaaataccttcgcataaaaactagacagaatcattctcagaaacttctttgtgatgtgtgtactccactcacagagttgaacatttcttttgatagaacagttttgaaacactcccttgtagaatctgcaggtgtatatttggacagctttgagtctttcattggaaacgggaatatcttctcataaaaactagacagaagcactctcagaaacttctctgtgatgtttgccttcaactctcagagttgatcattctttttcatagagcagttttgaaacactctttttgtagaatctgcaagcggacatttggagcactttgaggcctgtggtaaaaaaggaaatatcttcacatgaaaactagacaggagcattctaagaaacttatttgtgatgtgtacactaaactcacagagttgaacttttcatttgatagagcagttttgaaacactatttttgtgaatcctgcaagtggacatttggagcactttgaggtattctttggaaacgggaatatcttcacataaaaactagaaggaagcattctcagaaacttctttgtgaggtttgcattcaactcacagagttgaacttttcttttgataaagcagttttgaaacacactctgtagaatctgttaatggatatttggacagctttgaggttttcgttggaaacgggactatcttcacataaaaactagacagaagcattctcagaaacttctttgcgatgtttgcattcaactcacagagttgaagattccttttcatagagcagttttgatatgctctttttgtggaatctgcctgaggacattcggagtgctttgaggcctaaggtgaaaaaggaaatatcttcacataaaaactagacagaagcattctcagaaacttctttgtgaagtgtgtactcaactcacagagttaaacctttctttgatagagcagtcttgaaacacacttcttgtagaatttacaagtggatattaagacagctttcaggcttccattggaaatcctgaagatattcgctggaatatcttcacataaattgtagacagaagcattctcagaaacatctttgtgatgtgtgctgttggaactttcctttgatagagcagttttgaaacactctttttgtagaatttgcaagtggatatttggacagctttgaagccttcccaggaaatgagtttatcttcacataaaaactagacagaatcattctcagaaacttctttgtgatgtttggtttaactcacagagttgaacattccttttcatagagcagttttgaaaaacgctttttgtagaatctgcaagtggacatttgcagcgttttgggacccatagtgaataaggaaagatgttcacattaaaactagatagaagcattctcagaaacttctttgtgatgtgtgtactcaacacacagagttgaatctttgttttgacagagcagtttgaaacccttttttgtataatctgcaagtggatatttggatagctttgaggcttccgtcggaaacgggaatatcttcacataaaaactagagagaagcattcctcagaaacttctttgtgaagtttgccttcaactcacagagttgaacattccttttcatagagcagttttgaaacactctttttgtagaatctgcaa

SEQ ID NO: 45 - DNA, 304 bp, Homo sapiens
  source 1..304: mol_type = genomic DNA
caggcctgagcaatggcgggcacccctcccccagcctcgctgccgccttgcagtttgatctcagactgctgtgctagcaatgagcgaggcttcatgggcatggtaccctccgagccaggtgcgggttataatctcctggtgtgccgtttgccaagaccattggaaaagtgcagtattagggtgggagtgacccgattttccaggtgctgtctgtcacccctttctttgactaggaaaggaaattccctgaccccttgtacttcccaggtgaggcgatgcctcgccctgcttcggctcatgctcg

SEQ ID NO: 46 - DNA, 408 bp, Homo sapiens
  source 1..408: mol_type = genomic DNA
cacccaagtcacctcttgaatgctttgctgcttagacatttcttctgccagataccctaaataatctctctcaagttcaaagttccacaaatctctagggcaggggcaaaatgccaccagtctgtttgctaaaacataacaagagtcatctttgctccagttcccaacaaattccttatctccatctgagaccaccttatcctggatttcattgtccatatcattatcagcattttggtcaaagccactccacaagtccctaggaagttccaaactttcccacattttcctgtcttcttctgaggcctccaacctattccaccctctgtctgttacccagttccaaagttgcttctacattttcaggtgtcttttcagcagtgccacactctactggtactaatttac

SEQ ID NO: 47 - DNA, 178 bp, Homo sapiens
  source 1..178: mol_type = genomic DNA
aggcctggcacagtggctcatgcgtgtaatcccaacactgcgggaagctgaggcaggaggatcacttgagcccaggagctcatggctgtagtgagctgtgattgcaccactgcactgcactccagcctgggcaacagcaagaccctgtctccaaaaaaaaaaaacaaaacaaattaat

SEQ ID NO: 48 - DNA, 2122 bp, Homo sapiens
  source 1..2122: mol_type = genomic DNA
ttatggtggacatcattgaataattcatgggctttttcctaattctccttagtccttctagaacacaggtcaacagatgtttgtgactccagtccctatgatctgagttgaggtcccagtggggatccatactggggacagcttgctgaccagtagggaattttccctttctttggctgtcattttatcatttacttgactaagatgccaggtatctccaaactcttgggctgcagctaaagctgcattcttttcattaaaggccagggtttgatctaacaaaagcatgacatctttccaagtgagatcgaaggtttcccctagaccctgtaggacatctatgtacctatcaggatcatctgaaaactttcccaggtctgccttgatctgctttaaatcagagagggagaaggggacatgtacctgggttgggccaaattccccttcccctacagcttgaaggggacgtaaccgatagcccgggggtttttgtggtcctttggagatttctttgcttatttccttctgggcaggggagattagaggaggcttatcattaataggagggggagccatagggaggctaggatatgggactaagctgaaaggtcctcctgggggatgtaaattgcaagctttgcatagtcatgtattctccttcaatgaaaagagtttggacataaggtatttcactccatttgccttccctcttacagaaaaggtcaagctgcaggatagtattgtaatttatacttccctcaggtggccatttttccccatcagagagagaatattggggccaggctgtagggcagaaaaaaatgagccgcctctttttcagggtttgtgggtcaaattggtcccaatggcttaggatgcatttcaaaggtgagcctgttgatgcctgagtgtttcccatctgaaagacaaaaccacctgtggttttggtttgttttgtttctcccccacccacaaacctgcaacggtccctggaccctgctgatcggaatagttgtgctccctgacgcagcagcaaaaatactagttttcctcccagaccacaagggggactgagaaaggtcggatttagtggcccttaccaacgcattctcgaaaacctgcacccttgcctgtcctcctagaccacaaagaggaccgagaaaaatcggatttagtggcccttaccgacacattctcgaaaacccattagagtcctaagcattcacctgttagtattgggaccttaaccatgtcctataaagatgttatgccccccaaatgaagtggagtgccataccctgagggagggaagggatgtccagggttggaagagtgacaccttttgtccttacttatatgaataggaaggatacaatttctgaggctccccatattctagcttcaggaatagcttttgttaggcctgctagtctgaggagggatcctaaaattccagatagtcccccctacgatggggctttgggcaaaaattacgtctttctgattggtgagcctgggtgcctacagaaggtaacagaatcccaaatttatactagaaatcattcttataggagaaactagaaaagcaccagagacagggagtggtttttagaagcaggactagcctggagaagagaggcaaggggaagtttgtctggcaggcgttaggacccagggggcaatggtcagggtagatagaatagatgggcaagtctcgcttgggcgacatgactttgagagttccactcatggccacagggtcaaccaacttgttgttgggaccccggagctgaatggctttcctctctgtcgaccctgggctcagcccagaagtacaggaaaagcggaagctggttccaggcaaaccaacgctaccaactctgaagagtcagggattgttagaaaggcctttcccagaaagcctgacacctgtgtctttagtctggcagctgcgctagtagcttttaactggccgacaggtgcctggtatttagcccccgaattctaaggaaaaataggaaagaatagcaagcgaaaggggtccgatggtactcaccacttggcaataggcgatagtctcacttcttggcgataggcgatggtcccatctgggtcgccaaaa

SEQ ID NO: 49 - DNA, 292 bp, Homo sapiens
  source 1..292: mol_type = genomic DNA
ctttaacctccttgttacgtttgtctcttccagaatcgaagctgtaaaactacaaatggttcttcaaatggagccccagatgcagtccatgactaagatctaccgcggacccctggactggcctgctagcccatgctccaatgttgatgacattgaaggcacccctcccaaggaaatctcaactgcacgacccctactatgccccaattcagcatgaagcagttaagagcggttgtcagccaacctccccaacagcacttgggttttcctgttgagaggtgggactgagaga

SEQ ID NO: 50 - DNA, 338 bp, Homo sapiens
  source 1..338: mol_type = genomic DNA
cagtctttcaggaccagaagagatggattttaggttttccttccactcaatgtattctatatgtaccctaatcaacaaacatgcctggttgtcacaggtagaaggggtctctactctgaatcattcagagaagaaataatcatattctttcgatgctatcactaacacaggaccgtgatccgagacaaccaggttcagttcttgatatgtaaaggaatttgggaaaatatcatctctcaccacaaaatctgtatttttatgtatatttaagcaaaatcctgttttggtcatcattttctttacctttcttctatatccatcctaatctccctttcagc

SEQ ID NO: 51 - DNA, 388 bp, Homo sapiens
  source 1..388: mol_type = genomic DNA
cctatgttagaatagaaaatgtcttcacataaaatctagacagaagcaatctgagatacttctttgtggtgtgtcctttcatctcaaagagttaaaactttcttttgattgagcagttttcaaactctctttttgtagaatctgcaagtggacatttggagcgctttaaggcctgtggtggaaaaggaaatatcttcacctaaaaactagacagaagcaatctgagaaacttctttgtgatgtgtgaattcatttcacagagtaaaacctttcttttgatggagccgttttgaaactctctttttgtagaatctgcaaatggacatttggagttctttgaggcctatggtggaaaaggaaatatcttcacataaaaactagatagaag

SEQ ID NO: 52 - DNA, 770 bp, Homo sapiens
  source 1..770: mol_type = genomic DNA
tcatctcacagagttaaacctttcttttgattgagcagttttgaaactctctttttgtagaatctacaagtggacattgggagctctttgaggcctatgttagaaaaggaaatatcttcacataaaacctagatagaggcaatctgagaaacatctttgtgatgtgtgcattcatctcacggagttaaacctttcttttcgttgagcagtttttaaactgtctttttgtagaatgtgcaagtggacatttggagcgctttttgtcctatggtgggaaaggaagtatcttcacataaaaactagaccgaagaattctgagaaacttctttgtgacgtgtacgttcatctcactgagttgaacctttcttttgattgagcagtttggaaacactctttttgtagaatctgcaagtagacatttggagcgcttttcagcctacggtagaaaaggaaatatcttcacatcacaaagagagagtttcaaaactgctctatcaaaagaaaggtttaaccctgtgcgatgaatgcacatatcacaaagaagtttctcagattgcttctgtctagattttatgtgaagatattcctttttccaccataggcctcaaagcgctccaaatgtccacttgcagattctacaaaaagagtgcttccaaactgctcgatgaaaaaaatggttcaactctgtgagatgaatgcacgcatcacaaagaagtttctcagaatttttctatctagtatttatttgaagatatttcattttccaccat

SEQ ID NO: 53 - DNA, 129 bp, Homo sapiens
  source 1..129: mol_type = genomic DNA
tttcagatagagtcaccctctgtcgcccaggctggagtgcagtggcatgatcttggctcactgcaacctccacctcccaggttcaagcgattctcctgcctcagcctcctgagtagctgggattacaga

SEQ ID NO: 54 - DNA, 1603 bp, Homo sapiens
  source 1..1603: mol_type = genomic DNA
gaactaatttacactcccaccaacagtgtaaaagtgttcctatttttccacaacctctccagcatctgttgtttcctgacattttaatgattgccattctaactggcatgagatggtctcattgtggttttcatttgcatttctctaatgaccagtgatgataagcattttttcatacatctcttggctgcgtaaatgtcttcttttgaaaagtgtctgttcatatcctttgcccattttttgatggggttgttgccttttttcttgtaaatttctttcagttctttgtagcttctggatattagccctttgtcagatggacagattgcaaaaatcttctcccattctgtaggttgcctgttcactctgatgatgatgatagtttctttcgctgtgcagaagctctttagtttaattagatcccatttgtcaattttaactttttttgccattgtttttggtgttttagacatgaagccttttcccatgcctatgtcctgaatggtattgtctaggttttcttctaggatttttatggtcctaggtcttatgtttaagtctttgatcaatcttcagttgatttttgtataatgtgtaaggaaggggtccagtttcagttttctgcatatggccagccagctttcccaacaccatttattaaatagggaatcctttccccatttcttgtgtgtgtcaggtttgtcaaagatcagatggcagtaaatgtgtggtgttatttctgaggcctctgttctgttccattggtctatatatctgttttgataccagtgccatgctgttttggttactgtagacttgtagtatagtttgaagtcaggtagcatgatgcctccagctttgttcttcttgcccaggattttcttggctatgtgggcttttttttggttccatatgaagtttaaaatagttttttccaattctgtgaagaaagtcagtggtagctggatggggagagcattgaatctataaattactttgggcagtaaggccattttcatgatactgattcttcctatccatgagcatgcaaggtttttccatttgtttgtgtcctcttttatttccttgagcagtggtttttgaagaggtccttcatatcccttgtaagttgtattcctaggtattttattctctttgtagcaattgtgaatgagagttcactcatgatttggctctctgttgtctgttattggtgtataggaatgcttgtcattttcgcacattgattttgtatcctgagactttgctgaagttgcttatcagcttaaggaggttttgggctgagacaatgaggtttcctaaatatacaatcatgtcatctgcaaacagagacaatttgacttcctctcttcctatctgaataccctgtatttctttctcttgcctgattgccctggccagaacttccaatactatgttgaataggagtggtaagagagggcatccttgccttgtgctggttttcaaagggaatgcttccagttttttcccccattcagtatgatactggctgtgggtttgtcataaagagctcttattattttgagatatgttccatcaatacctagtttattgagagttt

SEQ ID NO: 55 - DNA, 301 bp, Homo sapiens
  source 1..301: mol_type = genomic DNA
gcggtggctcacgcttgtaatcccagcactttgggaggccgaggtgggcggatcacgaggtcaggagatcaagaccacggtgaaaccccatctctactaaaaatataaaaaattagccgggcgtggtggtgggcgcctgtaatcccagctactcagagaggctgaggcaggagaagggcgtgaacccgggaggcggagcttgtagtgagctgagatcgcgccactgcactccagcaccccagcctgggtgacagagcgagactccgtctcaaaaaaataataataataattaaataaataa

SEQ ID NO: 56 - DNA, 1853 bp, Homo sapiens
  source 1..1853: mol_type = genomic DNA
ccaagaagttcaaactggacagaacccaccacagctccacaaagccactgtagccagactgcctctctacattcctcctctctgggcaggccacctctgaaagaaaggcagcagccccagtcaggggcttatagataaaactcccatctccccgggacagagcacttgggggaaggggcatctgtgggtgcagcttcagcagacttaaacgttcctgtctgctggctctgaagagagcagcagatctcccagcacagcactccagctctcctaagggacagactgcctcctcaagtgtgtccctgaaccctgtgcctcctgactgggagacacctcccagcagggatcgacagacacctcatacaggagagctctggctggcatttggcgggtgcccctctgggacaaagcttccagaggaaagaacaggcagcaatctttgctgttctcagcctctgctggtgatacccatttaaacacagtctggagggggcctccagcaaactccagcagacctgcagaggagaggcctgactgttagaaggaaaactaacaaacagaaaggaatagcatcaacatccacaaaaaggacatccacacaaaaaccccatccaaaggtcaccaacatcaaagaccaaagatagataaatccatgaagatgaggaaaaaacagcgcaaaagggctgaaaattccaaaaaccagaatgcctctactcctcccaaggatcacaactccatgccagcaagggaacaaaactggacggagaatgaatttgaccaactgacagaagtaggtttcagaaggtgggtaacaacaaacccctctgagctaaaggagcatggtctaagccattgcaaggaagctaagaaccttgaaaaaagattagaggaattgctatctagaataaccaatttagagaagaatataaatgatctgatggagctgaaaaacacagcttgagaactttgtgaggcatacacaagattcaatagccaaatcgatcaagcagaagaaaggatatgagaattgaagttcaacttaatgaaataaaatctgaacattaaagtagagataaaagaatgaaaaggaacaaacaaagcctccaagaaatatgggacaatgtgaaaagaccaaacctacgtttcaatggtgtacctgaaagtcatgaagagaatggaaccaagttggaaaacactcttcaggatattatccaggaaaacttccccaacctagcaagagagcccaacactaaaattcaggatacacagagaacactacaaagatactcctcaagaagaccaaccccaagacacataatcatcagattcaccaaggttgaaaggaaggaaaaaatgttaagagcagccagagaaaagggtcaggttacccacaataggaaacccatcagactaacagctgatctctctgcagaaaacctacaagccagaagagagtagggccaatactcaacattcttaaacaaaagaatttttaacccaaaatttcatatccagtcaaactaagcttcataagcaaaggagaaataaaatctcttatggacaagcaaatgctgagagattttgtcaccatcaggcctgccttacaagagctcctgaggaagcactaaatataaaaaggaaaaactggtaccaaccactgcaaaaacataccaaattgtaaagaccatcaacaccatgaagaaatggcatcaaataaagggcaaaataaccaactaacatcataatgacaggatcaaattcacacataacaatattaaccttaaatgtaaatgggctaaatgccccaattaaaaaacacagactggcaaattggaaagagtcaagacaaattg

SEQ ID NO: 57 - DNA, 301 bp, Homo sapiens
  source 1..301: mol_type = genomic DNA
ttttttttttcccagacagaggctcactctgttgcccaggctggagtgcagtggcatgatctcggctcattgcaacctctgccttctagattcaagggattctcctgcctcagcctcccaagtagctgggattacaggcccacaccaccacgcctggctaatttttgtattttgagtagagacggcgtttcatcatgttggctaggcgggtctcgaattcctgacctcaagtgatctgcccgtctccgtctaccaaagtgctgggattacaggcgtgagccaccacgcccggcctgaagcc

SEQ ID NO: 58 - DNA, 361 bp, Homo sapiens
  source 1..361: mol_type = genomic DNA
tagatggagggggaaaaacagggtggggagcgggagggagggaaggttttgcagaaagacggcggttaaaaagtttatgggtagatggagggggaaaagagggtggcaagtgggaggaggaaagagggtgataagggggggaagtgggggtgagcagtagggagagaaggttttgtgaaaaaaatggggagaaaggttttggttagacgggagaaaagagggtaacaagttggggaaggaaaaagagggtagctaggaagaagacaaggttttgcgaaaagatagcggccagaaaagaaagacggtggagaaaggaaagacggtgggtaaaaagtgggtagatggagtggggaaagagggt

SEQ ID NO: 59 - DNA, 129 bp, Homo sapiens; identical to SEQ ID NO: 53
  source 1..129: mol_type = genomic DNA

SEQ ID NO: 60 - DNA, 759 bp, Homo sapiens
  source 1..759: mol_type = genomic DNA
attcagaagacaaatctcacatgcaaagacacacataggctcaaaataagtggatggaggaatatttaccaagcaaatggcaagcaaaaagaaaaagcaggggttgcaatcctagtatctgataaaacaggctttaaaccaagaaagatcaaaaaagacaaagaaggtcattacataatggtaaagggatcaatgcaacaagaagagctaactatcctaaatatatacgcacccaatacaggagcacccagattcataaagtaaattcttagagactacaaagagacttatactcccatacaataatagtgggagattttaacacctcattgttgatattagatcaatgagacagaaaattaacaaggatattcaggacttgaactcagcactggacaaagcagatttaatagacatctacagaactctctacctcagatcaacagaatatacattcttctcagcaccacatagcacttattctaaaattgaccacataattggaagtaaaacactcctcaacgaatgcaaaagaatggaaatcataacaaacagtctctcagaccacagtgcaatcaaattagaactcaggattacgaaacttactcaaaactgcacaactacatggaaactgaacaacctgctcctgaacgactactgggtaaataatgaaatgaaggcagaaataaataagtttttgaaaccaatgagaacaaagacacaacataccagaatctctgggacacagctaaaacagtg

SEQ ID NO: 61 - DNA, 270 bp, Homo sapiens
  source 1..270: mol_type = genomic DNA
tatttttaagcaaagacttagagagagagaggaagttagctatgcacattgtgggtgcggtgtggttagggtggccctgagatagggatatgcctggtgtgtttacggaacagctaagaggccagggaactagaggaaattgagtgggaaggagaagagtaggagattagttttgaaaagcaaatggtttgggaatatggatcatgtaagagcacatgagattttataaggatttcattttactcagggtgaaatgaagaagcactaaaa

SEQ ID NO: 62 - DNA, 284 bp, Homo sapiens
  source 1..284: mol_type = genomic DNA
gttcatgcctgtaatcccagtgcttagggaggccgaggtgggtggattgcttgagcccaggagttcgagaccagcctggacaaaatggcagaaactccatgtctacaaaaaatacaaaaattagccgggcatgatgttctgcgcctgtagtcccagctactcaggaggctgaggtgggaggatcgcttgagcccgggaggcggagtttgcagtgagctgagatgtcactgcattccagcctggcagacagagccagactctgtctcaaaagaaaaaaagaaaaa

SEQ ID NO: 63 - DNA, 384 bp, Homo sapiens
  source 1..384: mol_type = genomic DNA
atggaatactatgtagccctaaaaaaagatgagttcatgtcctttgcagggacatggatgaagctggaaaccatcattctcagcaaactaacacaggaacagaaatccaaacactgcatgttctcactcataagtgggagttgagcaatgagaaaacatggacacagggaggggaacatcacacactggggccttttggggagttgggggctaggggagggataacattaggagaaatacctaatgttatttctcctaatattatttcagcctaatgatgggttgatgggtgcagcaaaccagcatggcccatgtatacctatgtaacaaacctgcatgttctacatatgtatcacagaacttaaagtatgtatatataaaaaa

SEQ ID NO: 64 - DNA, 1243 bp, Homo sapiens
  source 1..1243: mol_type = genomic DNA
ttagtctgatgggcttccctttgtgggttacctgacctttctctctggctgcccttaacattttttccttcatttcaactttggtcagtctgacgattatgtgtcttggggttgctcttctcaaggagtattagtggtgttctctgtatttcctgaatttgaatattggcctgtcttgctacgttggggaggtcctctggataatatcctgaagagtgttttccaacttgattccattcttcccgtcactttcaggtacaccaatcagacgtaagtttggtcttttcacatagtcccatatttcttggagtccttgttcattctttttcattctttttatccctaatcttgtcctgatgctttatttcattaagttgatcttcaatctctgatatcctttcttccgcttgatcgattcggctattaatacttgtgtatgcttcacgaagttctcatgctatgtttttcagctccaccagctcatttatgttcttctctaaactggttactctagttagcaattcctctaaccttttttcaaggttcttagcttccttgcattggattagaacatgcccctttagctcagaggagtttgttattacccaccttctgaagcctacttctgtcaatttgtcaaactcattctctgtccagttttgttcccttgctggcaaggagttgtgatcctttgtagaggaggtattctattttttggaattttcagctattttgccctggtttttcctcatctttgtggatttatctacttttggtctttgatgctggtgaccttcggatggggtttttgtgtgtgtgtcccttttgttgatgttgatgctattcctttctgtttgttagttttccttctaactggcccgtctggtgcaggtctgctggagttttctggaggcccactccagaccctgtttgcctgagtatcaccagcggagcctgcaggacagcaaagattgctgcttgttcctttctctggaagctttatcccagaggggcacctgccagatggcagctggagctctcctgtatgaggtgtctgttgacccctgctgggaggtgtctgccagtcaggaggcacggggtcagggacccacttgaggaggcagtctgtcccttagcagagctcgagcactgtgctgggagatctgcttctcttttcagagccagaaggcaggaatgtttaagtctgtagaactgtgcccacagccgctcctttcccccaggt

SEQ ID NO: 65 - DNA, 298 bp, Homo sapiens
  source 1..298: mol_type = genomic DNA
gccgagcgcggtggctcaagcctgtaatcccaccactttgggaggcaaaggtgggtgaatcacctgaggtcaggagttcgagaccagcctgaccaacatggtgaaaccccatctctactaaaaatacaaaaattagccaggcctggtgtcgggtgcctgtaatcccagctactcaggaggctgagacaggagaatcacttgaacccaggaggtggaggttgcagtaagccgagaccgtgcccattgcactccaatctgggcgacgagagtgaaactccatcttaaaaaaaaaaaaaaa

SEQ ID NO: 66 - DNA, 195 bp, Homo sapiens
  source 1..195: mol_type = genomic DNA
gccattttcacgatattgattcttcctatccatgagcacggaatgtttttccattcgtttctgtcctctcttattgccttgagcagtggtttgtagatctccttgaagaggtccttcacatcctttgtaagttggattcctaggtattttattctctttgtagcaattgtgaatgggagttcactcatgatttca

SEQ ID NO: 67 - DNA, 114 bp, Homo sapiens
  source 1..114: mol_type = genomic DNA
ccttaatagttaatctcctattaataagtaagtgattctttaattaaactttcctcattcaaatggccatatgctttctgtctctcgactagatgctgagtgatatacccccca

SEQ ID NO: 68 - DNA, 290 bp, Homo sapiens
  source 1..290: mol_type = genomic DNA
acccggccttggacacgccattttcaactccgtggtgcgtttttttttttttttttttttttttgtaatggagtttcgctcttgttgcccaggatggagtgcaagggatcttggctcaccacagcctccgcctcttgggttcaagtgattcttctgcctcagcctcccaagtagctgggattataagcacccaccaccacgcccagctaattttgtattttttagaagagatggagtttctccagttggccaggatggtctgtatatcctgacctcatgatctgcccacc

SEQ ID NO: 69 - DNA, 202 bp, Homo sapiens
  source 1..202: mol_type = genomic DNA
ccaggatggagtgcaagggatcttggctcaccacagcctccgcctcttgggttcaagtgattcttctgcctcagcctcccaagtagctgggattataagcacccaccaccacgcccagctaattttgtattttttagaagagatggagtttctccagttggccaggatggtctgtatatcctgacctcatgatctgcccacc

SEQ ID NO: 70 - DNA, 114 bp, Homo sapiens
  source 1..114: mol_type = genomic DNA
gggattataagcacccaccaccacgcccagctaattttgtattttttagaagagatggagtttctccagttggccaggatggtctgtatatcctgacctcatgatctgcccacc

SEQ ID NO: 71 - DNA, 113 bp, Homo sapiens
  source 1..113: mol_type = genomic DNA
acccggccttggacacgccattttcaactccgtggtgcgtttttttttttttttttttttttttgtaatggagtttcgctcttgttgcccaggatggagtgcaagggatcttg

SEQ ID NO: 72 - DNA, 201 bp, Homo sapiens
  source 1..201: mol_type = genomic DNA
acccggccttggacacgccattttcaactccgtggtgcgtttttttttttttttttttttttttgtaatggagtttcgctcttgttgcccaggatggagtgcaagggatcttggctcaccacagcctccgcctcttgggttcaagtgattcttctgcctcagcctcccaagtagctgggattataagcacccaccaccacg

SEQ ID NO: 73 - DNA, 80 bp, Homo sapiens
  source 1..80: mol_type = genomic DNA
ccaggatggagtgcaagggatcttggctcaccacagcctccgcctcttgggttcaagtgattcttctgcctcagcctccc

SEQ ID NO: 74 - DNA, 113 bp, Homo sapiens
  source 1..113: mol_type = genomic DNA
ccaggatggagtgcaagggatcttggctcaccacagcctccgcctcttgggttcaagtgattcttctgcctcagcctcccaagtagctgggattataagcacccaccaccacg

SEQ ID NO: 75 - DNA, 158 bp, Homo sapiens
  source 1..158: mol_type = genomic DNA
ccaggatggagtgcaagggatcttggctcaccacagcctccgcctcttgggttcaagtgattcttctgcctcagcctcccaagtagctgggattataagcacccaccaccacgcccagctaattttgtattttttagaagagatggagtttctccagt

SEQ ID NO: 76 - DNA, 102 bp, Homo sapiens
  source 1..102: mol_type = genomic DNA
gtgattcttctgcctcagcctcccaagtagctgggattataagcacccaccaccacgcccagctaattttgtattttttagaagagatggagtttctccagt

SEQ ID NO: 77 - DNA, 146 bp, Homo sapiens
  source 1..146: mol_type = genomic DNA
gtgattcttctgcctcagcctcccaagtagctgggattataagcacccaccaccacgcccagctaattttgtattttttagaagagatggagtttctccagttggccaggatggtctgtatatcctgacctcatgatctgcccacc

SEQ ID NO: 78 - DNA, 290 bp, Homo sapiens
  source 1..290: mol_type = genomic DNA
acccggccttggacacgccattttcaactccgtggtgcgttttttttttttttttttttttttgtaatggagttttgctcttgttgcccaggatggagtgcaagggatcttggctcaccacagcctctgcctcctgggttcaagtgattcttctgcctcagcctcccaagtagctgggattataagcacccaccaccacgcccagctaattttgtattttttagaagagatggagtttctccagttggccaggatggtctgtatatcctgacctcatgatctgcccacca

SEQ ID NO: 79 - DNA, 14 bp, synthetic construct
  source 1..14: mol_type = other DNA
gcgctctgtgtgct

SEQ ID NO: 80 - DNA, 22 bp, Homo sapiens
  source 1..22: mol_type = genomic DNA
cmtctgataycatcacagccsc

SEQ ID NO: 81 - DNA, 18 bp, Homo sapiens
  source 1..18: mol_type = genomic DNA
ttttttttrrrtggagtc

SEQ ID NO: 82 - DNA, 18 bp, Homo sapiens
  source 1..18: mol_type = genomic DNA
mmagaaatggggaaagka

SEQ ID NO: 83 - DNA, 15 bp, Homo sapiens
  source 1..15: mol_type = genomic DNA
atcagaakgtgaggt

SEQ ID NO: 84 - DNA, 34 bp, Homo sapiens
  source 1..34: mol_type = genomic DNA
aagtaatcctccaccatgrccctccwaagtgctg

SEQ ID NO: 85 - DNA, 20 bp, Homo sapiens
  source 1..20: mol_type = genomic DNA
gsaaaggggccaaargagar

(19) United States
(12) Patent Application Publication
(10) Pub. No.: US 2025/0241939 A1
(43) Pub. Date: Jul. 31, 2025

(54) NUCLEIC ACID-BASED DELIVERY SYSTEMS AND USES THEREOF

(71) Applicants: Kodikaz Therapeutic Solutions, Inc., New York, NY (US); Emory University, Atlanta, GA (US)

(72) Inventors: Leon BERNAL-MIZRACHI, Atlanta, GA (US); Munevver CINAR, Brookhaven, GA (US); Pavan K. PUVVULA, Long Island City, NY (US); Lei FENG, Bronx, NY (US)

(21) Appl. No.: 19/035,421

(22) Filed: Jan. 23, 2025

Related U.S. Application Data
(63) Continuation of application No. PCT/US23/71229, filed on Jul. 28, 2023.
(60) Provisional application No. 63/506,800, filed on Jun. 7, 2023.
(60) Provisional application No. 63/369,913, filed on Jul. 29, 2022.

(51) Int. Cl. A61K 31/711; A61P 35/00; C12N 15/90

(52) U.S. Cl. CPC A61K 31/711; A61P 35/00; C12N 15/907; C12N 2800/90

(57) ABSTRACT

Compositions and methods including synthetic delivery systems that utilize polynucleotides are provided. Polynucleotides disclosed herein can, for example, facilitate binding, uptake, nuclear trafficking, and/or genomic integration of a component of the synthetic delivery system, for example, a cargo.

### CROSS REFERENCE

This application is a continuation of International Patent Application No. PCT/US23/71229, filed Jul. 28, 2023, which claims priority to and the benefit of U.S. Provisional Patent Application No. 63/506,800, filed Jun. 7, 2023, and U.S. Provisional Patent Application No. 63/369,913, filed Jul. 29, 2022, each of which is incorporated herein by reference in its entirety.

### SEQUENCE LISTING STATEMENT

The instant application contains a Sequence Listing which has been submitted electronically in XML format and is hereby incorporated by reference in its entirety. Said XML copy, created on Jan. 23, 2025, is named 54774_707_301_SL.txt and is 120,070 bytes in size.

### BACKGROUND

A transposable element (TE or transposon) is a DNA sequence that can change position within a genome. The relationship of transposable elements to the onset of disease is not well understood.

Tumor-derived cell-free DNA can contain genetic alterations relevant to tumorigenesis, suggesting circulating tumor-derived DNA (ctDNA) serves as a vehicle for genetic exchange between tumor cells. It has been suggested that ctDNA can transfer oncogenic gene mutations for reshaping the tumor microenvironment. However, the underlying process and mechanisms remain poorly understood.

### SUMMARY

Disclosed herein, in some embodiments, is a pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence consists essentially of a nucleotide sequence with at least 80% sequence identity to any one of SEQ ID NOs: 73-75.

Disclosed herein, in some embodiments, is a pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence comprises the nucleotide sequence of SEQ ID NO: 73.

Disclosed herein, in some embodiments, is a pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence comprises a nucleotide sequence with at least 80% sequence identity to at least 10 consecutive nucleotides of SEQ ID NO: 13 or 14.

Disclosed herein, in some embodiments, is a pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence comprises (a) a nucleotide sequence with at least 90% sequence identity to at least 60 consecutive nucleotides of SEQ ID NO: 33, or (b) a nucleotide sequence with at least 91% sequence identity to at least 50 consecutive nucleotides of SEQ ID NO: 33.

Disclosed herein, in some embodiments, is a pharmaceutical composition comprising a synthetic delivery system and a pharmaceutically-acceptable excipient, wherein the synthetic delivery system comprises a polynucleotide that comprises a recognition sequence, wherein the recognition sequence comprises an AluSp, AluSx, AluSg2, ERV2 superfamily, or ERV3 superfamily transposable element, or a functional fragment thereof that facilitates binding of the synthetic delivery system to a target cell or uptake of the synthetic delivery system or a component thereof by the target cell.

Disclosed herein, in some embodiments, is a pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the polynucleotide comprises a nucleotide sequence with at least 86% sequence identity to any one of SEQ ID NOs: 7, 16, 25, 29, and 31.

Disclosed herein, in some embodiments, is a method of delivering a cargo to a target cell, the method comprising contacting the target cell with a pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence comprises a nucleotide sequence with at least 80% sequence identity to at least 10 consecutive nucleotides of SEQ ID NO: 13 or 14.

Disclosed herein, in some embodiments, is a method of delivering a cargo to a target cell, the method comprising contacting the target cell with a pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence comprises (a) a nucleotide sequence with at least 90% sequence identity to at least 60 consecutive nucleotides of SEQ ID NO: 33, or (b) a nucleotide sequence with at least 91% sequence identity to at least 50 consecutive nucleotides of SEQ ID NO: 33.

Disclosed herein, in some embodiments, is a method of delivering a cargo to a target cell, the method comprising contacting the target cell with a pharmaceutical composition comprising a synthetic delivery system and a pharmaceutically-acceptable excipient, wherein the synthetic delivery system comprises a polynucleotide that comprises a recognition sequence, wherein the recognition sequence comprises an AluSp, AluSx, AluSg2, ERV2 superfamily, or ERV3 superfamily transposable element, or a functional fragment thereof that facilitates binding of the synthetic delivery system to a target cell or uptake of the synthetic delivery system or a component thereof by the target cell.

Disclosed herein, in some embodiments, is a method of delivering a cargo to a target cell, the method comprising contacting the target cell with a pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the polynucleotide comprises a nucleotide sequence with at least 86% sequence identity to any one of SEQ ID NOs: 7, 16, 25, 29, and 31.

Disclosed herein, in some embodiments, is a method of treating a condition in a subject in need thereof, the method comprising administering to the subject a pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence comprises a nucleotide sequence with at least 80% sequence identity to at least 10 consecutive nucleotides of SEQ ID NO: 13 or 14.

Disclosed herein, in some embodiments, is a method of treating a condition in a subject in need thereof, the method comprising administering to the subject a pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence comprises (a) a nucleotide sequence with at least 90% sequence identity to at least 60 consecutive nucleotides of SEQ ID NO: 33, or (b) a nucleotide sequence with at least 91% sequence identity to at least 50 consecutive nucleotides of SEQ ID NO: 33.

Disclosed herein, in some embodiments, is a method of treating a condition in a subject in need thereof, the method comprising administering to the subject a pharmaceutical composition comprising a synthetic delivery system and a pharmaceutically-acceptable excipient, wherein the synthetic delivery system comprises a polynucleotide that comprises a recognition sequence, wherein the recognition sequence comprises an AluSp, AluSx, AluSg2, ERV2 superfamily, or ERV3 superfamily transposable element, or a functional fragment thereof that facilitates binding of the synthetic delivery system to a target cell or uptake of the synthetic delivery system or a component thereof by the target cell.

Disclosed herein, in some embodiments, is a method of treating a condition in a subject in need thereof, the method comprising administering to the subject a pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the polynucleotide comprises a nucleotide sequence with at least 86% sequence identity to any one of SEQ ID NOs: 7, 16, 25, 29, and 31.

Disclosed herein, in some aspects, is a pharmaceutical composition comprising a synthetic delivery system and a pharmaceutically-acceptable excipient, wherein the synthetic delivery system comprises a polynucleotide, wherein the polynucleotide comprises a nucleotide sequence with at least 80% sequence identity to at least 10 consecutive nucleotides of any one of SEQ ID NOs: 1-78.

In some embodiments, the polynucleotide comprises a nucleotide sequence with at least 80% sequence identity to at least 15 consecutive nucleotides of any one of SEQ ID NOS: 1-78. In some embodiments, the polynucleotide comprises a nucleotide sequence with at least 80% sequence identity to at least 20 consecutive nucleotides of any one of SEQ ID NOs: 1-78. In some embodiments, the polynucleotide comprises a nucleotide sequence with at least 80% sequence identity to at least 30 consecutive nucleotides of any one of SEQ ID NOs: 1-78. In some embodiments, the polynucleotide comprises a nucleotide sequence with at least 80% sequence identity to at least 50 consecutive nucleotides of any one of SEQ ID NOs: 1-78. In some embodiments, the polynucleotide comprises a nucleotide sequence with at least 80% sequence identity to at least 75 consecutive nucleotides of any one of SEQ ID NOs: 1-78. In some embodiments, the polynucleotide comprises a nucleotide sequence with at least 80% sequence identity to at least 100 consecutive nucleotides of any one of SEQ ID NOs: 1-78. In some embodiments, the polynucleotide comprises a nucleotide sequence with at least 80% sequence identity to any one of SEQ ID NOs: 1-78. In some embodiments, the polynucleotide comprises a nucleotide sequence with at least 90% sequence identity to at least 10 consecutive nucleotides of any one of SEQ ID NOs: 1-78. In some embodiments, the polynucleotide comprises a nucleotide sequence with at least 90% sequence identity to at least 15 consecutive nucleotides of any one of SEQ ID NOs: 1-78. In some embodiments, the polynucleotide comprises a nucleotide sequence with at least 90% sequence identity to at least 20 consecutive nucleotides of any one of SEQ ID NOs: 1-78. In some embodiments, the polynucleotide comprises a nucleotide sequence with at least 90% sequence identity to at least 30 consecutive nucleotides of any one of SEQ ID NOs: 1-78. In some embodiments, the polynucleotide comprises a nucleotide sequence with at least 90% sequence identity to at least 50 consecutive nucleotides of any one of SEQ ID NOs: 1-78. In some embodiments, the polynucleotide comprises a nucleotide sequence with at least 90% sequence identity to at least 75 consecutive nucleotides of any one of SEQ ID NOs: 1-78. In some embodiments, the polynucleotide comprises a nucleotide sequence with at least 90% sequence identity to at least 100 consecutive nucleotides of any one of SEQ ID NOs: 1-78. In some embodiments, the polynucleotide comprises a nucleotide sequence with at least 90% sequence identity to any one of SEQ ID NOs: 1-78. In some embodiments, the polynucleotide comprises a nucleotide sequence with at least 80% sequence identity to any one of SEQ ID NOs: 1-78. In some embodiments, the polynucleotide comprises a nucleotide sequence with at least 90% sequence identity to any one of SEQ ID NOs: 1-78. In some embodiments, the polynucleotide comprises the nucleotide sequence of any one of SEQ ID NOs: 1-78.

In some embodiments, the disclosure provides a pharmaceutical composition comprising a synthetic delivery system and a pharmaceutically-acceptable excipient, wherein the synthetic delivery system comprises a polynucleotide, wherein the polynucleotide comprises a transposable element that is an AluSp, MER11C, AluY, 2L2a, ALluY, ALR/Alpha, ALU, AluJb, AluS1, AluSc8, AluSg, ALUSg2, AluSq, AluSq2, ALUSx, AluSx, AluSx1, AluSz, AluSz6, AluYc3, ASLUSq2, ERVK/LTR, ERVL, ERVL-MaLR, FLAM_C and AluY, HERV17-int, HERV9N-int, L1M1, L1MB3, L1ME4b, L1MEg, L1P1, L1P3, L1PA10, L1PA15, L1PA7, L1PB4, L2a, L2a/LTR40b/MLT1J2, L1NE/L1, LINE/L2, LTR, LTR/ERV1, LTR/ERVL, LTR/ERVL-MaLR, LTR/Gypsy, LTR41C, LTR81B, Mam_R4, Mamr4, Many, MER11B, MER41E, MIR, MIRB, MIRc, MIRc-part L2, MLT1J2, MLT2B1, MLT1J2, MLT2B4, parAluSp-FULLMTL1J2, REP522, Satellite/centr, SINE/Alu, SINE/MIR, THE1A, THE1B, THE1C, or Tigger3a transposable element.

In some embodiments, the disclosure provides a pharmaceutical composition comprising a synthetic delivery system and a pharmaceutically-acceptable excipient, wherein the synthetic delivery system comprises a polynucleotide that directs migration of the synthetic delivery system to target cell, wherein the polynucleotide comprises a nucleic acid sequence identified in circulating tumor DNA (ctDNA) from a substantially similar cell type as the target cell, wherein the nucleic acid sequence comprises a short interspersed nuclear elements (SINE), long interspersed nuclear elements (LINE), ERVL, or ERVK transposable element.

In some embodiments, the polynucleotide comprises an AluSp transposable element. In some embodiments, the polynucleotide comprises a MER11C transposable element. In some embodiments, the polynucleotide comprises a AluSx transposable element. In some embodiments, the polynucleotide comprises a MLT1J transposable element. In some embodiments, the polynucleotide comprises a AluSg2 transposable element. In some embodiments, the polynucleotide comprises a THE1A transposable element. In some embodiments, the polynucleotide comprises a AluJb transposable element. In some embodiments, the polynucleotide comprises a MLT2B4 transposable element. In some embodiments, the polynucleotide comprises a L2a transposable element. In some embodiments, the polynucleotide comprises a MLT1J2 transposable element. In some embodiments, the polynucleotide comprises a AluSq transposable element. In some embodiments, the polynucleotide comprises a L1MB3 transposable element. In some embodiments, the polynucleotide comprises a THE1C transposable element. In some embodiments, the polynucleotide comprises a AluY transposable element. In some embodiments, the polynucleotide is double stranded DNA. In some embodiments, the synthetic delivery system further comprises a cargo. In some embodiments, the cargo comprises a nucleic acid cargo. In some embodiments, the nucleic acid cargo is appended to a 3′ end of the polynucleotide. In some embodiments, the nucleic acid cargo is appended to a 5′ end of the polynucleotide. In some embodiments, the synthetic delivery system comprises a promoter. In some embodiments, the nucleic acid cargo encodes a tumor suppressor protein. In some embodiments, the cargo comprises a cytotoxic cargo. In some embodiments, the cargo comprises a therapeutic cargo. In some embodiments, the synthetic delivery system does not utilize a viral vector, a nanoparticle, a lipid nanoparticle, a liposome, an exosome, a dendrimer, a gene gun, or electroporation.

In some embodiments, the disclosure provides a method of delivering a cargo to a target cell, the method comprising contacting the target cell with the pharmaceutical composition or the synthetic delivery system of any one of the preceding embodiments.

In some embodiments, the cargo is delivered to a nucleus of the target cell. In some embodiments, the synthetic delivery system further comprises a transposon integration signal. In some embodiments, the cargo comprises a nucleic acid cargo. In some embodiments, the nucleic acid cargo is integrated into the target cell's genome. In some embodiments, the nucleic acid cargo is integrated into the target cell's genome at a location identified in TABLE 2, TABLE 4, TABLE 7, or TABLE 8. In some embodiments, the nucleic acid cargo is integrated into the target cells genome at Chr2: 32916224-32916626. In some embodiments, the nucleic acid cargo is integrated into the target cells genome at Ch16: 32628381-32629000. In some embodiments, the target cell is a leukocyte. In some embodiments, the target cell is a plasma cell. In some embodiments, the target cell is a cancer cell. In some embodiments, the target cell is a multiple myeloma cell. In some embodiments, the target cell is a pancreatic cell. In some embodiments, the target cell is a pancreatic cancer cell. In some embodiments, the target cell is a gastrointestinal cell. In some embodiments, the target cell is a colorectal cancer cell.

In some embodiments, the disclosure provides a method of treating a subject in need thereof, the method comprising administering the pharmaceutical composition of any one the preceding embodiments to the subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A shows gain of nucleotide variants in cells cocultured with ctDNA. Comparative SNV analysis was performed between cell genome, ctDNA, and ctDNA-cell coculture. The Venn diagrams display exclusive and shared SNVs between each experimental condition. The outlined area highlights SNVs commonly shared between ctDNA and ctDNA/cell coculture condition.

FIG. 1B shows stacked bar diagrams demonstrating the changes in allele depth of the variant (light gray) and reference (dark gray) allele in ctDNA, cell line genome, and coculture conditions. Cells under coculture conditions have more depth in the variant allele in several locations compared to the control cell genome.

FIG. 2A shows index IVG variant calls images and their allele frequency in pancreatic cancer experimental conditions. Horizontal bars represent the alternate nucleotide indicated underneath and light gray represents the reference nucleotide.

FIG. 2B shows index IVG variant calls images and their allele frequency in multiple myeloma experimental conditions. Horizontal bars represent the alternate nucleotide indicated underneath and light gray represents the reference nucleotide.

FIG. 2C shows index IVG variant calls images and their allele frequency in multiple myeloma experimental conditions. Horizontal bars represent the alternate nucleotide indicated underneath and light gray represents the reference nucleotide.

FIG. 2D shows index IVG variant calls images and their allele frequency in pancreatic cancer experimental conditions. Horizontal bars represent the alternate nucleotide indicated underneath and light gray represents the reference nucleotide.

FIG. 3A shows BLAST alignment images demonstrating the transition point of insertion between cell genome contigs (dark boxes) and a ctDNA contigs (light boxes) for pancreatic cancer and multiple myeloma ctDNA. Results were obtained after comparing the contigs carrying insertions in the coculture condition with the reference cell genome.

FIG. 3B shows BLAST alignment images demonstrating the transition point of insertion between cell genome contigs (dark boxes) and a ctDNA contigs (light boxes) for multiple myeloma ctDNA. Results were obtained after comparing the contigs carrying insertions in the coculture condition with the reference cell genome.

FIG. 3C shows BLAST alignment images demonstrating the transition point of insertion between cell genome contigs (dark boxes) and a ctDNA contigs (light boxes) for pancreatic cancer ctDNA. Results were obtained after comparing the contigs carrying insertions in the coculture condition with the reference cell genome.

FIG. 4 shows a summary of the distribution of contigs containing transposons and the fraction of transposons observed at 5′ or 3′ end in inserted vs. non inserted ctDNA fragments.

FIG. 5A shows illustrative common retrotransposons subfamilies and classes located at insertion points for pancreatic cancer ctDNA.

FIG. 5B shows illustrative common retrotransposons subfamilies and classes located at insertion points for multiple myeloma ctDNA.

FIG. 6A shows expression levels of selected transposable elements tumor samples.

FIG. 6B shows expression levels of selected transposable elements tumor samples.

FIG. 7 shows the effect of reverse transcriptase inhibitors or integrase inhibitor on ctDNA chromatid integration.

FIG. 8 shows an agarose gel of PCR products of transposons after incubation of the transposon constructs in complete media for 4 hours, followed by PCR.

FIG. 9A shows a time course of Cy5-AluSp and Cy5-control sequence treated MM1s cells (1 ug/mL). CY5 (+) cells were detected by flow cytometry.

FIG. 9B shows the results of a dose titration experiment of Cy5-AluSp and Cy5-control sequence treated MM1s cells. Before flow cytometry half of the samples were treated with trypsin to identify how much DNA as internalized.

FIG. 9C shows capture and internalization of different multiple myeloma retrotransposons and controls by MM1s cell after 4 hours of culture as evaluated by flow cytometry.

FIG. 9D shows capture and internalization of different multiple myeloma retrotransposons and controls by U266 cells after 4 hours in culture as evaluated by flow cytometry.

FIG. 10 provides illustrative flow cytometry scatterplots displaying the cell capture of AluSp, MER11, and control transposon sequence in plasma cells (CD138+) and non-plasma cells (CD138−) derived from bone marrow of multiple myeloma patients.

FIG. 11 shows the percentage of cells positive for fluorescently-labelled AluSp, MER11, control sequence, or a PC-specific transposon sequence after 14 hours of incubation with bone marrow of multiple myeloma patients or normal bone marrow. Data for plasma cells (CD138+) and non-plasma cells (CD138−) are separately shown.

FIG. 12A illustrates the effect of 5′ or 3′ deletions on internalization of Cy5 labeled AluSp by MM1s cells. Images were captured after culturing cells with the retrotransposons for 8 hours.

FIG. 12B provides gel images showing AluSp deletions.

FIG. 12C is a graphical display of the adenine (A)-thymine (T) and guanine (G)-cytosine (C) enriched regions or both identified by multiple sequence alignments of AluSp and other MM specific transposons.

FIG. 13 provides microscopy images of MM1s cells cultured with AluSp-CMV-mCherry, CMV-mCherry linear vector, or cells transfected with CMV-mCherry circular vector. Images were capture after 24 hours of coculture with DNA.

FIG. 14A is a bar chart displaying a summary of the number of mCherry genomic insertions identified in MM1s cells expressing high levels of mCherry, medium levels of mCherry, or no mCherry (n=5 cells per group).

FIG. 14B is an agarose gel showing a PCR band that indicates mCherry integration for chromatin extracted from cells treated with AluSp-CMV-mCherry cassette or control-CMV-mCherry cassette. * nonspecific band.

FIG. 14C displays the confidence of detection of the insertions identified versus the number of samples in which the specific site of insertion was detected.

FIG. 15 shows cell survival of 3 different cell lines cocultured with TE-HSV-Tk-GFP for 24 hours prior to adding ganciclovir (GCV). Apoptosis was measured at 96 hours after GVC addition. MM: multiple myeloma, CC: colon cancer and PC: pancreatic cancer, TE-CMV-GFP: transposon element joining to CMV-GFP, HSV-TK: herpes simplex virus thymidine kinase. Error bars in box and whiskers plot identify the standard deviation of triplicate experiments.

FIG. 16 shows the results of a cell viability assay measuring sensitivity to gemcitabine in pancreatic cancer cell lines (MIA and ASPC-1) cultured with plasma from patients resistant to gemcitabine, similar plasma pretreated with DNase I, or a control non-cancer patient. For the corresponding DNase I-treated samples, plasma was treated with DNase I for 10 minutes. GR: Gemcitabine resistant.

FIG. 17 shows a comparison of cell viability response to bortezomib in OPM1 cells cultured with plasma from a patient that failed to respond to bortezomib treatment, the combination of control plasma with ctDNA derived from the same patient resistant to bortezomib, and control plasma alone (non-cancer patient) (top left); viability response of RPMI to control plasma or control plasma with added ctDNA obtained from a patient that achieved a complete response to bortezomib (middle); cell viability assessment after bortezomib treatment of MM1s cells cultured with plasma from a bortezomib resistant patient (BR #2) alone or after treatment with DNase I or coculture with ctDNA from a different bortezomib-resistant (BR #1) patient (Right). BR: Bortezomib resistant, BS: Bortezomib sensitive. Error bars indicate the standard deviation of triplicate experiments.

FIG. 18A provides bar charts comparing the results of cell capture of synthesized, CY5-labelled retrotransposon sequences derived from multiple myeloma (MM) ctDNA to control. Data are grouped by cell line. In each case, the bars presented are, from left to right: control, followed by the synthesized MM-derived DNAs (MM01, MM02, MM03, MM04, and ZIP2). The horizontal line defines the control cutoff.

FIG. 18B provides bar charts comparing the results of cell capture of synthesized, CY5-labelled retrotransposon sequences derived from pancreatic ductal carcinoma (PDAC) ctDNA to control. Data are grouped by cell line. In each case, the bars presented are, from left to right: control, followed by the synthesized PDAC-derived DNAs (PC01, PC02, PC03, PC21, PC22, PC23, PC24). The horizontal line defines the control cutoff.

FIG. 19 provides fluorescence microcopy images showing uptake of fluorescently labelled synthetic DNA derived from a multiple myeloma ctDNA (MM2 (1-290), MM2 (89-290), MM2 (177-290), and MM2 (1-201), and MM2 (1-113)), or a control sequence.

FIG. 20 provides fluorescence microcopy images evaluating uptake of fluorescently labelled synthetic DNA derived from a multiple myeloma ctDNA (MM2 (1-290), MM2 (89-290), MM2 (177-290), and MM2 (1-201), and MM2 (1-113)) by multiple myeloma/B cell lines (MM1S and RPMI) and cell lines of other cell types (HFF—fibroblast, DU145—prostate cancer, MCF10A breast).

FIG. 21 provides microcopy images showing uptake of 5′ fluorescently labelled synthetic DNA fragments derived from a multiple myeloma ctDNA by MM cells (JK6L) or non-MM cells.

### DETAILED DESCRIPTION

Provided are compositions and methods including synthetic delivery systems that utilize polynucleotides disclosed herein. Polynucleotides disclosed herein can, for example, facilitate binding, uptake, nuclear trafficking, and/or genomic integration of a component of the synthetic delivery system, for example, a cargo.

Synthetic delivery systems disclosed herein can utilize polynucleotides that facilitate cell-specific horizontal gene transfer (HGT) between cells (e.g., human cancer cells). The transfer of genes between cells can play physiological and pathological roles in certain organisms. For example, HGT has been characterized in prokaryotes, in which HGT can provide survival and evolutionary advantages to environmental stressors. Evidence for HGT in mammals and humans is much more limited, as is understanding of potential mechanisms involved.

Identified herein are tissue-specific retrotransposons that can mediate a process whereby circulating tumor DNA (ctDNA) targets and transmits genetic material to cells that resemble the ctDNA's cell of origin. Compositions and methods provided herein include synthetic delivery systems that utilize polynucleotides to facilitate binding, uptake, nuclear trafficking, and/or genomic integration of a component of the synthetic delivery system, for example, a cargo. The compositions and methods can be used for delivery of a cargo to cells, including specific cell types and cancer cells.

### I. SYNTHETIC DELIVERY SYSTEM

Disclosed herein are synthetic delivery systems that can provide for cell-, tissue-, and/or organ-specific targeting, delivery, binding, uptake, nuclear localization and/or genomic integration, e.g., of a cargo. Compositions and methods provided herein include synthetic delivery systems that utilize polynucleotides to facilitate binding, uptake, nuclear trafficking, and/or genomic integration of a component of the synthetic delivery system, for example, a cargo. The compositions and methods can be used for delivery of a cargo to cells, including specific cell types and cancer cells.

A synthetic delivery system disclosed herein can comprise a polynucleotide. A synthetic delivery system can comprise, for example, a polynucleotide and a cargo. A polynucleotide can be or can comprise double stranded DNA (dsDNA). In some embodiments, a polynucleotide comprises single stranded DNA. In some embodiments, a polynucleotide comprises RNA.

A polynucleotide of the disclosure, sequence thereof, or a part thereof (for example, recognition and/or integration sequence) can be from or derived from a nucleic acid sequence of a biological sample (e.g., blood, plasma, or a tissue sample of a subject, such as a human). A polynucleotide of the disclosure, sequence thereof, or a part thereof (for example, recognition and/or integration sequence) can be from or derived from a cancerous cell or tissue. The polynucleotide can be recombinant or synthetic, e.g., not isolated from a biological sample.

A polynucleotide in a synthetic delivery system or a part thereof (for example, recognition and/or integration sequence) can be from or derived from a circulating tumor DNA (ctDNA), for example, from a human. A ctDNA can comprise double stranded DNA (dsDNA). In some embodiments, a ctDNA comprises single stranded DNA. Such a ctDNA or a fragment thereof can comprise a nucleic acid sequence that can have the ability to target a certain cell or cell population, and induce binding, uptake, nuclear localization, and/or genomic integration of a polynucleotide or cargo disclosed herein.

A polynucleotide in a synthetic delivery system that delivers a cargo to a target cell can comprise a nucleic acid sequence that is from or derived from ctDNA. The ctDNA can originate from the same cell type or tissue type as the target cell. A polynucleotide used to deliver a cargo to a target cell can comprise a nucleic acid sequence that is from or derived from ctDNA of substantially the same cell type or substantially the same tissue type as the target cell. A polynucleotide used to deliver a cargo to a target cell can comprise a nucleic acid sequence that is from or derived from ctDNA of a substantially similar cell type or substantially similar tissue type as the target cell. For example, a ctDNA from multiple myeloma or a component (e.g., subsequence or recognition sequence) therefrom can be used to generate a synthetic delivery system for delivery, binding, uptake, nuclear localization, and/or genomic integration of a cargo to a multiple myeloma target cell or a related target cell or target cell type. ctDNA from pancreatic cancer or a component (e.g., subsequence or recognition sequence) therefrom can be used to generate a synthetic delivery system for delivery, binding, uptake, nuclear localization, and/or genomic integration of a cargo to a pancreatic cancer target cell or a related target cell or target cell type. ctDNA from colorectal cancer or a component (e.g., subsequence or recognition sequence) therefrom can be used to generate a synthetic delivery system for delivery, binding, uptake, nuclear localization, and/or genomic integration of a cargo to a colorectal cancer target cell or a related target cell or target cell type. ctDNA from lung cancer or a component (e.g., subsequence or recognition sequence) therefrom can be used to generate a synthetic delivery system for delivery, binding, uptake, nuclear localization, and/or genomic integration of a cargo to a lung cancer target cell or a related target cell or target cell type.

A synthetic delivery system, polynucleotide, nucleic acid sequence, recognition sequence, integration sequence, or cargo can comprise a natural or unnatural nucleotide analogue or base or a combination thereof. A nucleotide analogue can comprise modification(s) at one or more of a ribose moiety, phosphate moiety, nucleoside moiety, or a combination thereof. A nucleotide analogue or base can comprise 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, locked nucleic acid (LNA), ethylene nucleic acid (ENA), peptide nucleic acid (PNA), 1′,5′-anhydrohexitol nucleic acids (HNA), morpholino, methylphosphonate nucleotides, thiolphosphonate nucleotides, or 2′-fluoro N3-P5′-phosphoramidites. A polynucleotide can comprise one or more abasic sites. The abasic site can be functionalized with a detectable moiety.

A synthetic delivery system, polynucleotide, first nucleic acid sequence, or second nucleic acid sequence can comprise a recognition sequence that can provide for cell-specific targeting and/or uptake of the polynucleotide, synthetic delivery system, or cargo. In some embodiments, the recognition sequence binds to a receptor that mediates or is associated with uptake of ctDNA and/or the synthetic delivery system. In some embodiments, the recognition sequence of a ctDNA, synthetic delivery system, or polynucleotide specifically or preferentially binds to a receptor that is associated with uptake of ctDNA and/or the synthetic delivery system.

In some embodiments, the recognition sequence facilitates preferential binding of a synthetic delivery system, polynucleotide, or cargo to a target cell as compared to a control cell. In some embodiments, the recognition sequence facilitates preferential uptake of a ctDNA, synthetic delivery system, polynucleotide, or cargo by a target cell as compared to a control cell. In some embodiments, the recognition sequence facilitates preferential binding, uptake, nuclear localization, and/or genomic integration of a ctDNA, synthetic delivery system, polynucleotide, or cargo by a target cell as compared to a control cell. The control cell can be, for example, a cell of a different cell type (e.g., a cell type disclosed herein). In some embodiments, the target cell is a cancer cell and the control cell is a non-cancer cell. In some embodiments, the target cell is a cancer cell of a first cell type and the control cell is a cancer cell of a second cell type. In some embodiments, the target cell is a cancer cell of a first cell type and the control cell is a non-cancer cell of a second cell type.

A recognition sequence can be or can comprise a functional fragment and/or variant of a sequence disclosed herein. For example, a recognition sequence can comprise a subset of nucleotides from a nucleotide sequence disclosed herein that facilitates preferential binding to a target cell as compared to a control cell, preferential uptake by the target cell as compared to the control cell, preferential nuclear localization as compared to the control cell, and/or preferential integration into the genome of the target cell as compared to the control cell. In some embodiments, the recognition sequence comprises a functional fragment and/or variant of a sequence disclosed herein that retains at least 10%, at least 20%, at least 30%, at least 40%, at least 50%, at least 60%, at least 70%, at least 80%, at least 90%, or at least 95% of the target cell binding, uptake, nuclear localization, and/or genomic integration activity of the nucleotide sequence from which the fragment and/or variant is derived.

For example, in some embodiments, the synthetic delivery system, polynucleotide, nucleic acid sequence, first nucleic acid sequence, second nucleic acid sequence, recognition sequence, or integration sequence disclosed herein is a functional fragment and/or variant of SEQ ID NO: 68, or SEQ ID NO: 78 (ACCCGGCCTTGGACACGCCATTTTCAACTCCGTGGTGCGTTTTTTTTTTTTTTTTT TTTTTTTGTAATGGAGTTTTGCTCTTGTTGCCCAGGATGGAGTGCAAGGGATCTTG GCTCACCACAGCCTCTGCCTCCTGGGTTCAAGTGATTCTTCTGCCTCAGCCTCCCA AGTAGCTGGGATTATAAGCACCCACCACCACGCCCAGCTAATTTTGTATTTTTTA GAAGAGATGGAGTTTCTCCAGTTGGCCAGGATGGTCTGTATATCCTGACCTCATG ATCTGCCCACCA), e.g., that comprises a subset of nucleotides from SEQ ID NO: 68 or SEQ ID NO: 78 that facilitate preferential binding to a target cell as compared to a control cell, preferential uptake by the target cell as compared to the control cell, preferential nuclear localization as compared to the control cell, and/or preferential integration into the genome of the target cell as compared to the control cell. In some embodiments, the recognition sequence comprises a functional fragment and/or variant of SEQ ID NO: 68 or SEQ ID NO: 78 that retains at least 10%, at least 20%, at least 30%, at least 40%, at least 50%, at least 60%, at least 70%, at least 80%, at least 90%, or at least 95% of the target cell binding, uptake, nuclear localization, and/or genomic integration activity of SEQ ID NO: 68 or SEQ ID NO: 78.

In some embodiments, the synthetic delivery system, polynucleotide, nucleic acid sequence, first nucleic acid sequence, second nucleic acid sequence, recognition sequence, or integration sequence does not contain a nucleotide sequence with at least 10%, at least 20%, at least 30%, at least 40%, at least 50%, at least 60%, at least 70%, at least 80%, at least 90%, or at least 95%, at least 97%, or at least 99% sequence identity to nucleotides 1-87 of SEQ ID NO: 68 or nucleotides 1-87 of SEQ ID NO: 78. In some embodiments, the synthetic delivery system, polynucleotide, nucleic acid sequence, first nucleic acid sequence, second nucleic acid sequence, recognition sequence, or integration sequence does not contain the nucleotide sequence of nucleotides 1-87 of SEQ ID NO: 68 or nucleotides 1-87 of SEQ ID NO: 78. In some embodiments, the synthetic delivery system or polynucleotide comprises the nucleotide sequence of nucleotides 1-87 of SEQ ID NO: 68, nucleotides 1-87 of SEQ ID NO: 78, or sequence with at least a minimum sequence identity thereto, but said sequence is not part of the recognition sequence, e.g., is not required for preferential binding to, uptake by, or nuclear localization in a target cell.

In some embodiments, the synthetic delivery system, polynucleotide, nucleic acid sequence, first nucleic acid sequence, second nucleic acid sequence, recognition sequence, or integration sequence does not contain a nucleotide sequence with at least 10%, at least 20%, at least 30%, at least 40%, at least 50%, at least 60%, at least 70%, at least 80%, at least 90%, or at least 95%, at least 97%, or at least 99% sequence identity to SEQ ID NO: 71. In some embodiments, the synthetic delivery system, polynucleotide, nucleic acid sequence, first nucleic acid sequence, second nucleic acid sequence, recognition sequence, or integration sequence does not contain the nucleotide sequence of SEQ ID NO: 71. In some embodiments, the synthetic delivery system or polynucleotide comprises SEQ ID NO: 71, or sequence with at least a minimum sequence identity thereto, but said sequence is not part of the recognition sequence, e.g., is not required for preferential binding to, uptake by, or nuclear localization in a target cell.

In some embodiments, the synthetic delivery system, polynucleotide, nucleic acid sequence, first nucleic acid sequence, second nucleic acid sequence, recognition sequence, or integration sequence comprises at most 10%, at most 20%, at most 30%, at most 40%, at most 50%, at most 60%, at most 70%, at most 80%, or at most 90% sequence identity to SEQ ID NO: 68 or SEQ ID NO: 78.

A recognition sequence can comprise, consist essentially of, or consist of a subset of consecutive nucleotides from a sequence disclosed herein, for example, at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at most about 10, at most about 20, at most about 30, at most about 40, at most about 50, at most about 60, at most about 70, at most about 80, at most about 90, at most about 100, at most about 110, at most about 120, at most about 130, at most about 140, at most about 150, at most about 160, at most about 170, at most about 180, at most about 190, at most about 200, at most about 250, at most about 300, at most about 350, at most about 400, at most about 450, at most about 500, about 10, about 15, about 20, about 25, about 30, about 40, about 50, about 60, about 70, about 80, about 90, about 100, about 110, about 120, about 130, about 140, about 150, about 160, about 170, about 180, about 190, about 200, about 250, about 300, about 350, about 400, about 450, about 500, about 5-10, about 5-20, about 5-30, about 5-40, about 5-50, about 5-60, about 5-70, about 5-80, about 5-90, about 5-100, about 5-150, about 5-200, about 5-300, about 5-400, about 5-500, about 10-20, about 10-30, about 10-40, about 10-50, about 10-60, about 10-70, about 10-80, about 10-90, about 10-100, about 10-150, about 10-200, about 10-300, about 10-400, about 10-500, about 15-20, about 15-30, about 15-40, about 15-50, about 15-60, about 15-70, about 15-80, about 15-90, about 15-100, about 15-150, about 15-200, about 15-300, about 15-400, about 15-500, about 20-30, about 20-40, about 20-50, about 20-60, about 20-70, about 20-80, about 20-90, about 20-100, about 20-150, about 20-200, about 20-300, about 20-400, about 20-500, about 30-40, about 30-50, about 30-60, about 30-70, about 30-80, about 30-90, about 30-100, about 30-150, about 30-200, about 30-300, about 30-400, about 30-500, about 40-50, about 40-60, about 40-70, about 40-80, about 40-90, about 40-100, about 40-150, about 40-200, about 40-300, about 40-400, about 40-500, about 50-60, about 50-70, about 50-80, about 50-90, about 50-100, about 50-150, about 50-200, about 50-300, about 50-400, about 50-500, about 100-150, about 100-200, about 100-300, about 100-400, about 100-500, about 200-300, about 200-400, or about 200-500 consecutive nucleotides from any one of SEQ ID NOs: 1-78.

A recognition sequence can comprise, consist essentially of, or consist of a nucleotide sequence with at least a minimum percentage of sequence identity to a subset of consecutive nucleotides from a sequence disclosed herein.

In some embodiments, a recognition sequence comprises at least 70% sequence identity to at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 consecutive nucleotides from any one of SEQ ID NOs: 1-78.

In some embodiments, a recognition sequence comprises at least 80% sequence identity to at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 consecutive nucleotides from any one of SEQ ID NOs: 1-78.

In some embodiments, a recognition sequence comprises at least 85% sequence identity to at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 consecutive nucleotides from any one of SEQ ID NOs: 1-78.

In some embodiments, a recognition sequence comprises at least 90% sequence identity to at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 consecutive nucleotides from any one of SEQ ID NOs: 1-78.

In some embodiments, a recognition sequence comprises at least 95% sequence identity to at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 consecutive nucleotides from any one of SEQ ID NOs: 1-78.

In some embodiments, a recognition sequence comprises at least 98% sequence identity to at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 consecutive nucleotides from any one of SEQ ID NOs: 1-78.

In some embodiments, a recognition sequence comprises at least 70% sequence identity to at least about 30 consecutive nucleotides of SEQ ID NO: 73. In some embodiments, a recognition sequence comprises at least 70% sequence identity to at least about 40 consecutive nucleotides of SEQ ID NO: 73. In some embodiments, a recognition sequence comprises at least 70% sequence identity to at least about 50 consecutive nucleotides of SEQ ID NO: 73. In some embodiments, a recognition sequence comprises at least 70% sequence identity to at least about 60 consecutive nucleotides of SEQ ID NO: 73. In some embodiments, a recognition sequence comprises at least 70% sequence identity to at least about 70 consecutive nucleotides of SEQ ID NO: 73. In some embodiments, a recognition sequence comprises at least 70% sequence identity to at least about 80 consecutive nucleotides of SEQ ID NO: 73.

In some embodiments, a recognition sequence comprises at least 70% sequence identity to at least about 30 consecutive nucleotides of SEQ ID NO: 74. In some embodiments, a recognition sequence comprises at least 70% sequence identity to at least about 40 consecutive nucleotides of SEQ ID NO: 74. In some embodiments, a recognition sequence comprises at least 70% sequence identity to at least about 50 consecutive nucleotides of SEQ ID NO: 74. In some embodiments, a recognition sequence comprises at least 70% sequence identity to at least about 60 consecutive nucleotides of SEQ ID NO: 74. In some embodiments, a recognition sequence comprises at least 70% sequence identity to at least about 70 consecutive nucleotides of SEQ ID NO: 74. In some embodiments, a recognition sequence comprises at least 70% sequence identity to at least about 80 consecutive nucleotides of SEQ ID NO: 74. In some embodiments, a recognition sequence comprises at least 70% sequence identity to at least about 90 consecutive nucleotides of SEQ ID NO: 74. In some embodiments, a recognition sequence comprises at least 70% sequence identity to at least about 100 consecutive nucleotides of SEQ ID NO: 74.

In some embodiments, a recognition sequence comprises at least 70% sequence identity to at least about 30 consecutive nucleotides of SEQ ID NO: 75. In some embodiments, a recognition sequence comprises at least 70% sequence identity to at least about 40 consecutive nucleotides of SEQ ID NO: 75. In some embodiments, a recognition sequence comprises at least 70% sequence identity to at least about 50 consecutive nucleotides of SEQ ID NO: 75. In some embodiments, a recognition sequence comprises at least 70% sequence identity to at least about 60 consecutive nucleotides of SEQ ID NO: 75. In some embodiments, a recognition sequence comprises at least 70% sequence identity to at least about 70 consecutive nucleotides of SEQ ID NO: 75. In some embodiments, a recognition sequence comprises at least 70% sequence identity to at least about 80 consecutive nucleotides of SEQ ID NO: 75. In some embodiments, a recognition sequence comprises at least 70% sequence identity to at least about 90 consecutive nucleotides of SEQ ID NO: 75. In some embodiments, a recognition sequence comprises at least 70% sequence identity to at least about 100 consecutive nucleotides of SEQ ID NO: 75. In some embodiments, a recognition sequence comprises at least 70% sequence identity to at least about 125 consecutive nucleotides of SEQ ID NO: 75. In some embodiments, a recognition sequence comprises at least 70% sequence identity to at least about 150 consecutive nucleotides of SEQ ID NO: 75.

In some embodiments, a recognition sequence comprises at least 80% sequence identity to at least about 30 consecutive nucleotides of SEQ ID NO: 73. In some embodiments, a recognition sequence comprises at least 80% sequence identity to at least about 40 consecutive nucleotides of SEQ ID NO: 73. In some embodiments, a recognition sequence comprises at least 80% sequence identity to at least about 50 consecutive nucleotides of SEQ ID NO: 73. In some embodiments, a recognition sequence comprises at least 80% sequence identity to at least about 60 consecutive nucleotides of SEQ ID NO: 73. In some embodiments, a recognition sequence comprises at least 80% sequence identity to at least about 70 consecutive nucleotides of SEQ ID NO: 73. In some embodiments, a recognition sequence comprises at least 80% sequence identity to at least about 80 consecutive nucleotides of SEQ ID NO: 73.

In some embodiments, a recognition sequence comprises at least 80% sequence identity to at least about 30 consecutive nucleotides of SEQ ID NO: 74. In some embodiments, a recognition sequence comprises at least 80% sequence identity to at least about 40 consecutive nucleotides of SEQ ID NO: 74. In some embodiments, a recognition sequence comprises at least 80% sequence identity to at least about 50 consecutive nucleotides of SEQ ID NO: 74. In some embodiments, a recognition sequence comprises at least 80% sequence identity to at least about 60 consecutive nucleotides of SEQ ID NO: 74. In some embodiments, a recognition sequence comprises at least 80% sequence identity to at least about 70 consecutive nucleotides of SEQ ID NO: 74. In some embodiments, a recognition sequence comprises at least 80% sequence identity to at least about 80 consecutive nucleotides of SEQ ID NO: 74. In some embodiments, a recognition sequence comprises at least 80% sequence identity to at least about 90 consecutive nucleotides of SEQ ID NO: 74. In some embodiments, a recognition sequence comprises at least 80% sequence identity to at least about 100 consecutive nucleotides of SEQ ID NO: 74.

In some embodiments, a recognition sequence comprises at least 80% sequence identity to at least about 30 consecutive nucleotides of SEQ ID NO: 75. In some embodiments, a recognition sequence comprises at least 80% sequence identity to at least about 40 consecutive nucleotides of SEQ ID NO: 75. In some embodiments, a recognition sequence comprises at least 80% sequence identity to at least about 50 consecutive nucleotides of SEQ ID NO: 75. In some embodiments, a recognition sequence comprises at least 80% sequence identity to at least about 60 consecutive nucleotides of SEQ ID NO: 75. In some embodiments, a recognition sequence comprises at least 80% sequence identity to at least about 70 consecutive nucleotides of SEQ ID NO: 75. In some embodiments, a recognition sequence comprises at least 80% sequence identity to at least about 80 consecutive nucleotides of SEQ ID NO: 75. In some embodiments, a recognition sequence comprises at least 80% sequence identity to at least about 90 consecutive nucleotides of SEQ ID NO: 75. In some embodiments, a recognition sequence comprises at least 80% sequence identity to at least about 100 consecutive nucleotides of SEQ ID NO: 75. In some embodiments, a recognition sequence comprises at least 80% sequence identity to at least about 125 consecutive nucleotides of SEQ ID NO: 75. In some embodiments, a recognition sequence comprises at least 80% sequence identity to at least about 150 consecutive nucleotides of SEQ ID NO: 75.

In some embodiments, a recognition sequence comprises at least 90% sequence identity to at least about 30 consecutive nucleotides of SEQ ID NO: 73. In some embodiments, a recognition sequence comprises at least 90% sequence identity to at least about 40 consecutive nucleotides of SEQ ID NO: 73. In some embodiments, a recognition sequence comprises at least 90% sequence identity to at least about 50 consecutive nucleotides of SEQ ID NO: 73. In some embodiments, a recognition sequence comprises at least 90% sequence identity to at least about 60 consecutive nucleotides of SEQ ID NO: 73. In some embodiments, a recognition sequence comprises at least 90% sequence identity to at least about 70 consecutive nucleotides of SEQ ID NO: 73. In some embodiments, a recognition sequence comprises at least 90% sequence identity to at least about 80 consecutive nucleotides of SEQ ID NO: 73.

In some embodiments, a recognition sequence comprises at least 90% sequence identity to at least about 30 consecutive nucleotides of SEQ ID NO: 74. In some embodiments, a recognition sequence comprises at least 90% sequence identity to at least about 40 consecutive nucleotides of SEQ ID NO: 74. In some embodiments, a recognition sequence comprises at least 90% sequence identity to at least about 50 consecutive nucleotides of SEQ ID NO: 74. In some embodiments, a recognition sequence comprises at least 90% sequence identity to at least about 60 consecutive nucleotides of SEQ ID NO: 74. In some embodiments, a recognition sequence comprises at least 90% sequence identity to at least about 70 consecutive nucleotides of SEQ ID NO: 74. In some embodiments, a recognition sequence comprises at least 90% sequence identity to at least about 80 consecutive nucleotides of SEQ ID NO: 74. In some embodiments, a recognition sequence comprises at least 90% sequence identity to at least about 90 consecutive nucleotides of SEQ ID NO: 74. In some embodiments, a recognition sequence comprises at least 90% sequence identity to at least about 100 consecutive nucleotides of SEQ ID NO: 74.

In some embodiments, a recognition sequence comprises at least 90% sequence identity to at least about 30 consecutive nucleotides of SEQ ID NO: 75. In some embodiments, a recognition sequence comprises at least 90% sequence identity to at least about 40 consecutive nucleotides of SEQ ID NO: 75. In some embodiments, a recognition sequence comprises at least 90% sequence identity to at least about 50 consecutive nucleotides of SEQ ID NO: 75. In some embodiments, a recognition sequence comprises at least 90% sequence identity to at least about 60 consecutive nucleotides of SEQ ID NO: 75. In some embodiments, a recognition sequence comprises at least 90% sequence identity to at least about 70 consecutive nucleotides of SEQ ID NO: 75. In some embodiments, a recognition sequence comprises at least 90% sequence identity to at least about 80 consecutive nucleotides of SEQ ID NO: 75. In some embodiments, a recognition sequence comprises at least 90% sequence identity to at least about 90 consecutive nucleotides of SEQ ID NO: 75. In some embodiments, a recognition sequence comprises at least 90% sequence identity to at least about 100 consecutive nucleotides of SEQ ID NO: 75. In some embodiments, a recognition sequence comprises at least 90% sequence identity to at least about 125 consecutive nucleotides of SEQ ID NO: 75. In some embodiments, a recognition sequence comprises at least 90% sequence identity to at least about 150 consecutive nucleotides of SEQ ID NO: 75.

In some embodiments, a recognition sequence comprises at least 95% sequence identity to at least about 30 consecutive nucleotides of SEQ ID NO: 73. In some embodiments, a recognition sequence comprises at least 95% sequence identity to at least about 40 consecutive nucleotides of SEQ ID NO: 73. In some embodiments, a recognition sequence comprises at least 95% sequence identity to at least about 50 consecutive nucleotides of SEQ ID NO: 73. In some embodiments, a recognition sequence comprises at least 95% sequence identity to at least about 60 consecutive nucleotides of SEQ ID NO: 73. In some embodiments, a recognition sequence comprises at least 95% sequence identity to at least about 70 consecutive nucleotides of SEQ ID NO: 73. In some embodiments, a recognition sequence comprises at least 95% sequence identity to at least about 80 consecutive nucleotides of SEQ ID NO: 73.

In some embodiments, a recognition sequence comprises at least 95% sequence identity to at least about 30 consecutive nucleotides of SEQ ID NO: 74. In some embodiments, a recognition sequence comprises at least 95% sequence identity to at least about 40 consecutive nucleotides of SEQ ID NO: 74. In some embodiments, a recognition sequence comprises at least 95% sequence identity to at least about 50 consecutive nucleotides of SEQ ID NO: 74. In some embodiments, a recognition sequence comprises at least 95% sequence identity to at least about 60 consecutive nucleotides of SEQ ID NO: 74. In some embodiments, a recognition sequence comprises at least 95% sequence identity to at least about 70 consecutive nucleotides of SEQ ID NO: 74. In some embodiments, a recognition sequence comprises at least 95% sequence identity to at least about 80 consecutive nucleotides of SEQ ID NO: 74. In some embodiments, a recognition sequence comprises at least 95% sequence identity to at least about 90 consecutive nucleotides of SEQ ID NO: 74. In some embodiments, a recognition sequence comprises at least 95% sequence identity to at least about 100 consecutive nucleotides of SEQ ID NO: 74.

In some embodiments, a recognition sequence comprises at least 95% sequence identity to at least about 30 consecutive nucleotides of SEQ ID NO: 75. In some embodiments, a recognition sequence comprises at least 95% sequence identity to at least about 40 consecutive nucleotides of SEQ ID NO: 75. In some embodiments, a recognition sequence comprises at least 95% sequence identity to at least about 50 consecutive nucleotides of SEQ ID NO: 75. In some embodiments, a recognition sequence comprises at least 95% sequence identity to at least about 60 consecutive nucleotides of SEQ ID NO: 75. In some embodiments, a recognition sequence comprises at least 95% sequence identity to at least about 70 consecutive nucleotides of SEQ ID NO: 75. In some embodiments, a recognition sequence comprises at least 95% sequence identity to at least about 80 consecutive nucleotides of SEQ ID NO: 75. In some embodiments, a recognition sequence comprises at least 95% sequence identity to at least about 90 consecutive nucleotides of SEQ ID NO: 75. In some embodiments, a recognition sequence comprises at least 95% sequence identity to at least about 100 consecutive nucleotides of SEQ ID NO: 75. In some embodiments, a recognition sequence comprises at least 95% sequence identity to at least about 125 consecutive nucleotides of SEQ ID NO: 75. In some embodiments, a recognition sequence comprises at least 95% sequence identity to at least about 150 consecutive nucleotides of SEQ ID NO: 75.

In some embodiments, a recognition sequence comprises, consists essentially of, or consists of, about, or at least about 30 consecutive nucleotides of SEQ ID NO: 73. In some embodiments, a recognition sequence comprises, consists essentially of, or consists of, about, or at least about 40 consecutive nucleotides of SEQ ID NO: 73. In some embodiments, a recognition sequence comprises, consists essentially of, or consists of, about, or at least about 50 consecutive nucleotides of SEQ ID NO: 73. In some embodiments, a recognition sequence comprises, consists essentially of, or consists of, about, or at least about 60 consecutive nucleotides of SEQ ID NO: 73. In some embodiments, a recognition sequence comprises, consists essentially of, or consists of, about, or at least about 70 consecutive nucleotides of SEQ ID NO: 73. In some embodiments, a recognition sequence comprises, consists essentially of, or consists of, about, or at least about 80 consecutive nucleotides of SEQ ID NO: 73.

In some embodiments, a recognition sequence comprises, consists essentially of, or consists of, about, or at least about 30 consecutive nucleotides of SEQ ID NO: 74. In some embodiments, a recognition sequence comprises, consists essentially of, or consists of, about, or at least about 40 consecutive nucleotides of SEQ ID NO: 74. In some embodiments, a recognition sequence comprises, consists essentially of, or consists of, about, or at least about 50 consecutive nucleotides of SEQ ID NO: 74. In some embodiments, a recognition sequence comprises, consists essentially of, or consists of, about, or at least about 60 consecutive nucleotides of SEQ ID NO: 74. In some embodiments, a recognition sequence comprises, consists essentially of, or consists of, about, or at least about 70 consecutive nucleotides of SEQ ID NO: 74. In some embodiments, a recognition sequence comprises, consists essentially of, or consists of, about, or at least about 80 consecutive nucleotides of SEQ ID NO: 74. In some embodiments, a recognition sequence comprises, consists essentially of, or consists of, about, or at least about 90 consecutive nucleotides of SEQ ID NO: 74. In some embodiments, a recognition sequence comprises, consists essentially of, or consists of, about, or at least about 100 consecutive nucleotides of SEQ ID NO: 74.

In some embodiments, a recognition sequence comprises, consists essentially of, or consists of, about, or at least about 30 consecutive nucleotides of SEQ ID NO: 75. In some embodiments, a recognition sequence comprises, consists essentially of, or consists of, about, or at least about 40 consecutive nucleotides of SEQ ID NO: 75. In some embodiments, a recognition sequence comprises, consists essentially of, or consists of, about, or at least about 50 consecutive nucleotides of SEQ ID NO: 75. In some embodiments, a recognition sequence comprises, consists essentially of, or consists of, about, or at least about 60 consecutive nucleotides of SEQ ID NO: 75. In some embodiments, a recognition sequence comprises, consists essentially of, or consists of, about, or at least about 70 consecutive nucleotides of SEQ ID NO: 75. In some embodiments, a recognition sequence comprises, consists essentially of, or consists of, about, or at least about 80 consecutive nucleotides of SEQ ID NO: 75. In some embodiments, a recognition sequence comprises, consists essentially of, or consists of, about, or at least about 90 consecutive nucleotides of SEQ ID NO: 75. In some embodiments, a recognition sequence comprises, consists essentially of, or consists of, about, or at least about 100 consecutive nucleotides of SEQ ID NO: 75. In some embodiments, a recognition sequence comprises, consists essentially of, or consists of, about, or at least about 125 consecutive nucleotides of SEQ ID NO: 75. In some embodiments, a recognition sequence comprises, consists essentially of, or consists of, about, or at least about 150 consecutive nucleotides of SEQ ID NO: 75.

In some embodiments, a recognition sequence is at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 nucleotides in length.

In some embodiments, a recognition sequence is at most about 10, at most about 20, at most about 30, at most about 40, at most about 50, at most about 60, at most about 70, at most about 80, at most about 90, at most about 100, at most about 110, at most about 120, at most about 130, at most about 140, at most about 150, at most about 160, at most about 170, at most about 180, at most about 190, at most about 200, at most about 250, at most about 300, at most about 350, at most about 400, at most about 450, or at most about 500 nucleotides in length.

In some embodiments, a recognition sequence is about 10, about 15, about 20, about 25, about 30, about 40, about 50, about 60, about 70, about 80, about 90, about 100, about 110, about 120, about 130, about 140, about 150, about 160, about 170, about 180, about 190, about 200, about 250, about 300, about 350, about 400, about 450, or about 500 nucleotides in length.

In some embodiments, a recognition sequence is about 5-10, about 5-20, about 5-30, about 5-40, about 5-50, about 5-60, about 5-70, about 5-80, about 5-90, about 5-100, about 5-150, about 5-200, about 5-300, about 5-400, about 5-500, about 10-20, about 10-30, about 10-40, about 10-50, about 10-60, about 10-70, about 10-80, about 10-90, about 10-100, about 10-150, about 10-200, about 10-300, about 10-400, about 10-500, about 15-20, about 15-30, about 15-40, about 15-50, about 15-60, about 15-70, about 15-80, about 15-90, about 15-100, about 15-150, about 15-200, about 15-300, about 15-400, about 15-500, about 20-30, about 20-40, about 20-50, about 20-60, about 20-70, about 20-80, about 20-90, about 20-100, about 20-150, about 20-200, about 20-300, about 20-400, about 20-500, about 30-40, about 30-50, about 30-60, about 30-70, about 30-80, about 30-90, about 30-100, about 30-150, about 30-200, about 30-300, about 30-400, about 30-500, about 40-50, about 40-60, about 40-70, about 40-80, about 40-90, about 40-100, about 40-150, about 40-200, about 40-300, about 40-400, about 40-500, about 50-60, about 50-70, about 50-80, about 50-90, about 50-100, about 50-150, about 50-200, about 50-300, about 50-400, about 50-500, about 100-150, about 100-200, about 100-300, about 100-400, about 100-500, about 200-300, about 200-400, or about 200-500 nucleotides in length.

In some embodiments, the recognition sequence of a synthetic delivery system or polynucleotide facilitates at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold higher binding to a target cell (e.g., population thereof) disclosed herein as compared to a control ctDNA, synthetic delivery system, or polynucleotide that lacks the recognition sequence, or comprises a control recognition sequence.

In some embodiments, the recognition sequence of a synthetic delivery system or polynucleotide facilitates at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold higher uptake of the synthetic delivery system, polynucleotide, or cargo by target cells as compared to a control ctDNA, synthetic delivery system, or polynucleotide that lacks the recognition sequence, or comprises a control recognition sequence.

In some embodiments, the recognition sequence of a synthetic delivery system or polynucleotide facilitates at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold higher nuclear localization of the synthetic delivery system, polynucleotide, or cargo in target cells as compared to a control ctDNA, synthetic delivery system, or polynucleotide that lacks the recognition sequence, or comprises a control recognition sequence.

In some embodiments, the recognition sequence of a synthetic delivery system or polynucleotide facilitates at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold higher genomic integration of the synthetic delivery system, polynucleotide, or cargo by target cells as compared to a control ctDNA, synthetic delivery system, or polynucleotide that lacks the recognition sequence, or comprises a control recognition sequence.

In an example, a multiple myeloma (MM)-derived recognition sequence can target binding and/or uptake of a synthetic delivery system, polynucleotide, and/or cargo by MM cells but not, e.g. pancreatic cells. A pancreatic cancer (PC)-derived recognition sequence can target binding and/or uptake of a synthetic delivery system, polynucleotide, and/or cargo by pancreatic cancer cells, but not, e.g., multiple myeloma cells.

A recognition sequence can be derived from a ctDNA. For example, a recognition sequence can be a fragment of a ctDNA identified by sequencing a ctDNA from a subject, e.g., from plasma of a human subject with a cancer. A recognition sequence can be derived from a ctDNA that originates from a lymphoma, leukemia, lung cancer, colon cancer (e.g., colorectal cancer), brain cancer, multiple myeloma, pancreatic cancer, hematologic cancer, solid cancer, or other cancer type disclosed herein.

A synthetic delivery system or polynucleotide can comprise any suitable number of recognition sequences, for example, one, two, three, four, or five recognition sequences. In some embodiments, a synthetic delivery system or polynucleotide comprises one recognition sequence. In some embodiments, a synthetic delivery system or polynucleotide comprises two recognition sequences, for example, a first recognition sequence at a 5′ region of the polynucleotide and a second recognition sequence at a 3′ region of the polynucleotide, optionally separated by a non-recognition sequence or component, such as a nucleic acid cargo.

In some embodiments, a synthetic delivery system or polynucleotide comprises at least two, at least three, at least four, at least five, or another suitable number of recognition sequences at either the 3′ or the 5′ end, or a combination thereof, of a polynucleotide. A composition of the disclosure can comprise a plurality of such polynucleotides, and each polynucleotide can target one or more cell type(s). Accordingly, the methods and compositions of the present disclosure can be used to target one or more different target cells, one or more different target cell population(s), and/or one or more different target tissue(s), e.g., simultaneously.

A synthetic delivery system, polynucleotide, or nucleic acid sequence (e.g., a recognition sequence, integration sequence, first nucleic acid sequence, or second nucleic acid sequence) can comprise a transposable element (TE) or a derivative thereof, for example, a fragment with a functional attribute disclosed herein. A transposable element can comprise a transposon or a derivative thereof. A transposable element can comprise a retrotransposon or a derivative thereof. A transposable element or transposon can be any suitable class, subclass, family, subfamily, type, or the like, including those disclosed herein. A transposable element or transposon can be a variant or derivative of a transposable element of a given class, subclass, family, subfamily, or type, including those disclosed herein.

A synthetic delivery system, polynucleotide, or nucleic acid sequence (e.g., a recognition sequence, integration sequence, first nucleic acid sequence, or second nucleic acid sequence) can comprise a transposable element or fragment thereof that is from or derived from, for example, a class I or class II transposon. In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from a short interspersed nuclear elements (SINE), long interspersed nuclear elements (LINE), ERVL, or ERVK transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from an AluSp, MER11C, AluY, 2L2a, ALluY, ALR/Alpha, ALU, AluJb, AluS1, AluSc8, AluSg, ALUSg2, AluSq, AluSq2, ALUSx, AluSx, AluSx1, AluSz, AluSz6, AluYc3, ASLUSq2, ERVK/LTR, ERVL, ERVL-MaLR, FLAM_C, AluY, FLAM_C and AluY, HERV17-int, HERV9N-int, L1M1, L1MB3, L1ME4b, L1MEg, L1P1, L1P3, L1PA10, L1PA15, L1PA7, L1PB4, L2a, L2a/LTR40b/MLT1J2, LINE/L1, LINE/L2, LTR, LTR/ERV1, LTR/ERVL, LTR/ERVL-MaLR, LTR/Gypsy, LTR41C, LTR81B, Mam_R4, Mamr4, Many, MER11B, MER41E, MIR, MIRB, MIRc, MIRc-part L2, MLT1J2, MLT2B1, MLT1J2, MLT2B4, parAluSp-FULLMTL1J2, REP522, Satellite/centr, SINE/Alu, SINE/MIR, THE1A, THE1B, THE1C, or Tigger3a transposable element, or a derivative thereof.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from a non-LTR retrotransposon transposable element, for example a SINE, LINE, or composite transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from a LINE class transposable element. In some embodiments, the transposable element or fragment thereof is or is derived from a LINE class transposable element of the L1 group, for example, a L1, L1HS, L1M1B_5, L1M1_5, L1M2A1_5, L1M2A_5, L1M2B_5, L1M2C_5, L1M2_5, L1M3A_5, L1M3B_5, L1M3C_5, L1M3DE_5, L1M3D_5, L1M4B, L1M6B_5end, L1M6_5end, L1M7_5end, L1MA1, L1MA10, L1MA2, L1MA3, L1MA4, L1MA4A, L1MA5, L1MASA, L1MA6, L1MA7, L1MA8, L1MA9, L1MA9_5, L1MB1, L1 MB2, L1 MB3, L1MB3_5, L1 MB4, L1MB4_5, L1 MB5, L1MB6_5, L1 MB7, L1 MB8, L1MC1, L1MC2, L1MC3, L1MC4, L1MC4B, L1MC4_5end, L1MC5, L1MCA_5, L1MCB_5, L1MCC_5, L1MD1, L1MD1_5, L1MD2, L1MD3, L1MDA_5, L1MDB_5, L1ME1, L1ME2, L1ME3, L1ME3A, L1ME3C_3end, L1ME3D_3end, L1ME3E_3end, L1ME3F_3end, L1ME4, L1ME4A, L1ME5, L1ME5_3end, L1MEA_5, L1MEB_5, L1MEC_5, L1MED_5, L1MEE_5, L1MEe_5end, L1MEf_5end, L1MEg_5end, L1ME_ORF2, L1P4a_5end, L1P4b_5end, L1P4c_5end, L1P4d_5end, L1P4e_5end, L1PA10, L1PA11, L1PA12, L1PA12_5, L1PA13, L1PA13_5, L1PA14, L1PA14_5, L1PA15, L1PA16, L1PA16_5, L1PA17_5, L1PA2, L1PA3, L1PA4, L1PA5, L1PA6, L1PA7, L1PA7_5, L1PA8, L1PB1, L1 PB2, L1PB2c, L1 PB3, L1 PB4, L1PBA1_5, L1PBA_5, L1PBB_5, L1PREC1, L1PREC2, L1P_MA2, HAL1, HAL1B (HAL1b), HAL1M8, IN25, MER25, or X9_LINE transposable element. In some embodiments, the transposable element or fragment thereof is or is derived from a LINE class transposable element of the L2 group, for example, an L2, L2A, L2B, L2C, L2D, X15_LINE, X24_LINE, UCON49, or UCON86 transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from a SINE class transposable element. In some embodiments, the transposable element or fragment thereof is or is derived from a SINE class transposable element of the SINE1/7S group, for example, an AluY lineage, AluS lineage, AluJ lineage, or monomeric Alu lineage transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from an AluY lineage transposable element, for example, an ALU, AluY, AluYa1, AluYa4, AluYa5, AluYa8, AluYb10, AluYb11, AluYb3a1, AluYb3a2, AluYb8, AluYb8a1, AluYb9, AluYbc3a, AluYc1, AluYc2, AluYc5, AluYd2, AluYd3, AluYd3a1, AluYd8, AluYe2, AluYe5, AluYf1, AluYf2, AluYf5, AluYg6, AluYh9, AluYi6, AluYk11, AluYk12, or AluYk13 transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from an AluS lineage transposable element, for example, an AluSc, AluSc5, AluSc8, AluSg, AluSg1, AluSg4, AluSg7, AluSp, AluSq, AluSq10, AluSq2, AluSq4, AluSx, AluSx3, AluSx4, AluSz, or AluSz6 transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from an AluJ lineage transposable element, for example, an AluJb, AluJo, AluJr, or AluJr4 transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from a monomeric Alu lineage transposable element, for example a FAM, FLAM, FRAM, or PB1D11 transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from a SINE class transposable element of the SINE2/tRNA group, for example, a MIR, MIR3, MIRb, MIRc, THER1, THER2, MARE3, UCON3, UCON55, LFSINE_Vert, LmeSINE1b, LmeSINE1c, or MamSINE1 transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from an LTR retrotransposon transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from an endogenous retrovirus (ERV) transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from an ERV1 superfamily transposable element, for example, an LTR06, LTR9, LTR24C, LTR26, LTR26B, LTR26C, LTR26D, LTR26E, LTR27, LTR27B, LTR27C, LTR29, LTR31, LTR34, LTR36, LTR44, LTR45, LTR45B, LTR45C, LTR48, LTR48B, LTR51, LTR54, LTR54B, LTR56, LTR59, LTR60, LTR60B, LTR64, LTR65, LTR68, LTR70, LTR72, LTR72B, LTR75_1, LTR78, LTR78B, LTR81A, LTR81AB, LTR81B, LTR81C, LTR2752, MER31A, MER31B, MER34A, MER34A1, MER34C, MER34C2, MER34D, MER39, MER39B, MER49, MER50B, MER50C, MER66A, MER66B, MER66D, MER72B, MER87, MER87B, MER88, MER90, MER90a_LTR (MER90a), MER92A, MER92B, MER92C, MER93, MER95, or MER101B transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from an ERV2 superfamily transposable element. In some embodiments, a transposable element is an ERV2 superfamily transposable element of the HML1, HML2, HML3, HML4, HML5, HML6, HML7, HML8, HML9, or HML10 group.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from a MER (Medium reiterated frequency repeats) family member.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from an HERV-K14I (HERVK14), LTR14A, LTR14B, HERVK, LTR5, LTR5A, HML3, HERVK9I (HERVK9), MER9a1, MER9a2, MER9a3, HML4, HERVK13I (HERVK13), LTR13, LTR13A, HML5, HERVK22I (HERVK22), LTR22A, LTR22B, LTR22B1, LTR22B2, LTR22C, LTR22C0, LTR22C2, HML6, HERVK3I, LTR3, LTR3A, LTR3B, HML7, HERVK11DI (HERVK11D), MER11D, HML8, HERVK11I (HERVK11), MER11A, MER11B, MER11C, HML9, HERV-K14CI (HERVK14C), LTR14C, HML10, HERVKC4, LTR14, LTR5B, LTR5_Hs, LTR22, LTR22E, MER9, MER9B, RLTR10B, or RLTR10C transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from an ERV3 superfamily transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from an ERV3 superfamily transposable element of the MaLR group, for example, a THE1_I, THE1A, THE1B, THE1C, THE1D, MLT1B, MLT1D, MLT1G, MLT1G1, MLT1G2, MLT1G3, MLT1H1, MLT1H2, MLT1I, MLT1J1, MLT1J2, MLT1K, MLT1L, MLT1M, MLT1N2, or MLT1O transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element or fragment thereof from or derived from an ERV3 superfamily transposable element of the ERVL lineage, for example, a HERVL, MLT2A1, MLT2A2, MLT2B3, MLT2C2, MLT2D, MLT2F, ERVL, MLT2B2, ERVL-B4, MLT2B4, ERVL-E, MLT2E, ERV3-16A3_I ERV3-16A3_LTR, LTR16A, LTR16A1, LTR16A2, LTR16B, LTR16B1, LTR16B2, LTR16C, LTR16D, LTR16D1, LTR16D2, LTR16E, LTR16E1, LTR16E2, HERV16, LTR16, ERVL47, LTR47B, LTR47B2, LTR47B3, or LTR47B4 transposable element.

In some embodiments, a synthetic delivery system, polynucleotide, or nucleic acid sequence (e.g., a recognition sequence, integration sequence, first nucleic acid sequence, or second nucleic acid sequence) comprises a transposable element that is an AluSp transposable element or a derivative thereof.

In some embodiments, a synthetic delivery system, polynucleotide, or nucleic acid sequence (e.g., a recognition sequence, integration sequence, first nucleic acid sequence, or second nucleic acid sequence) comprises a transposable element that is a MER11C transposable element or a derivative thereof.

In some embodiments, a synthetic delivery system, polynucleotide, or nucleic acid sequence (e.g., a recognition sequence, integration sequence, first nucleic acid sequence, or second nucleic acid sequence) comprises a transposable element that is a AluSx transposable element or a derivative thereof.

In some embodiments, a synthetic delivery system, polynucleotide, or nucleic acid sequence (e.g., a recognition sequence, integration sequence, first nucleic acid sequence, or second nucleic acid sequence) comprises a transposable element that is a MLT1J transposable element or a derivative thereof.

In some embodiments, a synthetic delivery system, polynucleotide, or nucleic acid sequence (e.g., a recognition sequence, integration sequence, first nucleic acid sequence, or second nucleic acid sequence) comprises a transposable element that is a AluSg2 transposable element or a derivative thereof.

In some embodiments, a synthetic delivery system, polynucleotide, or nucleic acid sequence (e.g., a recognition sequence, integration sequence, first nucleic acid sequence, or second nucleic acid sequence) comprises a transposable element that is a THE1A transposable element or a derivative thereof.

In some embodiments, a synthetic delivery system, polynucleotide, or nucleic acid sequence (e.g., a recognition sequence, integration sequence, first nucleic acid sequence, or second nucleic acid sequence) comprises a transposable element that is an AluJb transposable element or a derivative thereof.

In some embodiments, a synthetic delivery system, polynucleotide, or nucleic acid sequence (e.g., a recognition sequence, integration sequence, first nucleic acid sequence, or second nucleic acid sequence) comprises a transposable element that is a MTL2B4 or MLT2B4 transposable element or a derivative thereof.

In some embodiments, a synthetic delivery system, polynucleotide, or nucleic acid sequence (e.g., a recognition sequence, integration sequence, first nucleic acid sequence, or second nucleic acid sequence) comprises a transposable element that is a L2a transposable element or a derivative thereof.

In some embodiments, a synthetic delivery system, polynucleotide, or nucleic acid sequence (e.g., a recognition sequence, integration sequence, first nucleic acid sequence, or second nucleic acid sequence) comprises a transposable element that is a MLT1J2 transposable element or a derivative thereof.

In some embodiments, a synthetic delivery system, polynucleotide, or nucleic acid sequence (e.g., a recognition sequence, integration sequence, first nucleic acid sequence, or second nucleic acid sequence) comprises a transposable element that is an AluSq transposable element or a derivative thereof.

In some embodiments, a synthetic delivery system, polynucleotide, or nucleic acid sequence (e.g., a recognition sequence, integration sequence, first nucleic acid sequence, or second nucleic acid sequence) comprises a transposable element that is a L1MB3 transposable element or a derivative thereof.

In some embodiments, a synthetic delivery system, polynucleotide, or nucleic acid sequence (e.g., a recognition sequence, integration sequence, first nucleic acid sequence, or second nucleic acid sequence) comprises a transposable element that is a THE1C transposable element or a derivative thereof.

In some embodiments, a synthetic delivery system, polynucleotide, or nucleic acid sequence (e.g., a recognition sequence, integration sequence, first nucleic acid sequence, or second nucleic acid sequence) comprises a transposable element that is an AluY transposable element or a derivative thereof.

In some embodiments, upon contacting, arrival at, or internalization by the host cell, a transposable element disclosed herein co-opts the cellular retrotransposition machinery to integrate into the target (e.g., cancer) cell genome. In some embodiments, tissue-specific delivery, uptake, and/or integration can be determined by the retrotransposon's sequence and the host's expression of the retrotransposition machinery.

A synthetic delivery system, polynucleotide, or nucleic acid sequence can comprise an integration sequence that facilitates integration of the polynucleotide, synthetic delivery system, or cargo, into the genome of a target cell. The integration sequence can comprise, for example, a transposon integration signal. In some embodiments, a synthetic delivery system or polynucleotide lacks an integration sequence.

In some embodiments, the integration sequence facilitates preferential genomic integration of a ctDNA, synthetic delivery system, polynucleotide, or cargo by a target cell as compared to a control cell. The control cell can be, for example, a cell of a different cell type (e.g., a cell type disclosed herein). In some embodiments, the target cell is a cancer cell and the control cell is a non-cancer cell, e.g., of a same or substantially similar cell type. In some embodiments, the target cell is a cancer cell of a first cell type and the control cell is a cancer cell of a second cell type. In some embodiments, the target cell is a cancer cell of a first cell type and the control cell is a non-cancer cell of a second cell type.

An integration sequence can be or can comprise a functional fragment and/or variant of a sequence disclosed herein. For example, an integration sequence can comprise a subset of nucleotides from a nucleotide sequence disclosed herein that facilitates preferential integration into the genome of the target cell as compared to the control cell. In some embodiments, the integration sequence comprises a functional fragment and/or variant of a sequence disclosed herein that retains at least 10%, at least 20%, at least 30%, at least 40%, at least 50%, at least 60%, at least 70%, at least 80%, at least 90%, or at least 95% of the target cell genomic integration activity of the nucleotide sequence from which the fragment and/or variant is derived.

An integration sequence can comprise, consist essentially of, or consist of a subset of consecutive nucleotides from a sequence disclosed herein, for example, at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at most about 10, at most about 20, at most about 30, at most about 40, at most about 50, at most about 60, at most about 70, at most about 80, at most about 90, at most about 100, at most about 110, at most about 120, at most about 130, at most about 140, at most about 150, at most about 160, at most about 170, at most about 180, at most about 190, at most about 200, at most about 250, at most about 300, at most about 350, at most about 400, at most about 450, at most about 500, about 10, about 15, about 20, about 25, about 30, about 40, about 50, about 60, about 70, about 80, about 90, about 100, about 110, about 120, about 130, about 140, about 150, about 160, about 170, about 180, about 190, about 200, about 250, about 300, about 350, about 400, about 450, about 500, about 5-10, about 5-20, about 5-30, about 5-40, about 5-50, about 5-60, about 5-70, about 5-80, about 5-90, about 5-100, about 5-150, about 5-200, about 5-300, about 5-400, about 5-500, about 10-20, about 10-30, about 10-40, about 10-50, about 10-60, about 10-70, about 10-80, about 10-90, about 10-100, about 10-150, about 10-200, about 10-300, about 10-400, about 10-500, about 15-20, about 15-30, about 15-40, about 15-50, about 15-60, about 15-70, about 15-80, about 15-90, about 15-100, about 15-150, about 15-200, about 15-300, about 15-400, about 15-500, about 20-30, about 20-40, about 20-50, about 20-60, about 20-70, about 20-80, about 20-90, about 20-100, about 20-150, about 20-200, about 20-300, about 20-400, about 20-500, about 30-40, about 30-50, about 30-60, about 30-70, about 30-80, about 30-90, about 30-100, about 30-150, about 30-200, about 30-300, about 30-400, about 30-500, about 40-50, about 40-60, about 40-70, about 40-80, about 40-90, about 40-100, about 40-150, about 40-200, about 40-300, about 40-400, about 40-500, about 50-60, about 50-70, about 50-80, about 50-90, about 50-100, about 50-150, about 50-200, about 50-300, about 50-400, about 50-500, about 100-150, about 100-200, about 100-300, about 100-400, about 100-500, about 200-300, about 200-400, or about 200-500 consecutive nucleotides from any one of SEQ ID NOs: 1-78.

An integration sequence can comprise, consist essentially of, or consist of a nucleotide sequence with at least a minimum percentage of sequence identity to a subset of consecutive nucleotides from a sequence disclosed herein.

In some embodiments, an integration sequence comprises at least 70% sequence identity to at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 consecutive nucleotides from any one of SEQ ID NOs: 1-78.

In some embodiments, an integration sequence comprises at least 80% sequence identity to at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 consecutive nucleotides from any one of SEQ ID NOs: 1-78.

In some embodiments, an integration sequence comprises at least 85% sequence identity to at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 consecutive nucleotides from any one of SEQ ID NOs: 1-78.

In some embodiments, an integration sequence comprises at least 90% sequence identity to at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 consecutive nucleotides from any one of SEQ ID NOs: 1-78.

In some embodiments, an integration sequence comprises at least 95% sequence identity to at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 consecutive nucleotides from any one of SEQ ID NOs: 1-78.

In some embodiments, an integration sequence comprises at least 98% sequence identity to at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 consecutive nucleotides from any one of SEQ ID NOs: 1-78.

In some embodiments, an integration sequence is at least about 5, at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 nucleotides in length.

In some embodiments, an integration sequence is at most about 10, at most about 20, at most about 30, at most about 40, at most about 50, at most about 60, at most about 70, at most about 80, at most about 90, at most about 100, at most about 110, at most about 120, at most about 130, at most about 140, at most about 150, at most about 160, at most about 170, at most about 180, at most about 190, at most about 200, at most about 250, at most about 300, at most about 350, at most about 400, at most about 450, or at most about 500 nucleotides in length.

In some embodiments, an integration sequence is about 10, about 15, about 20, about 25, about 30, about 40, about 50, about 60, about 70, about 80, about 90, about 100, about 110, about 120, about 130, about 140, about 150, about 160, about 170, about 180, about 190, about 200, about 250, about 300, about 350, about 400, about 450, or about 500 nucleotides in length.

In some embodiments, an integration sequence is about 5-10, about 5-20, about 5-30, about 5-40, about 5-50, about 5-60, about 5-70, about 5-80, about 5-90, about 5-100, about 5-150, about 5-200, about 5-300, about 5-400, about 5-500, about 10-20, about 10-30, about 10-40, about 10-50, about 10-60, about 10-70, about 10-80, about 10-90, about 10-100, about 10-150, about 10-200, about 10-300, about 10-400, about 10-500, about 15-20, about 15-30, about 15-40, about 15-50, about 15-60, about 15-70, about 15-80, about 15-90, about 15-100, about 15-150, about 15-200, about 15-300, about 15-400, about 15-500, about 20-30, about 20-40, about 20-50, about 20-60, about 20-70, about 20-80, about 20-90, about 20-100, about 20-150, about 20-200, about 20-300, about 20-400, about 20-500, about 30-40, about 30-50, about 30-60, about 30-70, about 30-80, about 30-90, about 30-100, about 30-150, about 30-200, about 30-300, about 30-400, about 30-500, about 40-50, about 40-60, about 40-70, about 40-80, about 40-90, about 40-100, about 40-150, about 40-200, about 40-300, about 40-400, about 40-500, about 50-60, about 50-70, about 50-80, about 50-90, about 50-100, about 50-150, about 50-200, about 50-300, about 50-400, about 50-500, about 100-150, about 100-200, about 100-300, about 100-400, about 100-500, about 200-300, about 200-400, or about 200-500 nucleotides in length.

In some embodiments, the integration sequence of a synthetic delivery system or polynucleotide facilitates at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold higher genomic integration of the synthetic delivery system, polynucleotide, or cargo by target cells as compared to a control ctDNA, synthetic delivery system, or polynucleotide that lacks the integration sequence, or comprises a control integration sequence.

A synthetic delivery system, polynucleotide, or nucleic acid sequence can comprise a recognition sequence and an integration sequence. In some embodiments, the integration sequence and the recognition sequence are coupled, e.g., covalently coupled via phosphodiester bonds.

A synthetic delivery system, polynucleotide, or nucleic acid sequence can comprise a transposable element (e.g., transposon) sequence or a functional fragment thereof. A synthetic delivery system, polynucleotide, or nucleic acid sequence can comprise two or more transposable element (e.g., transposon) sequences or functional fragments thereof, e.g., from the same or different origin transposable elements. A transposable element (e.g., transposon) sequence or functional fragment thereof can be from or derived from, for example, a class I or class II transposon.

A polynucleotide can comprise a first nucleic acid sequence that was identified in, is from, or is derived from ctDNA. The ctDNA can originate from a cancer cell of the same, substantially the same, or a substantially similar cell type or tissue type as a target cell.

The first nucleic acid sequence can be in a 3′ region of the polynucleotide. The polynucleotide can further comprise a second nucleic acid sequence, for example, that was identified in, is from, or is derived from ctDNA. The ctDNA can be from or derived from the same, substantially the same, or a substantially similar cell type or tissue type as the target cell. The ctDNA can be the same ctDNA as that from which the first nucleic acid sequence is from or derived from. In some embodiments, the ctDNA is different than the ctDNA from which the first nucleic acid sequence is from or derived from. The second nucleic acid can be in a 5′ region of the polynucleotide. In some embodiments, the first nucleic acid sequence is a recognition sequence disclosed herein and the second nucleic acid sequence is an integration sequence disclosed herein. In some embodiments, the first nucleic acid sequence is an integration sequence disclosed herein and the second nucleic acid sequence is a recognition sequence disclosed herein. In some embodiments, the first nucleic acid sequence is first a recognition sequence disclosed herein and the second nucleic acid sequence is a second recognition sequence disclosed herein.

The first nucleic acid sequence can be or can comprise a nucleic acid sequence with at least 70%, at least 80%, at least 90%, at least 95%, or at least 99% sequence identity to any one or more of SEQ ID NOs: 1-78, or a functional fragment thereof disclosed herein (e.g., with at least a given number of consecutive nucleotides, with at least a given sequence identity to a given number of consecutive nucleotides, or with at least a given percentage of retained functional activity disclosed herein).

The second nucleic acid sequence can be or can comprise a nucleic acid sequence with at least 70%, at least 80%, at least 90%, at least 95%, or at least 99% sequence identity to any one or more of SEQ ID NOs: 1-78, or a functional fragment thereof disclosed herein (e.g., with at least a given number of consecutive nucleotides, with at least a given sequence identity to a given number of consecutive nucleotides, or with at least a given percentage of retained functional activity disclosed herein).

The first and/or the second nucleic acid sequence can comprise a functional fragment (for example, a recognition sequence or integration sequence) that is from or derived from any suitable sequence disclosed herein, for example, any one or more of SEQ ID NOs: 1-78.

The first nucleic acid sequence can be in a 5′ region of the polynucleotide. The polynucleotide can further comprise a second nucleic acid sequence, for example, that was identified in, is from, or is derived from ctDNA. The ctDNA can be from or derived from the same, substantially the same, or a substantially similar cell type or tissue type as the target cell. The ctDNA can be the same ctDNA as that from which the first nucleic acid sequence is from or derived from. In some embodiments, the ctDNA of origin for the second nucleic acid is different than the ctDNA from which the first nucleic acid sequence is from or derived from. The second nucleic acid can be in a 3′ region of the polynucleotide. The first and/or the second nucleic acid sequence can comprise a functional fragment (for example, a recognition sequence or integration sequence) that is from or derived from any suitable sequence disclosed herein.

In some embodiments, one portion (e.g., about half) of a nucleic acid sequence can be linked to the 5′ end of a nucleic acid cargo, gene, or sequence of interest, and the other portion (e.g., about half) of the sequence can be linked 3′ end of the nucleic acid cargo, gene, or sequence of interest. In some embodiments, a nucleic acid sequence can be linked directly or indirectly to a nucleic acid cargo, gene, or sequence of interest. For example, a first sequence can be linked directly to the 5′ end of a nucleic acid sequence comprising the nucleic acid cargo, gene, or sequence of interest, and a second sequence can be linked indirectly to the 3′ end of the nucleic acid sequence comprising the nucleic acid cargo, gene, or sequence of interest. In some embodiments, a composition of the present disclosure can comprise a guide sequence that is directly linked to a nucleic acid cargo, gene, or sequence of interest at one terminus (e.g., 3′ end) and directly linked to a recognition sequence or integration sequence at the other terminus (e.g., 5′ end), thereby indirectly connecting the integration or insertion sequence and the nucleic acid cargo, gene, or sequence of interest.

In some embodiments, a composition of the present disclosure comprises a first nucleic acid sequence (e.g., comprising a recognition and/or integration sequence) that is directly or indirectly linked to one end of a nucleic acid cargo, gene, or sequence of interest (e.g., 3′ end thereof), and a second nucleic acid sequence (e.g., comprising a recognition and/or integration sequence) that is directly or indirectly linked to the other end of the nucleic acid cargo, gene, or sequence of interest (e.g., a 5′ end thereof).

In some embodiments, a synthetic delivery system, polynucleotide, nucleic acid sequence (e.g., first nucleic acid sequence, second nucleic acid sequence, recognition sequence, or integration sequence) comprises a sequence disclosed herein, or a disclosed minimal degree of sequence identity to a sequence disclosed herein, or a fragment thereof. The fragment thereof can be a functional fragment, for example, that acts as a recognition sequence or integration sequence as disclosed herein.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 10 consecutive nucleotides of any one of SEQ ID NOs: 1-78.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 15 consecutive nucleotides of any one of SEQ ID NOs: 1-78.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 20 consecutive nucleotides of any one of SEQ ID NOs: 1-78.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 30 consecutive nucleotides of any one of SEQ ID NOs: 1-78.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 50 consecutive nucleotides of any one of SEQ ID NOs: 1-78.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 75 consecutive nucleotides of any one of SEQ ID NOs: 1-78.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 100 consecutive nucleotides of any one of SEQ ID NOs: 1-78.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 200 consecutive nucleotides of any one of SEQ ID NOs: 1-78.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 300 consecutive nucleotides of any one of SEQ ID NOs: 1-78.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to any one of SEQ ID NOs: 1-78.

In some embodiments, a synthetic delivery system, polynucleotide, nucleic acid sequence, first nucleic acid sequence, second nucleic acid sequence, recognition sequence, or integration sequence comprises, consists essentially of, or consists of the nucleotide sequence of any one of SEQ ID NOs: 1-78.

In some embodiments, a synthetic delivery system, polynucleotide, nucleic acid sequence, first nucleic acid sequence, second nucleic acid sequence, recognition sequence, or integration sequence comprises, consists essentially of, or consists of the nucleotide sequence of SEQ ID NO: 68.

In some embodiments, a synthetic delivery system, polynucleotide, nucleic acid sequence, first nucleic acid sequence, second nucleic acid sequence, recognition sequence, or integration sequence comprises, consists essentially of, or consists of the nucleotide sequence of SEQ ID NO: 73.

In some embodiments, a synthetic delivery system, polynucleotide, nucleic acid sequence, first nucleic acid sequence, second nucleic acid sequence, recognition sequence, or integration sequence comprises, consists essentially of, or consists of the nucleotide sequence of SEQ ID NO: 74.

In some embodiments, a synthetic delivery system, polynucleotide, nucleic acid sequence, first nucleic acid sequence, second nucleic acid sequence, recognition sequence, or integration sequence comprises, consists essentially of, or consists of the nucleotide sequence of SEQ ID NO: 75.

In some embodiments, a synthetic delivery system, polynucleotide, nucleic acid sequence, first nucleic acid sequence, second nucleic acid sequence, recognition sequence, or integration sequence comprises, consists essentially of, or consists of the nucleotide sequence of SEQ ID NO: 53.

In some embodiments, a synthetic delivery system, polynucleotide, nucleic acid sequence, first nucleic acid sequence, second nucleic acid sequence, recognition sequence, or integration sequence comprises, consists essentially of, or consists of the nucleotide sequence of SEQ ID NO: 13.

In some embodiments, a synthetic delivery system, polynucleotide, nucleic acid sequence, first nucleic acid sequence, second nucleic acid sequence, recognition sequence, or integration sequence comprises, consists essentially of, or consists of the nucleotide sequence of SEQ ID NO: 14.

In some embodiments, a synthetic delivery system, polynucleotide, nucleic acid sequence, first nucleic acid sequence, second nucleic acid sequence, recognition sequence, or integration sequence comprises, consists essentially of, or consists of the nucleotide sequence of SEQ ID NO: 33.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at least about 70%, at least about 80%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to SEQ ID NO: 53.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 50 consecutive nucleotides of SEQ ID NO: 53.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 75 consecutive nucleotides of SEQ ID NO: 53.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity to at least 100 consecutive nucleotides of SEQ ID NO: 53.

In some embodiments, a synthetic delivery system, polynucleotide, nucleic acid sequence, first nucleic acid sequence, second nucleic acid sequence, recognition sequence, or integration sequence comprises, consists essentially of, or consists of a nucleic acid sequence with at most about 70%, at most about 75%, at most about 80%, at most about 85%, at most about 90%, at most about 95%, at most about 97.5%, or at most about 99% sequence identity to any one of SEQ ID NOs: 1-78.

In some embodiments, a synthetic delivery system, polynucleotide, nucleic acid sequence, first nucleic acid sequence, second nucleic acid sequence, recognition sequence, or integration sequence comprises a nucleic acid sequence with one or more nucleotide insertions, deletions, or substitutions compared to any one of SEQ ID NOs: 1-78.

For example, the synthetic delivery system, polynucleotide, nucleic acid sequence, first nucleic acid sequence, second nucleic acid sequence, recognition sequence, or integration sequence can comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 nucleotide insertions relative to any one of SEQ ID NOs: 1-78.

In some embodiments, the synthetic delivery system, polynucleotide, nucleic acid sequence, first nucleic acid sequence, second nucleic acid sequence, recognition sequence, or integration sequence comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide insertions relative to any one of SEQ ID NOs: 1-78.

In some embodiments, the synthetic delivery system, polynucleotide, nucleic acid sequence, first nucleic acid sequence, second nucleic acid sequence, recognition sequence, or integration sequence comprises 1, 2, 3, 4, 5, 6, 7, 8, 9, 10, 11, 12, 13, 14, 15, 16, 17, 18, 19, 20, 25, 30, 35, 40, 45, or 50 nucleotide insertions relative to any one of SEQ ID NOs: 1-78.

The one or more insertions can be at the 5′ end, 3′ end, within the sequence, or a combination thereof. The one or more insertions can be contiguous, non-contiguous, or a combination thereof.

In some embodiments, the synthetic delivery system, polynucleotide, nucleic acid sequence, first nucleic acid sequence, second nucleic acid sequence, recognition sequence, or integration sequence comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 75, at least 100, at least 125, at least 150, at least 175, at least 200, at least 250, at least 300, at least 400, at least 500, at least 600, or at least 700 nucleotide deletions relative to any one of SEQ ID NOs: 1-78.

In some embodiments, the synthetic delivery system, polynucleotide, nucleic acid sequence, first nucleic acid sequence, second nucleic acid sequence, recognition sequence, or integration sequence comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or most 50, at most 75, at most 100, at most 125, at most 150, at most 175, at most 200, at most 250, at most 300, at most 400, at most 500, at most 600, or at most 700 nucleotide deletions relative to any one of SEQ ID NOs: 1-78.

In some embodiments, the synthetic delivery system, polynucleotide, nucleic acid sequence, first nucleic acid sequence, second nucleic acid sequence, recognition sequence, or integration sequence comprises 1, 2, 3, 4, 5, 6, 7, 8, 9, 10, 11, 12, 13, 14, 15, 16, 17, 18, 19, 20, 25, 30, 35, 40, 45, or 50 nucleotide deletions relative to any one of SEQ ID NOs: 1-78.

The one or more deletions can be at the 5′ end, 3′ end, within the sequence, or a combination thereof. The one or more deletions can be contiguous, non-contiguous, or a combination thereof.

In some embodiments, the synthetic delivery system, polynucleotide, nucleic acid sequence, first nucleic acid sequence, second nucleic acid sequence, recognition sequence, or integration sequence comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 nucleotide substitutions relative to any one of SEQ ID NOs: 1-78.

In some embodiments, the synthetic delivery system, polynucleotide, nucleic acid sequence, first nucleic acid sequence, second nucleic acid sequence, recognition sequence, or integration sequence comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 nucleotide substitutions relative to any one of SEQ ID NOs: 1-78.

In some embodiments, the synthetic delivery system, polynucleotide, nucleic acid sequence, first nucleic acid sequence, second nucleic acid sequence, recognition sequence, or integration sequence comprises 1, 2, 3, 4, 5, 6, 7, 8, 9, 10, 11, 12, 13, 14, 15, 16, 17, 18, 19, 20, 25, 30, 35, 40, 45, or 50 nucleotide substitutions relative to any one of SEQ ID NOs: 1-78.

The one or more substitutions can be at the 5′ end, 3′ end, within the sequence, or a combination thereof. The one or more substitutions can be contiguous, non-contiguous, or a combination thereof.

A polynucleotide or a nucleic acid sequence disclosed herein (e.g., recognition sequence, integration sequence, first nucleic acid sequence, or second nucleic acid sequence) can be from about 10 base pairs (bp) to about 1000 bp in length. A polynucleotide or a nucleic acid sequence disclosed herein can be from about 100 base pairs (bp) to about 900 bp in length. A polynucleotide or a nucleic acid sequence disclosed herein can be from about 200 bp to about 800 bp in length. A polynucleotide or a nucleic acid sequence disclosed herein can be from about 300 bp to about 700 bp in length. A polynucleotide or a nucleic acid sequence disclosed herein can be from about 400 bp to about 600 bp in length. A polynucleotide or a nucleic acid sequence disclosed herein can be at least about 50 bp in length. A polynucleotide or a nucleic acid sequence disclosed herein can be at least about 100 bp in length. A polynucleotide or a nucleic acid sequence disclosed herein can be at least about 200 bp in length. A polynucleotide or a nucleic acid sequence disclosed herein can be at least about 300 bp in length. A polynucleotide or a nucleic acid sequence disclosed herein can be at least about 400 bp in length. A polynucleotide or a nucleic acid sequence disclosed herein can be at least about 500 bp in length. A polynucleotide or a nucleic acid sequence disclosed herein can be at least about 1000 bp in length.

A polynucleotide or a nucleic acid sequence disclosed herein can be at most about 50 bp in length. A polynucleotide or a nucleic acid sequence disclosed herein can be at most about 100 bp in length. A polynucleotide or a nucleic acid sequence disclosed herein can be at most about 200 bp in length. A polynucleotide or a nucleic acid sequence disclosed herein can be at most about 300 bp in length. A polynucleotide or a nucleic acid sequence disclosed herein can be at most about 400 bp in length. A polynucleotide or a nucleic acid sequence disclosed herein can be at most about 500 bp in length. A polynucleotide or a nucleic acid sequence disclosed herein can be at most about 1000 bp in length. A polynucleotide or a nucleic acid sequence disclosed herein can be at most about 5000 bp in length. A polynucleotide or a nucleic acid sequence disclosed herein can be at most about 10000 bp in length.

In some embodiments, the polynucleotide or a nucleic acid sequence disclosed herein (e.g., recognition sequence, integration sequence, first nucleic acid sequence, or second nucleic acid sequence) is at least 10 base pairs in length. In some embodiments, the polynucleotide or a nucleic acid sequence disclosed herein is at least 20 base pairs in length. In some embodiments, the polynucleotide or a nucleic acid sequence disclosed herein is at least 50 base pairs in length. In some embodiments, the polynucleotide or a nucleic acid sequence disclosed herein is at least 100 base pairs in length. In some embodiments, the polynucleotide or a nucleic acid sequence disclosed herein is at least 200 base pairs in length. In some cases, the polynucleotide or a nucleic acid sequence disclosed herein can be at least 400 base pairs in length.

In some embodiments, the polynucleotide or a nucleic acid sequence disclosed herein (e.g., recognition sequence, integration sequence, first nucleic acid sequence, or second nucleic acid sequence) is from about 10 base pairs to about 100 base pairs in length. In some embodiments, the polynucleotide or a nucleic acid sequence disclosed herein can be from about 10 base pairs to about 200 base pairs in length. In some embodiments, the polynucleotide or a nucleic acid sequence disclosed herein can be from about 20 base pairs to about 100 base pairs in length. In some embodiments, the polynucleotide or a nucleic acid sequence disclosed herein can be from about 100 base pairs to about 200 base pairs in length. In some embodiments, the polynucleotide or a nucleic acid sequence disclosed herein can be from about 100 base pairs to about 500 base pairs in length. In some embodiments, the polynucleotide or a nucleic acid sequence disclosed herein can be from about 200 base pairs to about 800 base pairs in length. In some embodiments, the polynucleotide or a nucleic acid sequence disclosed herein can be from about 400 base pairs to about 2,000 base pairs in length. In some embodiments, the polynucleotide or a nucleic acid sequence disclosed herein can be from about 400 base pairs to about 5,000 base pairs in length. In some embodiments, the polynucleotide or a nucleic acid sequence disclosed herein can be from about 1,500 base pairs to about 7,200 base pairs in length. In some embodiments, the polynucleotide or a nucleic acid sequence disclosed herein can be from about 1,900 base pairs to about 5,800 base pairs in length. In some embodiments, the polynucleotide or a nucleic acid sequence disclosed herein can be from about 2,000 base pairs to about 10,000 base pairs in length. In some embodiments, the polynucleotide or a nucleic acid sequence disclosed herein can be from about 5,000 base pairs to about 15,000 base pairs in length. In some embodiments, the polynucleotide or a nucleic acid sequence disclosed herein can be from about 600 base pairs to about 16,900 base pairs in length. In some embodiments, the polynucleotide or a nucleic acid sequence disclosed herein can be from about 8,000 base pairs to about 18,000 base pairs in length. In some embodiments, the polynucleotide or a nucleic acid sequence disclosed herein can be from about 10,000 base pairs to about 20,000 base pairs in length. In some embodiments, the polynucleotide or a nucleic acid sequence disclosed herein can be from about 400 base pairs to about 20,000 base pairs in length.

In some embodiments, a synthetic delivery system, polynucleotide, nucleic acid sequence, first nucleic acid sequence, second nucleic acid sequence, recognition sequence, or integration sequence comprises from about 1 to about 20 insertions, deletions, and/or substitutions relative to any one of SEQ ID NOs: 1-78. In some embodiments, the polynucleotide or a nucleic acid sequence comprises from about 20 to about 100 insertions, deletions, and/or substitutions relative to any one of SEQ ID NOs: 1-78. In some embodiments, the polynucleotide or a nucleic acid sequence comprises from about 50 to about 250 insertions, deletions, and/or substitutions relative to any one of SEQ ID NOs: 1-78. In some embodiments, the polynucleotide or a nucleic acid sequence comprises from about 150 to about 500 insertions, deletions, and/or substitutions relative to any one of SEQ ID NOs: 1-78.

In some embodiments, a synthetic delivery system of the disclosure can be functional without requiring a commonly used delivery vector or delivery system, e.g., as “naked” DNA. For example, in some embodiments a synthetic delivery system does not require or utilize a viral vector, a nanoparticle, a lipid nanoparticle, a liposome, an exosome, a dendrimer, a gene gun, or electroporation.

In some embodiments, targeted cargo delivery via synthetic delivery systems disclosed herein can significantly reduce off-target and unwanted adverse effects associated with alternate delivery systems, for example, non-targeted delivery systems.

In some embodiments, a synthetic delivery system of the disclosure can be combined with or utilize a vector, such as a viral vector, a nanoparticle, a lipid nanoparticle, a liposome, an exosome, or a dendrimer.

In some embodiments, a candidate polynucleotide for use in a synthetic delivery system comprises a full length transposable element (e.g., retrotransposon). In some embodiments, candidate polynucleotide for use in a synthetic delivery system comprises a high rate of identified mutations, or more than one identified mutation (e.g., SNV), for example, favoring higher numbers of mutations relative to a reference sequence. The mutation can be shared between ctDNA-target cell coculture conditions and ctDNA alone, and not present or substantially not present in the genome of the untreated control cells. In some embodiments, a candidate polynucleotide for use in a synthetic delivery system is not located in an intron of a gene, or if present in an intron, is not close to an exon (e.g., is at least 3 kb, 5 kb, or 10 kb from an exon).

TABLE 1 provides sequences of illustrative polynucleotides that can be used in a synthetic delivery system disclosed herein, for example, for delivery to cancer cells, e.g., hematologic cancer cells, such as multiple myeloma cells, B cells, and/or plasma cells.

TABLE 1

| SEQ | | |
|---|---|---|
| ID NO | Description | Sequence |
| 1 | ALUSx | TTTTTCTTCTCTCTTTTTTTTTTATTTTTTGAGATGGAGTT |
| | (ASLUSq2) | TCACTCTGTTGCCCAGGCTGGAGTGCAATGGTGCAGTCT |
| | | CAGCTCACTGCAACCTCCACCTCCCGGGTTTGAGCAGTG |
| | | ATTCTGCCTCAGCCTCCCAAGTAGCTGGGATTACAGGCA |
| | | CCCACCACCATGCCTGGCTAATTTTTGTATTTTTAGTAGA |
| | | GATGGGGTTTCACCATGTTGGCCAGGCTGGTCTCGAACT |
| | | CCTGACCTCAGGTGATCCACCTGCCTCGGCCTCCCAAAG |
| | | TGCTGAAATTACAGGCATGAGCCACCGCACCTGGCC |
| 2 | L1MB3 | TTTTTGTATTTTTAGTAGAGATGGGGTTTCACCATGTTGG |
| | | CCAGGCTGGTCGAACTCCTGACCTCAGGTGATCCACCtG |
| | | CCTCGGCCTCCCAAAGTGCTGAaATTACAGGcaTGAGCCA |
| | | CCCCCAGAACTCTTCTCTTTCCCAAAACTAAAATTCTGC |
| | | ACCCATTGAACAGTAACTCCCCATTTCTCCTTTCCCCTAA |
| | | CTCCTGGCATCCACCATTCTGTTTTCTGTCTTTACGAATT |
| | | TTACTACCCTTGGTACTTCCTTTAAGTGGAATCATACAAT |
| | | ATTTTTCTTTTTCTGACTGGCTTATTTCACTAAGCATAAT |
| | | ATTTTTAAGGTTCAAGCATTTTGTAGCATATGTCAGAATT |
| | | TCCTTTCTTTTTAAAGCTCAATAATATTCCATTGTATGtAT |
| | | ATACCACATTTTACTTAACCATTCATCCgTCAGTGGACAC |
| | | TTGGGTTACTTCCAAcTTTTAGCTATTGTGAATAATGCTG |
| | | CTATGAACATGAATGTACAAATATCTnTTCGAGnCCCTGC |
| | | TTTCAATTCTTTTGGGTATATACCCAGAAGTGGAATTGCT |
| | | GGATCATATGGTAATTCTATTTTTAATTTTTTGAGGAACC |
| | | GCCATACTGTTTTCCACAGCGGCTGCACCATTTTACATTC |
| | | CCACCAGCAGTGCACAAGGGTTCCAATTTCTCCACATCC |
| | | TCGCCAACACTTGTTATTTTCTGTTTTTTTGATAGTAGCC |
| | | ATCCTAATGGGTGTGAGGTGGTATCTCATTGTGGTTTTG |
| | | ATTTGCATTTCCCTAATGATTAGTGATGTTGAGCATCTTT |
| | | TCATGTGCTTATTGGCCATTTGTATATCTTCTTTGGAGAA |
| | | ATGTCTATTCAAGTCCTTTGCCCATTTTTnAATCGGGTTG |
| | | TTTGTTTTTTTGTTGTTGAGTTGTAGGAGTTCTTTATATAT |
| | | TCTGGATATTAA |
| 3 | L2a | TCTCACCACCTCTACTACTGCCTATCTGCATCTGGGTGCA |
| | (2L2a) | CGTCCTCTGCTATTCCTTCTGTTTCTACAGAAGCTGTCCA |
| | | TATTTCTAAGGCTAATCCCTTGCATTTTGGTACCAGACTT |
| | | CCCTGACCTACACAAAGATGATCACCCCCCAAAATTGCC |
| | | TTTCTTTCCTGCATCAAGTTTTTTCTTCTCTACTGTATTAT |
| | | TCCTATTGCACATTAGCCCAAAAAATGCCGTTGTTTTTCC |
| | | ATCGGTAAAAAAAAAAAATAGAGAGAAAAAGAAAAAT |
| | | AATCCATTGCCGCCAATCTATCTTCTAGTCATTTTTCCAT |
| | | TTCTCTGTTCTCCATTCTGCATAAACCATTTTTACTTGAA |
| | | TTGTTTATACTTGCTGTTTACAACAAACCTCCTCTTTCTC |
| | | TCCACCCCAATCAGATTTTGGTCCACATTCCTCATGCAGT |
| | | GGTCAGTCCTTGGTCCTCAACCTAGTCTAGCCTGCTTGCT |
| | | GGTGGGTGTGCTCTCGGGGCATTTGCTTGTGTTTGCACG |
| | | ACAGCTCAGTCCCTCACTTCTCTCAAGTCTTTGCTAATAA |
| | | CACTCTTGCATCCAAGTCTTCCCTGACCTGCTACTTAAAA |
| | | ACAACTCACCCCCACCCCTAGAAATCCCTATCCTCTTCC |
| | | CTACGCTTTATTTTTCTCCATAATACTTATCACCATGTAG |
| | | CTGAACATGTATTTTACTCACTTTTTTTTATATTGTCCAT |
| | | CTTCCCTCAGTGAATTGAAAGCTCCATGAGGGCAGGAGG |
| | | TTTTGTTTATTGTGTTTACTGCTTTATCCCTAGCACCCAG |
| | | CACAGCATATGTGATATAGTAGACACTCAATACAAATCT |
| | | GATGTGTGTGTGTCT |
| 4 | THE1C | TGATATGGTTTGGCTGTGTCCCCACCTAAATCTCATCTTG |
| | (ERVL-MaLR) | AATTGTAGTTCCCATAATCCCCATGTGTCAtGGGAGGGA |
| | | CCCAGTGGGAGGTAATTTAATCCTGGGGGCAGTTACCCT |
| | | CCTGCTATGTGTGTGTGTGTGTGTGTGTTCTCACGAGATC |
| | | TGATGGTTTTATAAGGGGCTTTTCCCCCTTCGCTCGGCAC |
| | | TTCTCCTTCCTGCCGCCATGTGAAGAAGGACGTGTTTGC |
| | | TTCCCCTTCCGCCATGATTGTAAGTTTCCTGAGGCCTCCC |
| | | CAGCCATGCnGAACTGTGAGTCAATTAAACCTCTTTCCTT |
| | | TATAAATTACCCAGTCTCGGGTGTGTCTTTATTAGCAGC |
| | | GTGAGAACGGACTAATACA |
| 5 | ALUY (ALU) | TTTTGCTCTTCTTTTTTTTTTTTTTTTTGAGAGACGGAGTC |
| | | TCGCTCTGTCTCCCAGGCTGGAGTGCAGTGGCGCGATCT |
| | | CGGCTCACTGCAAGCTCCGCCTCCCGGGTTCACGCCATT |
| | | CTCCTGCCTCAGCCTCCCGAGTAGCTGGGACTACAGGCG |
| | | CCCGCCACCACGCCCGGCTAATTTTTTGTATTTTTAGTAG |
| | | AGACGGGGTTTCACCGTGTTAGCCAGGATGGTCTCGATC |
| | | TCCTGACCTCGTGATCCGCCCGCCTCGGCCTCCCAAAGT |
| | | GCTGGGATTACAGGCGTGAGCCACCGCGCCCGGCC |
| 6 | L1ME4b | TATTTACATTATTTTAATAGGTAATGCAATTACATCTTCT |
| | | AAAATTCAGAAAGCATAAAAGTACATAAGAGAAAAACT |
| | | CATCATCCAGCCCTTGCCCTTGAGGACGCTAGTTTCTAC |
| | | CCACACAGCTAAACCTTGTTACAAATTACTTGGGTGTAT |
| | | TACCAGTGGCATTGCCAGGTTTAACAAATAAAAACATAG |
| | | GATTCCTGATTAAGTTTAATTTTCAGATAAATAATTACAT |
| | | GCTTTTTAAGTAGAAGTATTTCCCAAATATTACAAGAGA |
| | | CAATATATTTAAAAAATTGTTTGATATCCAAATTTAACT |
| | | ATGCGTCCTATATTTTGTCTGGCAACCCTAATCCTTCATT |
| | | GTATGGATGTACCCCAGGTTCTTATGCAAATAGAGAAAA |
| | | TATGCACTCTTTTATTATCCTCCGTCCCTTTCTTTTACAG |
| | | ACAATGATAGAATGCTTTATTTAGTACATACGTCATTCT |
| | | GCACCTGTGCGAGTATTTTAGTTAACCACACTTTTGGGG |
| | | ATTGTTCTGCTATCATAAAAGATATTTCTCTTTTTTACCA |
| | | TAATGTATTTCATTAAACCCTCTTTCACAGGTTAGGTCCC |
| | | ATATTTTTCTTTTTTTTTTTTTTTTTGAGAGACGGAGTCTC |
| | | GCTCTGTCTCCCAGGCTGGAGTGCAGTGGCGCGATCTCG |
| | | GCTCACTGCAAGCCTGATAGGTGAAAAATGGTATCTCAG |
| | | TGTAGTTTTAATTTGCATTTCTCTTATTATGAGTGAGGTT |
| | | GAGCATCTTTTCATATGTTTAAGAGCCATTTGTATTTCCT |
| | | TTTCTGTGAACTGTCTGTCCGCCTCCCGGGTTCACGCCAT |
| | | TCTTGGGTTGTTGGTCTTTTTCTTATTGACCTGCCTCAGC |
| | | CTCCCGAGTAGCTGGGAAATCTA |
| 7 | ALUSx | GGTCAGTCGCGGTGGCTCATACTTGTAATCTCAGCACTT |
| | (ALUSg2) | TGGGAGGCCAAGGCGGGTGGATCACCTCAGGTCAGGAG |
| | | TTCAAGACCAGCCTGACCAATGTGGTAAAACCCTGTCTC |
| | | TACTAAAAATACAAAAATTAGCTGGGCGTGGTGGCACA |
| | | TGCCTGTAATCCCAGCTACTCAGGAGGCTGAGGCAGGA |
| | | GAATCGCTTGAACCCGGGAGGCAGAGGTTGCAGTGAGC |
| | | CAAGATAACACCACTGCACTCCAGCCTGGGTGACAGAG |
| | | CAAGACTCCGTCTCAAAAAAAAAAAAAAAAGATCCAGT |
| | | GATAGG |
| 8 | MIR | TATCTATTCACATTTATATGCTCTTAGTCAGAAAATAAA |
| | | ATACCTCTCTTGAAATGGGCCTGACTCCGGAGAGGGAGA |
| | | CCAGATTCACTGCAAGCTCCGCCTCCCGGGTTCCCGCCA |
| | | TTCTCCTGCCTCAGCCTCCCCAGTAGCTCTGACTACAGG |
| | | CGCCCGCCACCACGCCCGGCTAATTTTTTTATTTTTTAGT |
| | | AGAGACGGGGTTTCACCGTGTTAGCCAGGATGGTCTCGA |
| | | TCTCCCAACCTCGTGATCCGCCCGCCTCGGCCTCCCAAA |
| | | GTGCTGGGATTACAGGCATGAGCCACCGCGCCCGGCC |
| 9 | AluY | TTTTTTTTTCTTTTTTTTTTTTTTTCTTTTGAGACGGAGTCT |
| | (ALU) | TGCTCTGTCGCCCAGGCTGGAGTGCAGTGGCGCGATCTC |
| | | GGCTCACTGCAAGCTCCGCCTCCCAGGTTCATGCAATTC |
| | | TCCTGCCTCAGCCTCCCGAGTAGCTGGGACTACAGGCGC |
| | | CCGCCACCACGCCCGGCTAATTTTTTGTATTTTTAGTAGA |
| | | GACGGGGTTTCACCGTGTTAGCCAGGATGGTCTCGATCT |
| | | CCTGACCTCGTGATCCGCCCGCCTCGGCCTCCCAAAGTG |
| | | CTGGGATTACAGGCGTGAGCCACCGCGCCCGGCC |
| 10 | ALluY | TGCTGGGCATGGTGGCTCACGCCTGTAATCCCAGCACTT |
| | | TGGGAGGCCAAGGCGGGCAGATCACGAGGTCAGGAGTT |
| | | CGAGACCATCCTGGCTAACACAGTGAAACCCCGTCTCTA |
| | | CTAAAAATACAAAAAATTAGCCGGGCGTGGTGGCGGGT |
| | | GCCTGTAGTCCCAGCTACTCGGGAGGCTGAGGCAGGAG |
| | | AATGGCGTGAACCCAGGAGGCGGAGCTTGCAGTGAGCT |
| | | GAGATTGCGCCACTGCACTCCAGCCTGGGTGACAGAGC |
| | | GAGACTCCTCTTCAAAAAAAAAAAAAAAAAAAAAAAGA |
| | | CTCAT |
| 11 | AluJb | GGCCGGGCGCGGTGGCTCACGCCTGTAATCCCAGCACTT |
| | (ALU) | TGGGAGGCCGAGGCGGGnGGATCGCTTGAGCCCAGGAG |
| | | TTCGAGACCAGCCTGGGCAACATGGCGAAACCCCGTCTC |
| | | TACAAAAAATACAAAAATTAGCCGGGCGTGGTGGCGCG |
| | | CGCCTGTAGTCCCAGCTACTCGGGAGGCTGAGGCGGGA |
| | | GGATCGCTTGAGCCCGGGAGGTCGAGGCTTCAGTGAGCT |
| | | GAGATCAcgCCACTGCACTCCAGCCTGGGTGACAGAGcgA |
| | | GACTCtGTCTCAAAAAAAATAAATAAAAAATAAAAATAA |
| | | AA |
| 12 | AluSx | GGCCGGGCGCGGTGGCTCACGCCTGTAATCCCAGCACTT |
| | (AluSg) (ALU) | TGGGAGGCCGAGGCGGGCGGATCACCTGAGGTCAGGAG |
| | | TTCGAGACCAGCCTGGCCAACATGGTGAAACCCCGTCTC |
| | | TACTAAAAATACAAAAATTAGCCGGGCGTGGTGGCGCG |
| | | CGCCTGTAATCCCAGCTACTCGGGAGGCTGAGGCAGGA |
| | | GAATCGCTTGAACCCGGGAGGCGGAGGTTTCAGTGAGC |
| | | TGAGATCACGCCACTGCACTCCAGCCTGGGTGACAGAGC |
| | | GAGACTCTGTCTCAAAAATAAATAAATAAATAAAAAAT |
| | | AAAAA |
| 13 | MER11C | TATCGAAAATTCACCCCCGATATTCACGTAGGTTCTTTTC |
| | (LTR) | TATTTTCCCTAAGCGTCGGCAGGTTTGAGAAATAAAGGG |
| | (ERVK/LTR) | ACAGAGTACAAAAGAGAGAAATTTTAAAGCTGGGCGTC |
| | | CGGGGGAGACATCACATGTTGGTAGGTTCCATGATGCCC |
| | | CACAAGCCGTAAAAACCAGCAAGTTTTTATTAGTGATTT |
| | | TCAAAAGGGGAGGGAGTGTACGAATAGGTGTGGGTCAC |
| | | AGAGATCACGTGCTTCACAAGGTAATAGAATATCACAA |
| | | GGCAAATGGAGGCAGGGCGAGATCACAGGACCACAGGA |
| | | CCGGGGTGAAATTAAAATTGCTAATGAAGTTTTGGGCAC |
| | | CATTGTCATTGATAACATCTTATCAGGAGACAGGGTTTG |
| | | AGAGACAACCGGTCTGACCAAAATTTATTAGGTGGGAA |
| | | TTTCCTCGTCCTAATAAGCCTGGGAGTGCTATGGGAGAC |
| | | TGGGGCTTATTTCATCCCGTAGAGGCAACCATAAAAGAC |
| | | AGGCCCAAGCGGCCATTTAGAGCCTCCCAGGACATTCTC |
| | | TTTCTCAGGGTGTTCCTTGCTGAGAAAAAGAATTCAGAT |
| | | ATTTCTCCATTTGCTTTTGAAAGAAGAGAAATATGGCTC |
| | | TGTTCCCGGCTCACGGCGCAGATTTAAGGTTATCTCCTTT |
| | | TCCCTGAACAATTGCTGTTATCCTGTTCTTTTTTCAAGGT |
| | | GCCCAGATTTCATATTGTTCAAACACACATGCTTACAAT |
| | | TTGTGCAGTTAAGCAATATCACAGGGTCCTGAGGGACAT |
| | | ACATCCTCCTCAGCTGACAGGATTAAGAGATTAAAGTAA |
| | | AGACAGGCATAGGAAATCACAAGGGTATTGATTGGGGA |
| | | AGTGATAAGTGTCCATGAAATCTTCACAATTTATGTTTA |
| | | GAGATTGCAGTAAAGACAGGCATAAGAAATTATAAAAG |
| | | TATTAATTTGGGGAACTAATAAATGTCCATGAAATCTTC |
| | | ACAATCCACGTTCTTCTGCCATGGCTTCAGCCGGTCCCTC |
| | | CGTTCGGGGTCCCTGACTTCCCGCAACA |
| 14 | THE1A | TGATATGGCTTGGCTGTGTCCCCACCCAAATCTCATCTTG |
| | (LTR) | AATTGTACGTTCCATAATTCCCATGTGTTGTGGGAGGGA |
| | | CCCAGTGGGAGATAATTGAATCATGGGGAGCAGTTTCCC |
| | | CCATACTGTTCTCATGGTAGTGAATAAGTCTCATGAGAT |
| | | CTGATGGTTTTATCAGGGGTTTCTACTTTTGCATCTTCCT |
| | | CATTTTCCTCTTGCCACcACCATGTAAGAAGTGCCTTTCA |
| | | CCTCCCACCATGATTCTGAGGCCTCCCCAGCCATGTGGA |
| | | ACTGTAAGTCCAATTAAACTTCTTTTTCTTCCCAGTCTTG |
| | | GGTATATCTTTATCAGCAGCATGAAAATGGACTAATACA |
| 15 | AluY | TTTTTTTTTTTTTTTTTTTTTTTTTTTTTTGAGACGGAGTCT |
| | | CGCTCTGTCGCCCAGGCTGGAGTGCAGTGGCGCGATCTC |
| | | GGCTCACTGCAAGCTCCGCCTCCCGGGTTCACGCCATTC |
| | | TCCTGCCTCAGCCTCCCGAGTAGCTGGGACTACAGGCGC |
| | | CCGCCACCACGCCCGGCTAATTTTTTGTATTTTTAGTAGA |
| | | GACGGGGTTTCACCGTGTTAGCCAGGATGGTCTCGATCT |
| | | CCTGACCTCGTGATCCGCCCACCTCGGCCTCCCAAAGTG |
| | | CTGGGATTACAGGCGTGAGCCACCGCACCCAGCC |
| 16 | AluSx | TTTCTTTACTTCAACCTTCTTTTTTTTTTTGAGACAGAGTC |
| | | TGCTCTCGTTGCCCAGGCTGGAGTGCAATGGCGCAACCT |
| | | CAGCTCACTGCAACCTCTGCCTCCTGGGTTCAAGCAATT |
| | | CTCCTGCCTCAGCCTCCCAAGTAGCTGGGATTACAGGCA |
| | | TGCGCCACCACGCCCAGCTAATTTTGTATTTTTAGTAGA |
| | | GACGGGGTTTCACCATGTTGGCCAGGCTGGTCTTGAACT |
| | | CCTGACCTCAGGTGATCCACCCGCCTCAGCCTCTCAAAG |
| | | TGCTGGGATTACAGGCGTCAGCCACCGCACTCAGCC |
| 17 | AluY | GGCCGGGCGCGGTGGCTCACGCCTGTAATCCCAGCACTT |
| | (FLAM_C) | TGGGAGGCCGAGGCGGGCGGATCACGAGGTCAGGAGAT |
| | (ALU) | CGAGACCATCCTGGCTAACACGGTGAAACCCCGTCTCTA |
| | | CTAAAAATACAAAAAATTAGCCGGGCATGGTGGCAGGC |
| | | ACCTGTAGTCCCAGCTACTCAGGAGGCTGAGGCAGGAG |
| | | AATGGCGTGAACCCAGGAGGCGGAGCTTGCAGTGAGCC |
| | | GAGATCATGCCACTGCACTCCAGCCTGGGTGACAGAGTG |
| | | AGACTCCATCTCAAAAAAATGACACAAAATAAAATAAA |
| | | ATAA |
| 18 | AluSx | GGCCGGGCGTGGTGGCTCATGCCTGTAATCCTAGCACTT |
| | (AluSx1) | TGGGAGGCCGAGGCTAGCGGATCACCTGAGGTCAGGAG |
| | | TTTGAGACCAGCCTAGCCAACATGGCGAAACCCTGTCTC |
| | | TACTAAAAATACAAAAAATGGCTGGGCATGGTGGCAGA |
| | | CATCTGTAATCCCAGCTACTCAGGAGGTTGAGACAGGAG |
| | | AATTGCTTGAACTCAGGAAGCGGAGGTTGCAGTGAGCT |
| | | GAGATTGCGCCACTGCACTCCAGTCTGGGTGACAGAGCA |
| | | AGACTCTGTCTCAAAAAAATAAAATAAAATAAAAATAA |
| | | AATA |
| 19 | AluY | GGCCGGGCGTGGTGGCTCATGCCTGTAATCCTAGCACTT |
| | (AluSx1) | TGGGAGGCCGAGGCTAGCGGATCACGAGGTCAGGAGTT |
| | | TGAGACCAGCCTAGCCAACATGGCGAAACCCTGTCTCTA |
| | | CTAAAAATACCAAAAAATGGCTGGGCATGGTGGCAGAC |
| | | ATCTGTAATCCCAGCTACTCAGGAGGTTGAGACAGGAG |
| | | AATTGCTTGAACTCAGGAAGCGGAGGTTGCAGTGAGCT |
| | | GAGATTGCGCCACTGCACTCCAGTCTGGGTGACAGAGCA |
| | | AGACTCTGTCTCAAAAAAATAAAATAAAATAAAAATAA |
| | | AATA |
| 20 | ALR/Alpha | TTCTTCTCACGGAGTGAAACTTTTCCTTTGATTGAGCAGT |
| | (Satellite/centr) | TTCGAAACTCTCTTTTTGTAAAATCTGCAAGTGAACATTT |
| | | GGAGTGCTTTGTGGCCTATGATAGAAAAGGAAATATTTT |
| | | CACTTAAAATCTAGACAGAAGCAATCTGAGAAACTTCTT |
| | | TGTGATGTTTGTATTCATCTTACATAGTTAAACCTTTCTT |
| | | TTGATTGAGCAGTTTTGAAACTCTCTTTTTGTCGAATCTG |
| | | TAAGAGGACATTTGGAGTGCTTTCAGGCCTATGGTGAAA |
| | | AAGGAAGTATCTTCTCATGAAAACTAGACAGAAGAATT |
| | | CAGACAAACTTCTTTGTGATGTGTGCATTTATCTCACAG |
| | | ACTTGAACCTTTCTTTTGATTGAGCAGTTTGAAACACTCT |
| | | TTTTGTAGAATCTGCAAGTGGACATTTGGAGCACGTGGA |
| | | GGCCTATAGTGGAAATGGAAATGTCTTCACACTAAAAAC |
| | | TAGACAGAAGAATTCTGAGAAACTTCTTTGTGATCTTTG |
| | | CATTCATCTCACAGAGTTGAACCTTTCTTTTCATTGAGCA |
| | | GTTTGAAAACACTCTTTTTGTAGAATCGGCAAGTGGACA |
| | | TTTGGAACGCATTGCAGACTATGATGGAAAAGGAAATA |
| | | TCTCCA |
| 21 | Alu | CAGCGCAAGTAATATATCCTGGAATTGGAAGCAGAATG |
| | (SINE/Alu) | GGAACTATGGACTCAGTGAAGATTGTTCTGGACATTATT |
| | | TGATTCTTCTTTCTCGCTGCCAATTGGATACTTTTTTTTTT |
| | | TTTTTGAGACAGAGTCT |
| 22 | Alu | CAGCGCAAGTAATATATCCTGGAATTGGAAGCAGAATG |
| | (SINE/Alu) | GGAACTATGGACTCAGTGAAGATTGTTCTGGACATTATT |
| | | TGATTCTTCTTTCTCGCTGCCAATTGGATACTTTTTTTTTT |
| | | TTTTGAGACAGAGTCT |
| 23 | ALR/Alpha | TTCATCTTACAGAGATAAACATTTATCTTGATTCAGCAG |
| | (Satellite/centr) | TTTGGAAACACTGTTTTTATCCATTCTGTGAATGGACATT |
| | | TGGGAGTTCATTAAGGCACACATGAAAAGGTGTATATCC |
| | | CAGGGTAAAAACTAGAAGGAAGCTATCTGAGAAACCAC |
| | | TCTGTGGTGTGTGCATTGATCTCGCTGATTTAAAGCTTTC |
| | | TTTTGATTCAGCAGTTTGGAATCACTGTTTGTGCTCATTC |
| | | TGTAAATGGATATTTGGGAGCTCATTGAGGTCAATGGCA |
| | | AAAAAGTGAATAACTGAGGATAAAAACTAGAAATAAGC |
| | | TATCTGAGAAACGGATTTATGATGTGTGCATTCATCTCG |
| | | CAAAATTAAACATTTCTTTTCCTTCAACAGTTTGGAAAC |
| | | ACGGTTTTTATAGGATCTGCAAAGGGATATTTTGGAGCA |
| | | CATTGAGGTCTATGGTGTAAAAGAAAATATCTTCAGATA |
| | | AAACCTAGAAAGAAGCTTTCTGAGAAATTGCATTTTGAT |
| | | GTGTGCATTCATCTCACAGAGTTAATACTTTTTTTTGATT |
| | | CACCAGTGTGGATACACTGTTTTTGTCCATTCTGCTAATG |
| | | GACCTTTGGGGGCTCATTTAGGCCAATGGCAAAAAAGTG |
| | | AATATCCCAGGATAAAAACTAGAAGGAAGGTATCTGAG |
| | | AAACTGCTATGTGATGTGTGCCATCATCTCACAGTCTTA |
| | | AAAGTTTCTTTTCATTCACCAGTTTGGAAACAGTGTTTTT |
| | | GTAGAATCTGCAAAGTCATATTTTTGAGT |
| 24 | ALR/Alpha | GATGAATGCACATATCACAAAAAGTTTCTCAGATTGCTT |
| | (Satellite/centr) | CTGTCTAGATTTTATGTGAAGTTATTTCCTTTTCTACCAT |
| | | AGAACCCAAAGCACTTATAATGTCCCCTTGCAGATTCTA |
| | | CAAAAAGAATGTTTCAAAACTGCTCAATCAAAAGAAAG |
| | | GTTCAAATTTGTGAGATGAATGCTCGCATCACCAAGAAG |
| | | TTTCTCAAAATACTTCTGTCTAGTTTTTATGTGAAGACAT |
| | | TTCTTTTTCCGTGATAGGTCTCAAAATGATCAAAATTTCC |
| | | ACTTGCAAATTCTACAAAAAGAGAGTTTCAAAGCTGCTC |
| | | AATCAAAAGAAAGGTTTATATCTGTGAGATGAACACAC |
| | | ACTTCACAAAGCAGTTTCTAAGATTTCTTCTGCCTAGATT |
| | | TTATGTAATGATATTTCCTTTTCTACCATACTTTGCAAAC |
| | | TGCTCCAAATGTCCACCTGTAGATTCTACAAAAACAGTG |
| | | TTTCCAAACTGCTCAATCAAAAGAAAGGTTAACTCTGTG |
| | | AGATGAACTCACACATCACAAAGAAGTTTCTCAGAATTC |
| | | TTCTGTCTAGTTCTAATGTGAAGATATTTCCTTTTGGACC |
| | | AGAGGCCCCAAGGCTATCGAAATGTCCCCTTGTAGATTC |
| | | TACAAAAAGAGTATTTCAAAACTGGTCCATCAAGAGAA |
| | | AGGCTCAACTCTGGGAGATGAATGCACACATCACAAAG |
| | | AAGTTTCTCAGAATGCTTCTATCTAGTTTTTACATGAAGC |
| | | TATTTCCTTTTCCACCATAAGCCTCAAAGCGCTCCAAAT |
| | | GTCCACTTGCAGGTTCTGCAAAAAGAGAGTTTCCAAAGT |
| | | TCTCAATCAAAAGAAACGGTTAACTCTGTGAGATAAATG |
| | | CACACATCACAAAGAAGTTTCTCAGAATTTTTCTGTCTA |
| | | GTTTTTGTGTGAAGATATTTCATTTTC |

TABLE 2 provides certain details of illustrative polynucleotides that can be used in a synthetic delivery system disclosed herein, including, e.g., identified genomic insertion sites and/or a number of Single nucleotide variants (SNVs or SNPs) identified in experiments disclosed herein. Certain provided polynucleotides can correspond to the sequences provided in TABLE 1 and can be used in a synthetic delivery system disclosed herein, for example, for delivery to cancer cells, e.g., hematologic cancer cells, such as multiple myeloma cells, B cells, and/or plasma cells.

TABLE 2

| SEQ | | | | | |
|---|---|---|---|---|---|
| ID | | No. | | | Chr |
| NO | Description | SNP | Location | Gene | position |
| 1 | ALUSx | 1 | Chr2 | CHMP3 | |
| | (ASLUSq2) | | | | |
| 2 | L1MB3 | Many | Chr2 | CHMP3 | |
| 3 | L2a | Many | Chr4 | AKAP6 | |
| | (2L2a) | | | | |
| 4 | THE1C | 2 | Chr4 | PI4K2B | |
| | (ERVL-MaLR) | | | | |
| 5 | ALUY (ALU) | 8 | Ch13 | none | |
| 6 | L1ME4b | 6 | Chr13 | none | |
| 7 | ALUSx | 0 | Chr17 | CHMP3 | |
| | (ALUSg2) | | | | |
| 8 | MIR | 2 | Chr12 | LMO3 | |
| 9 | AluY | 9 | Chr8/16 | none | |
| | (ALU) | | | | |
| 10 | ALluY | 4 | Chr3 | none | |
| 11 | AluJb | 10 | Ch10/16 | none | |
| | (ALU) | | | | |
| 12 | AluSx | 5 | Chr10/ | none | |
| | (AluSg) (ALU) | | | | |
| 13 | MER11C | 4 | Chr14/ | none | |
| | (LTR) | | Chr3 | | |
| | (ERVK/LTR) | | | | |
| 14 | THE1A | 6 | Chr14/ | none | |
| | (LTR) | | Chr9 | | |
| 15 | AluY | 7 | Chr2 | OSBPL6 | |
| 16 | AluSx | 4 | Chr3 | none | |
| 17 | AluY | 4 | Chr6 | LINC01227 | |
| | (FLAM_C) | | | | |
| | (ALU) | | | | |
| 18 | AluSx | 0 | Chr5 | GTF2H2 | |
| | (AluSx1) | | | | |
| 19 | AluY | 0 | Chr5 | GTF2H2 | |
| | (AluSx1) | | | | |
| 20 | ALR/Alpha | 24 | Chr21 | AK311573 | 10752909 |
| | (Satellite/centr) | | | | |
| 21 | Alu | 1 | Chr3 | TBL1XR1 | 176878250 |
| | (SINE/Alu) | | | | |
| 22 | Alu | 1 | Chr3 | TBL1XR1 | 176878250 |
| | (SINE/Alu) | | | | |
| 23 | ALR/Alpha | 9 | Chr16 | | 32534349 |
| | (Satellite/centr) | | | | |
| 24 | ALR/Alpha | 49 | Chr21 | AK311573 | 10749086 |
| | (Satellite/centr) | | | | |

TABLE 3 provides sequences of illustrative polynucleotides that can be used in a synthetic delivery system disclosed herein, for example, for delivery to cancer cells, such as pancreatic cancer cells.

TABLE 3

| SEQ | | |
|---|---|---|
| ID NO | Description | Sequence |
| 25 | AluSx | AGCCAGGTGCAGTGGCTCACACCTGTAATCCCAGCACTT |
| | (AluSx1) | TGGGAAGCTGAGGCGGGTGGATCACTTGAGGTCAGGAG |
| | | TTCGAGACCAGCCTGGCCAACATGGTGAAACCCCGTCTC |
| | | TACTAAAAAAGTAAAAATTAGCCAGGTGTGGTGGCGGG |
| | | CACCTGTAATCCCAGCTACTCAGGAGGCTGAGGCAGGA |
| | | GAATTGCTTGAACCCGGGAGGCGGAGGTTGCAGTGAGC |
| | | TGAGATTACACCACTGCACTCCAGCCTGGGCAAGAGAG |
| | | CGAGAATCCGTCTCAAAAAAAAAAAAACAAAAAAAACA |
| | | AAAAAC |
| 26 | MIRc | AATTACAACAACCCTTAATATTTGTAAAGCGCTTGATAA |
| | (MIRB) | TTCAGGGAGGGCTTTTAAAAACTTCTATCCATCTCTTCCC |
| | (MIR) | CCCAGCAATCCTATGAGGTAGGCAGGGCTGGAATCATTT |
| | | GCTGCATTTTATCGAATAGGAAGCCATGACTCGTAGAGG |
| | | TCTGGTGACTTAACTGAGGTCACCCACTGAGGACGTGGC |
| | | AGAGCTGGGTCCTGAATCTGTGGCCCTCAGGCTCTTTGC |
| | | CTGGCCCTGCCTCACATAGACTACGCTGCCTCT |
| 27 | AluJb | TTTCTTACTTTTTTTATTTATTTTTTTCTTGAGACAGGGTC |
| | (AluSx1) | TCACTTGGTCACCCACGCTGAAGTGCAGTGGCgTGATCT |
| | | TAGCCCACTGCAACCTCTACCTCCCGGGCTCAAGcGATC |
| | | CTCCCTCCTCAGCCTCCTGAGTAGCTGGGACCACAGGCA |
| | | TCTGCCACCACACCCAGCTAATTTTTGTATTTTTAGTAGA |
| | | GATGGGGTTTCACCATGTTGGTCAGGCTGGTCTCAAACT |
| | | CCTGgCTTCAaGTGATCTGCCCACCTCAGCCTCCCAAAGT |
| | | GCTGGGATTACAGATGTGAGCCACTGCGCCTGGCC |
| 28 | AluSx1 | TTGATTTTTTTTTCTTTTATCTTTTTTTTTGAGACAGAGTT |
| | (AluSq) | TTGCTCTGTTGCCCAGGCTGGAGTGCAGTGGCGTGATCT |
| | (ALU) | TGGCTCACTGCAGTGTCCGCCTCCTGGGTTCAAGCGATT |
| | | CTCCTGCCTCAGCCTCCTGAGTAGCTGGGATTACAGGCA |
| | | TGCACCACCACGCCCGGCTTCATTTTTTATTTTTAGTAGA |
| | | GACAGGGTTTCACCATGTTGGTCAGGCTGGTCTCGAACT |
| | | CCTGACCTCAGGTGATCCACCCGCCTTGGCCTCCCAAAG |
| | | TGCTGGGATTACAGGCGTGAGCCACCGTGCCCGGCC |
| 29 | AluSx | GGCTGGGCATGGTGGTTCACGCCTATAATCCCAGCACTT |
| | (AluSp) | TGGGAGGCCGAGGCAGGCAGATCACCTGAGGTCGGGAG |
| | (Alu) | TTCGAGACCAGCCTGACCAATATGGTGAAACCCCGTCTC |
| | | TACTAAAAATACAAAAATTAGCCGGGCGTGGTGGTGCA |
| | | TGCCTGTAATCCCAGCTACTTGGGAGGCTGAGGCAGGAG |
| | | AATCGCTTGAACCCAGGAGGCGGAGGTTGCAGTGAGCC |
| | | GAGATTGCGCCACTGCACTCCAGCCTGGGTAACAGAGC |
| | | GAAACTCCGTCTCAGAAAAAAAAAAAGGCAGATATGAA |
| | | AAAGG |
| 30 | MLT2B4 | AATATTAATTTGGGCTCTCCAGAGAAACAGAACCAATAG |
| | (MLT2B4) | ATTACACATAGATATATAGAAAGACAGTTATTACAAGG |
| | (ERVL) | AATTGGCTCACAGGATTATGTAGGCTGAGAAGTCCCATG |
| | | ACCTGCCGTCTGCAAGCTGGAGACCCAGGAAAGCCGGT |
| | | GGTGTAGTTCCAGTCCGAGTTGGGAGGTCTGAGAATCGG |
| | | GAGAGCTGACAGAGTAAGCTCTAGTTGCAGCCTGGAGT |
| | | CCAGAGGGCAAAGAACACCTATGTCCCATCTCAAGCAC |
| | | GGTCAGGCGTCCCAGCTCAAGCAGAGAGAAAGAAGACT |
| | | TTCCTGCTCAGCCTCCTTGCTCTAAGCAGCCTCCCGACG |
| | | GATTGGATGAAGCTCACCCACGCTGGGGAGGGCAGCCA |
| | | CCTTCACTCAGTCCACCAATTCAAATGCTAATCTCATCC |
| | | AGAAACACCCTCACAGGCACACCCAGAAATAATGTTTA |
| | | GCCACATATCTGGGCATCCTGAGGACCAGTCAAATTGAC |
| | | TCATAAGTCTCACCATCATA |
| 31 | AluSx1 | TTATGACTATTATTTCTTTTTTTTTTTTTTGAGATGGAGTC |
| | | TCGCTCTATTGCCCAGGCTGGAGTGCAGTGGCATGATCT |
| | | CGGCTCACTGCAACCTCTGCCTCCCGGGTTCAAGCGATT |
| | | CTCCTGCCTCAGCCTCCTGAGTAGCTGGGATTACAGGTG |
| | | CCTGCCACCACACCCGGCTAATTTTTGTATTTTTAGTAGA |
| | | GATGGGGTTTCACTGTGTTGGCCAGGATGGTCTTAAACT |
| | | CCTGACCTCAAGTGATCCACCCACCTCGGCCTCCCAAAG |
| | | TGTTGGGATTACAGGCGTGAGCCACCATGCCCGGCC |
| 32 | L2a | CTCCATGTTCTGACCTCAGGTGATCtGCCCgCCTCCGCCT |
| | (L2a/LTR40b/ML | CCCATAAGTTCCTTCTGCACCTATTCCATTCCATCTGCAA |
| | T1J2) | TGTCATCCTGGCCAACCTGTGAATGGGTATAGCAGGATA |
| | | GTAAGTTAGTCCCAGTGTTTAGGCAGACTCCATAGACCA |
| | | GATTTAACGTGTTTGTATAAACCGCATGGCTCGCTCTTA |
| | | CTCAGCCTTCGGGTCGGCCTTAAGATACACAGTTTTAAG |
| | | AAAAGCATTGTCTTCACTCTTAGTGTAGATCAGGTCCTC |
| | | CTCnCCCCCAnCACGTTATGTCCCTGTCATTGAACATTAT |
| | | ACTTTTATGAAGAATTGTGAGCTGTCTGAGATTTTACCTT |
| | | ACTTGCAAaCTAACAAGTGGGCCTGCCAtAGTTTCATGTC |
| | | TGGCAGAAGACTCCAACAGGTCAGAGATTTTCATCTGTC |
| | | TTGTTCACTATTGTCTACATAGTGCCTGGAAAAATGCCT |
| | | GGCACACAGTAAACCCTCAATCAGTATTTTATGAATGAG |
| | | TGAAC |
| 33 | MLT1J2 | TGTGTTAGTCAGGATAGGCTAACCTCTGTAACACACAAC |
| | (parAluSp- | CCCAATTTTTCAGTGGCTCAGAACAATAGGAGTTTCTTT |
| | FULLMTL1J2) | CTTACTCATGTAAAGTCCAGGTGTGGCAGAAACTTGCGG |
| | (LTR) | GGGAGAAAGTCTACTCCACAGCATCACTCAGGGACCCA |
| | | GGCTCCTGCCTTACGGTGGTTCCGCCATTTTCCAGGGCCT |
| | | CGGAGTCCTCCATCCCATCTCCTCCATTCAGCTGGTGGA |
| | | TGTGGGAGGAGAGCGTGTATGAAAGACCACACAGAAGC |
| | | TTTTTTTGGGCCAGGCTTTGAGACGGCGCACATCAGTTT |
| | | CgCTCTTGTTGCCCAGGCTGGAGTGCAATGGcGCAATCTC |
| | | GGCTCACCGCAAAAGACACTGGGAAATTTGGTCTGGCC |
| | | ATGTGTCTAGGAAGCAGAGGAGATGGGGTTTCAtAAACT |
| | | CCTGAACTCTCTCTACCACG |
| 34 | AluSx | GGCCGGGTGCAGTGGCTCATGCCTGTAATCCCAGCACTT |
| | (AluY) | TGGGAGGCCGAGGCGGGTGGATCACCTGAGGTCAGGAG |
| | | ATCAAGACCATCCTGGCTAACACGGTGAAACCCCATCTC |
| | | TACTAAAAATACAAAAATTAGCTGGGCGTGGTGGCAGG |
| | | CGCCTGTAGTCCCAGCTACTCGGGAGGCTGAGGCAGGA |
| | | GAATGGCGTGAACCCAGGAGGCAGAGCTTGCAGTGAGC |
| | | CGAGATTGAGCCACTGCACTCCAGCCTGGGGGACAGAG |
| | | CAAGACTCTGCCTCAAAAAAAAAAAAAAATGGAAAAAA |
| | | AAAAAA |
| 35 | (MIRB) | AAGATGGGGAGGATGGTGGTGGAGCAGGCTGGGGAGAT |
| | (MIRc- part L2) | GAGGAGCTTGAGGGGCCTAGGAGCTAACCACCCATTCA |
| | (MIRc) | CTGTTTATGGGTGGGGTGGCTCCGGGCAGGTGGCCTGAC |
| | | CCCTCTAAACCTCAGTTTCCCCATCTGTAaAATGGgAGTA |
| | | aTTGTACCTGCCTCACGGGCATGTAGCAGGTGGGAGAGA |
| | | GAGTAATACGGGCAAGACGTCTAGTGCaGTGGCTGGAAC |
| | | ATAACAAGCACCCAGCAAGTGGTGGCCCCATTGCT |
| 36 | AluSx | GGCCGGGCACGGTGGCTCAAACCTATAATCCCAGCACTT |
| | AluSq | TAGAAGGCCAAGGCGGGCAGATCACCTGAGGTCAGGAG |
| | | TTCAAGACCAGCCTGGCCAACATGGTGAAACCCCGTCTC |
| | | TACTAAAAATACAAAAATTAGCCAGGTGTGGTGGCGGG |
| | | CACCTGTAATCCCAGCTACTCGGGAGGCTGAGGCAGGA |
| | | GAATCACTTGAACCCAGGAGGCGGAGGTTGCAGTGAGC |
| | | CGAGATCATGCCACTGCACTCCAGCCTGGGCAACAGAGT |
| | | GAAACTCTGTCTCAAAAAAAAAAAAAAAAAAAAAAAAA |
| | | AAAAA |
| 37 | MamR4 | AAATCGAATTCTCTGTCTCCAATTGCTTCTTGCTTTCAAG |
| | | GGACCCAAGCAGGAGGCTGCTCCGCCACATATAAATGT |
| | | AGGACCTGAAGAAGCAGTCTGCAGCTCAAGGCTGGAAT |
| | | TGGCCTAGATCCTCCCTGGAGGCCCAGATTCACAGTGTA |
| | | ATCGTTCCAAGCCATTAATCACAGGCACCCGGCGGGACT |
| | | GTGTCTCGTCCCAAAGGGCATCTCAAGAGGTTTTGCAGA |
| | | GTTTCATGTAAATGATCATTACACTACTAATAAATATGG |
| | | TTAGTGACTTTGAGCCACATCAGGTGCCTTGGGCCAAGG |
| | | AGCTGGCGGAAGGAGTATCTAGGCTTCCTTAAGTGAAG |
| | | GCACTGAACAGCCAGCAGAATTAATCCATCAACTGCTCT |
| | | CCTTTCTTCCCCACTTCTCATACACACACACACGCACAC |
| | | GCGCGCATGTACACACACGCTGGCGAGAGTTTTAGACCA |
| | | GAGCACCAAGCCCTCCAATGCCCATGTTAAGAAAATATG |
| | | AAGTTAATATGCTCGGCTTCCTGTTGAAGTCCGCCTCATT |
| | | CCCACTAACTGATCAGGATACACTCACAGTTCACCCGCA |
| | | AGAGCAGACAACAAATGAGGAGAGATAGGGCAACGGC |
| | | ACCTCTGTCTGCAGCATTTTTAAAAATATAGATACCTAC |
| | | TCTTTCAAACAGATTTGAAGTGAGAATATGGTTTCTATG |
| | | ATAATACCCAAAAATGAACTGCATAAACCATTGCCTAAT |
| | | GCTGAACAGCACTCCTCGATTCTTCCTAAATCTATACTC |
| | | GCAGTTCTTATGCAAGAAAAAGGTAACAACATCCCTGTT |
| | | CTCCATGCTTCCATTTTACTTCACATCTGGACTGTCACTG |
| | | CAAACAGCTGAATCTGATTTTAAAAAGGTACACATATAC |
| | | CACTTTCATTATCAATGTCTTATACAAATTACCTGATACA |
| | | TGTAGTTACTTAGAAATTATTTCCCATTGCTTCAATATCT |
| | | ATGAACAAAAAGTAAACACTACATAAGATAAAATAAAC |
| | | CAAAATATTAATGAGGCTCACAGAACTACTGATGAATA |
| | | AGCTCAGAAAACTGTTAGTATTTTTTATACTATGTGAAA |
| | | CCAAAGACCGATCAAGAAAATGTATTTACTAACTGTATT |
| | | TAATAACCACTGCAATAAGACTGAATGACTTAAGCAAA |
| | | GATAAATTGATAAAAGGGCCGGGCACGGTGGCTCAaaCC |
| | | TATAATCCCAGCACTTTaGAATGGCCaAGGCGGGCaGATC |
| | | ACCTGAGGTCAGGAGTTCaAGACCAGCCTGGCCAACATG |
| | | GTGAAACCCCGTCTCTACTAAAAATACAAAAAAAATTA |
| | | GCCAGGtGTGGTGGCGGTGCaCCTGTAaTCCCAGCTACTT |
| | | CgGGAGGCTGATCAAGTGGCAGGAGAATCACTTGAACCC |
| | | aGGAGGCGGAGGTTGCAGTGAGCCGAGAACTGCACTCC |
| | | AGCCTGGGCAACAAGAGTGAaACTCTGTCTCAAAAAAAA |
| | | AAAAAAAAAAAAAAAActgaagcaatcaaagcggctagtAAAAGAC |
| | | TTACAGAAAAAGTTGATTTGTAATAGAGGGTCAATCTGG |
| | | CACAACTGTAGATTACCTATAAATGTTTAGAATTATCCT |
| | | CTCTTCTAAAATGCAAACAGAATCAGCATGCAAAATAAC |
| | | TTTGGCTTACATGATTCAATTCTAGTCATCTAACAAAAG |
| | | AATTTACTCCAAATCCGGGAAGTTATGGCCAGCCTGGAG |
| | | GGCTAAGAAGAATCTGAAATAGAAAGAAATTTTTATTAT |
| | | TTAAATACTTCCCCTCTCTTCTGCTAAACAATCTCTTTAG |
| | | GCTTGATGGGCAAAAAGGATGTCGCAAGTTTCTACATTC |
| | | TTCTAAAAAACTCAGTACTTTAAAAGCTGATAGAGAAAT |
| | | TCAGTCACCCAAAGGAGTCAGCTGTCACAGAAAATGTTA |
| | | TCTAATAGTGGAAAGATTACATAATGCCGATCTGCACTC |
| | | GTATCTAATAGAGATTTTGAAGAACTAAGGAATTAGGCC |
| | | TCCTAAGATAAAATCTTTGCAGCATGCAATGAGCAAGTA |
| | | TAAAGATCTCATTCTCTTCCCAGGTGACTGGACAAGGGA |
| | | AAGATTCAAGCAAGCCTCTGGGAAGCGGGGAATATTTC |
| | | AAGGAGACTTACTTATTTCAACAAACATTTACTTGGGAC |
| | | TGGACCATCTATGCTGGGCCTAGGACTGTGCTAGGAGCT |
| | | GATGATACATGGATGAACAGGATTAACATCATATAAGA |
| | | AATGATTTGTTCTACGTGGACGTACTGAATCTCTAAGCC |
| | | AACACTAAAATCAAATTTCATATACTGCCAAAAATCACC |
| | | AAGAACTTTAGTGATGATATACAAATGGAGTTTGATATG |
| | | GAAAACTGGAGGCCAATAAAAATGAGGAAAGAACAACT |
| | | TCCTATAACAGATTAGTTCCCTAGTAGGAAAAACCATGT |
| | | TATTAAAGCTCTAAGTTAGGGTAACATTTAGGAGTATCT |
| | | AGGCTTCCTTGAGCCAAAACAGGTTAATCTCATCAGTTT |
| | | TAGATCATAACTTAAGAAAATATGAAGTTAATATGCTTA |
| | | GAAAGTACTAAAACCACACATTAAAAGAGGCAATAAGA |
| | | CCAAGGCAATAAATTTACTTGTAGTACCGGTTCCGAGAC |
| | | ACTTATTTGGTATTAGAAAGCCATCTAATAAAGGTTAAA |
| | | ATATTAGAGAACATAAACTATAAATGTCAATGACACAA |
| | | AATCTAATCTATCATCTTAAAGCTGCAATCGAGAGGATC |
| | | ACTCTTACAAATGATAGAATTAAAAAGAGGGTAATTAG |
| | | CATCAAAAATTCATTTTTTTTCCTGAATTTAAAACTATAG |
| | | GGCTATTTTTACAGCAAATCAAATGTAGAGCGGTGAAAA |
| | | ATAGGCCCCAAAACCGATCnCAGTGTTATGCCAGCAGAA |
| | | CCTGAAGAAACACATCATGGTCCTCTGGTAAGTAAGCTG |
| | | ATGACCTAACAGCAAATTCAGACACAGAAACAAAAGGA |
| | | ACTTTCAGGTGTCTACACACATATCATTAAGAAAGATTA |
| | | TGTGGCAGATGCTACATCACATTTACGTCTCAGGAAAGA |
| | | CCATGTGGCCCAGAGATGAGGGATGGTTTTTATGTCAAT |
| | | TCAAGTAAAAGTGGTTGCCCCTAAAAATAACCACATCTG |
| | | TATACATAAAGAAAGCTTGTCTGATGATACATGCAGAAG |
| | | ATGAGAAAAGGCTTAAGTGGAAAATGAATATAAAATTG |
| | | GATAATGTGTCACCTTGGCTGACATGGATTACACCAACA |
| | | AGCATAGTAATTCTGCTAAGATTACATACCAATAATTAG |
| | | CTTTCCAGTGTGGGACTGATAATGGATTAACATAGTTCC |
| | | ATTAACGTTAACTCAAGATTATTTTAGAAAATAACTGTA |
| | | AACGCCTGCTCAGATTCAGCCATATAACAGGATACTAGC |
| | | AAGTTCTTACTAACAGACCAGACATTCTGCTGCTTGATT |
| | | CATCTTTTAATTTGGTAGAACTTACTAAAATAACCTTTTC |
| | | TCTTAAATGGCACATCCAAAAACCCTCACAGAAAAAAA |
| | | TaTTTAAAAGTATGTAATCCCCAGTGAGATTCAAAATATT |
| | | GTGGAGATAATAACCAAACAGAACAAATGCACCAACCA |
| | | TCTCGGCGATCACAATAAACACAAGATTAAAAATCAAA |
| | | AACATAACTTTGCAGTAACCTTGTGACTAGCTGGTATCA |
| | | GAGCAGCACGCAAATCTGACAGAAAACAGCAATTAAAC |
| | | AAAGGCATTTCTGTCTTGACAACAGACTTCTATAAAACA |
| | | AGGAACTAAGGGCCGTTTTGGTAAGAAATAGAAAGGCC |
| | | AGATTAATCAGAGGAATGAAATTAATATTTA |
| 38 | AluSx | ACAGTGGCACACGCCTGTAGTCCCAGCACCTTTGGAGGC |
| | (SINE/Alu) | TGAGGTGGGTGGATCACCTGAGTCCAGGAGTTCGAGAC |
| | | CAGCCTGGGCAACATGGCATGACCCTGTCTCTGCAAAAA |
| | | ATATGAAAATTAGCCAGGCATGGTGGTGCGTGCCTGTAG |
| | | TCCTAGCTACTTGGGAGGCTGAAATAGGAGGATCGCTTG |
| | | AGCCCAAGAGTTGAAGGTTGCTGTGAGCCAAGATCGCA |
| | | CCACTACAATCCAGCTTGGGTGTCAGAGTAAGACCCTGT |
| | | CTCAAAACAAAAAGATAAAT |
| 39 | MIR | GTATTTAGATTCCCAGACTTTGGAGCCAGATCGGCTGAG |
| | (SINE/MIR) | TTCAAACCCTGTCTGTGCAACTATCAGCTGTTCAGCTGT |
| | | GTGACCTTGGGCAAGTTACTTAACCTCTCTGAACCCTGA |
| | | TTCCTCCTGCAAACGGTGGGAAAAATAATAGAACCAATC |
| | | TCTCTGGGTTGCTGTAAGGATTAAATTAATTTACAGTGC |
| | | ATGTAACATGCTTACAGCAATGTCTGGCACACAGTAAGC |
| | | ACTAAGTATTTGTTGTTATTTGTCATC |
| 40 | L1M1 | AGGATTCATCACCTTCTGACTTAGGAGCTGCTGGTCTCT |
| | (LINE/L1) | GAATAACCTACAGTGGTACCCCAATAACACATCATGAAC |
| | | ATTGGACTCCGAGATGTGCTGGCTTCAGGTGTGACCCAG |
| | | AACATTTCCCACTGTGGTGGGTATAGTGAAAGACACCTT |
| | | CTGTTTGAGCAAAGCAGAGGAAAAAGTAAATGGGATTC |
| | | TGTCTTGCACCTGAGGTACCAGCTTGGCCACAGTGGGGT |
| | | AGAGCACCAAGCAGACTCTTAGGGTCCCTGAGTCAGCAT |
| | | TTCTGGACCTGCCCTGCACCAAAGAGTAAGTCCCAGTTG |
| | | TGGAAGCAGTCATCATAAGCTGACTGAAGAGTGCTTGG |
| | | GTGTTGAGTGAACATCAGTGATAGCCCAGTGGAAACCCC |
| | | TGTGGGCTGGTGGTGGTGGCAGACACATTGTGAGGCTCC |
| | | TCTGCCTGCAGAAAGGGGAGGGAGGAGTGGTAAGGATG |
| | | TTGTCTTGTAATTTGAATGCCAGCTTAGCTGCAGTAGAA |
| | | TAGAACACGAGATAAATAAGGCTTCTGACTTCAATACCT |
| | | GGCTCCCACACAATATCTCTGAACCTACCTGGGGCCAAA |
| | | GGGAACTCACTGCCCTGAGGAGACACAAACCTGGCTGG |
| | | TTACCACCTGCTGATTGTAGAGGCCTAAGGTCTTGAGTA |
| | | AACTTAGGTGGTAGCCAGGCAGTAATTACACTGGGCCTT |
| | | GGGTGACACCCAGTGCTGTGCTCACTTCAAGTCTAAATT |
| | | AGCACAGTCCCAGTGGTGGGGGCAACAGGGAAGCTTAT |
| | | GTCATCCCACCCCAGCTCCAGGTAGCTCAGCATAGAGAA |
| | | AGAAATTCCATTCGTCTGGAAGAAACTAAGGGAAGAGA |
| | | ACAAGAGTCTCTGCTTGGTAATCCAGATAATTCATCGAT |
| | | TTTAACCAAGACCAACCACATGGTACCTCTATGAGTCTG |
| | | CAAGAACAACGTTATTGAGTTTGAGATCCAAGTACCTTC |
| | | AAATTCTGGAAAGCCTTCCCAACAAGGGTGGACACAAA |
| | | GCCCAGACTGCGAAGGCTACAATAAATGCCTAACTCTTC |
| | | AATGCCAAGACACAGATGAACATCTACACATTAAGACC |
| | | ATCCAGTAAAATATGGCCTCACCTAACAAACTGAATAAG |
| | | GCATAAGAGGCCAATCCTGGAGAAACAAAATAAGTAAC |
| | | TTTTCAGACAGATGATTCAACATAGTTGTGTTGAGGAAA |
| | | CAAATTCAAGATAAAATAGAGAAGGAATTCAGATTCTA |
| | | TCAGATAAATCAAACAAAGAGGTTGAAATAATTAAAAG |
| | | GAATCAAGCAGAAATTGAAGTTAAAAAAGCAATTGGCA |
| | | TACTGAAGAATGCATCAGTCTCTTAATAGCAAAACTGAT |
| | | CAGGCAAAAGAAAAAATTATTGAGCTTGAACATAGGCA |
| | | ATTTGAAAATATGTAGAGAAGACAAAAAAGAATGAAGT |
| | | ATGCCTACAAGATTTAGAAAACACCCTGAAAAGGGCAA |
| | | ATCTAAGAGTTGTTAGCCTTAAAGAGAGAAAGAGAAAG |
| | | GGGTAGAAAGTTTCAAGAATACGATCAGAGAACTTCCC |
| | | AAATCTATAAAGATATCAATATCCAAGTACAACCAGGTT |
| | | ACAGAACACAAAGCAGATTGAACCCAAAGACGACTACC |
| | | TCAAGACATTTAATAATCAAACTCCCAAAAATTAAGAAT |
| | | AAAGGATCCTAAAAGCAGCAAGAAAAAAGAAACAGCAT |
| | | ACAATGAAACTCCAGTACGTCTGGCAGCAGATTTTCAGT |
| | | GGAAATCTTACAGGCCAGGAGAGAATAGCATGACATAT |
| | | TTAAAGTGCTGAAGTAGCAAAGCTTTTACACTAGAATAG |
| | | TCTATCCCGTGAAAATATTCCATAGCCATGAAGGAGAAA |
| | | AAAATGACCTTCCCAGACAAACACTGAGAGATTTCATTA |
| | | AAACCAGACCTGTCCTACAAGTAATACTAAGGGGCATTC |
| | | TCTTCCATCTGTAAGAAAAGAATGTTAATGAACAATAAG |
| | | AAGTCATCTGAAGGTACAGAACTCATTGGTAATAGCACA |
| | | GAGAAAAACTGAGTATTATAATATGGTAACTGTGGTGTG |
| | | TAAAATACTCAAGTAGAAATACTCAATGATGACTCAATA |
| | | CAACTACAACAATGTTTCAAGACAAAGACAGTACAATA |
| | | AGACATAAAGAGAACCAACAAAAAGTTGAAAAATGGGG |
| | | AAGCAAAGTTAAAGGTAGAGTTTTTACTAGTTTTCTTATT |
| | | GCTTATTTGTTCACTTAGGCAATCAGTGTGAAGTTGTCA |
| | | GCAGTTAAAACAATAGGTTATAAGATAGTATTTGTAAGT |
| | | TTCATGGTAACCTCAAATAAAAAAACATACAACAGACA |
| | | CAGAAACCAAAAAGCAAGAAATTAAATCATACCACCAA |
| | | GAAAAATCATGTTCACTAAAATGAAGACAGAAAGGAAG |
| | | GACAAAAGAAAGAGAAGATGCAAAAACAAATAACAAA |
| | | ATGGCAGGAGTGAACACATACTTATCAATAATAATGCTG |
| | | AATGGAAATGCACTAAACACTCCAATTAGATGACATAG |
| | | AATGGGTGAAAAAAAGAAAAAGGAAGACCCAATTATCT |
| | | GCTGCCCACAAGAAATACACTTCATCTATAAAGATACAC |
| | | ATAGACTGAAAAATAAAAGAATGGAAAAATGTTCCATG |
| | | CCAATGAAAACAAAAAAAAAGAACCAAAGTAGCTATAC |
| | | TTACATCAGATAAAATATATTTCAAGATAAAAACTGTGA |
| | | AGACAGAACAAATGTCATTTTATGATAAAGGGATCAATT |
| | | CAGTCAGAGAATATAACAATTTTAAATATCCATGCACCC |
| | | TACACTAAAGCACTGAGACATAGAGTATTAGAGCTAAA |
| | | GAGAGAGGCCCCAATACAATAATAATTGAGACTTCAAC |
| | | ACACCACTTTCAGCATTGTACAGATCTTACAGATAGAAA |
| | | ATCATGAAAGAAACATCAGACTTAATCTGTGCTATAAAC |
| | | CAAATATACCTAATAAATATTTGCAGAACATTTTATCTA |
| | | ATGGCTACAGAACACACATTCTTCTCAGCACATAGATAA |
| | | TTTTCAGATACACAACATTTTAGGTTACAAAACAAGTCT |
| | | TAAAACATTCAGAAAAGTGAAATAATATCAAACATCTCT |
| | | GCCCACAATGGAATATAACTAGAAATCAGTAACAAGAA |
| | | TTTTGGAAACTATACAAACAAATGTAAATTAAACAATAC |
| | | GCTCCTCAATGAAAATTGGATCAGTAAAGAAATTAAGA |
| | | ACGAAATTGAAAAATTTCTTCAAACAAATAATGAAAAA |
| | | ACACATACCAAAACCTATGGGATACAGCAAAAGCAGTA |
| | | CTGAGAAATGTATAGATATATGTGCTTACATCAAAAAAG |
| | | AAAAAAATCATATAACCTAATGTTGCATCTTAAAGAAAA |
| | | AACAGCAAAACAAAAATTATAATAAACATCACAGCAGA |
| | | AATAAAATTCAAATAAAAAATACAAAAAAAATCAATGA |
| | | AATGAAAAGTTGATTTTTTTAAGCAGATAAATAAAATTT |
| | | ACAAACCTTTAGCTTGACTAAGAAAAAAAGAAGACCCA |
| | | CATAAAACCAGAGATGAAAAAGGAGACATTATAACTGA |
| | | CACTGCAGAAATTCAAAAGATTATTAGTGGCTATTATTA |
| | | ACAACTGTATGCCAACTAATTGGAAAATCTAACAAAAAT |
| | | GGGTAAGTTCCCAGATATATACAACTTACCAAGATTAAA |
| | | CAACGAAGAAATCCAAAACATGAACAGACCAATAAGAA |
| | | GTAACAAGTTCAAAGCCATAATAAAAAGTCTCCCAGTA |
| | | AAGAAAAGCCCAGGACCTGATGGCTTCACCACTAAATTC |
| | | TACCCAACATTTAAAGATCTAATACCAGCTCTACTCAAA |
| | | AACTTCCAAAACACAGGAGGAAGGAATACTTCCCAACT |
| | | CATTCTTTGAGGCCAGTATTACCATAATACCCAAATCAG |
| | | AAAAAGATACATCAAAAGATGAAAACTGTACGCCAATA |
| | | TCTATAATAAATATTTATGTAAAAATAACAAAGTGTTAG |
| | | CAAACAGAATTCAACAACACATTAAAAGATTGTTCATTA |
| | | TGACAAAGTGGGATTTATCCCAGGGATGCAAGAATGGTT |
| | | CATCATATGCAATCAATCAGTATGATACATCAACAGAAT |
| | | GAAGGACAAAATCATATGATCATTTCAACTGCTGCTGAA |
| | | AAAAGGATTTAATAAAATTCAACATCCTTTGATAATAAA |
| | | AACTCTCAAAAAGCTGGGTATAGAAAGAACATACCTCA |
| | | AAATAATAAAAACCATATATGACAGATCCACAGCTAGT |
| | | ATCATCCTGAATGGAGGAAAAAAATCTGAAAGTCTTTTC |
| | | TCTAAGATCTGGAACATAACAAGGATGTCCACTTTCAAC |
| | | ACTGTTATTCAACATAGTACTGGAAGTGCTAGCTACAGC |
| | | AGCAGTAAAACGAGAAAAAACTAAATGGCATCCAAATT |
| | | GGAAAGGAAGTCGTCAAATTATTCTTGTTTGCAGATGGT |
| | | ACAGTCTTATATTAAGAAAAATCTAAAGACTCCACCAAA |
| | | AAACAGTAAGAACTGATAAAGTTGCAGGATATAAAATG |
| | | AACATACAAACATCAATAGCATTTCTATATGCCAACAGA |
| | | GAATCTGAAAAAAGAGATTAGAAAGTAATTCCATTTAC |
| | | AGTAGCCACAAATAAAATTAAATATCTAGACATTAACTT |
| | | AACCAAAGAAGTGAAAGATCTGTACAATGAAAACTATA |
| | | AAACATGAATGAAAGACATTGAAGAGGATGGCTGGG |
| 41 | LIMEg | CCCTTGAACTTATTAAAATCTTTTATAATGTAGTACATTT |
| | (LINE/L1) | ACTGACTGCTTTCAGGCAGGCATGAGGACCTTTCACTCC |
| | | ATTTGCTCCATTGTCCCTTTTGAAGGATTCTTGTCATATA |
| | | TATAAAACCTCTTAAGACATAATGTTATTGTTTTATATAC |
| | | TCTTTAATCACCTACATCTTTGCACTTTAATATTTCCTAA |
| | | ATTTGCATTCTTCTCTCTGGGACTAGAAGACCTCAGCTTA |
| | | GCGGTTCTTTTCATGTGGGCCTGAGAAATTTTCTCACTTT |
| | | TTGTTTCACTTTCATTCTTGAAGGAACGTTTCACTGGTTA |
| | | TAGGATGCAAGGTTAGCGGTTATTCTCTTCTGCATCTTCC |
| | | CACTGCATTGTCTTCTGAATCTTATAATTTATGTAAAAAC |
| | | ATTAGTTGTCTGTCTTCCCATTGCTTCTGTGAAAATAATG |
| | | CATGTTTTCCTCTGGTTGTTTTTGACATTTTCTTTGTGTCT |
| | | GTTTCTCAGCAATTTTTCCATGATATATGCTCGGAGCGG |
| | | GCTTACTTATCCTACACCCTGAATCTGTAGCTTGACGTCT |
| | | TTTTGTCAACTTTGGAAAGTTTTTGGCATTATCTCATCAA |
| | | ATATTTCCCCTGTCCTGTTCTTGCTCTTCTCTTGCTGGAG |
| | | TGCAGTCGTTCCTGTATTAGACCTTTGCATCATGTTCTAG |
| | | GTATTTTTATCT |
| 42 | AluSx | GCGGTGGCTCACGCCTGTAATCCCAGCACTTTGGGAGGC |
| | (SINE/Alu) | TGAGGCGGGTGGATCACTTGAGGTCAGGAGTTCGAGAC |
| | | CAGCCTGGCCAACATGGAGAAACCCCATCTCTACTAAAA |
| | | ATACAAAAATTAGCTGGGCGTGGTGGCTCATGCCTGTAA |
| | | TCCCAGCTACTTGGGAGGCTGAGACAGGAGAATTGCTTG |
| | | AACCCAGGAGGTGGAGGTTGCAGTGAGCCAAGAACACA |
| | | CTATTGCACTGCAGCCTGGGTGACAGAGCCAGACTCTGC |
| | | CTGAAAAGAAAAGAAAAGAAAAAGAATATG |
| 43 | AluSz6 | TTTTTCTTTGAGGTAGAGTCTCACTCTGTCACCTAGGCTG |
| | (SINE/Alu) | GAGTACAGTGGTGCAATCTCAGCTCACTGCAACCTCTAC |
| | | TTCCCAGGTTCAAGTGATTTTCGTGCCTCAGCCACCCAA |
| | | GTAGCTGGGATTATAGCCATGCGCCACCACACACAGCTA |
| | | ATTTTTTTGTATTTTTTAGTAGAGATGGGGTTCGCCATGT |
| | | TGGCTAGGCTGGTCTCAAACTCCTGGCCTTAAATGATCC |
| | | ACCCACCTCAGCCTCCCAAATTGCTGGGATTGCAAGCAT |
| | | GAGCCATCGCACCTGGCGCAGAAC |
| 44 | ALR/Alpha | TCAACTCACAGAGTTGAACAATCCTTTTCATACAGCAGT |
| | (Satellite/centr) | TTTGAATCTCTCTTTTTGTAGAATCTCCAATGGACATTTG |
| | | GAGCACTTTGGGGCCTTCGCTCGAAACGGGAATATCTTC |
| | | CCATAAAAACTGGACAGAAGCTTTATCAGAAACTACTTT |
| | | GGGATATGTGCTTTCAACTCACATAGTTGAACCCTTCTTT |
| | | TGATAGAGCAGTTTTGAAACACTCTCATTGTAGAATCTG |
| | | CAAGTGGACATCTGGTGTGCTTTGAGGCCTATGGAGAAA |
| | | AAGGAAATATCTTCACATAAAAACTAGACAGAAGCATT |
| | | CTCAGAAACTTATTTTTGATGTGTGTACTCAACTGACTCA |
| | | GTTACACCTTTCTTTTGATAGAGCAGTTTTGAAACACTCT |
| | | TCTTGCAGAATTTATAAGTGGATATTAGGAAAGCTTTGA |
| | | GGCTTTTGTTGGAAACGGGAATGTCTTCACATAAAAACC |
| | | AGACAGAAGCATTCTCAGGAACATCTTTGTTTTACGCGC |
| | | ATTCATCTCACAGACTTGAAACTTTCTTTTGATAGAGCA |
| | | GCTTTGAAACAAACTTTCTGTAAAATTTGCAAGTGGATA |
| | | TTTGGACAGCTTTGAAGCCTTTGCTGGAAACGGGAGTAT |
| | | CTTCACATAAAAACTAGATGGAAGGATTCTCAGACACTT |
| | | CCTTGGGAGGTTTGCATTCAACTCAAAGAGTTGAACATT |
| | | CCTTTTCATAGAGCTGTTTTGAAACACTCTTTTTGTAGAA |
| | | TCTGCTAGTGGACATTTGGAGCAACTTGAGGCCTGTGGT |
| | | GAAAAATGAAATACCTTCGCATAAAAACTAGACAGAAT |
| | | CATTCTCAGAAACTTCTTTGTGATGTGTGTACTCCACTCA |
| | | CAGAGTTGAACATTTCTTTTGATAGAACAGTTTTGAAAC |
| | | ACTCCCTTGTAGAATCTGCAGGTGTATATTTGGACAGCT |
| | | TTGAGTCTTTCATTGGAAACGGGAATATCTTCTCATAAA |
| | | AACTAGACAGAAGCACTCTCAGAAACTTCTCTGTGATGT |
| | | TTGCCTTCAACTCTCAGAGTTGATCATTCTTTTTCATAGA |
| | | GCAGTTTTGAAACACTCTTTTTGTAGAATCTGCAAGCGG |
| | | ACATTTGGAGCACTTTGAGGCCTGTGGTAAAAAAGGAA |
| | | ATATCTTCACATGAAAACTAGACAGGAGCATTCTAAGAA |
| | | ACTTATTTGTGATGTGTACACTAAACTCACAGAGTTGAA |
| | | CTTTTCATTTGATAGAGCAGTTTTGAAACACTATTTTTGT |
| | | GAATCCTGCAAGTGGACATTTGGAGCACTTTGAGGTATT |
| | | CTTTGGAAACGGGAATATCTTCACATAAAAACTAGAAG |
| | | GAAGCATTCTCAGAAACTTCTTTGTGAGGTTTGCATTCA |
| | | ACTCACAGAGTTGAACTTTTCTTTTGATAAAGCAGTTTTG |
| | | AAACACACTCTGTAGAATCTGTTAATGGATATTTGGACA |
| | | GCTTTGAGGTTTTCGTTGGAAACGGGACTATCTTCACAT |
| | | AAAAACTAGACAGAAGCATTCTCAGAAACTTCTTTGCGA |
| | | TGTTTGCATTCAACTCACAGAGTTGAAGATTCCTTTTCAT |
| | | AGAGCAGTTTTGATATGCTCTTTTTGTGGAATCTGCCTGA |
| | | GGACATTCGGAGTGCTTTGAGGCCTAAGGTGAAAAAGG |
| | | AAATATCTTCACATAAAAACTAGACAGAAGCATTCTCAG |
| | | AAACTTCTTTGTGAAGTGTGTACTCAACTCACAGAGTTA |
| | | AACCTTTCTTTGATAGAGCAGTCTTGAAACACACTTCTT |
| | | GTAGAATTTACAAGTGGATATTAAGACAGCTTTCAGGCT |
| | | TCCATTGGAAATCCTGAAGATATTCGCTGGAATATCTTC |
| | | ACATAAATTGTAGACAGAAGCATTCTCAGAAACATCTTT |
| | | GTGATGTGTGCTGTTGGAACTTTCCTTTGATAGAGCAGT |
| | | TTTGAAACACTCTTTTTGTAGAATTTGCAAGTGGATATTT |
| | | GGACAGCTTTGAAGCCTTCCCAGGAAATGAGTTTATCTT |
| | | CACATAAAAACTAGACAGAATCATTCTCAGAAACTTCTT |
| | | TGTGATGTTTGGTTTAACTCACAGAGTTGAACATTCCTTT |
| | | TCATAGAGCAGTTTTGAAAAACGCTTTTTGTAGAATCTG |
| | | CAAGTGGACATTTGCAGCGTTTTGGGACCCATAGTGAAT |
| | | AAGGAAAGATGTTCACATTAAAACTAGATAGAAGCATT |
| | | CTCAGAAACTTCTTTGTGATGTGTGTACTCAACACACAG |
| | | AGTTGAATCTTTGTTTTGACAGAGCAGTTTGAAACCCTTT |
| | | TTTGTATAATCTGCAAGTGGATATTTGGATAGCTTTGAG |
| | | GCTTCCGTCGGAAACGGGAATATCTTCACATAAAAACTA |
| | | GAGAGAAGCATTCCTCAGAAACTTCTTTGTGAAGTTTGC |
| | | CTTCAACTCACAGAGTTGAACATTCCTTTTCATAGAGCA |
| | | GTTTTGAAACACTCTTTTTGTAGAATCTGCAA |
| 45 | L1P1 | CAGGCCTGAGCAATGGCGGGCACCCCTCCCCCAGCCTCG |
| | (LINE/L1) | CTGCCGCCTTGCAGTTTGATCTCAGACTGCTGTGCTAGC |
| | | AATGAGCGAGGCTTCATGGGCATGGTACCCTCCGAGCCA |
| | | GGTGCGGGTTATAATCTCCTGGTGTGCCGTTTGCCAAGA |
| | | CCATTGGAAAAGTGCAGTATTAGGGTGGGAGTGACCCG |
| | | ATTTTCCAGGTGCTGTCTGTCACCCCTTTCTTTGACTAGG |
| | | AAAGGAAATTCCCTGACCCCTTGTACTTCCCAGGTGAGG |
| | | CGATGCCTCGCCCTGCTTCGGCTCATGCTCG |
| 46 | THE1B | CACCCAAGTCACCTCTTGAATGCTTTGCTGCTTAGACATT |
| | (LTR/ERVL- | TCTTCTGCCAGATACCCTAAATAATCTCTCTCAAGTTCAA |
| | MaLR) | AGTTCCACAAATCTCTAGGGCAGGGGCAAAATGCCACC |
| | | AGTCTGTTTGCTAAAACATAACAAGAGTCATCTTTGCTC |
| | | CAGTTCCCAACAAATTCCTTATCTCCATCTGAGACCACC |
| | | TTATCCTGGATTTCATTGTCCATATCATTATCAGCATTTT |
| | | GGTCAAAGCCACTCCACAAGTCCCTAGGAAGTTCCAAAC |
| | | TTTCCCACATTTTCCTGTCTTCTTCTGAGGCCTCCAACCT |
| | | ATTCCACCCTCTGTCTGTTACCCAGTTCCAAAGTTGCTTC |
| | | TACATTTTCAGGTGTCTTTTCAGCAGTGCCACACTCTACT |
| | | GGTACTAATTTAC |
| 47 | AluSg | AGGCCTGGCACAGTGGCTCATGCGTGTAATCCCAACACT |
| | (SINE/Alu) | GCGGGAAGCTGAGGCAGGAGGATCACTTGAGCCCAGGA |
| | | GCTCATGGCTGTAGTGAGCTGTGATTGCACCACTGCACT |
| | | GCACTCCAGCCTGGGCAACAGCAAGACCCTGTCTCCAAA |
| | | AAAAAAAAACAAAACAAATTAAT |
| 48 | HER V9N-int | TTATGGTGGACATCATTGAATAATTCATGGGCTTTTTCCT |
| | (LTR/ERV1) | AATTCTCCTTAGTCCTTCTAGAACACAGGTCAACAGATG |
| | | TTTGTGACTCCAGTCCCTATGATCTGAGTTGAGGTCCCA |
| | | GTGGGGATCCATACTGGGGACAGCTTGCTGACCAGTAG |
| | | GGAATTTTCCCTTTCTTTGGCTGTCATTTTATCATTTACTT |
| | | GACTAAGATGCCAGGTATCTCCAAACTCTTGGGCTGCAG |
| | | CTAAAGCTGCATTCTTTTCATTAAAGGCCAGGGTTTGAT |
| | | CTAACAAAAGCATGACATCTTTCCAAGTGAGATCGAAG |
| | | GTTTCCCCTAGACCCTGTAGGACATCTATGTACCTATCA |
| | | GGATCATCTGAAAACTTTCCCAGGTCTGCCTTGATCTGC |
| | | TTTAAATCAGAGAGGGAGAAGGGGACATGTACCTGGGT |
| | | TGGGCCAAATTCCCCTTCCCCTACAGCTTGAAGGGGACG |
| | | TAACCGATAGCCCGGGGGTTTTTGTGGTCCTTTGGAGAT |
| | | TTCTTTGCTTATTTCCTTCTGGGCAGGGGAGATTAGAGG |
| | | AGGCTTATCATTAATAGGAGGGGGAGCCATAGGGAGGC |
| | | TAGGATATGGGACTAAGCTGAAAGGTCCTCCTGGGGGA |
| | | TGTAAATTGCAAGCTTTGCATAGTCATGTATTCTCCTTCA |
| | | ATGAAAAGAGTTTGGACATAAGGTATTTCACTCCATTTG |
| | | CCTTCCCTCTTACAGAAAAGGTCAAGCTGCAGGATAGTA |
| | | TTGTAATTTATACTTCCCTCAGGTGGCCATTTTTCCCCAT |
| | | CAGAGAGAGAATATTGGGGCCAGGCTGTAGGGCAGAAA |
| | | AAAATGAGCCGCCTCTTTTTCAGGGTTTGTGGGTCAAAT |
| | | TGGTCCCAATGGCTTAGGATGCATTTCAAAGGTGAGCCT |
| | | GTTGATGCCTGAGTGTTTCCCATCTGAAAGACAAAACCA |
| | | CCTGTGGTTTTGGTTTGTTTTGTTTCTCCCCCACCCACAA |
| | | ACCTGCAACGGTCCCTGGACCCTGCTGATCGGAATAGTT |
| | | GTGCTCCCTGACGCAGCAGCAAAAATACTAGTTTTCCTC |
| | | CCAGACCACAAGGGGGACTGAGAAAGGTCGGATTTAGT |
| | | GGCCCTTACCAACGCATTCTCGAAAACCTGCACCCTTGC |
| | | CTGTCCTCCTAGACCACAAAGAGGACCGAGAAAAATCG |
| | | GATTTAGTGGCCCTTACCGACACATTCTCGAAAACCCAT |
| | | TAGAGTCCTAAGCATTCACCTGTTAGTATTGGGACCTTA |
| | | ACCATGTCCTATAAAGATGTTATGCCCCCCAAATGAAGT |
| | | GGAGTGCCATACCCTGAGGGAGGGAAGGGATGTCCAGG |
| | | GTTGGAAGAGTGACACCTTTTGTCCTTACTTATATGAAT |
| | | AGGAAGGATACAATTTCTGAGGCTCCCCATATTCTAGCT |
| | | TCAGGAATAGCTTTTGTTAGGCCTGCTAGTCTGAGGAGG |
| | | GATCCTAAAATTCCAGATAGTCCCCCCTACGATGGGGCT |
| | | TTGGGCAAAAATTACGTCTTTCTGATTGGTGAGCCTGGG |
| | | TGCCTACAGAAGGTAACAGAATCCCAAATTTATACTAGA |
| | | AATCATTCTTATAGGAGAAACTAGAAAAGCACCAGAGA |
| | | CAGGGAGTGGTTTTTAGAAGCAGGACTAGCCTGGAGAA |
| | | GAGAGGCAAGGGGAAGTTTGTCTGGCAGGCGTTAGGAC |
| | | CCAGGGGGCAATGGTCAGGGTAGATAGAATAGATGGGC |
| | | AAGTCTCGCTTGGGCGACATGACTTTGAGAGTTCCACTC |
| | | ATGGCCACAGGGTCAACCAACTTGTTGTTGGGACCCCGG |
| | | AGCTGAATGGCTTTCCTCTCTGTCGACCCTGGGCTCAGC |
| | | CCAGAAGTACAGGAAAAGCGGAAGCTGGTTCCAGGCAA |
| | | ACCAACGCTACCAACTCTGAAGAGTCAGGGATTGTTAGA |
| | | AAGGCCTTTCCCAGAAAGCCTGACACCTGTGTCTTTAGT |
| | | CTGGCAGCTGCGCTAGTAGCTTTTAACTGGCCGACAGGT |
| | | GCCTGGTATTTAGCCCCCGAATTCTAAGGAAAAATAGGA |
| | | AAGAATAGCAAGCGAAAGGGGTCCGATGGTACTCACCA |
| | | CTTGGCAATAGGCGATAGTCTCACTTCTTGGCGATAGGC |
| | | GATGGTCCCATCTGGGTCGCCAAAA |
| 49 | HERV17-int | CTTTAACCTCCTTGTTACGTTTGTCTCTTCCAGAATCGAA |
| | (LTR/ERV1) | GCTGTAAAACTACAAATGGTTCTTCAAATGGAGCCCCAG |
| | | ATGCAGTCCATGACTAAGATCTACCGCGGACCCCTGGAC |
| | | TGGCCTGCTAGCCCATGCTCCAATGTTGATGACATTGAA |
| | | GGCACCCCTCCCAAGGAAATCTCAACTGCACGACCCCTA |
| | | CTATGCCCCAATTCAGCATGAAGCAGTTAAGAGCGGTTG |
| | | TCAGCCAACCTCCCCAACAGCACTTGGGTTTTCCTGTTG |
| | | AGAGGTGGGACTGAGAGA |
| 50 | LTR81B | CAGTCTTTCAGGACCAGAAGAGATGGATTTTAGGTTTTC |
| | (LTR/Gypsy) | CTTCCACTCAATGTATTCTATATGTACCCTAATCAACAA |
| | | ACATGCCTGGTTGTCACAGGTAGAAGGGGTCTCTACTCT |
| | | GAATCATTCAGAGAAGAAATAATCATATTCTTTCGATGC |
| | | TATCACTAACACAGGACCGTGATCCGAGACAACCAGGTT |
| | | CAGTTCTTGATATGTAAAGGAATTTGGGAAAATATCATC |
| | | TCTCACCACAAAATCTGTATTTTTATGTATATTTAAGCAA |
| | | AATCCTGTTTTGGTCATCATTTTCTTTACCTTTCTTCTATA |
| | | TCCATCCTAATCTCCCTTTCAGC |
| 51 | ALR/Alpha | CCTATGTTAGAATAGAAAATGTCTTCACATAAAATCTAG |
| | (Satellite/centr) | ACAGAAGCAATCTGAGATACTTCTTTGTGGTGTGTCCTT |
| | | TCATCTCAAAGAGTTAAAACTTTCTTTTGATTGAGCAGTT |
| | | TTCAAACTCTCTTTTTGTAGAATCTGCAAGTGGACATTTG |
| | | GAGCGCTTTAAGGCCTGTGGTGGAAAAGGAAATATCTTC |
| | | ACCTAAAAACTAGACAGAAGCAATCTGAGAAACTTCTTT |
| | | GTGATGTGTGAATTCATTTCACAGAGTAAAACCTTTCTTT |
| | | TGATGGAGCCGTTTTGAAACTCTCTTTTTGTAGAATCTGC |
| | | AAATGGACATTTGGAGTTCTTTGAGGCCTATGGTGGAAA |
| | | AGGAAATATCTTCACATAAAAACTAGATAGAAG |
| 52 | ALR/Alpha | TCATCTCACAGAGTTAAACCTTTCTTTTGATTGAGCAGTT |
| | (Satellite/centr) | TTGAAACTCTCTTTTTGTAGAATCTACAAGTGGACATTG |
| | | GGAGCTCTTTGAGGCCTATGTTAGAAAAGGAAATATCTT |
| | | CACATAAAACCTAGATAGAGGCAATCTGAGAAACATCT |
| | | TTGTGATGTGTGCATTCATCTCACGGAGTTAAACCTTTCT |
| | | TTTCGTTGAGCAGTTTTTAAACTGTCTTTTTGTAGAATGT |
| | | GCAAGTGGACATTTGGAGCGCTTTTTGTCCTATGGTGGG |
| | | AAAGGAAGTATCTTCACATAAAAACTAGACCGAAGAAT |
| | | TCTGAGAAACTTCTTTGTGACGTGTACGTTCATCTCACTG |
| | | AGTTGAACCTTTCTTTTGATTGAGCAGTTTGGAAACACT |
| | | CTTTTTGTAGAATCTGCAAGTAGACATTTGGAGCGCTTTT |
| | | CAGCCTACGGTAGAAAAGGAAATATCTTCACATCACAA |
| | | AGAGAGAGTTTCAAAACTGCTCTATCAAAAGAAAGGTTT |
| | | AACCCTGTGCGATGAATGCACATATCACAAAGAAGTTTC |
| | | TCAGATTGCTTCTGTCTAGATTTTATGTGAAGATATTCCT |
| | | TTTTCCACCATAGGCCTCAAAGCGCTCCAAATGTCCACT |
| | | TGCAGATTCTACAAAAAGAGTGCTTCCAAACTGCTCGAT |
| | | GAAAAAAATGGTTCAACTCTGTGAGATGAATGCACGCA |
| | | TCACAAAGAAGTTTCTCAGAATTTTTCTATCTAGTATTTA |
| | | TTTGAAGATATTTCATTTTCCACCAT |
| 53 | AluSc8 | TTTCAGATAGAGTCACCCTCTGTCGCCCAGGCTGGAGTG |
| | (SINE/Alu) | CAGTGGCATGATCTTGGCTCACTGCAACCTCCACCTCCC |
| | | AGGTTCAAGCGATTCTCCTGCCTCAGCCTCCTGAGTAGC |
| | | TGGGATTACAGA |
| 54 | L1P3 | GAACTAATTTACACTCCCACCAACAGTGTAAAAGTGTTC |
| | (LINE/L1) | CTATTTTTCCACAACCTCTCCAGCATCTGTTGTTTCCTGA |
| | | CATTTTAATGATTGCCATTCTAACTGGCATGAGATGGTC |
| | | TCATTGTGGTTTTCATTTGCATTTCTCTAATGACCAGTGA |
| | | TGATAAGCATTTTTTCATACATCTCTTGGCTGCGTAAATG |
| | | TCTTCTTTTGAAAAGTGTCTGTTCATATCCTTTGCCCATT |
| | | TTTTGATGGGGTTGTTGCCTTTTTTCTTGTAAATTTCTTTC |
| | | AGTTCTTTGTAGCTTCTGGATATTAGCCCTTTGTCAGATG |
| | | GACAGATTGCAAAAATCTTCTCCCATTCTGTAGGTTGCC |
| | | TGTTCACTCTGATGATGATGATAGTTTCTTTCGCTGTGCA |
| | | GAAGCTCTTTAGTTTAATTAGATCCCATTTGTCAATTTTA |
| | | ACTTTTTTTGCCATTGTTTTTGGTGTTTTAGACATGAAGC |
| | | CTTTTCCCATGCCTATGTCCTGAATGGTATTGTCTAGGTT |
| | | TTCTTCTAGGATTTTTATGGTCCTAGGTCTTATGTTTAAG |
| | | TCTTTGATCAATCTTCAGTTGATTTTTGTATAATGTGTAA |
| | | GGAAGGGGTCCAGTTTCAGTTTTCTGCATATGGCCAGCC |
| | | AGCTTTCCCAACACCATTTATTAAATAGGGAATCCTTTC |
| | | CCCATTTCTTGTGTGTGTCAGGTTTGTCAAAGATCAGAT |
| | | GGCAGTAAATGTGTGGTGTTATTTCTGAGGCCTCTGTTCT |
| | | GTTCCATTGGTCTATATATCTGTTTTGATACCAGTGCCAT |
| | | GCTGTTTTGGTTACTGTAGACTTGTAGTATAGTTTGAAGT |
| | | CAGGTAGCATGATGCCTCCAGCTTTGTTCTTCTTGCCCAG |
| | | GATTTTCTTGGCTATGTGGGCTTTTTTTTGGTTCCATATG |
| | | AAGTTTAAAATAGTTTTTTCCAATTCTGTGAAGAAAGTC |
| | | AGTGGTAGCTGGATGGGGAGAGCATTGAATCTATAAATT |
| | | ACTTTGGGCAGTAAGGCCATTTTCATGATACTGATTCTTC |
| | | CTATCCATGAGCATGCAAGGTTTTTCCATTTGTTTGTGTC |
| | | CTCTTTTATTTCCTTGAGCAGTGGTTTTTGAAGAGGTCCT |
| | | TCATATCCCTTGTAAGTTGTATTCCTAGGTATTTTATTCT |
| | | CTTTGTAGCAATTGTGAATGAGAGTTCACTCATGATTTG |
| | | GCTCTCTGTTGTCTGTTATTGGTGTATAGGAATGCTTGTC |
| | | ATTTTCGCACATTGATTTTGTATCCTGAGACTTTGCTGAA |
| | | GTTGCTTATCAGCTTAAGGAGGTTTTGGGCTGAGACAAT |
| | | GAGGTTTCCTAAATATACAATCATGTCATCTGCAAACAG |
| | | AGACAATTTGACTTCCTCTCTTCCTATCTGAATACCCTGT |
| | | ATTTCTTTCTCTTGCCTGATTGCCCTGGCCAGAACTTCCA |
| | | ATACTATGTTGAATAGGAGTGGTAAGAGAGGGCATCCTT |
| | | GCCTTGTGCTGGTTTTCAAAGGGAATGCTTCCAGTTTTTT |
| | | CCCCCATTCAGTATGATACTGGCTGTGGGTTTGTCATAA |
| | | AGAGCTCTTATTATTTTGAGATATGTTCCATCAATACCTA |
| | | GTTTATTGAGAGTTT |
| 55 | AluYc3 | GCGGTGGCTCACGCTTGTAATCCCAGCACTTTGGGAGGC |
| | (SINE/Alu) | CGAGGTGGGCGGATCACGAGGTCAGGAGATCAAGACCA |
| | | CGGTGAAACCCCATCTCTACTAAAAATATAAAAAATTAG |
| | | CCGGGCGTGGTGGTGGGCGCCTGTAATCCCAGCTACTCA |
| | | GAGAGGCTGAGGCAGGAGAAGGGCGTGAACCCGGGAG |
| | | GCGGAGCTTGTAGTGAGCTGAGATCGCGCCACTGCACTC |
| | | CAGCACCCCAGCCTGGGTGACAGAGCGAGACTCCGTCTC |
| | | AAAAAAATAATAATAATAATTAAATAAATAA |
| 56 | L1PA10 | CCAAGAAGTTCAAACTGGACAGAACCCACCACAGCTCC |
| | (LINE/L1) | ACAAAGCCACTGTAGCCAGACTGCCTCTCTACATTCCTC |
| | | CTCTCTGGGCAGGCCACCTCTGAAAGAAAGGCAGCAGC |
| | | CCCAGTCAGGGGCTTATAGATAAAACTCCCATCTCCCCG |
| | | GGACAGAGCACTTGGGGGAAGGGGCATCTGTGGGTGCA |
| | | GCTTCAGCAGACTTAAACGTTCCTGTCTGCTGGCTCTGA |
| | | AGAGAGCAGCAGATCTCCCAGCACAGCACTCCAGCTCTC |
| | | CTAAGGGACAGACTGCCTCCTCAAGTGTGTCCCTGAACC |
| | | CTGTGCCTCCTGACTGGGAGACACCTCCCAGCAGGGATC |
| | | GACAGACACCTCATACAGGAGAGCTCTGGCTGGCATTTG |
| | | GCGGGTGCCCCTCTGGGACAAAGCTTCCAGAGGAAAGA |
| | | ACAGGCAGCAATCTTTGCTGTTCTCAGCCTCTGCTGGTG |
| | | ATACCCATTTAAACACAGTCTGGAGGGGGCCTCCAGCAA |
| | | ACTCCAGCAGACCTGCAGAGGAGAGGCCTGACTGTTAG |
| | | AAGGAAAACTAACAAACAGAAAGGAATAGCATCAACAT |
| | | CCACAAAAAGGACATCCACACAAAAACCCCATCCAAAG |
| | | GTCACCAACATCAAAGACCAAAGATAGATAAATCCATG |
| | | AAGATGAGGAAAAAACAGCGCAAAAGGGCTGAAAATTC |
| | | CAAAAACCAGAATGCCTCTACTCCTCCCAAGGATCACAA |
| | | CTCCATGCCAGCAAGGGAACAAAACTGGACGGAGAATG |
| | | AATTTGACCAACTGACAGAAGTAGGTTTCAGAAGGTGG |
| | | GTAACAACAAACCCCTCTGAGCTAAAGGAGCATGGTCT |
| | | AAGCCATTGCAAGGAAGCTAAGAACCTTGAAAAAAGAT |
| | | TAGAGGAATTGCTATCTAGAATAACCAATTTAGAGAAG |
| | | AATATAAATGATCTGATGGAGCTGAAAAACACAGCTTG |
| | | AGAACTTTGTGAGGCATACACAAGATTCAATAGCCAAAT |
| | | CGATCAAGCAGAAGAAAGGATATGAGAATTGAAGTTCA |
| | | ACTTAATGAAATAAAATCTGAACATTAAAGTAGAGATA |
| | | AAAGAATGAAAAGGAACAAACAAAGCCTCCAAGAAATA |
| | | TGGGACAATGTGAAAAGACCAAACCTACGTTTCAATGGT |
| | | GTACCTGAAAGTCATGAAGAGAATGGAACCAAGTTGGA |
| | | AAACACTCTTCAGGATATTATCCAGGAAAACTTCCCCAA |
| | | CCTAGCAAGAGAGCCCAACACTAAAATTCAGGATACAC |
| | | AGAGAACACTACAAAGATACTCCTCAAGAAGACCAACC |
| | | CCAAGACACATAATCATCAGATTCACCAAGGTTGAAAG |
| | | GAAGGAAAAAATGTTAAGAGCAGCCAGAGAAAAGGGTC |
| | | AGGTTACCCACAATAGGAAACCCATCAGACTAACAGCT |
| | | GATCTCTCTGCAGAAAACCTACAAGCCAGAAGAGAGTA |
| | | GGGCCAATACTCAACATTCTTAAACAAAAGAATTTTTAA |
| | | CCCAAAATTTCATATCCAGTCAAACTAAGCTTCATAAGC |
| | | AAAGGAGAAATAAAATCTCTTATGGACAAGCAAATGCT |
| | | GAGAGATTTTGTCACCATCAGGCCTGCCTTACAAGAGCT |
| | | CCTGAGGAAGCACTAAATATAAAAAGGAAAAACTGGTA |
| | | CCAACCACTGCAAAAACATACCAAATTGTAAAGACCAT |
| | | CAACACCATGAAGAAATGGCATCAAATAAAGGGCAAAA |
| | | TAACCAACTAACATCATAATGACAGGATCAAATTCACAC |
| | | ATAACAATATTAACCTTAAATGTAAATGGGCTAAATGCC |
| | | CCAATTAAAAAACACAGACTGGCAAATTGGAAAGAGTC |
| | | AAGACAAATTG |
| 57 | AluSz | TTTTTTTTTTCCCAGACAGAGGCTCACTCTGTTGCCCAGG |
| | (SINE/Alu) | CTGGAGTGCAGTGGCATGATCTCGGCTCATTGCAACCTC |
| | | TGCCTTCTAGATTCAAGGGATTCTCCTGCCTCAGCCTCCC |
| | | AAGTAGCTGGGATTACAGGCCCACACCACCACGCCTGG |
| | | CTAATTTTTGTATTTTGAGTAGAGACGGCGTTTCATCATG |
| | | TTGGCTAGGCGGGTCTCGAATTCCTGACCTCAAGTGATC |
| | | TGCCCGTCTCCGTCTACCAAAGTGCTGGGATTACAGGCG |
| | | TGAGCCACCACGCCCGGCCTGAAGCC |
| 58 | REP522 | TAGATGGAGGGGGAAAAACAGGGTGGGGAGCGGGAGG |
| | | GAGGGAAGGTTTTGCAGAAAGACGGCGGTTAAAAAGTT |
| | | TATGGGTAGATGGAGGGGGAAAAGAGGGTGGCAAGTGG |
| | | GAGGAGGAAAGAGGGTGATAAGGGGGGGAAGTGGGGG |
| | | TGAGCAGTAGGGAGAGAAGGTTTTGTGAAAAAAATGGG |
| | | GAGAAAGGTTTTGGTTAGACGGGAGAAAAGAGGGTAAC |
| | | AAGTTGGGGAAGGAAAAAGAGGGTAGCTAGGAAGAAG |
| | | ACAAGGTTTTGCGAAAAGATAGCGGCCAGAAAAGAAAG |
| | | ACGGTGGAGAAAGGAAAGACGGTGGGTAAAAAGTGGGT |
| | | AGATGGAGTGGGGAAAGAGGGT |
| 59 | AluSc8 | TTTCAGATAGAGTCACCCTCTGTCGCCCAGGCTGGAGTG |
| | (SINE/Alu) | CAGTGGCATGATCTTGGCTCACTGCAACCTCCACCTCCC |
| | | AGGTTCAAGCGATTCTCCTGCCTCAGCCTCCTGAGTAGC |
| | | TGGGATTACAGA |
| 60 | L1P3 | ATTCAGAAGACAAATCTCACATGCAAAGACACACATAG |
| | (LINE/L1) | GCTCAAAATAAGTGGATGGAGGAATATTTACCAAGCAA |
| | | ATGGCAAGCAAAAAGAAAAAGCAGGGGTTGCAATCCTA |
| | | GTATCTGATAAAACAGGCTTTAAACCAAGAAAGATCAA |
| | | AAAAGACAAAGAAGGTCATTACATAATGGTAAAGGGAT |
| | | CAATGCAACAAGAAGAGCTAACTATCCTAAATATATAC |
| | | GCACCCAATACAGGAGCACCCAGATTCATAAAGTAAAT |
| | | TCTTAGAGACTACAAAGAGACTTATACTCCCATACAATA |
| | | ATAGTGGGAGATTTTAACACCTCATTGTTGATATTAGAT |
| | | CAATGAGACAGAAAATTAACAAGGATATTCAGGACTTG |
| | | AACTCAGCACTGGACAAAGCAGATTTAATAGACATCTAC |
| | | AGAACTCTCTACCTCAGATCAACAGAATATACATTCTTC |
| | | TCAGCACCACATAGCACTTATTCTAAAATTGACCACATA |
| | | ATTGGAAGTAAAACACTCCTCAACGAATGCAAAAGAAT |
| | | GGAAATCATAACAAACAGTCTCTCAGACCACAGTGCAA |
| | | TCAAATTAGAACTCAGGATTACGAAACTTACTCAAAACT |
| | | GCACAACTACATGGAAACTGAACAACCTGCTCCTGAAC |
| | | GACTACTGGGTAAATAATGAAATGAAGGCAGAAATAAA |
| | | TAAGTTTTTGAAACCAATGAGAACAAAGACACAACATA |
| | | CCAGAATCTCTGGGACACAGCTAAAACAGTG |
| 61 | L2a | TATTTTTAAGCAAAGACTTAGAGAGAGAGAGGAAGTTA |
| | (LINE/L2) | GCTATGCACATTGTGGGTGCGGTGTGGTTAGGGTGGCCC |
| | | TGAGATAGGGATATGCCTGGTGTGTTTACGGAACAGCTA |
| | | AGAGGCCAGGGAACTAGAGGAAATTGAGTGGGAAGGA |
| | | GAAGAGTAGGAGATTAGTTTTGAAAAGCAAATGGTTTG |
| | | GGAATATGGATCATGTAAGAGCACATGAGATTTTATAAG |
| | | GATTTCATTTTACTCAGGGTGAAATGAAGAAGCACTAAA |
| | | A |
| 62 | AluJb | GTTCATGCCTGTAATCCCAGTGCTTAGGGAGGCCGAGGT |
| | (SINE/Alu) | GGGTGGATTGCTTGAGCCCAGGAGTTCGAGACCAGCCTG |
| | | GACAAAATGGCAGAAACTCCATGTCTACAAAAAATACA |
| | | AAAATTAGCCGGGCATGATGTTCTGCGCCTGTAGTCCCA |
| | | GCTACTCAGGAGGCTGAGGTGGGAGGATCGCTTGAGCC |
| | | CGGGAGGCGGAGTTTGCAGTGAGCTGAGATGTCACTGC |
| | | ATTCCAGCCTGGCAGACAGAGCCAGACTCTGTCTCAAAA |
| | | GAAAAAAAGAAAAA |
| 63 | L1PA7 | ATGGAATACTATGTAGCCCTAAAAAAAGATGAGTTCATG |
| | (LINE/L1) | TCCTTTGCAGGGACATGGATGAAGCTGGAAACCATCATT |
| | | CTCAGCAAACTAACACAGGAACAGAAATCCAAACACTG |
| | | CATGTTCTCACTCATAAGTGGGAGTTGAGCAATGAGAAA |
| | | ACATGGACACAGGGAGGGGAACATCACACACTGGGGCC |
| | | TTTTGGGGAGTTGGGGGCTAGGGGAGGGATAACATTAG |
| | | GAGAAATACCTAATGTTATTTCTCCTAATATTATTTCAGC |
| | | CTAATGATGGGTTGATGGGTGCAGCAAACCAGCATGGC |
| | | CCATGTATACCTATGTAACAAACCTGCATGTTCTACATA |
| | | TGTATCACAGAACTTAAAGTATGTATATATAAAAAA |
| 64 | L1PA10 | TTAGTCTGATGGGCTTCCCTTTGTGGGTTACCTGACCTTT |
| | (LINE/L1) | CTCTCTGGCTGCCCTTAACATTTTTTCCTTCATTTCAACTT |
| | | TGGTCAGTCTGACGATTATGTGTCTTGGGGTTGCTCTTCT |
| | | CAAGGAGTATTAGTGGTGTTCTCTGTATTTCCTGAATTTG |
| | | AATATTGGCCTGTCTTGCTACGTTGGGGAGGTCCTCTGG |
| | | ATAATATCCTGAAGAGTGTTTTCCAACTTGATTCCATTCT |
| | | TCCCGTCACTTTCAGGTACACCAATCAGACGTAAGTTTG |
| | | GTCTTTTCACATAGTCCCATATTTCTTGGAGTCCTTGTTC |
| | | ATTCTTTTTCATTCTTTTTATCCCTAATCTTGTCCTGATGC |
| | | TTTATTTCATTAAGTTGATCTTCAATCTCTGATATCCTTT |
| | | CTTCCGCTTGATCGATTCGGCTATTAATACTTGTGTATGC |
| | | TTCACGAAGTTCTCATGCTATGTTTTTCAGCTCCACCAGC |
| | | TCATTTATGTTCTTCTCTAAACTGGTTACTCTAGTTAGCA |
| | | ATTCCTCTAACCTTTTTTCAAGGTTCTTAGCTTCCTTGCA |
| | | TTGGATTAGAACATGCCCCTTTAGCTCAGAGGAGTTTGT |
| | | TATTACCCACCTTCTGAAGCCTACTTCTGTCAATTTGTCA |
| | | AACTCATTCTCTGTCCAGTTTTGTTCCCTTGCTGGCAAGG |
| | | AGTTGTGATCCTTTGTAGAGGAGGTATTCTATTTTTTGGA |
| | | ATTTTCAGCTATTTTGCCCTGGTTTTTCCTCATCTTTGTGG |
| | | ATTTATCTACTTTTGGTCTTTGATGCTGGTGACCTTCGGA |
| | | TGGGGTTTTTGTGTGTGTGTCCCTTTTGTTGATGTTGATG |
| | | CTATTCCTTTCTGTTTGTTAGTTTTCCTTCTAACTGGCCCG |
| | | TCTGGTGCAGGTCTGCTGGAGTTTTCTGGAGGCCCACTC |
| | | CAGACCCTGTTTGCCTGAGTATCACCAGCGGAGCCTGCA |
| | | GGACAGCAAAGATTGCTGCTTGTTCCTTTCTCTGGAAGC |
| | | TTTATCCCAGAGGGGCACCTGCCAGATGGCAGCTGGAGC |
| | | TCTCCTGTATGAGGTGTCTGTTGACCCCTGCTGGGAGGT |
| | | GTCTGCCAGTCAGGAGGCACGGGGTCAGGGACCCACTT |
| | | GAGGAGGCAGTCTGTCCCTTAGCAGAGCTCGAGCACTGT |
| | | GCTGGGAGATCTGCTTCTCTTTTCAGAGCCAGAAGGCAG |
| | | GAATGTTTAAGTCTGTAGAACTGTGCCCACAGCCGCTCC |
| | | TTTCCCCCAGGT |
| 65 | AluSq2 | GCCGAGCGCGGTGGCTCAAGCCTGTAATCCCACCACTTT |
| | (SINE/Alu) | GGGAGGCAAAGGTGGGTGAATCACCTGAGGTCAGGAGT |
| | | TCGAGACCAGCCTGACCAACATGGTGAAACCCCATCTCT |
| | | ACTAAAAATACAAAAATTAGCCAGGCCTGGTGTCGGGT |
| | | GCCTGTAATCCCAGCTACTCAGGAGGCTGAGACAGGAG |
| | | AATCACTTGAACCCAGGAGGTGGAGGTTGCAGTAAGCC |
| | | GAGACCGTGCCCATTGCACTCCAATCTGGGCGACGAGA |
| | | GTGAAACTCCATCTTAAAAAAAAAAAAAAA |
| 66 | L1P3 | GCCATTTTCACGATATTGATTCTTCCTATCCATGAGCACG |
| | (LINE/L1) | GAATGTTTTTCCATTCGTTTCTGTCCTCTCTTATTGCCTTG |
| | | AGCAGTGGTTTGTAGATCTCCTTGAAGAGGTCCTTCACA |
| | | TCCTTTGTAAGTTGGATTCCTAGGTATTTTATTCTCTTTGT |
| | | AGCAATTGTGAATGGGAGTTCACTCATGATTTCA |
| 67 | LTR41C | CCTTAATAGTTAATCTCCTATTAATAAGTAAGTGATTCTT |
| | (LTR/ERVL) | TAATTAAACTTTCCTCATTCAAATGGCCATATGCTTTCTG |
| | | TCTCTCGACTAGATGCTGAGTGATATACCCCCCA |

TABLE 4 provides certain details of illustrative polynucleotides that can be used in a synthetic delivery system disclosed herein, including, e.g., identified genomic insertion sites and/or a number of Single nucleotide variants (SNVs or SNPs) identified in experiments disclosed herein. Certain provided polynucleotides can correspond to the sequences provided in TABLE 3 and can be used in a synthetic delivery system disclosed herein, for example, for delivery to cancer cells, such as pancreatic cancer cells.

TABLE 4

| SEQ | | | | | |
|---|---|---|---|---|---|
| ID | | No. | | | Chr |
| NO | Description | SNP | Location | Gene | location |
| 25 | AluSx | 5 | Chr20 | | |
| | (AluSx1) | | | | |
| 26 | MIRc | 0 | Chr16 | PPAC | |
| | (MIRB) | | | | |
| | (MIR) | | | | |
| 27 | AluJb | 2 | Chr7 | MAGI2 | |
| | (AluSx1) | | | | |
| 28 | AluSx1 | 2 | Chr8 | | |
| | (AluSq) | | | | |
| | (ALU) | | | | |
| 29 | AluSx | 2 | Chr3 | RAB7A | |
| | (AluSp) | | | | |
| | (Alu) | | | | |
| 30 | MLT2B4 | 1 | Chr3 | RAB7A | |
| | (MTL2B4) | | | | |
| | (ERVL) | | | | |
| 31 | AluSx1 | 5 | Chr21 | | |
| 32 | L2a | 5 | Chr21 | | |
| | (L2a/LTR40b/ | | | | |
| | MLT1J2) | | | | |
| 33 | MLT1J2 | 4 | Chr21 | | |
| | (parAluSp- | | | | |
| | FULLMTL1J2) | | | | |
| | (LTR) | | | | |
| 34 | AluSx | 2 | Chr7 | | |
| | (AluY) | | | | |
| 35 | (MIRB) | 0 | Chr14 | | |
| | (MIRc- part L2) | | | | |
| | (MIRc) | | | | |
| 36 | AluSx | 2 | Chr9 | | |
| | AluSq | | | | |
| 37 | MamR4 | 0 | Chr9 | | |
| 38 | AluSx | 1 | Chr 5 | | 178415731 |
| | (SINE/Alu) | | | | |
| 39 | MIR | 1 | Chr 2 | | 27942978 |
| | (SINE/MIR) | | | | |
| 40 | L1M1 | 6 | Chr 16 | ZNF267 | 31922257 |
| | (LINE/L1) | | | | |
| 41 | L1MEg | 1 | Chr 7 | GRB10 | 50660129 |
| | (LINE/L1) | | | | |
| 42 | AluSx | 2 | Chr 16 | DNAAF1 | 84209602 |
| | (SINE/Alu) | | | | |
| 43 | AluSz6 | 2 | Chr 2 | | 27951382 |
| | (SINE/Alu) | | | | |
| 44 | ALR/Alpha | 81 | Chr 9 | | 66797787 |
| | (Satellite/centr) | | | | |
| 45 | L1P1 | 3 | Chr 10 | | 57016162 |
| | (LINE/L1) | | | | |
| 46 | THE1B | 2 | Chr 5 | | 107782747 |
| | (LTR/ERVL- | | | | |
| | MaLR) | | | | |
| 47 | AluSg | 4 | Chr 10 | LINE1/Alu | 14247220 |
| | (SINE/Alu) | | | | |
| 48 | HERV9N-int | 9 | Chr 2 | WDPCP | 63548334 |
| | (LTR/ERV1) | | | | |
| 49 | HERV17-int | 5 | Chr 6 | AIG1 | 143409567 |
| | (LTR/ERV1) | | | | |
| 50 | LTR81B | 3 | Chr 7 | HDAC9, | 18828526 |
| | (LTR/Gypsy) | | | uncommon | |
| | | | | isoform | |
| 51 | ALR/Alpha | 7 | Chr 21 | | 14359298 |
| | (Satellite/centr) | | | | |
| 52 | ALR/Alpha | 55 | Chr 7 | | 57988111 |
| | (Satellite/centr) | | | | |
| 53 | AluSc8 | 1 | Chr18 | | 9092284 |
| | (SINE/Alu) | | | | |
| 54 | L1P3 | 1 | Chr 1 | | 160552759 |
| | (LINE/L1) | | | | |
| 55 | AluYc3 | 1 | Chr 14 | | 103067410 |
| | (SINE/Alu) | | | | |
| 56 | L1PA10 | 1 | Chr 2 | | 40056057 |
| | (LINE/L1) | | | | |
| 57 | AluSz | 1 | Chr 6 | | 153049622 |
| | (SINE/Alu) | | | | |
| 58 | REP522 | 1 | Chr 13 | | 19693028 |
| 59 | AluSc8 | 1 | Chr 18 | | 9092284 |
| | (SINE/Alu) | | | | |
| 60 | L1P3 | 1 | Chr 4 | | 36939485 |
| | (LINE/L1) | | | | |
| 61 | L2a | 1 | Chr 14 | | 21413450 |
| | (LINE/L2) | | | | |
| 62 | AluJb | 1 | Chr 18 | | 14432555 |
| | (SINE/Alu) | | | | |
| 63 | L1PA7 | 1 | Chr 2 | | 153758182 |
| | (LINE/L1) | | | | |
| 64 | L1PA10 | 1 | Chr 10 | | 87038517 |
| | (LINE/L1) | | | | |
| 65 | AluSq2 | 1 | Chr 1 | | 231207459 |
| | (SINE/Alu) | | | | |
| 66 | L1P3 | 1 | Chr 6 | | 72066634 |
| | (LINE/L1) | | | | |
| 67 | LTR41C | 1 | Chr 19 | | 40372997 |
| | (LTR/ERVL) | | | | |

In some embodiments, a synthetic delivery system disclosed herein does not elicit, substantially does not elicit, or elicits a reduced immunogenic response compared to an alternate delivery system, such as a viral vector.

In some embodiments, a synthetic delivery system disclosed herein comprises a conjugation moiety. The conjugation moiety can be attached at the 5′ end, the 3′ end, or at an internal site along the length of a polynucleotide. The conjugation moiety can be a nucleotide analogue (such as bromodeoxyuridine). The conjugation moiety can be a conjugating functional group. The conjugating functional group can be an azido group or an alkyne group. A conjugation moiety can be a modified nucleotide that facilitates conjugation to chemical compound. A conjugation moiety can be a modified nucleotide comprising a functional group that can be conjugated to a chemical compound. In some embodiments, a synthetic delivery system disclosed herein comprises a plurality of conjugation moieties. In some embodiments, the plurality of conjugation moieties are the same. In some embodiments, the plurality of conjugation moieties are different. In some embodiments, a synthetic delivery system or polynucleotide comprises a first conjugation moiety and a second conjugation moiety.

A conjugation moiety can be or can comprise a single modified nucleotide of choice (e.g., modified A, C, G, U, or T containing an azide at the 2′-position) that is incorporated site-specifically under optimized conditions (e.g., via solid-phase chemical synthesis). The conjugation moiety can be or can comprise a plurality of nucleotides containing an azide at the 2′-position that are incorporated, for example, by substituting a nucleotide during an in vitro transcription reaction (e.g., substituting UTP for 5-azido-C3-UTP). Non-limiting examples of conjugation moieties include modified UTP analogs, 5-Azidomethyl-UTP, 5-Azido-C3-UTP, 5-Azido-PEG4-UTP, 5-Ethynyl-UTP, DBCO-PEG4-UTP, Vinyl-UTP, 8-Azido-ATP, 3′-Azido-2′,3′-ddATP, 5-Azido-PEG4-CTP, 5-DBCO-PEG4-CTP, N6-Azidohexyl-3′-dATP, 5-azidopropyl-UTP, and 5-DBCO-PEG4-dCpG.

A conjugation moiety can further be derivatized through a chemical reaction such as click chemistry or any other bioconjugation reaction. A conjugation moiety can be conjugated via a chemical reaction, e.g., using click chemistry or a Staudinger reaction to chemical compound. The click chemistry can comprise a copper (I)-catalyzed [3+2]-Huisgen 1,3-dipolar cyclo-addition of alkynes and azides leading to 1,2,3-triazoles. The click chemistry can be a copper free variant of a reaction. The click chemistry can be an inverse electron-demand Diels-Alder reaction between a trans-cyclooctadiene and a tetrazine.

A polynucleotide can be conjugated to a conjugation partner (e.g., cargo) using a copper-catalyzed click reaction, e.g., copper-catalyzed 1,3-dipolar azide-alkyne cycloaddition (CuAAC) of an alkyne-functionalized conjugation partner (e.g., cargo, such as a small molecule) and an azide-functionalized nucleic acid. A linear polynucleotide can be modified at its 3′-end by a poly (A) polymerase with an azido-derivatized nucleotide. The azide can be conjugated to a conjugation partner via copper-catalyzed or strain-promoted azide-alkyne click reaction.

A polynucleotide can be conjugated to a conjugation partner (e.g., cargo) using a Staudinger reaction. For example, a polynucleotide comprising azide-functionalized nucleotide(s) can be conjugated with an alkyne-functionalized small molecule in the presence of triphenylphosphine-3,3′,3″-trisulfonic acid (TPPTS).

A polynucleotide can be conjugated to a conjugation partner (e.g., cargo) using a Suzuki-Miyaura reaction. For example, a polynucleotide comprising a halogenated nucleotide analog can be subjected to Suzuki-Miyaura reaction in the presence of a cognate reactive conjugation partner (e.g., cargo). A polynucleotide comprising 5-Iodouridine triphosphate (IUTP), for example, can be used in a catalytic system with Pd(OAc)₂ and 2-aminopyrimidine-4,6-diol (ADHP) or dimethylamino-substituted ADHP (DMADHP) to functionalize iodouridine-labeled polynucleotide in the presence of various suitable boronic acid and ester substrates. In another example, a polynucleotide comprising 8-bromoguanosine can be reacted with arylboronic acids in the presence of a catalytic system made of Pd(OAc)₂ and a water-soluble triphenylphosphan-3,3′,3″-trisulfonate ligand.

A polynucleotide conjugate can be generated using Michael addition, for example, via reaction of an electron-rich Michael Donor with an α,β-unsaturated compound (Michael Acceptor).

In some embodiments, the conjugation moiety as used herein can comprise a hapten group. A hapten group can include digoxigenin, 2,4-dinitrophenyl, biotin, avidin, or be selected from azoles, nitroaryl compounds, benzofurazans, triterpenes, ureas, thioureas, rotenones, oxazoles, thiazoles, coumarins, cyclolignans, heterobiaryl compounds, azoaryl compounds or benzodiazepines. A hapten group can include biotin. A nucleic acid comprising the conjugating moiety can further be linked to a second nucleic acid, a fluorescent moiety (such as a dye such as a quantum dot), or a conjugating partner such as a polymer (such as PEG), a macromolecule (such as a carbohydrate, a lipid, a polypeptide), for example.

In some embodiments, a synthetic delivery system disclosed herein comprises a polynucleotide conjugated to a cargo. A polynucleotide can be conjugated to, for example, a chemical compound (e.g., a small molecule), an antibody or fragment thereof, a peptide, a protein, an aptamer, a drug, a second polynucleotide, or a combination thereof.

In some embodiments, a polynucleotide is conjugated to a conjugation partner (e.g., cargo) using a chelator.

In some embodiments, a synthetic delivery system disclosed herein comprises a first conjugation moiety that is conjugated to a first conjugation partner (e.g., cargo) and a second conjugation moiety that is conjugated to a second conjugation partner (e.g., the same or a different conjugation partner and/or cargo).

### II. CARGO

In some embodiments, the present disclosure provides compositions and methods for cell-, tissue-, and/or organ-specific binding, targeting, uptake, nuclear localization and/or genomic integration of a cargo. A synthetic delivery system disclosed herein can be used to deliver a cargo to a target cell. For example, a recognition sequence of a synthetic delivery system can bind to a component (e.g., receptor) of a target cell, thereby facilitating binding, uptake, nuclear localization, and/or genomic integration of the cargo of the synthetic delivery system.

A cargo can be delivered to a membrane or surface of a target cell. A cargo can be delivered to the cytoplasm of a target cell. A cargo can be delivered to the cytosol of a target cell. A cargo can be delivered to the nucleus of a target cell.

A cargo can be or can comprise, consist essentially of, or consist of a nucleic acid cargo. A cargo can be or can comprise a DNA, such as a dsDNA or a ssDNA. A cargo can be, comprise, or encode an RNA, such as an mRNA, rRNA, tRNA, siRNA, shRNA, or ncRNA. A cargo can be or can comprise a transgene.

A cargo can be or can comprise a nucleic acid that encodes a protein. A cargo can be or can comprise a nucleic acid that encodes a therapeutic protein. A cargo can be or can comprise a nucleic acid that encodes, for example, a cytotoxic polypeptide (e.g., a caspase, such as a self-activating, inducible, or non-inducible caspase, a protein that induces p53-mediated apoptotic cell killing CASP3, CASP8, CASP9, BAX, DNA fragmentation factor (DFF) 40, or Herpes Simplex Virus Thymidine Kinase (HSV-TK)), a tumor suppressor, an antigenic peptide, an antibody or antigen-binding fragment thereof, an enzyme, a cytokine, a signaling molecule, a microbial (e.g., bacterial or viral) antigen or epitope, a polypeptide that increases an immune response, or a polypeptide that reduces an immune response. A cargo can be or can comprise a nucleic acid that encodes a diagnostic protein. A cargo can be or can comprise a diagnostic protein. A cargo can be, comprise, or encode an immunomodulatory factor. A cargo can be, comprise, or encode an immune checkpoint modulator, such as an immune checkpoint inhibitor or activator. A cargo can be, comprise, or encode a one or more neoantigens or tumor associated antigens. A cargo can be, comprise, or encode a factor that alters sensitivity of a target cell to a drug. A cargo can be a nucleic acid that encodes an immune receptor, such as a chimeric antigen receptor (CAR).

A cargo gene that encodes a protein can be operatively linked to and/or under regulatory control of a promoter, such as a promoter that is also part of the cargo. The promoter can be, for example, constitutive, inducible, and/or cell type-specific for expression of an RNA and/or protein in the target cell. A cargo can be or can comprise a nucleic acid that does not encode a protein.

In some embodiments, a cargo encodes an endogenous protein or functional RNA molecule that is either lacking or functionally impaired in a subject. In some embodiments, a subject carries a mutation in a gene, such as cancerous mutation, a loss-of function mutation, or a gain-of-function mutation that can be treated by supplying a therapeutic cargo, for example, a transgene encoding the lacking or functionally impaired protein or RNA, or to inhibit a pathogenic protein or RNA.

In some embodiments, a cargo is a nucleic acid that inserts into the genome and causes genetic or genomic instability and/or cell death (e.g., of a cancer target cell).

In some embodiments, a cargo is a nucleic acid that includes a natural or unnatural nucleotide analogue or base.

A cargo can be or can comprise a polypeptide. The polypeptide can be a therapeutic polypeptide. The polypeptide can be a diagnostic polypeptide. The polypeptide can be an anti-cancer polypeptide. The polypeptide can be a cytotoxic peptide, for example, for inducing cell death, of the target cell, such as apoptosis, necroptosis, or necrosis. A cargo can be or can comprise, for example, a tumor suppressor, an antigenic peptide, an antibody or antigen-binding fragment thereof, an enzyme, a cytokine, a signaling molecule, a polypeptide that increases an immune response, or a polypeptide that reduces an immune response.

A cargo can be or can comprise a small or organic molecule (e.g., small molecule therapeutic, or fluorescent dye). The small or organic molecule can be or can comprise a therapeutic agent. The small or organic molecule can be or can comprise a diagnostic agent. The small or organic molecule can be or can comprise a cytotoxic agent. In some embodiments, the small or organic molecule is or comprises a radiolabeled or radioactive cytotoxic molecule. The small or organic molecule can be an anti-cancer drug. The small or organic molecule can be a kinase inhibitor. A small molecule can be a compound (e.g., organic compound) with a molecular weight of, for example, at most 500, at most 600, at most 700, at most 800, at most 900, at most 1000, at most 1100, at most 1200, or at most 1500 daltons.

A cargo can be or can comprise a therapeutic agent. Non-limiting examples of therapeutic agents that a cargo can be or can comprise include vaccines (e.g., mRNA vaccines), AKT inhibitors, alkylating agents, anti-angiogenic agents, antibiotic agents, antifolates, anti-hormone therapies, anti-inflammatory agents, antimetabolites, anti-VEGF agents, apoptosis promoting agents, aromatase inhibitors, ATM regulators, biologic agents, BRAF inhibitors, BTK inhibitors, a chimeric antigen receptor (e.g., a nucleotide encoding a CAR), CDK inhibitors, cell growth arrest inducing-agents, chemotherapeutic agents, cytokines, cytotoxic drugs, demethylating agents, differentiation-inducing agents, estrogen receptor antagonists, gene therapy agents, growth factor inhibitors, growth factor receptor inhibitors, HDAC inhibitors, heat shock protein inhibitors, hematopoietic stem cell transplantation (HSCT), hormones, hydrazine, immune checkpoint inhibitors, immumomodulators, immunosuppressants, kinase inhibitors, KRAS inhibitors, matrix metalloproteinase inhibitors, MEK inhibitors, mitotic inhibitors, mTOR inhibitors, multi-specific (e.g., bispecific) immune cell engagers, multi-specific (e.g., bispecific) killer cell engagers, multi-specific (e.g., bispecific) T cell engagers, nitrogen mustards, oncolytic viruses, oxazaphosphorines, p53 reactivating agents, plant alkaloids, platinum-based agents, proteasome inhibitors, purine analogs, purine antagonists, pyrimidine antagonists, radiation therapy, ribonucleotide reductase inhibitors, signal transduction inhibitors, RNA silencing (e.g., RNAi) agents, gene editing agents, a CRISPR/Cas systems or a component thereof, an RNA replacement therapy, a protein replacement therapy, a gene therapy, antibody drug conjugates, surgery, taxanes, therapeutic antibodies, topoisomerase inhibitors, tyrosine kinase inhibitors, vinca alkaloids, and nucleic acids encoding any of the applicable preceding agents. A cargo can be, for example, an anti-cancer therapeutic agent.

A cargo can be or can comprise a drug. A cargo can be or can comprise a non-cancer therapeutic, for example, a therapeutic for a metabolic disease, autoimmune disease, neurological disease, or degenerative disease. A cargo can be or can comprise, for example, a vaccine (e.g., cancer vaccine), a drug, an immunotherapy, an immune checkpoint inhibitor, a kinase inhibitor, a small molecule, a chemotherapeutic agent, a radiotherapy, a biologic, or any combination thereof.

A cargo that is or comprises a therapeutic agent can modulate (e.g., increase or decrease) activity of a target gene (e.g., an aberrantly expressed gene), or a product encoded by the target gene, such as a protein or RNA. A cargo that is or comprises a therapeutic agent can modulate (e.g., increase or decrease) expression of a target gene (e.g., an aberrantly expressed gene). A cargo that is or comprises a therapeutic agent can modulate (e.g., increase or decrease) activity of a ligand or receptor of a target gene (e.g., an aberrantly expressed gene). In some embodiments, a cargo that is or comprises a therapeutic agent can alter the gene product of an aberrantly-expressed gene, e.g., by targeting the gene product, the transcript of the gene, or epigenetic factors that influence a property of the gene (e.g., expression). Non-limiting examples include targeting the protein that the gene encodes, reducing expression levels of the gene using gene therapy or RNAi, and using RNA vaccines to establish an immune response.

In some embodiments, a cargo can be or can comprise a dye (e.g., a fluorescent dye), a radioactive isotope, or a contrast agent for use in diagnostic imaging in vivo.

In some embodiments, a cargo is a detectable label (e.g., a fluorescent label, quantum dot, a Quasar Dye, or a radioactive label). A detectable label can be a radioactive label (e.g., a radioactive isotope) that facilitates the visualization of distribution of the herein disclosed constructs via positron emission tomography (PET) or single photon emission computed tomography (SPECT). A detectable moiety can comprise a radioactive isotope of at least one element.

A cargo can be or can comprise a radionucleotide. A cargo can be or can comprise a polymer. A cargo can be or can comprise a nanoparticle. In some embodiments a cargo can cause DNA damage either from outside the target cell or from within the target cell.

A cargo can be covalently or noncovalently attached to a polynucleotide or synthetic delivery system.

In some embodiments, a synthetic delivery system does not include a cargo. For example, in some embodiments a polynucleotide disclosed herein can directly exert an effect on a target cell, e.g., by inducing cell death after binding, uptake, nuclear trafficking, and/or genomic integration of the polynucleotide. In some embodiments, a synthetic delivery system acts as an antagonist to reduce uptake of ctDNA.

### III. TARGET CELLS

Compositions and methods disclosed herein can be used for delivering a cargo to a target cell. For example, uptake of the synthetic delivery system or cargo by the target cell can be induced by contacting the target cell with a synthetic delivery system and/or polynucleotide disclosed herein. Methods disclosed herein can comprise delivering a cargo to a target cell.

A target cell can be a primary cell. A target cell can be a cancer cell, for example, a hematologic tumor cell or a solid tumor cell. A target cell can be a cell line.

A target cell can be a eukaryotic cell. A target cell can be a mammalian cell. A target cell can be a human cell.

A target cell can be a leukocyte, such as a lymphoid or myeloid cell. A target cell can be a plasma cell. A target cell can be a multiple myeloma cell. A target cell can be a B cell. A target cell can be a T cell. A target cell can be a lymphocyte. A target cell can be a monocyte or a macrophage. A target cell can be a neoplastic myeloid cell. A target cell can be a neoplastic lymphoid cell. A target cell can be a multiple myeloma cell. A target cell can be a leukemia cell. A target cell can be a lymphoma cell.

A target cell can be a pancreatic cell. A target cell can be an exocrine pancreatic cell. A target cell can be an endocrine pancreatic cell. A target cell can be a pancreatic cancer cell. A target cell can be a bile duct cell. A target cell can be a gallbladder cell.

A target cell can be a gastrointestinal cell. A target cell can be, for example, an intestine cell, such as a colon cell. A target cell can be a colorectal cancer cell.

A target cell can be, for example, a lymphoma, leukemia, lung cancer, colon cancer, or brain cancer cell.

In some embodiments, a target cell is a hematologic or hematopoietic cell. In some embodiments, a target cell is a bone marrow-derived cell.

In some embodiments, a target cell is a solid tumor cell, a liquid tumor cell, a hematologic cancer cell, a leukemia cell, a lymphoma cell, a B cell, a bladder cancer cell, brain cancer (e.g., astrocytoma, glioblastoma, meningioma, or oligodendroglioma) cell, breast cancer (e.g., ER+, PR+, HER2+, or triple-negative breast cancer) cell, bone cancer cell, cervical cancer cell, colon cancer cell, colorectal cancer cell, esophageal cancer cell, head and neck cancer cell, kidney cancer cell, liver cancer cell, lung cancer cell, medullary thyroid cancer cell, mouth cancer cell, nose cancer cell, ovarian cancer (e.g., mucinous, endometrioid, clear cell, or undifferentiated) cell, pancreatic cancer cell, renal cancer cell, skin cancer cell, stomach cancer cell, throat cancer cell, thyroid cancer cell, uterus cancer cell, carcinoma cell, sarcoma cell, adenoma cell, adenocarcinoma cell, cancer cell of unknown primary tissue, or neuroendocrine tumor cell.

In some embodiments, a synthetic delivery system, polynucleotide, nucleic acid sequence, first nucleic acid sequence, second nucleic acid sequence, recognition sequence, or integration sequence disclosed herein, or a functional fragment thereof, can specifically or preferentially mediate binding, uptake, and/or genomic integration of a target cell. For example, in some embodiments, a synthetic delivery system disclosed herein can bind to, enter, localize to the nucleus of, and/or integrate into the genome of a target cell disclosed herein at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold more than a control cell. The control cell can be, for example, a different cell or tissue type. The control cell can be, for example, a corresponding cell type that is a non-cancer cell, as compared to a target cell that is a cancer cell.

In some embodiments, specificity for a target cell can reduce off-target and unwanted side effects, for example, as compared to conventional cargo delivery system (e.g., nanoparticles or viral vectors).

### IV. PHARMACEUTICAL COMPOSITIONS

Compositions disclosed herein can be pharmaceutical compositions (e.g., formulations), and methods can utilize pharmaceutical compositions. For example, in some embodiments provided is a pharmaceutical composition comprising a compound, such as a synthetic delivery system, polynucleotide, cargo, therapeutic agent, and/or diagnostic agent and a pharmaceutically-acceptable excipient, carrier, vehicle, or diluent. In some embodiments, the disclosure provides a pharmaceutical composition comprising a synthetic delivery system or polynucleotide disclosed herein and a pharmaceutically-acceptable excipient, carrier, vehicle, or diluent.

An active agent or compound (e.g., synthetic delivery system, polynucleotide, cargo, therapeutic agent, and/or diagnostic agent) described herein can be combined with other chemical and/or pharmaceutically acceptable components, such as carriers, stabilizers, diluents, dispersing agents, suspending agents, thickening agents, antioxidants, solubilizers, buffers, osmolytes, salts, surfactants, amino acids, encapsulating agents, bulking agents, cryoprotectants, and/or excipients, to form a pharmaceutical composition. The composition can facilitate administration of any active agent (e.g., polynucleotide or synthetic delivery system) described herein to an organism. Compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, and topical administration (e.g., for use in melanoma). A pharmaceutical composition can be administered in a local or systemic manner, for example, via injection of the nucleic acid molecules as described herein directly into an organ, optionally in a depot.

Parenteral injections can be formulated for bolus injection or continuous infusion. The pharmaceutical compositions as described herein can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a compound, such as a synthetic delivery system, polynucleotide, cargo, therapeutic agent, and/or diagnostic agent described herein in water soluble form. Suspensions of the active compound(s) can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents that increase the solubility and/or reduce the aggregation of compounds to allow for the preparation of highly concentrated solutions. Alternatively or additionally, the compounds can be lyophilized or in powder form for re-constitution with a suitable vehicle, e.g., sterile pyrogen-free water, before use. In some cases, a compound, such as a synthetic delivery system, polynucleotide, cargo, therapeutic agent, and/or diagnostic agent is injected directly into a tissue. A compound, such as a synthetic delivery system, polynucleotide, cargo, therapeutic agent, and/or diagnostic agent described herein can be administered to a subject, home, target, migrate to, penetrate an organ, e.g., the pancreas, or a cell, e.g. multiple myeloma cell.

A compound, such as a synthetic delivery system, polynucleotide, cargo, therapeutic agent, and/or diagnostic agent of the disclosure can be applied directly to an organ, or an organ tissue or cells, such as the lung or pancreas, during a surgical procedure. The compound, such as a synthetic delivery system, polynucleotide, cargo, therapeutic agent, and/or diagnostic agent can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers, and preservatives.

In practicing the methods of treatment or use provided herein, therapeutically-effective amounts of the compound, such as a synthetic delivery system, polynucleotide, cargo, therapeutic agent, and/or diagnostic agent described herein can be administered in pharmaceutical compositions to a subject having a disease or condition to be treated (e.g., cancer). In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which can facilitate processing of the active compounds into preparations that can be used pharmaceutically. Formulation can be modified and adapted depending upon the route of administration chosen. Pharmaceutical compositions comprising a polynucleotide or synthetic delivery system described herein can be manufactured, for example, by expressing the polynucleotide in a recombinant system, purifying the polynucleotide or a complex comprising the polynucleotide, lyophilizing the nucleic acid molecule, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form. Pharmaceutical compositions can contain solubilizers, stabilizers, tonicity enhancing agents, buffers, and preservatives.

Methods for the preparation of compositions comprising the compounds described herein (e.g., synthetic delivery systems, polynucleotides, nucleic acids, or a polynucleotide attached to a cargo) as described herein include formulating the compounds with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of auxiliary substances, such as wetting or emulsifying agents, pH buffering agents, and other pharmaceutically-acceptable additives.

Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions.

The disclosed compositions can optionally comprise pharmaceutically-acceptable preservatives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Non-limiting examples of pharmaceutically-acceptable carriers include saline, Ringer's solution, and dextrose solution. In some embodiments, the pH of the solution can be from about 5 to about 8, from about 7 to 8, or from about 7 to about 7.5. Further carriers include sustained release preparations such as semipermeable matrices of solid hydrophobic polymers containing the compound. The matrices can be in the form of shaped articles, for example, films, liposomes, microparticles, or microcapsules.

A pharmaceutical composition described herein can be in a unit dosage form suitable for a single administration of a precise dosage. In unit dosage form, the formulation can be divided into unit doses containing appropriate quantities of one or more synthetic delivery systems, antagonists, or therapeutic agents. The unit dosage can be in the form of a package containing discrete quantities of the formulation. Non-limiting examples are packaged injectables, vials, and ampoules. An aqueous suspension composition disclosed herein can be packaged in a single-dose non-reclosable container. Multiple-dose reclosable containers can be used, for example, in combination with or without a preservative. A formulation for injection disclosed herein can be present in a unit dosage form, for example, in ampoules, or in multi dose containers with a preservative.

### V. METHODS

Disclosed herein, in some embodiments, is a method of delivering a cargo to a target cell. Various assays can be used to demonstrate receptor binding, uptake, nuclear localization, and/or genomic integration of a cargo, synthetic delivery system, or polynucleotide.

The disclosure provides methods for the identification, characterization, isolation, synthesis, in vitro testing, and in vivo testing of cell- and tissue-type specific synthetic delivery systems.

Methods disclosed herein can comprise administering a therapeutic agent to a subject. The subject can be a subject in need thereof, for example, that has a condition to be treated. A therapeutic agent administered to the subject can be or can comprise a synthetic delivery system disclosed herein, e.g., for delivery of a cargo.

In some embodiments, a method utilizing a synthetic delivery system disclosed herein can be used to treat cancer, an inflammatory disease, or an autoimmune disease. In some embodiments, a method of delivering a cargo disclosed herein utilizing a synthetic delivery system can be used to administer a vaccine, for example, the cargo can comprise a vaccine or component thereof, for example, an antigen and/or adjuvant, or a nucleic acid sequence encoding an antigen and/or adjuvant.

A synthetic delivery system disclosed herein can be used to treat a solid tumor. A synthetic delivery system disclosed herein can be used to treat a liquid tumor, e.g., a hematologic cancer. A synthetic delivery system disclosed herein can be used to treat any type of cancer disclosed herein.

Methods of the disclosure can be used to treat a condition in a subject in need thereof, such as a cancer. The cancer can be a solid tumor or a liquid cancer, e.g., leukemia or lymphoma. In some embodiments, the cancer is a solid tumor. In some embodiments, the cancer is a hematological cancer, for example, a B cell cancer, such as multiple myeloma. In some embodiments, the cancer is a pancreatic cancer. In some embodiments, the cancer is colon cancer or colorectal cancer. In some embodiments, the cancer is a lung cancer.

In some embodiments, the cancer comprises bladder cancer, brain cancer (e.g., astrocytoma, glioblastoma, meningioma, or oligodendroglioma), breast cancer (e.g., ER+, PR+, HER2+, or triple-negative breast cancer), bone cancer, cervical cancer, colon cancer, colorectal cancer, esophageal cancer, head and neck cancer, kidney cancer, liver cancer, lung cancer, medullary thyroid cancer, mouth cancer, nose cancer, ovarian cancer (e.g., mucinous, endometrioid, clear cell, or undifferentiated), pancreatic cancer, renal cancer, skin cancer, stomach cancer, throat cancer, thyroid cancer, or uterus cancer. In some embodiments, the cancer comprises bladder cancer, brain cancer, breast cancer, colon cancer, colorectal cancer, lung cancer, or ovarian cancer. In some embodiments, the cancer is lung cancer. In some embodiments, the cancer is brain cancer. In some embodiments, the cancer is breast cancer, e.g., triple-negative breast cancer. In some embodiments, the cancer is ovarian cancer. In some embodiments, the cancer is bladder cancer.

In some embodiments, the cancer is a carcinoma. In some embodiments, the cancer is a sarcoma. In some embodiments, the cancer is an adenoma. In some embodiments, the cancer is an adenocarcinoma.

In some embodiments, the cancer is of unknown primary tissue. In some embodiments, a method disclosed herein is used to identify the primary tissue type. In some embodiments, the cancer is a neuroendocrine tumor.

A treatment can comprise administering to a subject a synthetic delivery system. A treatment can comprise administering to a subject a synthetic delivery system of the disclosure intravenously, subcutaneously, intramuscularly, by inhalation, dermally, intradermally, topically, orally, sublingually, intrathecally, transdermally, intranasally, intracerebrally, intraspinally, intraarticularly, ophthalmically, rectally, via a peritoneal route, or directly into the brain, e.g., via an intracerebral ventricle route. A synthetic delivery system can be administered locally. A synthetic delivery system can be administered systemically. A synthetic delivery system can be administered parenterally. A synthetic delivery system or antagonist can be administered via absorption through epithelial or mucocutaneous linings (e.g., oral mucosa, rectal and/or intestinal mucosa).

A synthetic delivery system or therapeutic agent described herein can be administered before, during, or after the occurrence of a disease or condition, and the timing of administering a composition containing the synthetic delivery system or therapeutic agent can vary. For example, the composition can be used as a prophylactic and can be administered continuously to subjects with a propensity to conditions or diseases in order to lessen a likelihood of the occurrence of the disease or condition. The composition can be administered to a subject already suffering from a disease or condition, in an amount sufficient to cure or at least partially arrest the symptoms of the disease or condition, or to cure, heal, improve, or ameliorate the condition. The composition can be administered to a subject during or as soon as practical after the onset of the symptoms.

Multiple synthetic delivery systems or therapeutic agents disclosed herein can be administered in any order or simultaneously. If simultaneously, the multiple synthetic delivery systems or therapeutic agents can be provided in a single, unified form, or in multiple forms, for example, as multiple separate injections or infusions. The synthetic delivery systems or therapeutic agents can be packed together or separately, in a single package or in a plurality of packages. One or all of the synthetic delivery systems or therapeutic agents can be given in multiple doses.

In some embodiments, a nucleic acid cargo or a component of a polynucleotide is integrated into a target cell genome at a location identified in TABLE 2, TABLE 4, TABLE 7, or TABLE 8. In some embodiments, a nucleic acid cargo or a component of a polynucleotide is integrated into a target cell genome at Chr2: 32916224-32916626. In some embodiments, a nucleic acid cargo or a component of a polynucleotide is integrated into a target cell genome at Ch16: 32628381-32629000.

The methods and compositions of the present disclosure can be useful for altering the phenotype of a target cell and/or a target cell population. In some cases, phenotypic alteration of target cells in a subject can improve the therapeutic and/or clinical response of the subject to a therapeutic intervention. In some embodiments, methods and compositions of the disclosure can be used to alter sensitivity of a target cell or target cell population to a drug, for example, increase sensitivity of cancer cells to an anti-cancer drug, or limiting or reducing a decrease in sensitivity of cancer cells to the anti-cancer drug.

A composition or method of the present disclosure can be used to elicit an immune response in a subject, e.g., upon administration of a synthetic delivery system. For example, an immune response can be elicited by administering nucleic acid constructs that comprise a cargo nucleic acid sequence coding for one or more antigenic or immunogenic peptides or proteins, and such immunogenic peptides or proteins can elicit an immune response in the subject upon expression. In some embodiments, a synthetic delivery system elicits an immune response based on the presence of cytosolic nucleic acid (e.g., DNA). In some embodiments, a synthetic delivery system elicits an immune response based on expression of a transgene from a nucleic acid cargo, for example, encoding a cytokine, antigen, or other immunomodulatory agent.

In some embodiments, the high specificity and/or efficiency of delivery to target cells (e.g., multiple myeloma cells) by a synthetic delivery system disclosed herein facilitates use of the synthetic delivery system as a therapeutic vector. For example, in some embodiments a synthetic delivery system facilitates preferential delivery to target cells (e.g., hematologic cancer cells, such as multiple myeloma cells), with lower or substantially no delivery non-target cells (e.g., bone marrow cells that are not hematologic cancer cells, multiple myeloma cells, precursors thereof, cancer stem cells, or a combination thereof).

In some embodiments, a nucleic acid cargo or a component of a polynucleotide is integrated into a target cell genome via homologous recombination or homology-directed repair. In some embodiments, the sequence of a polynucleotide disclosed herein can, to a substantial degree, define the directionality of insertion. In some embodiments, the polynucleotide includes one or more guide nucleic acid sequences to facilitate insertion of at a specific insertion site within the genome of a target cell.

In some embodiments, a synthetic delivery system or polynucleotide of the present disclosure can be used for diagnostic and monitoring purposes in various chronic, infectious or inherited (e.g., genetic) diseases, including cancer and certain disorders related to, for example, blood cells (e.g., anemia, thalassemia, hemophilia, or platelet disorders). In some cases, the presence of a cell and/or tissue specific recognition sequence as disclosed herein can be used as a biomarker for a particular disease or condition and/or can be used to monitor response to a particular therapeutic intervention (e.g., chemotherapy, targeted therapy, immunotherapy, cell therapy, and/or gene therapy). In some embodiments, a synthetic delivery system or polynucleotide disclosed herein can be used as a diagnostic agent or in a diagnostic method. For example, the integration of a polynucleotide or a part thereof into a genome of a cell can be used as a measurement or marker to determine the degree of integration of a cargo nucleic acid sequence (e.g., a therapeutic gene sequence) into a genome. In some embodiments, cell targeting and genomic integration of a polynucleotide, nucleic acid sequence, cargo, or synthetic delivery system, or component thereof described herein can be used as a marker for a particular biological effect. In an example, the degree of uptake or genomic integration of a cargo, polynucleotide, or part thereof can be a marker or measurement for a therapeutic effect, e.g., cell killing, cargo delivery, or expression of a nucleic acid cargo.

In some embodiments, a synthetic delivery system is used to deliver therapeutic and/or diagnostic cargos to a specific cell, cell type, tissue, tissue type, or organ of interest.

In some embodiments, a synthetic delivery system is used to visualize and/or track a disease or condition (e.g., cancer) in vivo, e.g., by delivering a chemical dye (e.g., a fluorescent dye) or a radioactive isotope to one or more cells associated with the disease or conditions. In another example, tumor cells can be visualized and tracked in vivo by delivering, for example, a chemical dye (e.g., a fluorescent dye), a radioactive isotope, or contrast agent to the tumor site(s) (e.g., primary tumor site and/or metastatic sites) by using a synthetic delivery system disclosed herein.

In some embodiments, the modular nature of synthetic delivery systems disclosed herein facilitates use to prevent, treat, and/or diagnose a variety of diseases and conditions that can include chronic, metabolic, and infectious diseases, or conditions such as cancer. A method can comprise administering an effective amount of a synthetic delivery system, polynucleotide, or pharmaceutical composition to a subject. In some embodiments, an effective amount is an amount sufficient to relieve (e.g., partially or fully relieve) one or more of the signs or symptoms of the disease or condition being treated.

A synthetic delivery system, polynucleotide, or pharmaceutical composition can be administered in combination with one or more other therapeutic agents (e.g., small molecule drugs, immunotherapeutic agents (e.g., immune check point blocker), therapeutic antibodies, or any other therapeutic agent disclosed herein.

Non-limiting examples therapeutic agents that can be administered to a subject in combination with a synthetic delivery system or polynucleotide disclosed herein include anti-cancer therapeutic agents, vaccines (e.g., mRNA vaccines), cancer vaccines, AKT inhibitors, alkylating agents, anti-angiogenic agents, antibiotic agents, antifolates, anti-hormone therapies, anti-inflammatory agents, antimetabolites, anti-VEGF agents, apoptosis promoting agents, aromatase inhibitors, ATM regulators, biologic agents, BRAF inhibitors, BTK inhibitors, CAR-T cells, CAR-NK cells, CDK inhibitors, cell growth arrest inducing-agents, cell therapies, chemotherapy, cytokine therapies, cytotoxic drugs, demethylating agents, differentiation-inducing agents, estrogen receptor antagonists, gene therapy agents, growth factor inhibitors, growth factor receptor inhibitors, HDAC inhibitors, heat shock protein inhibitors, hematopoietic stem cell transplantation (HSCT), hormones, hydrazine, immune checkpoint inhibitors (e.g., immune checkpoint inhibitors targeting the PD-1/PD-L1 axis, or other immune checkpoints such as cytotoxic T lymphocyte-associated molecule 4 (CTLA-4) or LAG-3, TIM-3, TIGIT, VISTA, or B7/H3), immunomodulators, kinase inhibitor, KRAS inhibitors, matrix metalloproteinase inhibitors, MEK inhibitors, mitotic inhibitors, mTOR inhibitors, multi-specific (e.g., bispecific) immune cell engagers, multi-specific (e.g., bispecific) killer cell engagers, multi-specific (e.g., bispecific) T cell engagers, nitrogen mustards, oncolytic viruses, oxazaphosphorines, p53 reactivating agents, plant alkaloids, platinum-based agents, proteasome inhibitors, purine analogs, purine antagonists, pyrimidine antagonists, radiation therapy, ribonucleotide reductase inhibitors, signal transduction inhibitors, RNA silencing (e.g., RNAi) agents, gene editing agents, a CRISPR/Cas systems or a component thereof, an RNA replacement therapy, a protein replacement therapy, a gene therapy, antibody drug conjugates, surgery, taxanes, therapeutic antibodies, topoisomerase inhibitors, transgenic T cells, tyrosine kinase inhibitors, vinca alkaloids, and nucleic acids encoding any of the applicable preceding agents.

The methods and compositions of this disclosure can be used to prevent, treat, arrest, reverse, or ameliorate the symptoms of a condition. The treatment can comprise treating a subject (e.g., an individual, a domestic animal, a wild animal, or a lab animal afflicted with a disease or condition) with a synthetic delivery system, polynucleotide, or pharmaceutical composition disclosed herein. The disease can be or comprise a cancer or tumor. In treating the condition, the delivery system, polynucleotide, or pharmaceutical composition of the present disclosure can be contacted to the tumor or cancerous cells. The subject can be a mammal. The subject can be a human. Subjects can be, for example, humans; non-human primates such as chimpanzees, and other apes and monkey species; farm animals, cattle, horses, sheep, goats, and swine; or domestic animals such as rabbits, dogs, and cats; laboratory animals including rodents, such as rats, mice, and guinea pigs. In some embodiments, the subject is a non-rodent subject. A subject can be a female subject. A subject can be a male subject. Treatment can be provided to the subject before, at, or after clinical onset of disease.

Disclosed herein, in some embodiments, is a method of delivering a cargo to a target cell. The method can comprise contacting a target cell or a receptor on the target cell with a synthetic delivery system disclosed herein that comprises a polynucleotide.

Compositions and methods disclosed herein can utilize receptors. Methods of the disclosure can comprise, for example, delivering a cargo to a target cell by contacting a receptor with a synthetic delivery system or a polynucleotide that binds to the receptor, and/or administering an agent that binds to and/or induces uptake via a receptor. A receptor can be expressed by a target cell disclosed herein. In some embodiments, expression of the receptor is specific to a target cell, target cell type, or target cell population, thereby facilitating specific delivery of a cargo to the target cell, target cell type, or target cell population. In some embodiments, expression of the receptor is relatively higher on a target cell, target cell type, or target cell population than a control cell, control cell type, or control cell population, thereby facilitating delivery of a cargo to the target cell, target cell type, or target cell population. In some embodiments, expression of the receptor is not specific to a target cell, target cell type, or target cell population. Binding of a synthetic delivery system or a polynucleotide to a receptor disclosed herein can induce uptake of the synthetic delivery system, polynucleotide, or cargo associated therewith by, for example, endocytosis, receptor-mediated endocytosis, clathrin-dependent endocytosis, caveolae-dependent endocytosis, CLIC/GEEC pathway uptake, lipid-raft associated endocytosis, phagocytosis, macropinocytosis, micropinocytosis, pinocytosis, or endosomal recycling.

A receptor can be or can comprise a MHC protein or a component thereof. A receptor can be or can comprise a human leukocyte antigen or a component thereof. A receptor can be or can comprise a class I MHC protein or a component thereof. For example, a receptor can be or can comprise HLA-A or a component thereof. A receptor can be or can comprise a protein that interacts with MHC class I molecules, for example, APLP2.

A receptor can be or can comprise a G-protein coupled receptor (GPCR) or a component thereof. The GPCR can be a class A (rhodopsin-like), class B (e.g., secretin and adhesion subfamilies), class C (glutamate), or class F (Frizzled) family or subfamily GPCR. A GPCR can be a GPCR that elicits a cyclic adenosine 3,5-monophosphate (cAMP) response, calcium mobilization, or phosphorylation of extracellular regulated protein kinases 1/2 (pERK1/2). For example, a receptor can be or can comprise CD97 or a component thereof.

A receptor can be or can comprise an adhesion protein or a component thereof. For example, a receptor can be or can comprise CD97 or a component thereof, PTK7 or a component thereof, ITGB5 or a component thereof, ITGA2B or a component thereof, or αVβ5 integrin or a component thereof.

A receptor can be or can comprise an integrin or a component thereof. A receptor can be or can comprise, for example, ITGB5 or a component thereof, ITGA2B or a component thereof, or αVβ5 integrin or a component thereof.

A receptor can be or can comprise a tyrosine phosphatase or a component thereof. A receptor can be or can comprise a protein tyrosine phosphatase or a component thereof. A receptor can be or can comprise a protein tyrosine phosphatase receptor or a component thereof. A receptor can be or can comprise, for example, PTPRF or a component thereof.

A receptor can be or can comprise an ion channel or a component thereof. A receptor can comprise a sensory neuron ion channel, such as a nociceptor ion channel. A receptor can comprise a function in mechanosensation. A receptor can be or can comprise, for example, TMEM120A or a component thereof.

A receptor can be or can comprise a mineral transporter or a component thereof. A receptor can be or can comprise a ferroxidase or a component thereof. A receptor can be or can comprise, for example, ceruloplasmin or a component thereof.

A receptor can be or can comprise a metalloprotein or a component thereof. A receptor can be or can comprise, for example, ceruloplasmin or a component thereof.

A receptor can be or can comprise a nutrient transporter or a component thereof. A receptor can be or can comprise a monocarboxylate transporter or a component thereof. A receptor can be or can comprise, for example, SLC16A1 or a component thereof. A receptor can be or can comprise an amino acid transporter or a component thereof. A receptor can be or can comprise, for example, SLC7A5 or a component thereof.

A receptor can be or can comprise a receptor of fibronectin, fibrinogen, plasminogen, prothrombin, thrombospondin, vitronectin, or a combination thereof. For example, a receptor can be or can comprise ITGA2B or a component thereof.

A receptor can be or can comprise an ATPase or a component thereof. A receptor can be or can comprise a sodium/potassium transporting ATPase or a component thereof. A receptor can be or can comprise, for example, ATP1B1 or a component thereof. A receptor can be or can comprise a calcium transporter or a component thereof. A receptor can be or can comprise an ion transporting ATPase or a component thereof. A receptor can be, for example, ATP2B4 or a component thereof.

A receptor can be or can comprise an Armadillo-like protein, for example, comprising one or more armadillo repeats. A receptor can be or can comprise a member of a p120(ctn)/plakophilin subfamily of Armadillo-like proteins, including for example CTNND1, CTNND2, PKP1, PKP2, PKP4, or ARVCF. In some embodiments a receptor is PKP4.

A receptor can be or can comprise an APP (amyloid precursor protein) family member, for example, APP, APLP1, or APLP2. In some embodiments, a receptor is APLP2.

A receptor can be or can comprise a kinase. A receptor can be or can comprise a lipid kinase. A receptor can be or can comprise a Phosphatidylinositol kinase. A receptor can be or can comprise a phosphatidylinositol-5-phosphate 4-kinase family member. A receptor can be capable of catalyzing the phosphorylation of phosphatidylinositol-5-phosphate on the fourth hydroxyl of the myo-inositol ring to form phosphatidylinositol-5,4-bisphosphate. A receptor can be or can comprise PIP4K2A.

A receptor can be or can comprise P142A or a component thereof.

In some embodiments, a particular cargo or type of cargo disclosed herein can be delivered by targeting a synthetic delivery system to a receptor or type of receptor disclosed herein, for example, targeting via a polynucleotide and/or recognition sequence.

In some embodiments, targeting a synthetic delivery system to a receptor or type of receptor disclosed herein can increase specific binding, uptake, nuclear localization, and/or genomic integration of the synthetic delivery system, polynucleotide, and/or cargo by target cells relative to control cells.

In some embodiments, a receptor comprises, consists essentially of, or consists of a single protein, receptor, or subunit disclosed herein. In some embodiments, a receptor comprises, consists essentially of, or consists of two proteins or subunits disclosed herein, or two receptors, proteins, or subunits contribute to uptake. In some embodiments, a receptor comprises, consists essentially of, or consists of three proteins or subunits disclosed herein, or three receptors, proteins, or subunits contribute to uptake.

Methods disclosed herein can include measuring an expression level of a receptor that is associated with uptake of a ctDNA, polynucleotide, transposable element, or synthetic delivery system. For example, a biological sample can be assayed to determine whether the receptor is expressed in the biological sample at a level that is associated with uptake of the ctDNA, polynucleotide, transposable element, or synthetic delivery system. The assay can measure expression of the receptor at protein level and/or mRNA level, e.g., can quantify or qualify an expression level of the receptor protein, or an mRNA encoding the receptor. Any suitable assay can be used to determine the expression level of the receptor. Non-limiting examples of assays that can be used include immunohistochemistry, ELISA, multiplex immunoassay, mass spectrometry (e.g., targeted or untargeted), fluorescence microscopy, flow cytometry, quantitative PCR (e.g., quantitative reverse transcriptase real time PCR), digital PCR, and RNA sequencing. In some embodiments, the assay is quantitative. In some embodiments, the assay is semi-quantitative, for example, provides an expression level relative to a control. A biological sample can comprise or can be a liquid. A biological sample can be a liquid biopsy. A biological sample can be or can comprise, for example, blood (e.g., whole blood). A biological sample can be or can comprise a solid. A biological sample can be or can comprise a solid tissue sample from any organ or tissue. A biological sample can be or can comprise a biopsy that comprises tumor tissue or is suspected to comprise tumor tissue. A biological sample can comprise tumor tissue, for example, of any cancer or tumor type disclosed herein. A biological sample can comprise cancer cells, for example, of any cancer or tumor type disclosed herein. A biological sample can comprise predominantly cells from a specific organ or from a tissue within a specific organ.

The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length, maximizing the number of matches and minimizes the number of gaps. Default settings can be used.

### VI. EMBODIMENTS

Embodiment 1. A pharmaceutical composition comprising a synthetic delivery system and a pharmaceutically-acceptable excipient, wherein the synthetic delivery system comprises a polynucleotide, wherein the polynucleotide comprises a nucleotide sequence with at least 80% sequence identity to at least 10 consecutive nucleotides of any one of SEQ ID NOs: 1-78.

Embodiment 2. The pharmaceutical composition of embodiment 1, wherein the polynucleotide comprises a nucleotide sequence with at least 80% sequence identity to at least 15 consecutive nucleotides of any one of SEQ ID NOs: 1-78.

Embodiment 3. The pharmaceutical composition of embodiment 1, wherein the polynucleotide comprises a nucleotide sequence with at least 80% sequence identity to at least 20 consecutive nucleotides of any one of SEQ ID NOs: 1-78.

Embodiment 4. The pharmaceutical composition of embodiment 1, wherein the polynucleotide comprises a nucleotide sequence with at least 80% sequence identity to at least 30 consecutive nucleotides of any one of SEQ ID NOs: 1-78.

Embodiment 5. The pharmaceutical composition of embodiment 1, wherein the polynucleotide comprises a nucleotide sequence with at least 80% sequence identity to at least 50 consecutive nucleotides of any one of SEQ ID NOs: 1-78.

Embodiment 6. The pharmaceutical composition of embodiment 1, wherein the polynucleotide comprises a nucleotide sequence with at least 80% sequence identity to at least 75 consecutive nucleotides of any one of SEQ ID NOs: 1-78.

Embodiment 7. The pharmaceutical composition of embodiment 1, wherein the polynucleotide comprises a nucleotide sequence with at least 80% sequence identity to at least 100 consecutive nucleotides of any one of SEQ ID NOs: 1-78.

Embodiment 8. The pharmaceutical composition of any one of embodiments 1-7, wherein the polynucleotide comprises a nucleotide sequence with at least 80% sequence identity to any one of SEQ ID NOs: 1-78.

Embodiment 9. The pharmaceutical composition of embodiment 1, wherein the polynucleotide comprises a nucleotide sequence with at least 90% sequence identity to at least 10 consecutive nucleotides of any one of SEQ ID NOs: 1-78.

Embodiment 10. The pharmaceutical composition of embodiment 1, wherein the polynucleotide comprises a nucleotide sequence with at least 90% sequence identity to at least 15 consecutive nucleotides of any one of SEQ ID NOs: 1-78.

Embodiment 11. The pharmaceutical composition of embodiment 1, wherein the polynucleotide comprises a nucleotide sequence with at least 90% sequence identity to at least 20 consecutive nucleotides of any one of SEQ ID NOs: 1-78.

Embodiment 12. The pharmaceutical composition of embodiment 1, wherein the polynucleotide comprises a nucleotide sequence with at least 90% sequence identity to at least 30 consecutive nucleotides of any one of SEQ ID NOs: 1-78.

Embodiment 13. The pharmaceutical composition of embodiment 1, wherein the polynucleotide comprises a nucleotide sequence with at least 90% sequence identity to at least 50 consecutive nucleotides of any one of SEQ ID NOs: 1-78.

Embodiment 14. The pharmaceutical composition of embodiment 1, wherein the polynucleotide comprises a nucleotide sequence with at least 90% sequence identity to at least 75 consecutive nucleotides of any one of SEQ ID NOs: 1-78.

Embodiment 15. The pharmaceutical composition of embodiment 1, wherein the polynucleotide comprises a nucleotide sequence with at least 90% sequence identity to at least 100 consecutive nucleotides of any one of SEQ ID NOs: 1-78.

Embodiment 16. The pharmaceutical composition of any one of the preceding embodiments, wherein the polynucleotide comprises a nucleotide sequence with at least 90% sequence identity to any one of SEQ ID NOs: 1-78.

Embodiment 17. The pharmaceutical composition of any one of the preceding embodiments, wherein the polynucleotide comprises a nucleotide sequence with at least 95% sequence identity to any one of SEQ ID NOs: 1-78.

Embodiment 18. The pharmaceutical composition of any one of the preceding embodiments, wherein the polynucleotide comprises a nucleotide sequence with at least 97% sequence identity to any one of SEQ ID NOs: 1-78.

Embodiment 19. The pharmaceutical composition of embodiment 1, wherein the polynucleotide comprises the nucleotide sequence of any one of SEQ ID NOs: 1-78.

Embodiment 20. A pharmaceutical composition comprising a synthetic delivery system and a pharmaceutically-acceptable excipient, wherein the synthetic delivery system comprises a polynucleotide, wherein the polynucleotide comprises a transposable element that is an AluSp, MER11C, AluY, 2L2a, ALluY, ALR/Alpha, ALU, AluJb, AluS1, AluSc8, AluSg, ALUSg2, AluSq, AluSq2, ALUSx, AluSx, AluSx1, AluSz, AluSz6, AluYc3, ASLUSq2, ERVK/LTR, ERVL, ERVL-MaLR, FLAM_C and AluY, HERV17-int, HERV9N-int, L1M1, L1MB3, L1ME4b, L1MEg, L1P1, L1P3, L1PA10, L1PA15, L1PA7, L1PB4, L2a, L2a/LTR40b/MLT1J2, LINE/L1, LINE/L2, LTR, LTR/ERV1, LTR/ERVL, LTR/ERVL-MaLR, LTR/Gypsy, LTR41C, LTR81B, Mam_R4, Mamr4, Many, MER11B, MER41E, MIR, MIRB, MIRc, MIRc-part L2, MLT1J2, MLT2B1, MLT1J2, MLT2B4, parAluSp-FULLMTLIJ2, REP522, Satellite/centr, SINE/Alu, SINE/MIR, THE1A, THE1B, THE1C, or Tigger3a transposable element.

Embodiment 21. A pharmaceutical composition comprising a synthetic delivery system and a pharmaceutically-acceptable excipient, wherein the synthetic delivery system comprises a polynucleotide that comprises a recognition sequence, wherein the recognition sequence facilitates binding or uptake of the synthetic delivery system by a target cell, wherein the recognition sequence comprises a nucleic acid sequence identified in circulating tumor DNA (ctDNA) from a substantially similar cell type as the target cell, wherein the polynucleotide comprises a short interspersed nuclear elements (SINE), long interspersed nuclear elements (LINE), ERVL, or ERVK transposable element.

Embodiment 22. The pharmaceutical composition of any one of embodiments 1-21, wherein the polynucleotide comprises an AluSp transposable element.

Embodiment 23. The pharmaceutical composition of any one of embodiments 1-21, wherein the polynucleotide comprises a MER11C transposable element.

Embodiment 24. The pharmaceutical composition of any one of embodiments 1-21, wherein the polynucleotide comprises an AluSx transposable element.

Embodiment 25. The pharmaceutical composition of any one of embodiments 1-21, wherein the polynucleotide comprises an MLT1J transposable element.

Embodiment 26. The pharmaceutical composition of any one of embodiments 1-21, wherein the polynucleotide comprises an AluSg2 transposable element.

Embodiment 27. The pharmaceutical composition of any one of embodiments 1-21, wherein the polynucleotide comprises a THE1A transposable element.

Embodiment 28. The pharmaceutical composition of any one of embodiments 1-21, wherein the polynucleotide comprises an AluJb transposable element.

Embodiment 29. The pharmaceutical composition of any one of embodiments 1-21, wherein the polynucleotide comprises an MLT2B4 transposable element.

Embodiment 30. The pharmaceutical composition of any one of embodiments 1-21, wherein the polynucleotide comprises an L2a transposable element.

Embodiment 31. The pharmaceutical composition of any one of embodiments 1-21, wherein the polynucleotide comprises a MLT1J2 transposable element.

Embodiment 32. The pharmaceutical composition of any one of embodiments 1-21, wherein the polynucleotide comprises an AluSq transposable element.

Embodiment 33. The pharmaceutical composition of any one of embodiments 1-21, wherein the polynucleotide comprises a L1MB3 transposable element.

Embodiment 34. The pharmaceutical composition of any one of embodiments 1-21, wherein the polynucleotide comprises a THE1C transposable element.

Embodiment 35. The pharmaceutical composition of any one of embodiments 1-21, wherein the polynucleotide comprises an AluY transposable element.

Embodiment 36. The pharmaceutical composition of any one of the preceding embodiments, wherein the polynucleotide comprises double stranded DNA.

Embodiment 37. The pharmaceutical composition of any one of the preceding embodiments, wherein the synthetic delivery system further comprises a cargo.

Embodiment 38. The pharmaceutical composition of embodiment 37, wherein the cargo comprises a nucleic acid cargo.

Embodiment 39. The pharmaceutical composition of embodiment 38, wherein the nucleic acid cargo is appended to a 3′ end of the polynucleotide.

Embodiment 40. The pharmaceutical composition of embodiment 38, wherein the nucleic acid cargo is appended to a 5′ end of the polynucleotide.

Embodiment 41. The pharmaceutical composition of any one of the preceding embodiments, wherein the synthetic delivery system comprises a promoter.

Embodiment 42. The pharmaceutical composition of any one of embodiments 38-41, wherein the nucleic acid cargo encodes a tumor suppressor protein.

Embodiment 43. The pharmaceutical composition of any one of embodiments 37-42, wherein the cargo comprises a cytotoxic cargo.

Embodiment 44. The pharmaceutical composition of any one of embodiments 37-43, wherein the cargo comprises a therapeutic cargo.

Embodiment 45. The pharmaceutical composition of any one of the preceding embodiments, wherein the synthetic delivery system does not utilize a viral vector, a nanoparticle, a lipid nanoparticle, a liposome, an exosome, a dendrimer, a gene gun, or electroporation.

Embodiment 46. A method of delivering a cargo to a target cell, the method comprising contacting the target cell with the pharmaceutical composition or the synthetic delivery system of any one of the preceding embodiments.

Embodiment 47. The method of embodiment 46, wherein the cargo is delivered to a nucleus of the target cell.

Embodiment 48. The method of embodiment 46 or embodiment 47, wherein the synthetic delivery system further comprises an integration sequence that facilitates integration of the polynucleotide, synthetic delivery system, or cargo, into the genome of the target cell.

Embodiment 49. The method of any one of embodiments 46-48, wherein the cargo comprises a nucleic acid cargo.

Embodiment 50. The method of embodiment 49, wherein the nucleic acid cargo is integrated into the target cell's genome.

Embodiment 51. The method of embodiment 49, wherein the nucleic acid cargo is integrated into the target cell's genome at a location identified in TABLE 2, TABLE 4, TABLE 7, or TABLE 8.

Embodiment 52. The method of embodiment 49, wherein the nucleic acid cargo is integrated into the target cells genome at Chr2: 32916224-32916626.

Embodiment 53. The method of embodiment 49, wherein the nucleic acid cargo is integrated into the target cells genome at Ch16: 32628381-32629000.

Embodiment 54. The method of any one of embodiments 46-53, wherein the target cell is a leukocyte.

Embodiment 55. The method of any one of embodiments 46-53, wherein the target cell is a plasma cell.

Embodiment 56. The method of any one of embodiments 46-53, wherein the target cell is a cancer cell.

Embodiment 57. The method of any one of embodiments 46-53, wherein the target cell is a multiple myeloma cell.

Embodiment 58. The method of any one of embodiments 46-53, wherein the target cell is a pancreatic cell.

Embodiment 59. The method of any one of embodiments 46-53, wherein the target cell is a pancreatic cancer cell.

Embodiment 60. The method of any one of embodiments 46-53, wherein the target cell is a gastrointestinal cell.

Embodiment 61. The method of any one of embodiments 46-53, wherein the target cell is a colorectal cancer cell.

Embodiment 62. A method of treating a condition in a subject in need thereof, the method comprising administering the pharmaceutical composition of any one of embodiments 1-45 to the subject.

Embodiment 63. A pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence comprises a nucleotide sequence with at least 80% sequence identity to at least 10 consecutive nucleotides of SEQ ID NO: 13 or 14.

Embodiment 64. The pharmaceutical composition of embodiment 63, wherein the recognition sequence comprises a nucleotide sequence with at least 80% sequence identity to at least 10 consecutive nucleotides of SEQ ID NO: 13.

Embodiment 65. The pharmaceutical composition of embodiment 63, wherein the recognition sequence comprises a nucleotide sequence with at least 80% sequence identity to at least 25 consecutive nucleotides of SEQ ID NO: 13.

Embodiment 66. The pharmaceutical composition of embodiment 63, wherein the recognition sequence comprises a nucleotide sequence with at least 80% sequence identity to at least 50 consecutive nucleotides of SEQ ID NO: 13.

Embodiment 67. The pharmaceutical composition of embodiment 63, wherein the recognition sequence comprises a nucleotide sequence with at least 80% sequence identity to at least 100 consecutive nucleotides of SEQ ID NO: 13.

Embodiment 68. The pharmaceutical composition of embodiment 63, wherein the recognition sequence comprises a nucleotide sequence with at least 80% sequence identity to at least 200 consecutive nucleotides of SEQ ID NO: 13.

Embodiment 69. The pharmaceutical composition of embodiment 63, wherein the recognition sequence comprises a nucleotide sequence with at least 90% sequence identity to at least 10 consecutive nucleotides of SEQ ID NO: 13.

Embodiment 70. The pharmaceutical composition of embodiment 63, wherein the recognition sequence comprises a nucleotide sequence with at least 90% sequence identity to at least 25 consecutive nucleotides of SEQ ID NO: 13.

Embodiment 71. The pharmaceutical composition of embodiment 63, wherein the recognition sequence comprises a nucleotide sequence with at least 90% sequence identity to at least 50 consecutive nucleotides of SEQ ID NO: 13.

Embodiment 72. The pharmaceutical composition of embodiment 63, wherein the recognition sequence comprises a nucleotide sequence with at least 90% sequence identity to at least 100 consecutive nucleotides of SEQ ID NO: 13.

Embodiment 73. The pharmaceutical composition of embodiment 63, wherein the recognition sequence comprises a nucleotide sequence with at least 90% sequence identity to at least 200 consecutive nucleotides of SEQ ID NO: 13.

Embodiment 74. The pharmaceutical composition of embodiment 63, wherein the recognition sequence comprises a nucleotide sequence with at least 90% sequence identity to SEQ ID NO: 13.

Embodiment 75. The pharmaceutical composition of embodiment 63, wherein the recognition sequence comprises a nucleotide sequence with at least 95% sequence identity to SEQ ID NO: 13.

Embodiment 76. The pharmaceutical composition of embodiment 63, wherein the recognition sequence comprises the nucleotide sequence of SEQ ID NO: 13.

Embodiment 77. The pharmaceutical composition of embodiment 63, wherein the recognition sequence comprises a nucleotide sequence with at least 80% sequence identity to at least 10 consecutive nucleotides of SEQ ID NO: 14.

Embodiment 78. The pharmaceutical composition of embodiment 63, wherein the recognition sequence comprises a nucleotide sequence with at least 80% sequence identity to at least 25 consecutive nucleotides of SEQ ID NO: 14.

Embodiment 79. The pharmaceutical composition of embodiment 63, wherein the recognition sequence comprises a nucleotide sequence with at least 80% sequence identity to at least 50 consecutive nucleotides of SEQ ID NO: 14.

Embodiment 80. The pharmaceutical composition of embodiment 63, wherein the recognition sequence comprises a nucleotide sequence with at least 80% sequence identity to at least 100 consecutive nucleotides of SEQ ID NO: 14.

Embodiment 81. The pharmaceutical composition of embodiment 63, wherein the recognition sequence comprises a nucleotide sequence with at least 80% sequence identity to at least 200 consecutive nucleotides of SEQ ID NO: 14.

Embodiment 82. The pharmaceutical composition of embodiment 63, wherein the recognition sequence comprises a nucleotide sequence with at least 90% sequence identity to at least 10 consecutive nucleotides of SEQ ID NO: 14.

Embodiment 83. The pharmaceutical composition of embodiment 63, wherein the recognition sequence comprises a nucleotide sequence with at least 90% sequence identity to at least 25 consecutive nucleotides of SEQ ID NO: 14.

84. The pharmaceutical composition of embodiment 63, wherein the recognition sequence comprises a nucleotide sequence with at least 90% sequence identity to at least 50 consecutive nucleotides of SEQ ID NO: 14.

Embodiment 85. The pharmaceutical composition of embodiment 63, wherein the recognition sequence comprises a nucleotide sequence with at least 90% sequence identity to at least 100 consecutive nucleotides of SEQ ID NO: 14.

Embodiment 86. The pharmaceutical composition of embodiment 63, wherein the recognition sequence comprises a nucleotide sequence with at least 90% sequence identity to at least 200 consecutive nucleotides of SEQ ID NO: 14.

Embodiment v87. The pharmaceutical composition of embodiment 63, wherein the recognition sequence comprises a nucleotide sequence with at least 90% sequence identity to SEQ ID NO: 14.

Embodiment 88. The pharmaceutical composition of embodiment 63, wherein the recognition sequence comprises a nucleotide sequence with at least 95% sequence identity to SEQ ID NO: 14.

Embodiment 89. The pharmaceutical composition of embodiment 63, wherein the recognition sequence comprises the nucleotide sequence of SEQ ID NO: 14.

Embodiment 90. A pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence comprises (a) a nucleotide sequence with at least 90% sequence identity to at least 60 consecutive nucleotides of SEQ ID NO: 33, or (b) a nucleotide sequence with at least 91% sequence identity to at least 50 consecutive nucleotides of SEQ ID NO: 33.

Embodiment 91. The pharmaceutical composition of embodiment 90, wherein the recognition sequence comprises the nucleotide sequence with at least 90% sequence identity to at least 60 consecutive nucleotides of SEQ ID NO: 33.

Embodiment 92. The pharmaceutical composition of embodiment 90, wherein the recognition sequence comprises the nucleotide sequence with at least 91% sequence identity to at least 50 consecutive nucleotides of SEQ ID NO: 33.

Embodiment 93. The pharmaceutical composition of embodiment 90, wherein the recognition sequence comprises a nucleotide sequence with at least 90% sequence identity to at least 75 consecutive nucleotides of SEQ ID NO: 33.

Embodiment 94. The pharmaceutical composition of embodiment 90, wherein the recognition sequence comprises a nucleotide sequence with at least 90% sequence identity to at least 100 consecutive nucleotides of SEQ ID NO: 33.

Embodiment 95. The pharmaceutical composition of embodiment 90, wherein the recognition sequence comprises a nucleotide sequence with at least 90% sequence identity to at least 200 consecutive nucleotides of SEQ ID NO: 33.

Embodiment 96. The pharmaceutical composition of embodiment 90, wherein the recognition sequence comprises a nucleotide sequence with at least 95% sequence identity to at least 50 consecutive nucleotides of SEQ ID NO: 33.

Embodiment 97. The pharmaceutical composition of embodiment 90, wherein the recognition sequence comprises a nucleotide sequence with at least 90% sequence identity to SEQ ID NO: 33.

Embodiment 98. The pharmaceutical composition of embodiment 90, wherein the recognition sequence comprises a nucleotide sequence with at least 95% sequence identity to SEQ ID NO: 33.

Embodiment 99. The pharmaceutical composition of embodiment 90, wherein the recognition sequence comprises the nucleotide sequence of SEQ ID NO: 33.

Embodiment 100. A pharmaceutical composition comprising a synthetic delivery system and a pharmaceutically-acceptable excipient, wherein the synthetic delivery system comprises a polynucleotide that comprises a recognition sequence, wherein the recognition sequence comprises an AluSp, AluSx, AluSg2, ERV2 superfamily, or ERV3 superfamily transposable element, or a functional fragment thereof that facilitates binding of the synthetic delivery system to a target cell or uptake of the synthetic delivery system or a component thereof by the target cell.

Embodiment 101. The pharmaceutical composition of embodiment 100, wherein the recognition sequence comprises the AluSp transposable element or the functional fragment thereof.

Embodiment 102. The pharmaceutical composition of embodiment 100, wherein the recognition sequence comprises the AluSx transposable element or the functional fragment thereof.

Embodiment 103. The pharmaceutical composition of embodiment 100, wherein the recognition sequence comprises the AluSg2 transposable element or the functional fragment thereof.

Embodiment 104. The pharmaceutical composition of embodiment 100, wherein the recognition sequence comprises the ERV2 superfamily transposable element or the functional fragment thereof.

Embodiment 105. The pharmaceutical composition of embodiment 104, wherein the ERV2 superfamily transposable element or the functional fragment thereof comprises an HML8 group transposable element of functional fragment thereof that facilitates binding of the synthetic delivery system to the target cell or uptake of the synthetic delivery system or component thereof by the target cell.

Embodiment 106. The pharmaceutical composition of embodiment 104 or 105, wherein the ERV2 superfamily transposable element or the functional fragment thereof comprises a MER family transposable element of functional fragment thereof that facilitates binding of the synthetic delivery system to the target cell or uptake of the synthetic delivery system or component thereof by the target cell.

Embodiment 107. The pharmaceutical composition of any one of embodiments 104-106, wherein the ERV2 superfamily transposable element or the functional fragment thereof comprises a MER11C transposable element of functional fragment thereof that facilitates binding of the synthetic delivery system to the target cell or uptake of the synthetic delivery system or component thereof by the target cell.

Embodiment 108. The pharmaceutical composition of embodiment 100, wherein the recognition sequence comprises the ERV3 superfamily transposable element or the functional fragment thereof.

Embodiment 109. The pharmaceutical composition of embodiment 108, wherein the ERV3 superfamily transposable element or the functional fragment thereof comprises a MaLR group transposable element of functional fragment thereof that facilitates binding of the synthetic delivery system to the target cell or uptake of the synthetic delivery system or component thereof by the target cell.

Embodiment 110. The pharmaceutical composition of embodiment 108 or 109, wherein the ERV3 superfamily transposable element or the functional fragment thereof comprises a THE1A transposable element of functional fragment thereof that facilitates binding of the synthetic delivery system to the target cell or uptake of the synthetic delivery system or component thereof by the target cell.

Embodiment 111. The pharmaceutical composition of embodiment 108 or 109, wherein the ERV3 superfamily transposable element or the functional fragment thereof comprises a MLT1J2 transposable element of functional fragment thereof that facilitates binding of the synthetic delivery system to the target cell or uptake of the synthetic delivery system or component thereof by the target cell.

Embodiment 112. A pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the polynucleotide comprises a nucleotide sequence with at least 86% sequence identity to any one of SEQ ID NOs: 7, 16, 25, 29, and 31.

Embodiment 113. The pharmaceutical composition of embodiment 112, wherein the polynucleotide comprises a nucleotide sequence with at least 90% sequence identity to SEQ ID NO: 7.

Embodiment 114. The pharmaceutical composition of embodiment 112, wherein the polynucleotide comprises a nucleotide sequence with at least 90% sequence identity to SEQ ID NO: 16.

Embodiment 115. The pharmaceutical composition of embodiment 112, wherein the polynucleotide comprises a nucleotide sequence with at least 90% sequence identity to SEQ ID NO: 25.

Embodiment 116. The pharmaceutical composition of embodiment 112, wherein the polynucleotide comprises a nucleotide sequence with at least 90% sequence identity to SEQ ID NO: 29.

Embodiment 117. The pharmaceutical composition of embodiment 112, wherein the polynucleotide comprises a nucleotide sequence with at least 90% sequence identity to SEQ ID NO: 31.

Embodiment 118. The pharmaceutical composition of embodiment 112, wherein the polynucleotide comprises a nucleotide sequence with at least 95% sequence identity to SEQ ID NO: 7.

Embodiment 119. The pharmaceutical composition of embodiment 112, wherein the polynucleotide comprises a nucleotide sequence with at least 95% sequence identity to SEQ ID NO: 16.

Embodiment 120. The pharmaceutical composition of embodiment 112, wherein the polynucleotide comprises a nucleotide sequence with at least 95% sequence identity to SEQ ID NO: 25.

Embodiment 121. The pharmaceutical composition of embodiment 112, wherein the polynucleotide comprises a nucleotide sequence with at least 95% sequence identity to SEQ ID NO: 29.

Embodiment 122. The pharmaceutical composition of embodiment 112, wherein the polynucleotide comprises a nucleotide sequence with at least 95% sequence identity to SEQ ID NO: 31.

Embodiment 123. The pharmaceutical composition of embodiment 112, wherein the polynucleotide comprises a nucleotide sequence with at least 98% sequence identity to SEQ ID NO: 7.

Embodiment 124. The pharmaceutical composition of embodiment 112, wherein the polynucleotide comprises a nucleotide sequence with at least 98% sequence identity to SEQ ID NO: 16.

Embodiment 125. The pharmaceutical composition of embodiment 112, wherein the polynucleotide comprises a nucleotide sequence with at least 98% sequence identity to SEQ ID NO: 25.

Embodiment 126. The pharmaceutical composition of embodiment 112, wherein the polynucleotide comprises a nucleotide sequence with at least 98% sequence identity to SEQ ID NO: 29.

Embodiment 127. The pharmaceutical composition of embodiment 112, wherein the polynucleotide comprises a nucleotide sequence with at least 98% sequence identity to SEQ ID NO: 31.

Embodiment 128. The pharmaceutical composition of embodiment 112, wherein the polynucleotide comprises the nucleotide sequence of SEQ ID NO: 7.

Embodiment 129. The pharmaceutical composition of embodiment 112, wherein the polynucleotide comprises the nucleotide sequence of SEQ ID NO: 16.

Embodiment 130. The pharmaceutical composition of embodiment 112, wherein the polynucleotide comprises the nucleotide sequence of SEQ ID NO: 25.

Embodiment 131. The pharmaceutical composition of embodiment 112, wherein the polynucleotide comprises the nucleotide sequence of SEQ ID NO: 29.

Embodiment 132. The pharmaceutical composition of embodiment 112, wherein the polynucleotide comprises the nucleotide sequence of SEQ ID NO: 31.

Embodiment 133. The pharmaceutical composition of any one of the preceding embodiments, wherein the recognition sequence facilitates binding of the synthetic delivery system to the target cell.

Embodiment 134. The pharmaceutical composition of any one of the preceding embodiments, wherein the recognition sequence facilitates at least 50% higher binding of the synthetic delivery system to the target cell as compared to a control synthetic delivery system that lacks the recognition sequence.

Embodiment 135. The pharmaceutical composition of any one of the preceding embodiments, wherein the recognition sequence facilitates at least 50% higher binding of the synthetic delivery system to the target cell as compared to a control cell of a different cell type.

Embodiment 136. The pharmaceutical composition of any one of the preceding embodiments, wherein the recognition sequence facilitates uptake of the synthetic delivery system or component thereof by the target cell.

Embodiment 137. The pharmaceutical composition of any one of the preceding embodiments, wherein the recognition sequence facilitates at least 50% higher uptake of the synthetic delivery system by the target cell as compared to a control synthetic delivery system that lacks the recognition sequence.

Embodiment 138. The pharmaceutical composition of any one of the preceding embodiments, wherein the recognition sequence facilitates at least 50% higher uptake of the synthetic delivery system by the target cell as compared to a control cell of a different cell type.

Embodiment 139. The pharmaceutical composition of any one of the preceding embodiments, wherein the recognition sequence facilitates at least 50% higher nuclear localization of the synthetic delivery system within the target cell as compared to a control synthetic delivery system that lacks the recognition sequence.

Embodiment 140. The pharmaceutical composition of any one of the preceding embodiments, wherein the recognition sequence facilitates at least 50% higher nuclear localization of the synthetic delivery system within the target cell as compared to a control cell of a different cell type.

Embodiment 141. The pharmaceutical composition of any one of the preceding embodiments, wherein the recognition sequence facilitates binding of the synthetic delivery system to the target cell and uptake of the synthetic delivery system or component thereof by the target cell.

Embodiment 142. The pharmaceutical composition of any one of the preceding embodiments, wherein the recognition sequence comprises a nucleic acid sequence identified in circulating tumor DNA (ctDNA) from a substantially similar cell type as the target cell.

Embodiment 143. The pharmaceutical composition of any one of the preceding embodiments, wherein the polynucleotide comprises double stranded DNA.

Embodiment 144. The pharmaceutical composition of any one of the preceding embodiments, wherein the synthetic delivery system further comprises a cargo.

Embodiment 145. The pharmaceutical composition of embodiment 144, wherein the cargo comprises an anti-cancer therapeutic agent.

Embodiment 146. The pharmaceutical composition of embodiment 144 or 145, wherein the cargo comprises a nucleic acid cargo.

Embodiment 147. The pharmaceutical composition of embodiment 147, wherein the nucleic acid cargo encodes a tumor suppressor protein

Embodiment 148. The pharmaceutical composition of any one of embodiments 144-147, wherein the cargo is appended to a 3′ end of the polynucleotide.

Embodiment 149. The pharmaceutical composition of any one of embodiments 144-148, wherein the cargo is appended to a 5′ end of the polynucleotide.

Embodiment 150. The pharmaceutical composition of any one of embodiments 144-149, wherein the cargo comprises a cytotoxic cargo.

Embodiment 151. The pharmaceutical composition of any one of embodiments 144-150, wherein the cargo comprises a therapeutic cargo.

Embodiment 152. The pharmaceutical composition of any one of embodiments 144-151, wherein the cargo comprises a polypeptide.

Embodiment 153. The pharmaceutical composition of any one of embodiments 144-152, wherein the cargo comprises a small molecule.

Embodiment 154. The pharmaceutical composition of any one of the preceding embodiments, wherein the synthetic delivery system further comprises a promoter.

Embodiment 155. The pharmaceutical composition of any one of the preceding embodiments, wherein the synthetic delivery system further comprises an integration sequence that facilitates integration of the polynucleotide, synthetic delivery system, cargo, or a component thereof into the genome of the target cell.

Embodiment 156. The pharmaceutical composition of embodiment 155, wherein the integration sequence facilitates at least 50% higher genomic integration of the synthetic delivery system, polynucleotide, cargo, or component thereof in target cells as compared to a control synthetic delivery system that lacks the integration sequence.

Embodiment 157. The pharmaceutical composition of any one of embodiments 155-156, wherein the integration sequence facilitates at least 50% higher genomic integration of the synthetic delivery system, polynucleotide, cargo, or component thereof by target cells as compared to control cells of a different cell type.

Embodiment 158. The pharmaceutical composition of any one of the preceding embodiments, wherein the synthetic delivery system does not utilize a viral vector, a nanoparticle, a lipid nanoparticle, a liposome, an exosome, a dendrimer, a gene gun, or electroporation for delivery to the target cell.

Embodiment 159. The pharmaceutical composition of any one of embodiments 62-158, wherein the target cell is a leukocyte.

Embodiment 160. The pharmaceutical composition of any one of embodiments 62-159, wherein the target cell is a plasma cell.

Embodiment 161. The pharmaceutical composition of any one of embodiments 62-160, wherein the target cell is a cancer cell.

Embodiment 162. The pharmaceutical composition of any one of embodiments 62-161, wherein the target cell is a multiple myeloma cell.

Embodiment 163. The pharmaceutical composition of any one of embodiments 62-158 and 161, wherein the target cell is a pancreatic cell.

Embodiment 164. The pharmaceutical composition of any one of embodiments 62-158, wherein the target cell is a pancreatic cancer cell.

Embodiment 165. The pharmaceutical composition of any one of embodiments 62-158, wherein the target cell is a gastrointestinal cell.

Embodiment 166. The pharmaceutical composition of any one of embodiments 62-158, wherein the target cell is a colorectal cancer cell.

Embodiment 167. A method of delivering a cargo to a target cell, the method comprising contacting the target cell with the pharmaceutical composition or the synthetic delivery system of any one of the preceding embodiments.

Embodiment 168. The method of embodiment 167, wherein the cargo is delivered to an intracellular compartment of the target cell.

Embodiment 169. The method of embodiment 167 or 168, wherein the cargo is delivered to a nucleus of the target cell.

Embodiment 170. The method of any one of embodiments 167-169, wherein the cargo comprises a nucleic acid cargo, wherein the nucleic acid cargo is integrated into the target cell's genome.

Embodiment 171. The method of embodiment 170, wherein the nucleic acid cargo is integrated into the target cell's genome at a location identified in TABLE 2, TABLE 4, TABLE 7, or TABLE 8.

Embodiment 172. The method of embodiment 170, wherein the nucleic acid cargo is integrated into the target cell's genome at Chr2: 32916224-32916626.

Embodiment 173. The method of embodiment 170, wherein the nucleic acid cargo is integrated into the target cell's genome at Ch16: 32628381-32629000.

Embodiment 174. A method of treating a condition in a subject in need thereof, the method comprising administering to the subject the pharmaceutical composition of any one of the preceding embodiments.

Embodiment 175. The method of embodiment 174, wherein the subject is mammalian.

Embodiment 176. The method of any one of embodiment 174, wherein the subject is human.

Embodiment 177. The method of any one of embodiments 174-176, wherein the condition is a cancer.

Embodiment 178. The method of embodiment 177, wherein the cancer is a hematologic cancer.

Embodiment 179. The method of embodiment 177, wherein the cancer is a multiple myeloma.

Embodiment 180. The method of embodiment 177, wherein the cancer is a solid tumor.

Embodiment 181. The method of embodiment 177 or 180, wherein the cancer is a pancreatic cancer.

Embodiment 182. The method of embodiment 177 or 180, wherein the cancer is colorectal cancer.

Embodiment 183. A pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence consists essentially of a nucleotide sequence with at least 80% sequence identity to any one of SEQ ID NOs: 73-75.

Embodiment 184. The pharmaceutical composition of embodiment 183, wherein the recognition sequence consists essentially of a nucleotide sequence with at least 80% sequence identity to SEQ ID NO: 73.

Embodiment 185. The pharmaceutical composition of embodiment 183, wherein the recognition sequence consists essentially of a nucleotide sequence with at least 90% sequence identity to SEQ ID NO: 73.

Embodiment 186. The pharmaceutical composition of embodiment 183, wherein the recognition sequence consists essentially of a nucleotide sequence with at least 95% sequence identity to SEQ ID NO: 73.

Embodiment 187. The pharmaceutical composition of embodiment 183, wherein the recognition sequence consists essentially of SEQ ID NO: 73.

Embodiment 188. The pharmaceutical composition of embodiment 183, wherein the recognition sequence consists essentially of a nucleotide sequence with at least 80% sequence identity to SEQ ID NO: 74.

Embodiment 189. The pharmaceutical composition of embodiment 183, wherein the recognition sequence consists essentially of a nucleotide sequence with at least 90% sequence identity to SEQ ID NO: 74.

Embodiment 190. The pharmaceutical composition of embodiment 183, wherein the recognition sequence consists essentially of a nucleotide sequence with at least 95% sequence identity to SEQ ID NO: 74.

Embodiment 191. The pharmaceutical composition of embodiment 183, wherein the recognition sequence consists essentially of SEQ ID NO: 74.

Embodiment 192. The pharmaceutical composition of embodiment 183, wherein the recognition sequence consists essentially of a nucleotide sequence with at least 80% sequence identity to SEQ ID NO: 75.

Embodiment 193. The pharmaceutical composition of embodiment 183, wherein the recognition sequence consists essentially of a nucleotide sequence with at least 90% sequence identity to SEQ ID NO: 75.

Embodiment 194. The pharmaceutical composition of embodiment 183, wherein the recognition sequence consists essentially of a nucleotide sequence with at least 95% sequence identity to SEQ ID NO: 75.

Embodiment 195. The pharmaceutical composition of embodiment 183, wherein the recognition sequence consists essentially of the nucleotide sequence of SEQ ID NO: 75.

Embodiment 196. The pharmaceutical composition of any one of embodiments 183-195, wherein the recognition sequence does not contain a nucleotide sequence with at least 50% sequence identity to SEQ ID NO: 71.

Embodiment 197. The pharmaceutical composition of any one of embodiments 183-195, wherein the recognition sequence does not contain a nucleotide sequence with at least 80% sequence identity to SEQ ID NO: 71.

Embodiment 198. The pharmaceutical composition of any one of embodiments 183-195, wherein the recognition sequence does not contain a nucleotide sequence with at least 90% sequence identity to SEQ ID NO: 71.

Embodiment 199. The pharmaceutical composition of any one of embodiments 183-195, wherein the recognition sequence does not contain the nucleotide sequence of SEQ ID NO: 71.

Embodiment 200. The pharmaceutical composition of any one of embodiments 183-195, wherein the recognition sequence does not contain a nucleotide sequence with at least 50% sequence identity to nucleotides 1-87 of SEQ ID NO: 68.

Embodiment 201. The pharmaceutical composition of any one of embodiments 183-195, wherein the recognition sequence does not contain a nucleotide sequence with at least 80% sequence identity to nucleotides 1-87 of SEQ ID NO: 68.

Embodiment 202. The pharmaceutical composition of any one of embodiments 183-195, wherein the recognition sequence does not contain a nucleotide sequence with at least 90% sequence identity to nucleotides 1-87 of SEQ ID NO: 68.

Embodiment 203. The pharmaceutical composition of any one of embodiments 183-195, wherein the recognition sequence does not contain the nucleotide sequence of nucleotides 1-87 of SEQ ID NO: 68.

Embodiment 204. A pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence comprises the nucleotide sequence of SEQ ID NO: 73.

Embodiment 205. The pharmaceutical composition of embodiment 204, wherein the recognition sequence comprises a nucleotide sequence with at least 80% sequence identity to SEQ ID NO: 74.

Embodiment 206. The pharmaceutical composition of embodiment 204, wherein the recognition sequence comprises a nucleotide sequence with at least 90% sequence identity to SEQ ID NO: 74.

Embodiment 207. The pharmaceutical composition of embodiment 204, wherein the recognition sequence comprises the nucleotide sequence of SEQ ID NO: 74.

Embodiment 208. The pharmaceutical composition of embodiment 204, wherein the recognition sequence comprises a nucleotide sequence with at least 80% sequence identity to SEQ ID NO: 75.

Embodiment 209. The pharmaceutical composition of embodiment 204, wherein the recognition sequence comprises a nucleotide sequence with at least 90% sequence identity to SEQ ID NO: 75.

Embodiment 210. The pharmaceutical composition of embodiment 204, wherein the recognition sequence comprises the nucleotide sequence of SEQ ID NO: 75.

Embodiment 211. The pharmaceutical composition of any one of embodiments 204-210, wherein the recognition sequence does not contain a nucleotide sequence with at least 50% sequence identity to SEQ ID NO: 71.

Embodiment 212. The pharmaceutical composition of any one of embodiments 204-210, wherein the recognition sequence does not contain a nucleotide sequence with at least 80% sequence identity to SEQ ID NO: 71.

Embodiment 213. The pharmaceutical composition of any one of embodiments 204-210, wherein the recognition sequence does not contain a nucleotide sequence with at least 90% sequence identity to SEQ ID NO: 71.

Embodiment 214. The pharmaceutical composition of any one of embodiments 204-210, wherein the recognition sequence does not contain the nucleotide sequence of SEQ ID NO: 71.

Embodiment 215. The pharmaceutical composition of any one of embodiments 204-210, wherein the recognition sequence does not contain a nucleotide sequence with at least 50% sequence identity to nucleotides 1-87 of SEQ ID NO: 68.

Embodiment 216. The pharmaceutical composition of any one of embodiments 204-210, wherein the recognition sequence does not contain a nucleotide sequence with at least 80% sequence identity to nucleotides 1-87 of SEQ ID NO: 68.

Embodiment 217. The pharmaceutical composition of any one of embodiments 204-210, wherein the recognition sequence does not contain a nucleotide sequence with at least 90% sequence identity to nucleotides 1-87 of SEQ ID NO: 68.

Embodiment 218. The pharmaceutical composition of any one of embodiments 204-210, wherein the recognition sequence does not contain the nucleotide sequence of nucleotides 1-87 of SEQ ID NO: 68.

Embodiment 219. A pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence comprises a nucleotide sequence with at least 80% sequence identity to at least 10 consecutive nucleotides of SEQ ID NO: 13 or 14.

Embodiment 220. The pharmaceutical composition of embodiment 219, wherein the recognition sequence comprises a nucleotide sequence with at least 80% sequence identity to at least 10 consecutive nucleotides of SEQ ID NO: 13.

Embodiment 221. The pharmaceutical composition of embodiment 219, wherein the recognition sequence comprises a nucleotide sequence with at least 80% sequence identity to at least 25 consecutive nucleotides of SEQ ID NO: 13.

Embodiment 222. The pharmaceutical composition of embodiment 219, wherein the recognition sequence comprises a nucleotide sequence with at least 80% sequence identity to at least 50 consecutive nucleotides of SEQ ID NO: 13.

Embodiment 223. The pharmaceutical composition of embodiment 219, wherein the recognition sequence comprises a nucleotide sequence with at least 80% sequence identity to at least 100 consecutive nucleotides of SEQ ID NO: 13.

Embodiment 224. The pharmaceutical composition of embodiment 219, wherein the recognition sequence comprises a nucleotide sequence with at least 80% sequence identity to at least 200 consecutive nucleotides of SEQ ID NO: 13.

Embodiment 225. The pharmaceutical composition of embodiment 219, wherein the recognition sequence comprises a nucleotide sequence with at least 90% sequence identity to at least 10 consecutive nucleotides of SEQ ID NO: 13.

Embodiment 226. The pharmaceutical composition of embodiment 219, wherein the recognition sequence comprises a nucleotide sequence with at least 90% sequence identity to at least 25 consecutive nucleotides of SEQ ID NO: 13.

Embodiment 227. The pharmaceutical composition of embodiment 219, wherein the recognition sequence comprises a nucleotide sequence with at least 90% sequence identity to at least 50 consecutive nucleotides of SEQ ID NO: 13.

Embodiment 228. The pharmaceutical composition of embodiment 219, wherein the recognition sequence comprises a nucleotide sequence with at least 90% sequence identity to at least 100 consecutive nucleotides of SEQ ID NO: 13.

Embodiment 229. The pharmaceutical composition of embodiment 219, wherein the recognition sequence comprises a nucleotide sequence with at least 90% sequence identity to at least 200 consecutive nucleotides of SEQ ID NO: 13.

Embodiment 230. The pharmaceutical composition of embodiment 219, wherein the recognition sequence comprises a nucleotide sequence with at least 90% sequence identity to SEQ ID NO: 13.

Embodiment 231. The pharmaceutical composition of embodiment 219, wherein the recognition sequence comprises a nucleotide sequence with at least 95% sequence identity to SEQ ID NO: 13.

Embodiment 232. The pharmaceutical composition of embodiment 219, wherein the recognition sequence comprises the nucleotide sequence of SEQ ID NO: 13.

Embodiment 233. The pharmaceutical composition of embodiment 219, wherein the recognition sequence comprises a nucleotide sequence with at least 80% sequence identity to at least 10 consecutive nucleotides of SEQ ID NO: 14.

Embodiment 234. The pharmaceutical composition of embodiment 219, wherein the recognition sequence comprises a nucleotide sequence with at least 80% sequence identity to at least 25 consecutive nucleotides of SEQ ID NO: 14.

Embodiment 235. The pharmaceutical composition of embodiment 219, wherein the recognition sequence comprises a nucleotide sequence with at least 80% sequence identity to at least 50 consecutive nucleotides of SEQ ID NO: 14.

Embodiment 236. The pharmaceutical composition of embodiment 219, wherein the recognition sequence comprises a nucleotide sequence with at least 80% sequence identity to at least 100 consecutive nucleotides of SEQ ID NO: 14.

Embodiment 237. The pharmaceutical composition of embodiment 219, wherein the recognition sequence comprises a nucleotide sequence with at least 80% sequence identity to at least 200 consecutive nucleotides of SEQ ID NO: 14.

Embodiment 238. The pharmaceutical composition of embodiment 219, wherein the recognition sequence comprises a nucleotide sequence with at least 90% sequence identity to at least 10 consecutive nucleotides of SEQ ID NO: 14.

Embodiment 239. The pharmaceutical composition of embodiment 219, wherein the recognition sequence comprises a nucleotide sequence with at least 90% sequence identity to at least 25 consecutive nucleotides of SEQ ID NO: 14.

Embodiment 240. The pharmaceutical composition of embodiment 219, wherein the recognition sequence comprises a nucleotide sequence with at least 90% sequence identity to at least 50 consecutive nucleotides of SEQ ID NO: 14.

Embodiment 241. The pharmaceutical composition of embodiment 219, wherein the recognition sequence comprises a nucleotide sequence with at least 90% sequence identity to at least 100 consecutive nucleotides of SEQ ID NO: 14.

Embodiment 242. The pharmaceutical composition of embodiment 219, wherein the recognition sequence comprises a nucleotide sequence with at least 90% sequence identity to at least 200 consecutive nucleotides of SEQ ID NO: 14.

Embodiment 243. The pharmaceutical composition of embodiment 219, wherein the recognition sequence comprises a nucleotide sequence with at least 90% sequence identity to SEQ ID NO: 14.

Embodiment 244. The pharmaceutical composition of embodiment 219, wherein the recognition sequence comprises a nucleotide sequence with at least 95% sequence identity to SEQ ID NO: 14.

Embodiment 245. The pharmaceutical composition of embodiment 219, wherein the recognition sequence comprises the nucleotide sequence of SEQ ID NO: 14.

Embodiment 246. A pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence comprises (a) a nucleotide sequence with at least 90% sequence identity to at least 60 consecutive nucleotides of SEQ ID NO: 33, or (b) a nucleotide sequence with at least 91% sequence identity to at least 50 consecutive nucleotides of SEQ ID NO: 33.

Embodiment 247. The pharmaceutical composition of embodiment 246, wherein the recognition sequence comprises the nucleotide sequence with at least 90% sequence identity to at least 60 consecutive nucleotides of SEQ ID NO: 33.

Embodiment 248. The pharmaceutical composition of embodiment 246, wherein the recognition sequence comprises the nucleotide sequence with at least 91% sequence identity to at least 50 consecutive nucleotides of SEQ ID NO: 33.

Embodiment 249. The pharmaceutical composition of embodiment 246, wherein the recognition sequence comprises a nucleotide sequence with at least 90% sequence identity to at least 75 consecutive nucleotides of SEQ ID NO: 33.

Embodiment 250. The pharmaceutical composition of embodiment 246, wherein the recognition sequence comprises a nucleotide sequence with at least 90% sequence identity to at least 100 consecutive nucleotides of SEQ ID NO: 33.

Embodiment 251. The pharmaceutical composition of embodiment 246, wherein the recognition sequence comprises a nucleotide sequence with at least 90% sequence identity to at least 200 consecutive nucleotides of SEQ ID NO: 33.

Embodiment 252. The pharmaceutical composition of embodiment 246, wherein the recognition sequence comprises a nucleotide sequence with at least 95% sequence identity to at least 50 consecutive nucleotides of SEQ ID NO: 33.

Embodiment 253. The pharmaceutical composition of embodiment 246, wherein the recognition sequence comprises a nucleotide sequence with at least 90% sequence identity to SEQ ID NO: 33.

Embodiment 254. The pharmaceutical composition of embodiment 246, wherein the recognition sequence comprises a nucleotide sequence with at least 95% sequence identity to SEQ ID NO: 33.

Embodiment 255. The pharmaceutical composition of embodiment 246, wherein the recognition sequence comprises the nucleotide sequence of SEQ ID NO: 33.

Embodiment 256. A pharmaceutical composition comprising a synthetic delivery system and a pharmaceutically-acceptable excipient, wherein the synthetic delivery system comprises a polynucleotide that comprises a recognition sequence, wherein the recognition sequence comprises an AluSp, AluSx, AluSg2, ERV2 superfamily, or ERV3 superfamily transposable element, or a functional fragment thereof that facilitates binding of the synthetic delivery system to a target cell or uptake of the synthetic delivery system or a component thereof by the target cell.

Embodiment 257. The pharmaceutical composition of embodiment 256, wherein the recognition sequence comprises the AluSp transposable element or the functional fragment thereof.

Embodiment 258. The pharmaceutical composition of embodiment 256, wherein the recognition sequence comprises the AluSx transposable element or the functional fragment thereof.

Embodiment 259. The pharmaceutical composition of embodiment 256, wherein the recognition sequence comprises the AluSg2 transposable element or the functional fragment thereof.

Embodiment 260. The pharmaceutical composition of embodiment 256, wherein the recognition sequence comprises the ERV2 superfamily transposable element or the functional fragment thereof.

Embodiment 261. The pharmaceutical composition of embodiment 260, wherein the ERV2 superfamily transposable element or the functional fragment thereof comprises an HML8 group transposable element of functional fragment thereof that facilitates binding of the synthetic delivery system to the target cell or uptake of the synthetic delivery system or component thereof by the target cell.

Embodiment 262. The pharmaceutical composition of embodiment 260, wherein the ERV2 superfamily transposable element or the functional fragment thereof comprises a MER family transposable element of functional fragment thereof that facilitates binding of the synthetic delivery system to the target cell or uptake of the synthetic delivery system or component thereof by the target cell.

Embodiment 263. The pharmaceutical composition of embodiment 260, wherein the ERV2 superfamily transposable element or the functional fragment thereof comprises a MER11C transposable element of functional fragment thereof that facilitates binding of the synthetic delivery system to the target cell or uptake of the synthetic delivery system or component thereof by the target cell.

Embodiment 264. The pharmaceutical composition of embodiment 256, wherein the recognition sequence comprises the ERV3 superfamily transposable element or the functional fragment thereof.

Embodiment 265. The pharmaceutical composition of embodiment 264, wherein the ERV3 superfamily transposable element or the functional fragment thereof comprises a MaLR group transposable element of functional fragment thereof that facilitates binding of the synthetic delivery system to the target cell or uptake of the synthetic delivery system or component thereof by the target cell.

Embodiment 266. The pharmaceutical composition of embodiment 264, wherein the ERV3 superfamily transposable element or the functional fragment thereof comprises a THE1A transposable element of functional fragment thereof that facilitates binding of the synthetic delivery system to the target cell or uptake of the synthetic delivery system or component thereof by the target cell.

Embodiment 267. The pharmaceutical composition of embodiment 264, wherein the ERV3 superfamily transposable element or the functional fragment thereof comprises a MLT1J2 transposable element of functional fragment thereof that facilitates binding of the synthetic delivery system to the target cell or uptake of the synthetic delivery system or component thereof by the target cell.

Embodiment 268. A pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the polynucleotide comprises a nucleotide sequence with at least 86% sequence identity to any one of SEQ ID NOs: 7, 16, 25, 29, and 31.

Embodiment 269. The pharmaceutical composition of embodiment 268, wherein the polynucleotide comprises a nucleotide sequence with at least 90% sequence identity to SEQ ID NO: 7.

Embodiment 270. The pharmaceutical composition of embodiment 268, wherein the polynucleotide comprises a nucleotide sequence with at least 90% sequence identity to SEQ ID NO: 16.

Embodiment 271. The pharmaceutical composition of embodiment 268, wherein the polynucleotide comprises a nucleotide sequence with at least 90% sequence identity to SEQ ID NO: 25.

Embodiment 272. The pharmaceutical composition of embodiment 268, wherein the polynucleotide comprises a nucleotide sequence with at least 90% sequence identity to SEQ ID NO: 29.

Embodiment 273. The pharmaceutical composition of embodiment 268, wherein the polynucleotide comprises a nucleotide sequence with at least 90% sequence identity to SEQ ID NO: 31.

Embodiment 274. The pharmaceutical composition of embodiment 268, wherein the polynucleotide comprises a nucleotide sequence with at least 95% sequence identity to SEQ ID NO: 7.

Embodiment 275. The pharmaceutical composition of embodiment 268, wherein the polynucleotide comprises a nucleotide sequence with at least 95% sequence identity to SEQ ID NO: 16.

Embodiment 276. The pharmaceutical composition of embodiment 268, wherein the polynucleotide comprises a nucleotide sequence with at least 95% sequence identity to SEQ ID NO: 25.

Embodiment 277. The pharmaceutical composition of embodiment 268, wherein the polynucleotide comprises a nucleotide sequence with at least 95% sequence identity to SEQ ID NO: 29.

Embodiment 278. The pharmaceutical composition of embodiment 268, wherein the polynucleotide comprises a nucleotide sequence with at least 95% sequence identity to SEQ ID NO: 31.

Embodiment 279. The pharmaceutical composition of embodiment 268, wherein the polynucleotide comprises a nucleotide sequence with at least 98% sequence identity to SEQ ID NO: 7.

Embodiment 280. The pharmaceutical composition of embodiment 268, wherein the polynucleotide comprises a nucleotide sequence with at least 98% sequence identity to SEQ ID NO: 16.

Embodiment 281. The pharmaceutical composition of embodiment 268, wherein the polynucleotide comprises a nucleotide sequence with at least 98% sequence identity to SEQ ID NO: 25.

Embodiment 282. The pharmaceutical composition of embodiment 268, wherein the polynucleotide comprises a nucleotide sequence with at least 98% sequence identity to SEQ ID NO: 29.

Embodiment 283. The pharmaceutical composition of embodiment 268, wherein the polynucleotide comprises a nucleotide sequence with at least 98% sequence identity to SEQ ID NO: 31.

Embodiment 284. The pharmaceutical composition of embodiment 268, wherein the polynucleotide comprises the nucleotide sequence of SEQ ID NO: 7.

Embodiment 285. The pharmaceutical composition of embodiment 268, wherein the polynucleotide comprises the nucleotide sequence of SEQ ID NO: 16.

Embodiment 286. The pharmaceutical composition of embodiment 268, wherein the polynucleotide comprises the nucleotide sequence of SEQ ID NO: 25.

Embodiment 287. The pharmaceutical composition of embodiment 268, wherein the polynucleotide comprises the nucleotide sequence of SEQ ID NO: 29.

Embodiment 288. The pharmaceutical composition of embodiment 268, wherein the polynucleotide comprises the nucleotide sequence of SEQ ID NO: 31.

Embodiment 289. The pharmaceutical composition of any one of embodiments 183-288, wherein the recognition sequence facilitates binding of the synthetic delivery system to the target cell.

Embodiment 290. The pharmaceutical composition of any one of embodiments 183-289, wherein the recognition sequence facilitates at least 50% higher binding of the synthetic delivery system to the target cell as compared to a control synthetic delivery system that lacks the recognition sequence.

Embodiment 291. The pharmaceutical composition of any one of embodiments 183-290, wherein the recognition sequence facilitates at least 50% higher binding of the synthetic delivery system to the target cell as compared to a control cell of a different cell type.

Embodiment 292. The pharmaceutical composition of any one of embodiments 183-291, wherein the recognition sequence facilitates uptake of the synthetic delivery system or component thereof by the target cell.

Embodiment 293. The pharmaceutical composition of any one of embodiments 183-292, wherein the recognition sequence facilitates at least 50% higher uptake of the synthetic delivery system by the target cell as compared to a control synthetic delivery system that lacks the recognition sequence.

Embodiment 294. The pharmaceutical composition of any one of embodiments 183-293, wherein the recognition sequence facilitates at least 50% higher uptake of the synthetic delivery system by the target cell as compared to a control cell of a different cell type.

Embodiment 295. The pharmaceutical composition of any one of embodiments 183-294, wherein the recognition sequence facilitates at least 50% higher nuclear localization of the synthetic delivery system within the target cell as compared to a control synthetic delivery system that lacks the recognition sequence.

Embodiment 296. The pharmaceutical composition of any one of embodiments 183-295, wherein the recognition sequence facilitates at least 50% higher nuclear localization of the synthetic delivery system within the target cell as compared to a control cell of a different cell type.

Embodiment 297. The pharmaceutical composition of any one of embodiments 183-296, wherein the recognition sequence facilitates binding of the synthetic delivery system to the target cell and uptake of the synthetic delivery system or component thereof by the target cell.

Embodiment 298. The pharmaceutical composition of any one of embodiments 183-297, wherein the recognition sequence comprises a nucleic acid sequence identified in circulating tumor DNA (ctDNA) from a substantially similar cell type as the target cell.

Embodiment 299. The pharmaceutical composition of any one of embodiments 183-298, wherein the polynucleotide comprises double stranded DNA.

Embodiment 300. The pharmaceutical composition of any one of embodiments 183-299, wherein the synthetic delivery system further comprises a cargo.

Embodiment 301. The pharmaceutical composition of embodiment 300, wherein the cargo comprises an anti-cancer therapeutic agent.

Embodiment 302. The pharmaceutical composition of embodiment 300 or 301, wherein the cargo comprises a nucleic acid cargo.

Embodiment 303. The pharmaceutical composition of embodiment 302, wherein the nucleic acid cargo encodes a tumor suppressor protein

Embodiment 304. The pharmaceutical composition of any one of embodiments 300-303, wherein the cargo is appended to a 3′ end of the polynucleotide.

Embodiment 305. The pharmaceutical composition of any one of embodiments 300-304, wherein the cargo is appended to a 5′ end of the polynucleotide.

Embodiment 306. The pharmaceutical composition of any one of embodiments 300-305, wherein the cargo comprises a cytotoxic cargo.

Embodiment 307. The pharmaceutical composition of any one of embodiments 300-306, wherein the cargo comprises a therapeutic cargo.

Embodiment 308. The pharmaceutical composition of any one of embodiments 300-307, wherein the cargo comprises a polypeptide.

Embodiment 309. The pharmaceutical composition of any one of embodiments 300-308, wherein the cargo comprises a small molecule.

Embodiment 310. The pharmaceutical composition of any one of embodiments 183-309, wherein the synthetic delivery system further comprises a promoter.

Embodiment 311. The pharmaceutical composition of any one of embodiments 183-310, wherein the synthetic delivery system further comprises an integration sequence that facilitates integration of the polynucleotide, synthetic delivery system, cargo, or a component thereof into the genome of the target cell.

Embodiment 312. The pharmaceutical composition of embodiment 311, wherein the integration sequence facilitates at least 50% higher genomic integration of the synthetic delivery system, polynucleotide, cargo, or component thereof in target cells as compared to a control synthetic delivery system that lacks the integration sequence.

Embodiment 313. The pharmaceutical composition of any one of embodiments 311-312, wherein the integration sequence facilitates at least 50% higher genomic integration of the synthetic delivery system, polynucleotide, cargo, or component thereof by target cells as compared to control cells of a different cell type.

Embodiment 314. The pharmaceutical composition of any one of embodiments 183-313, wherein the synthetic delivery system does not utilize a viral vector, a nanoparticle, a lipid nanoparticle, a liposome, an exosome, a dendrimer, a gene gun, or electroporation for delivery to the target cell.

Embodiment 315. The pharmaceutical composition of any one of embodiments 183-314, wherein the target cell is a leukocyte.

Embodiment 316. The pharmaceutical composition of any one of embodiments 183-315, wherein the target cell is a plasma cell.

Embodiment 317. The pharmaceutical composition of any one of embodiments 183-316, wherein the target cell is a cancer cell.

Embodiment 318. The pharmaceutical composition of any one of embodiments 183-317, wherein the target cell is a multiple myeloma cell.

Embodiment 319. The pharmaceutical composition of any one of embodiments 183-314, wherein the target cell is a pancreatic cell.

Embodiment 320. The pharmaceutical composition of any one of embodiments 183-314, wherein the target cell is a pancreatic cancer cell.

Embodiment 321. The pharmaceutical composition of any one of embodiments 183-314, wherein the target cell is a gastrointestinal cell.

Embodiment 322. The pharmaceutical composition of any one of embodiments 183-314, wherein the target cell is a colorectal cancer cell.

Embodiment 323. A method of delivering a cargo to a target cell, the method comprising contacting the target cell with the pharmaceutical composition or the synthetic delivery system of any one of the preceding embodiments.

Embodiment 324. The method of embodiment 323, wherein the cargo is delivered to an intracellular compartment of the target cell.

Embodiment 325. The method of any one of embodiments 323-324, wherein the cargo is delivered to a nucleus of the target cell.

Embodiment 326. The method of any one of embodiments 323-325, wherein the cargo comprises a nucleic acid cargo, wherein the nucleic acid cargo is integrated into the target cell's genome.

Embodiment 327. The method of any one of embodiments 323-326, wherein the nucleic acid cargo is integrated into the target cell's genome at a location identified in TABLE 2, TABLE 4, TABLE 7, or TABLE 8.

Embodiment 328. The method of any one of embodiments 323-327, wherein the nucleic acid cargo is integrated into the target cell's genome at Chr2: 32916224-32916626.

Embodiment 329. The method of any one of embodiments 323-327, wherein the nucleic acid cargo is integrated into the target cell's genome at Ch16: 32628381-32629000.

Embodiment 330. A method of treating a condition in a subject in need thereof, the method comprising administering to the subject the pharmaceutical composition of any one of the preceding embodiments.

Embodiment 331. The method of embodiment 330, wherein the subject is mammalian.

Embodiment 332. The method of any one of embodiments 330-331, wherein the subject is human.

Embodiment 333. The method of any one of embodiments 330-332, wherein the condition is a cancer.

Embodiment 334. The method of embodiment 333, wherein the cancer is a hematologic cancer.

Embodiment 335. The method of any one of embodiments 333-334, wherein the cancer is a multiple myeloma.

Embodiment 336. The method of embodiment 333, wherein the cancer is a solid tumor.

Embodiment 337. The method of embodiment 333, wherein the cancer is a pancreatic cancer.

Embodiment 338. The method of any one of embodiment 333, wherein the cancer is colorectal cancer.

### VII. EXAMPLES

### Example 1: Identification of Genomic Integration Sites of ctDNA

The integration of ctDNAs into the host cell genome was evaluated. ctDNA was extracted from patient plasma, cultured for 24 hours with cells of a matching cancer cell type or non-matching cancer cell type, and whole genome sequencing was performed. The cell lines used were MM1s (multiple myeloma, MM) and MIA (pancreatic cancer, PC).

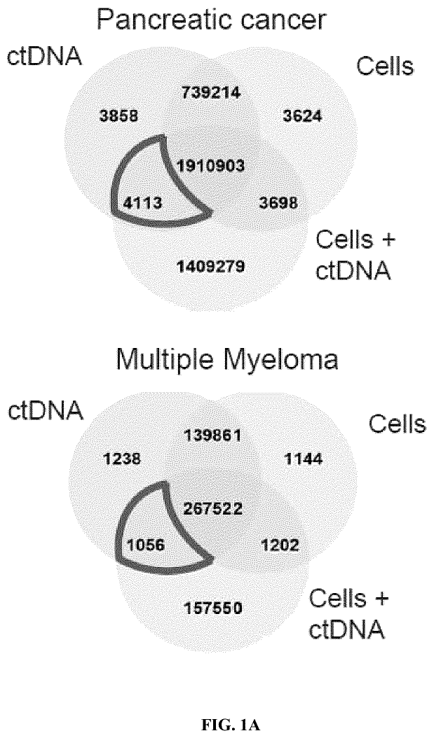

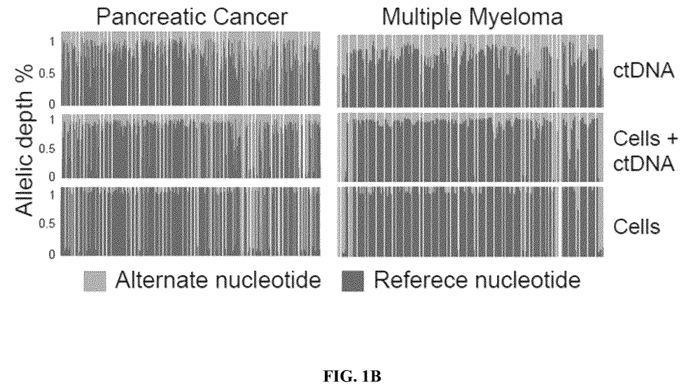

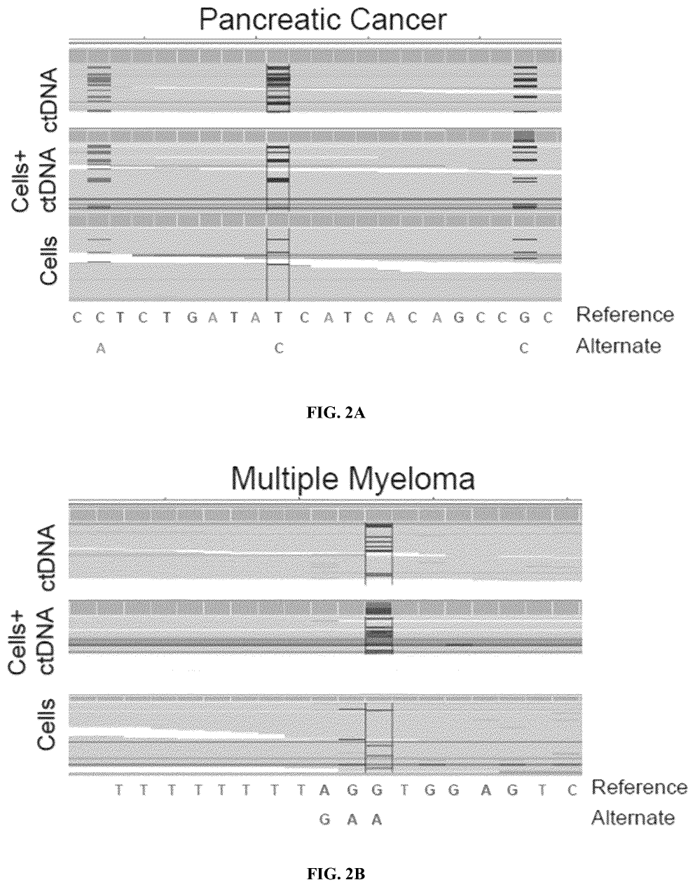

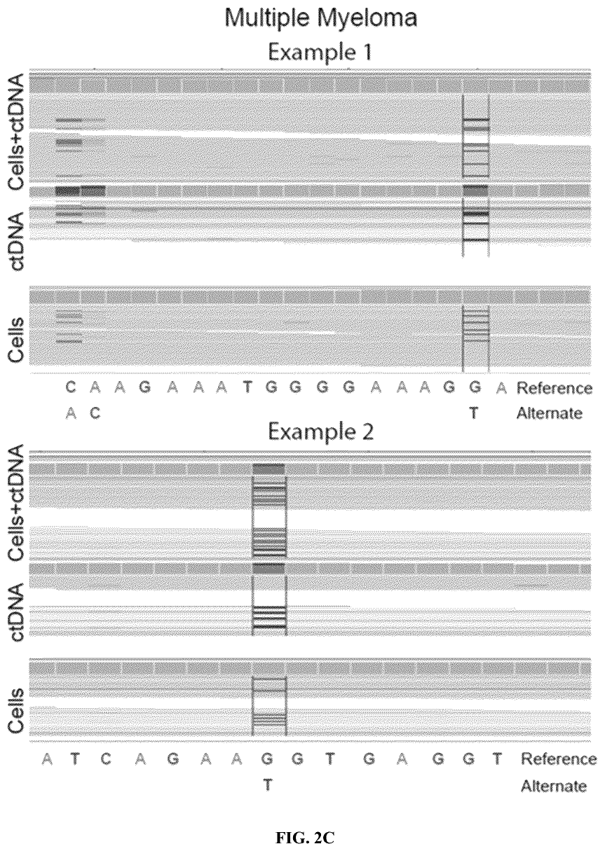

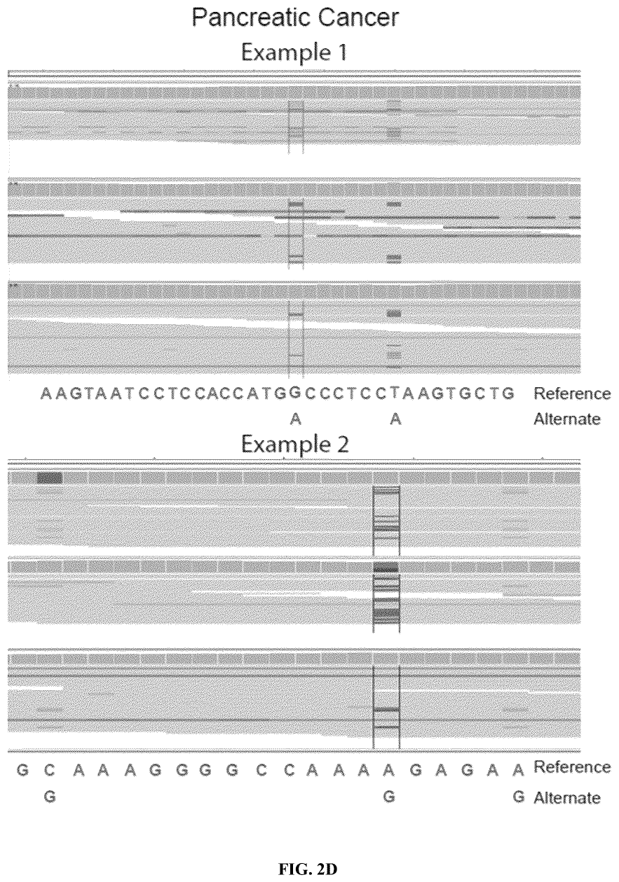

Single nucleotide variants (SNVs) unique to the ctDNA that were found in the host cell genome under matching coculture conditions were identified. Comparing the sequence from each experimental condition after alignment to the reference human genome (Hg38) resulted in identification of multiple SNVs shared between coculture conditions and ctDNA that were not present in the genome of the untreated control cells (here called “SNVs of interest,” FIG. 1A). Further detailing of these SNVs of interest demonstrated that cells cocultured with ctDNA showed skewing of the variant allele frequency towards that of the variant ctDNA allele compared to cells alone (FIG. 1B). Examples of SNVs exhibiting ctDNA ‘skewing’ in the variant allele frequency (VAF) after coculture with ctDNA are shown in FIGS. 2A-2D.

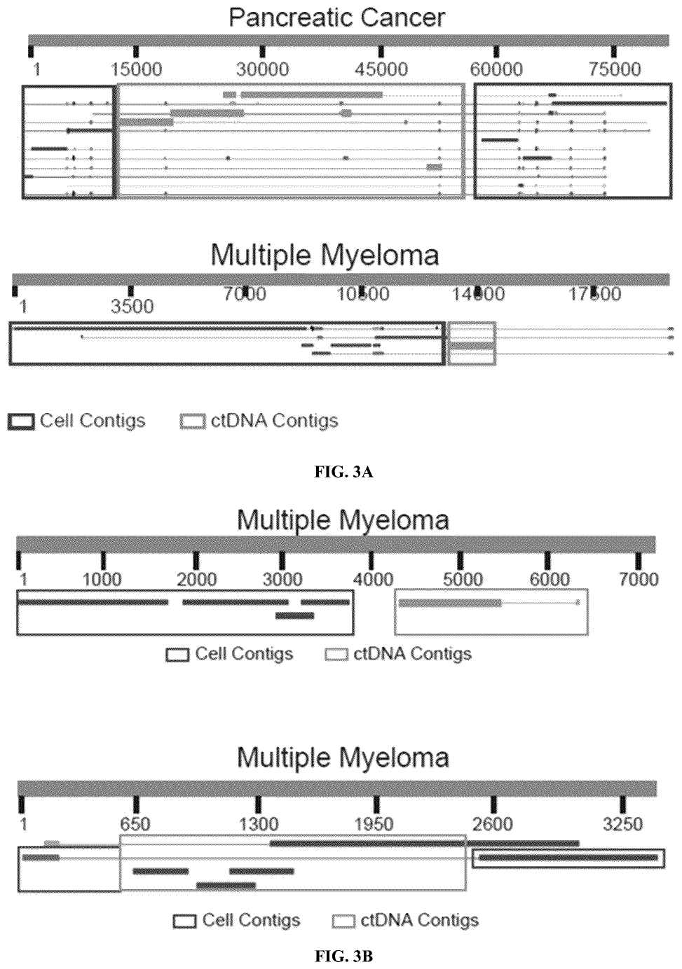

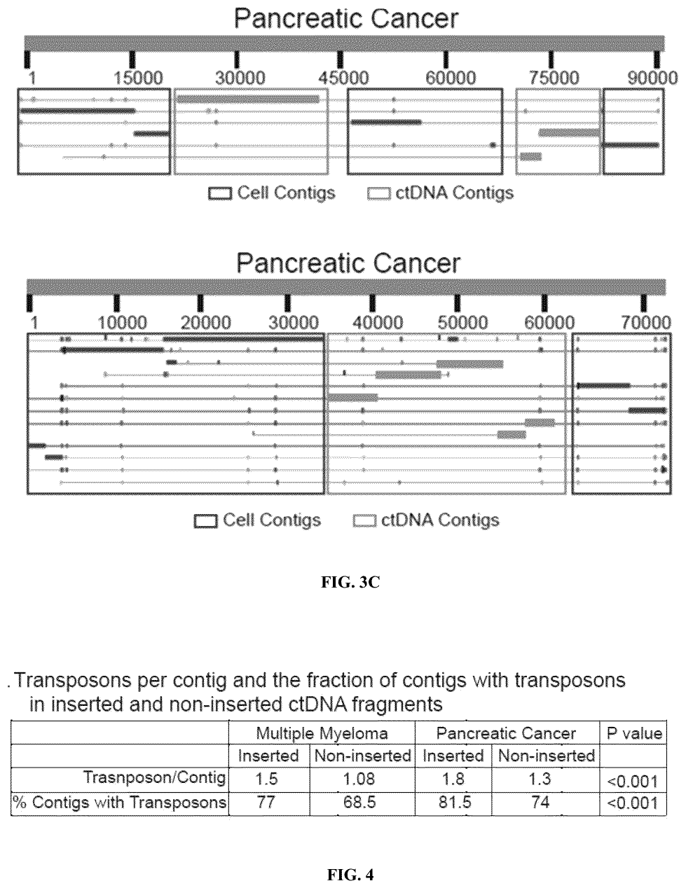

The sequence data was used to reconstruct genomic contigs for each experimental condition by de novo assembly. Comparative analysis of the contig sequences between the cell line, ctDNA, and coculture conditions described above resulted in detection of ctDNA fragments integrated into the cell genome under coculture conditions. Coculture contigs carrying a ctDNA insertion were identified using NucDiff analysis. BLAST alignment analysis further validated the gain of ctDNA fragments in coculture conditions and identified the transition points of ctDNA insertion (FIGS. 3A-3C). Analysis of incorporated ctDNA fragments and genome-insertion sites identified from both tumor models indicated that most (˜67%) of the inserted ctDNA fragments originated from chromosomes 3 and 7. Moreover, about 80% of the ctDNA fragments targeted cellular chromosomal regions near the genomic location from which ctDNA originated, suggesting a high potential for insertion of ctDNA in homologous target regions. The remaining 20% inserted into a chromosomal location distinct from their site of origin.

### Example 2: Identification of Transposable Elements in ctDNA

Experiments were designed to identify the presence of transposons in ctDNA fragments. To map ctDNA genomic junctions more precisely, a PACBIO probe was ligated to multiple myeloma and pancreatic cancer ctDNA samples to label their 5′ and 3′ ends. Whole-genome sequencing of DNA from cells with ctDNA genomic integration was done using the Illumina platform. De novo assembly was performed on the sequencing output. ctDNA contigs containing PACBIO adaptor were then matched to those contigs containing inserted ctDNA sequences identified in the coculture experiments. Transposable element on ctDNA sequences at the insertion points (<100 nucleotides from transition point between cell and ctDNA) were detected and classified using RepeatMaster. This analysis demonstrated that the ctDNA fragments integrated into the cell genome contained more transposable elements than non-integrated ctDNA fragments (FIG. 4). These findings suggested that retrotransposons mediate horizontal gene transfer in cancer cells.

A search was conducted for transposons that preferentially target cells of matching tumor types. The lists of transposons identified at the ctDNA insertion point in matching and mismatched coculture conditions were compared (e.g., MM ctDNA with MM cells or PC cells). Transposons were selected that were uniquely or preferentially inserted in the matching conditions.

Class I transposons ERV-L, LTRs, SINE, and LINEs comprised the majority of transposable elements at the transition points in pancreatic cancer or multiple myeloma.

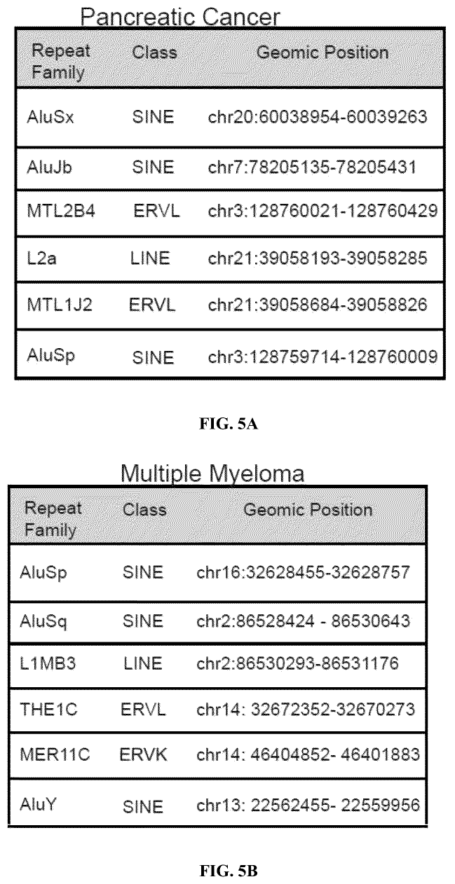

Within matching PC ctDNA, AluSx, MIRc, and MTL1J were among the most common retrotransposons subfamilies located at insertion points (FIG. 5A and TABLE 8).

In multiple myeloma, AluSp, MER11, MER11C, AluJb, and L2a, among other subfamilies of retrotransposons were identified at the MM ctDNA insertion sites (FIG. 5B and TABLE 7).

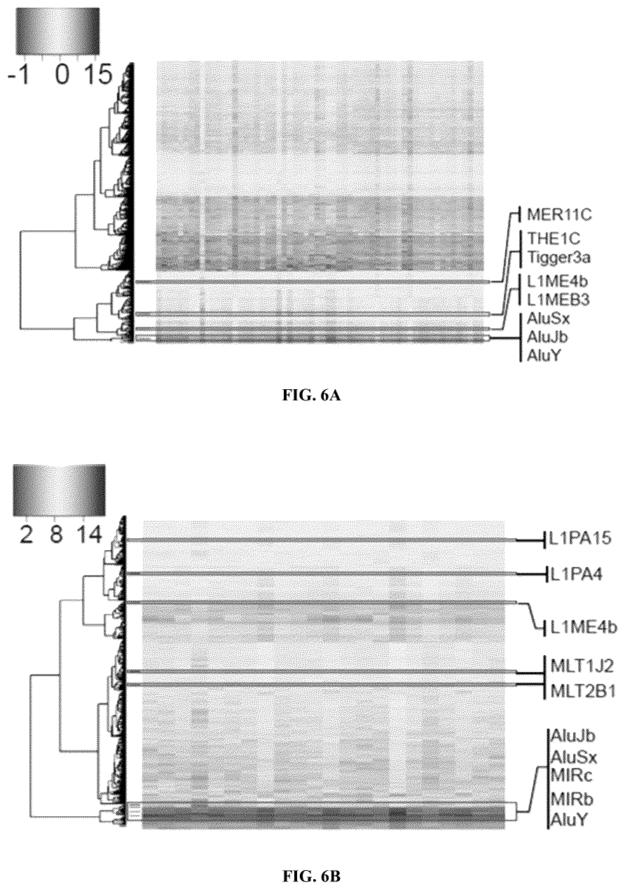

Activation of short interspersed nuclear elements (SINEs) and long interspersed nuclear elements (LINEs) have been observed in cancer, leaving open the possibility of retrotransposition. Expression of transposable elements was evaluated in RNA sequencing data of 60 MM and 23 PC patient samples. Batch-normalized raw data was used to measure the expression variability. The analysis confirmed several tissue-specific retrotransposons to be highly expressed across pancreatic cancer or multiple myeloma. These findings demonstrate that the retrotransposons identified at insertion sites were transcriptionally active in these cancer types (FIGS. 6A and 6B).

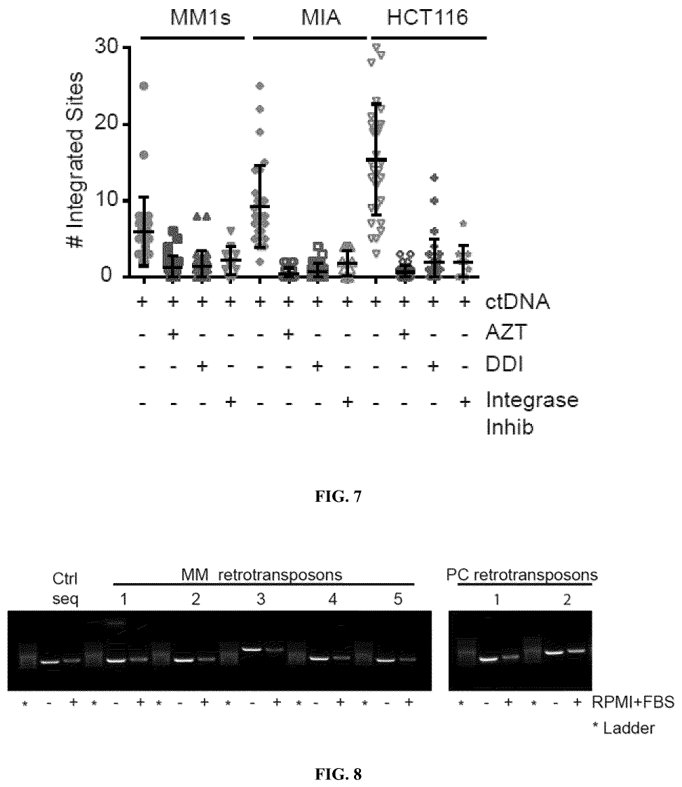

To evaluate functionally the role of retrotransposons in horizontal gene transfer of ctDNA, experiments were conducted to test the impact of inhibiting reverse transcriptases or integrases on ctDNA integration. Multiple myeloma (MM1s), pancreatic cancer (MIA), and colon cancer (HCT116) cell lines were treated with the reverse-transcriptase inhibitors zidovudine or didanosine, and with the integrase inhibitor raltegravir prior to adding the ctDNA to culture media. Treatment with the reverse transcriptase inhibitors or integrase inhibitor significantly reduced ctDNA chromatid integration (FIG. 7) compared to the no treatment control, providing evidence that retrotransposons play an important role in ctDNA horizontal gene transfer.

### Example 3: Evaluation of Synthesized Transposable Elements and Cargo Delivery

Several MM retrotransposons containing point mutations unique to the tumor type were synthesized and tested for capacity for integration. Retrotransposons were selected that could be synthetized via the gBlock gene fragment method (including AluSp, AluSg2, MER11C, THE1A and AluSx). For controls, a PC retrotransposon (AluSx1) and a synthetic sequence with similar length and GC content that did not contain a retrotransposon sequence were included.

The retrotransposons' ability to resist the degradation induced by DNAses contained in complete culture media was evaluated. Agarose gel electrophoresis demonstrated that all fragments remain intact under culture conditions (FIG. 8).

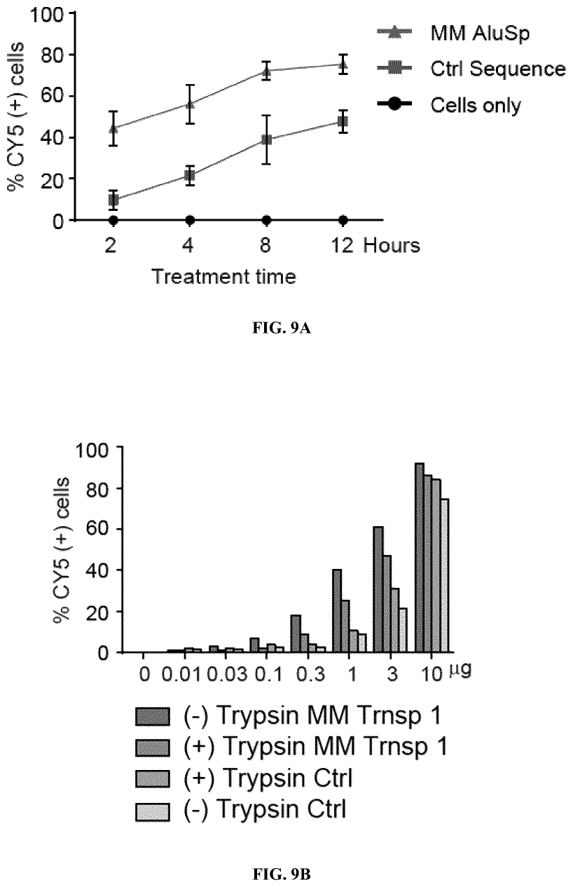

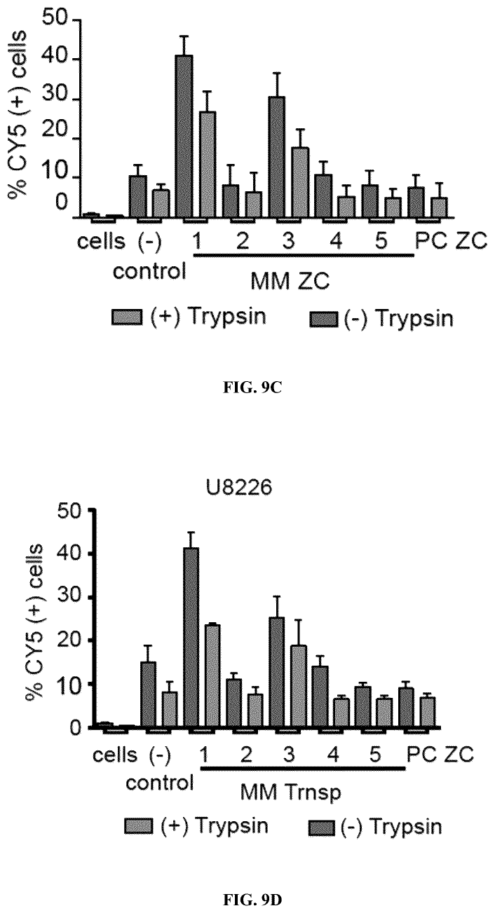

The retrotransposons were labelled with CY5 to determine uptake by MM cell lines using flow cytometry methods. The kinetics of retrotransposon capture were monitored by flow cytometry. Results showed that the cell capture of CY5-AluSp increased over time more efficiently than the control sequence (FIG. 9A).

Dose titration experiments were performed to evaluate cell capture and internalization. DNA was considered internalized if the DNA was not removed from the cells by trypsin treatment. This experiment delineated a dose-dependent CY5-AluSp cell capture and internalization that was more efficient that than that observed in control sequence (FIG. 9B).

The variability of cell capture of several transposons was tested in two MM cell lines at 4 hours of culture. These experiments identified that CY5-AluSp and CY5-MER11C cell capture was higher than that of the other transposons, the control sequence, and PC transposons (FIG. 9C and FIG. 9D).

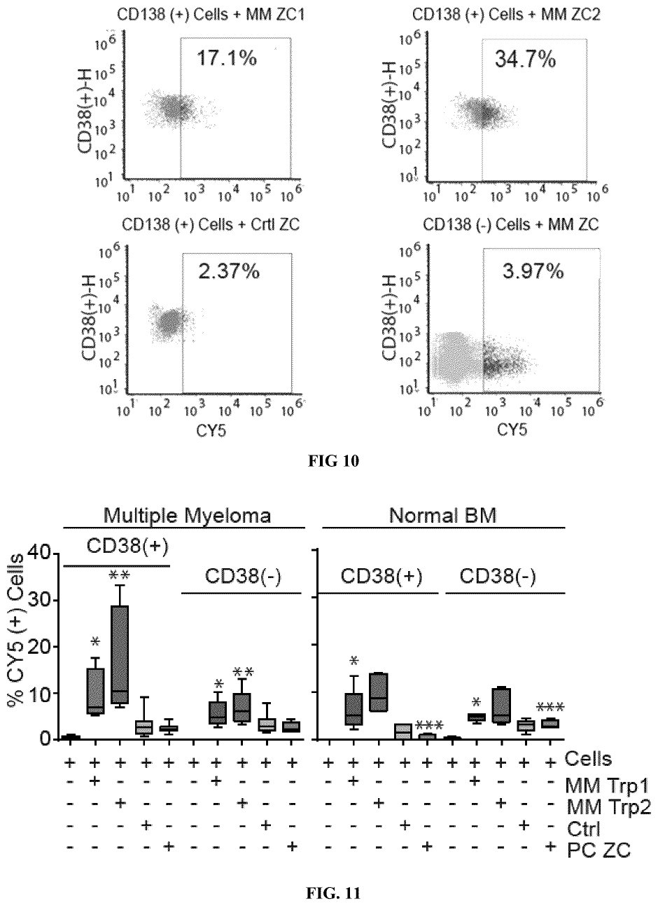

Cell targeting of the AluSp and MER11C sequences was tested in bone marrow samples derived from patients with and without multiple myeloma. After 14 hours of cell culture, more CY5 positive plasma cells were observed in samples cultured with CY5-AluSp or CY5-MER11C (FIG. 10, upper panels) than with a control sequence or PC specific sequence (FIG. 10, lower left panel). Interestingly, the levels of plasma cell targeting were higher in plasma cells derived from MM patients than those without MM. This result suggests that malignant plasma cells are more suitable for capturing these retrotransposons (FIG. 11). AluSp and MER11C showed higher levels of signal in the plasma cells of MM patients than in the non-MM CD138 (−) cells compartment, validating their tissue-specific cell targeting capability (FIG. 10, lower right panel, and FIG. 11).

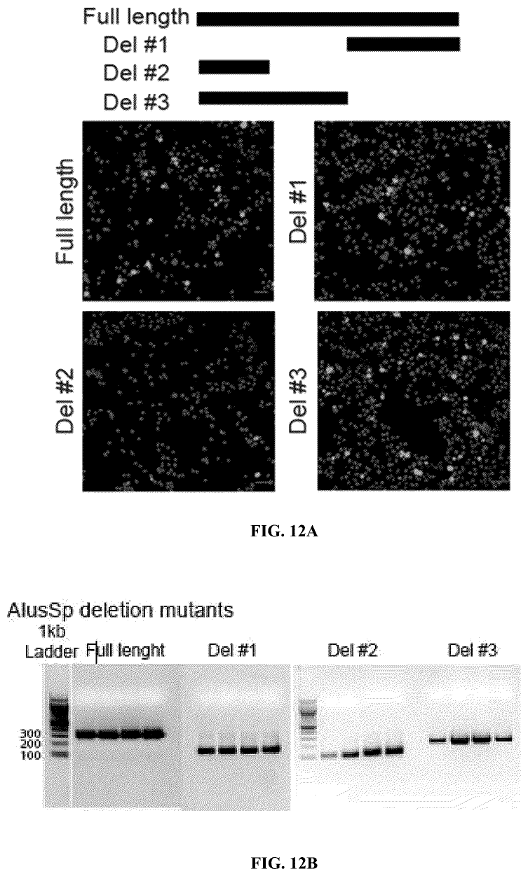

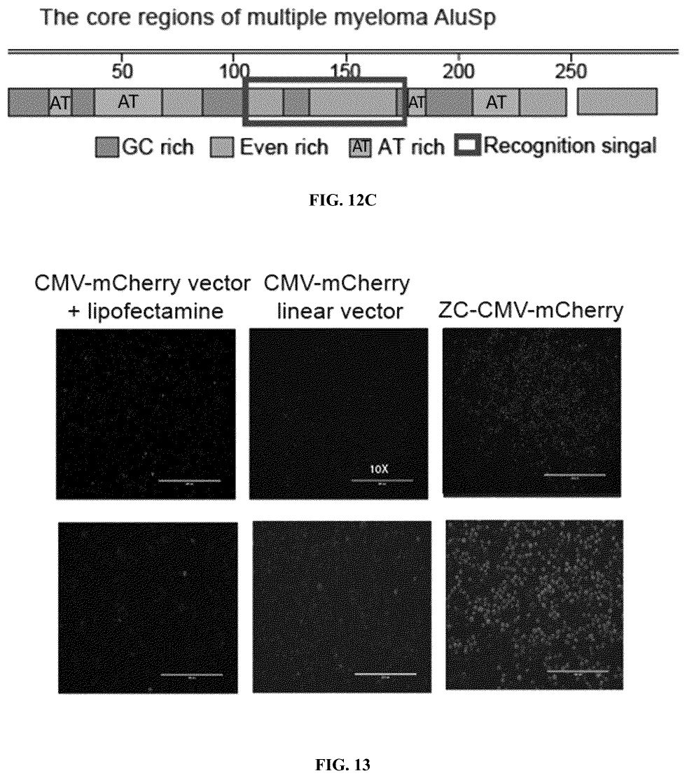

To test that the retrotransposon sequence contains a cell recognition signal, several deletion mutants of the AluSp sequence were generated and evaluated for impact of the deletion on cell capture. The results showed that lacking the 3′ end of the AluSp sequence reduced MM cell capture, suggesting that the last ˜230 based pairs contain an MM cell recognition sequence (FIGS. 12A and 12B). Multiple sequence alignments of AluSp and all MM specific transposons revealed several conserved nucleotides and two GC and AT rich regions towards the 3′ end (FIG. 12C).

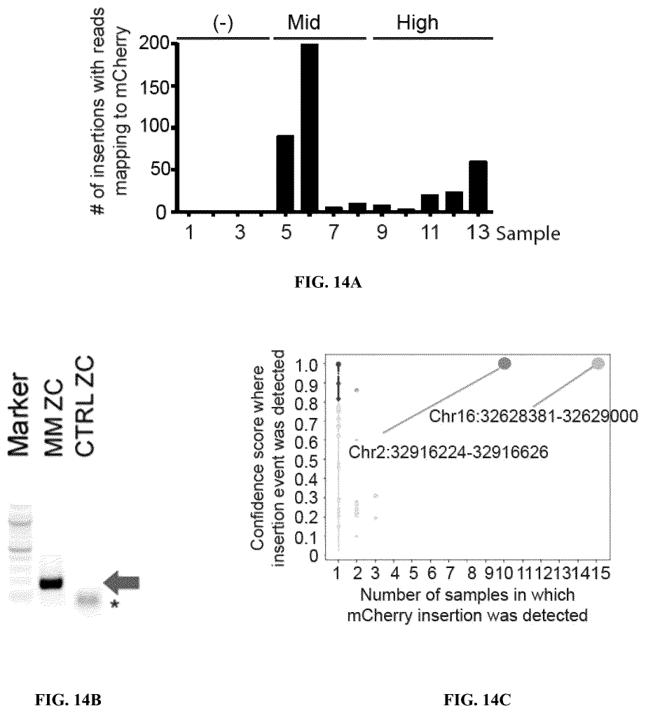

Experiments were designed to test ability of the AluSp sequence for transferring genetic material between cells. Delivery of an mCherry reporter cassette to MM cell lines by the AluSp sequence was tested. To do so, AluSp was ligated to both ends of a linearized CMVpromoter-mCherry cassette. This AluSp-CMV-mCherry ligation product was then added to the culture media of MM1s cells for 24 hours and mCherry expression evaluated. Microscopy experiments demonstrated a higher number of mCherry expressing cells in AluSp-CMV-mCherry treated cells versus those treated with the linearized vector alone or after transfection with the circularized CMV-mCherry vector (FIG. 13). The expression of mCherry in MM1s cells culture with AluSp-CMV-mCherry was also detected by flow cytometry.

To evaluate genomic integration the AluSp-CMV-mCherry cassette, whole-genome single-cell sequencing was performed on MM1s cells expressing various levels of mCherry. mCherry insertions were identified by recognizing sequences with one read aligned to the cell genome while the mate aligned to the CMV or mCherry sequence. This analysis detected various levels of mCherry insertions in cells with mid or high expression levels, while no insertions were identified in cells that failed to express mCherry (FIG. 14A). In contrast, when cells were cultured with control-mCherry sequence, only a few were found with high levels of mCherry insertion and expression. mCherry integration was confirmed by mCherry PCR of cells cultured with AluSp-mCherry vector but not in control-mCherry treated cells (FIG. 14B). The AluSp-CMV-mCherry vector integrated with high confidence into two specific regions in the genome (Chr2: 32916224-32916626 and Ch16: 32628381-32629000) that are enriched for simple repeats and AluSp, respectively (FIG. 14C).

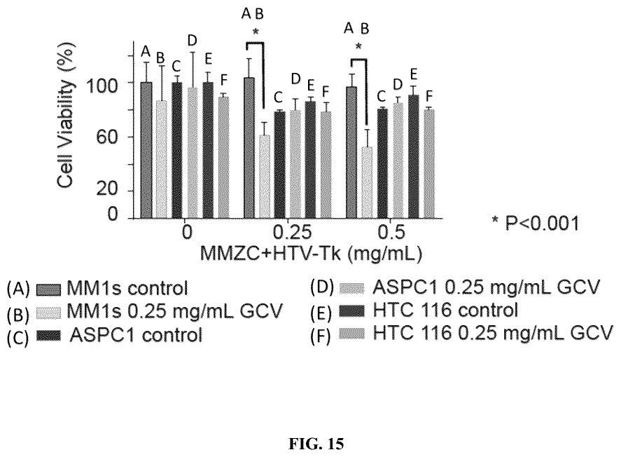

The tissue-specific cell targeting capacity of the AluSp and ability to deliver a payload were evaluated by ligating herpes simplex virus tyrosine kinase (HSV-Tk) cytotoxic gene between AluSp sequences (AluSp-HSV-Tk-GFP) and testing the construct's effect on the viability of MM, PC, and CC cell lines. Cells were cultured for 24 hours with the AluSp-HSV-Tk-GFP, followed by a 96-hour treatment with ganciclovir (GCV), which is converted into a toxic product by HSV-Tk. GCV specifically reduced cell viability of MM cells when treated with MM-AluSp-HSV-Tk (FIG. 15). In contrast, AluSp-HSV-Tk/GCV treatment caused no or substantially no changes in viability in PC or CC cell lines.

These results demonstrated that the synthesized AluSp could transfer and integrate genetic cargo material into specific cells, and could be combined with a cytotoxic cargo to target cancer cells for killing.

### Example 4: ctDNA Alters the Drug Response Phenotype of MM or PC Cell Lines

The effect of ctDNA horizontal gene transfer on cell drug sensitivity was evaluated.

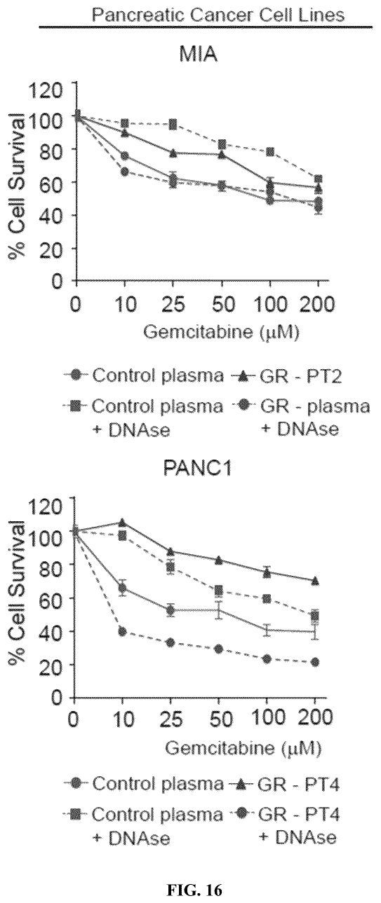

Gemcitabine (GEM)-sensitive pancreatic cancer cell lines (MIA Paca-2 [MIA] and PANC1) were cultured for 24 h with DNase-treated or non-treated plasma from (GEM)-resistant (GR) patients or control plasma. Compared to control serum, both cell lines became resistant to GEM when cultured with non-DNase-treated GR plasma. Exposure to the DNase-treated GR plasma restored the inherent sensitivity of the cells to the treatment (FIG. 16). In contrast, treatment of control serum with DNase resulted in an increased resistance of PC cells to gemcitabine.

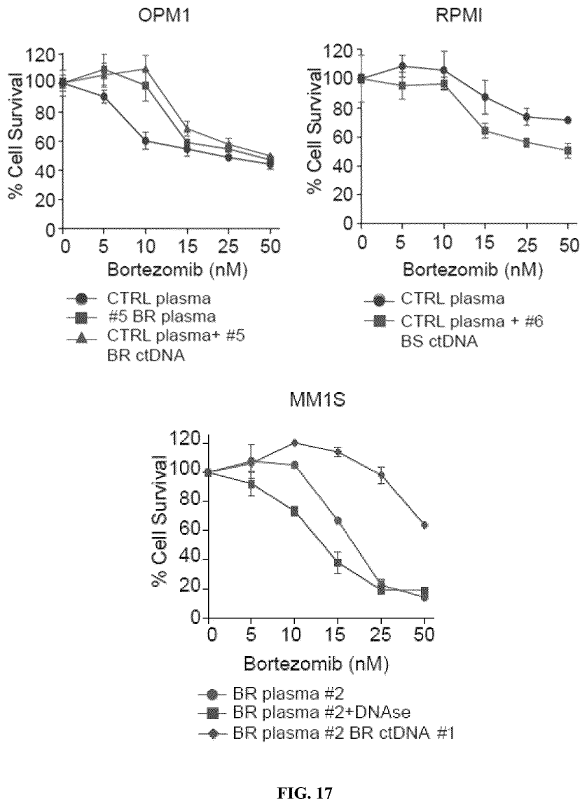

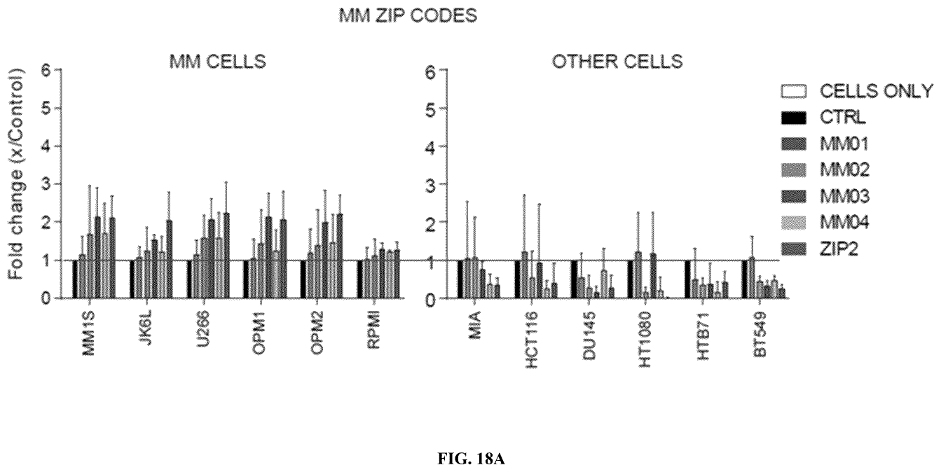

To validate further the contribution of ctDNA to drug-response transmission, cell-free ctDNA was extracted from bortezomib-resistant (BR) or-sensitive (BS) patients and added to media containing plasma of a patient without cancer or bortezomib-resistance. The result showed that the addition of ctDNA derived from a BR patient to non-cancer patient plasma decreased the sensitivity of MM1s and OPM1 to bortezomib (FIG. 17). In addition, resistance to bortezomib was further increased when ctDNA of a BR patient was added to plasma of a BR patient. In contrast, the addition of ctDNA of a BS patient to RPMI cells cultured with control plasma from non-cancer patients resulted in increased sensitivity to bortezomib. These findings suggest that ctDNA can transmit genetic material that confers drug sensitivity or resistance from one cell to another.

### Example 5: Additional Materials and Methods

Clinical specimens and sample preparation: Retrospective plasma samples were obtained from 10 multiple myeloma (MM), 10 pancreatic cancer (PC), 3 colon cancer (CC) and 2 lung cancer (LC) patients from samples stored in a Tissue and Acquisition. The 10 patients with newly diagnosed MM were treated with bortezomib-containing regimens (among them were 5 responders and 5 non-responders). Of the 10 PC patients, 7 with advanced staged cancer were treated with gemcitabine (at the time of obtaining the plasma sample 2 patients were in partial response and 5 had progressive disease) and 3 early-stage patients had undergone surgical resection. Response in MM was determined using the International Uniform Response Criteria for Multiple Myeloma, and PC patients were evaluated using the Response evaluation criteria in solid tumors (RECIST) criteria. Plasma was isolated after centrifuging blood samples at 1500 rpm for 10 minutes. The plasma supernatant was collected for storage. Plasma from non-cancer patients was purchased from a commercial vendor.

Cell lines and reagents: The following cell lines were grown in Roswell Park Memorial Institute (RPMI)-1640 medium: Four multiple myelomas (OPM1, RPMI, JK6L, and MM1S); one PC (ASPC-1); and one LC (A549). Colon cancer cell lines (HCT-116 and HT-29) were cultured in McCoy media. Pancreatic cancer cell lines (PANC1 and MIA Paca-2 [MIA]) were cultured in DMEM media, and CC cell line RKO was cultured in EMEM. All culture media were supplemented with 10% fetal bovine plasma, 1% L-glutamine, 1 mM sodium pyruvate, and 50 μg/ml penicillin-streptomycin. In MIA cells, 2.5% horse serum was also added to the culture medium.

Cell viability: Cell viability assays were performed in 96 well, black, clear-bottom microplates. For MM cell line studies, 3×10\hat{ }4 cells were cultured for 24 hours with media containing 10% human plasma of a bortezomib-resistant or-sensitive patient or a control non-cancer patient. Cells were then cultured for 24 hours with titrating concentrations of bortezomib (doses: 0, 5, 10, 15, 50 nM; Sigma). For PC cell lines viability studies, 1×10\hat{ }4 cells were incubated for 24 hours with media containing plasma of a gemcitabine-resistant patient or non-cancer patient. Subsequently, titrating concentrations of gemcitabine (doses: 0, 10, 25, 50, 100, 200 μM; Sigma-Aldrich, MO) were added to the culture media and cells were then incubated for 96 hours. CellTiter-Blue Cell Viability Assay was used to evaluate cell viability according to the manufacturer's instructions (Promega). Cell viability was measured by a fluorescent protocol in an microplate reader. Experiments were performed in triplicate of 3 independent studies.

ctDNA extraction and immunofluorescence labeling: Isolation of ctDNA was performed following QIAamp MinElute ccfDNA Kits protocol (Qiagen, Cambridge, MA). The ctDNA was fluorescently labeled with either Cx-rhodamine or CY5 using the Label IT® Nucleic Acid Labeling kit (Mirus Bio LLC, WI).

Image acquisition: 1×10⁶ cells cultured in 1 ml of culture media were incubated with rhodamine- or CY5-labeled DNA at different time points. Adherent cells (2.5×10\hat{ }5 cells) were grown on a coverslip prior to processing. After cells were attached to slides, slides were washed with PBS twice, and the cells were then counterstained with 4, 6-diamidino-2-phenylindole (DAPI) for nuclear detection (ThermoFisher MA). For live-cell imaging, the plasma membrane was labeled following the cellLight Plasma Membrane-green fluorescent protein (GFP), Bacman 2.0 protocol (ThermoFisher MA). The presented images are from triplicate experiments. Images were acquired using a Leica SP8 LIGHTING confocal microscope.

Xenograft experiments: Pilot and validation xenograft studies are performed to evaluate the accumulation of ctDNA in tumors in mice. For the pilot time-course study, 3 mice bearing pancreatic cancer MIA cell xenografts are generated by injecting 1×10\hat{ }6 cells in the dorsum of J: NU (007850) outbred nude mice. After tumors reach a volume of 0.5 cm, mice are assigned to a specific experimental arm: some mice undergo tail vein injection with rhodamine-labeled ctDNA or synthetic delivery system and others receive a tail-vein injection with PBS as a control. Tumors from tail vein ctDNA-injected mice are harvested 24 and 48 hours post-injection. For mice in the control group, tumor is harvested 48 hours post-injection. Based on the results of these experiments, a harvest time point is selected, e.g., of 48 hours post-injection. For the validation study, xenograft models are developed using human-derived PC (MIA), MM (MM1s), and CC (HCT-116) cell lines. Following pilot study protocol, 3 mice per tumor xenograft are then dosed to assess tumor localization of labeled ctDNA. At harvest time, tumors and selected organs (liver, lung, large bowel, pancreas, and spleen) undergo frozen section dissection. Each slide is fixed with paraformaldehyde 4% and stained with DAPI before mounting the coverslip.

Whole-genome sequencing and Whole exon sequencing: ctDNA was extracted from 10 MM and 10 PC patients. DNA from CD138 (+) cells and from cell lines used in in vitro experiments (MM1s and MIA) was extracted using Blood & Cell Culture DNA Mini Kit (Quiagen, MD). DNA from fresh frozen paraffin embedded pancreatic tumors was obtained using QIAamp DNA FFPE Tissue Kit Print (Qiagen, MD).

After extraction, ctDNA was ligated to the PACBIO adaptor (GCGCTCTGTGTGCT) using the ABM DNA Library Prep Kit for Illumina Sequencing (Applied Biological Materials Inc. Canada). PACBio-labeled ctDNA and regular ctDNA were subjected to standard methods for library preparation and sequencing using Illumina and Agilent protocols, respectively. Applied Biological Materials Inc. prepared the libraries and performed whole-exon and -genome sequencing. The average target coverage was 50×.

Nucleotide variance concordance between tumor and ctDNA: The raw sequence data was subjected to quality control checks using FastQC (https://www.bioinformatics.babraham.ac.uk/projects/fastqc/). Samples that failed the QC checks were trimmed using BBDuk (https://jgi.doe.gov/data-and-tools/bbtools/bb-tools-user-guide/bbduk-guide/) in the adapter trimming mode for paired reads. The sequence reads were then aligned to the human genome GRCh38 assembly (https://www.ncbi.nlm.nih.gov/assembly/GCF_000001405.39) using the Falcon Accelerated Genomics Pipeline (https://github.com/falconcomputing/falcon-genome). This program is an accelerated version of the GATK Best Practices Pipeline. Beginning with paired-end FASTQ sequence files, the first step mapped the sequences to the reference. The resulting mapped BAM file was sorted and duplicates marked. GATK 4.1.3 best practice somatic mutation pipeline was run with base recalibration, with read orientation filtering to account for damage seen when using FFPE samples (https://www.intel.com/content/dam/www/programmable/us/en/pdf/literature/wp/wp-accelerating-genomics-open1-fpgas.pdf, https://www.intel.com/content/www/us/en/healthcare-it/solutions/genomicscode-gatk.html and https://pdfs.semanticscholar.org/e85d/4f927d91e9f25b7c20de71f91c78250771bb.pdf).

Variant Calling And Annotation: Variant calling was done using VarDict (https://academic.oup.com/nar/article/44/11/e108/2468301), a versatile variant caller for next-generation sequencing in cancer research. An allele frequency threshold of 0.01 was used. Variants for 6 of the samples (for which a control samples were not available) were called in single sample mode (https://github.com/AstraZeneca-NGS/VarDict). For the samples where controls were available, paired mode was run to distinguish between somatic and germline variants. The called variants were annotated using SnpEff (http://snpeff.sourceforge.net/SnpEff.html), which is a variant annotation and effect prediction tool.

Analysis for concordance of ctDNA and primary tumor sample: Single nucleotide variant concordance between ctDNA and corresponding tumor samples was analyzed using bcftools isec (http://samtools.github.io/bcftools/bcftools.html) to obtain concordant variants.

Identification of transposable elements and its nucleotide variants: To determine the locations of transposable-like regions in contigs, sequences were analyzed and transposable elements (TEs) were identified and classified using RepeatMasker version 4.1.0 and the Dfam database (release 3.1) of repetitive DNA families was used as a reference for identifying repeats in ctDNA contig sequences that were part of qualifying structural events. For each repeat sequence identified by RepeatMasker, the overall frequency of the specific repeat (e.g., for AluSp or L1) and their parent class (e.g., SINE) was computed.

Once the TE elements were identified, hit repeats/TE sequences, unique contigs for all samples, and insertion sequence were aligned with MAFFT software version. Mutations in TE sequences were identified by comparison of nucleotides at each position in the multiple sequence alignment with the “Biostrings” R package (version 4.1). A position was considered to contain mutation if the substitution was present in contigs of all samples. In the case of ambiguous nucleotides in contigs introduced by short read alignment (putative heterozygosity in the sample), a non-matched nucleotide was considered as a mutation if the non-matched nucleotide was present in all samples. Based on the identified mutations, the mutated transposon sequences for each unique contig was constructed (for MM1S and MIA datasets).

Transposon linearized vector: A polynucleotide comprising sequences corresponding to the transposon that contained mutations shared by all the MM samples was generated by Integrated DNA Technologies, Inc (IDT). Similar methods were used to generate deletion mutants from AluSp.

### Example 6: Cell Type-Specific Uptake of Retrotransposon-Derived Synthetic Delivery System DNA

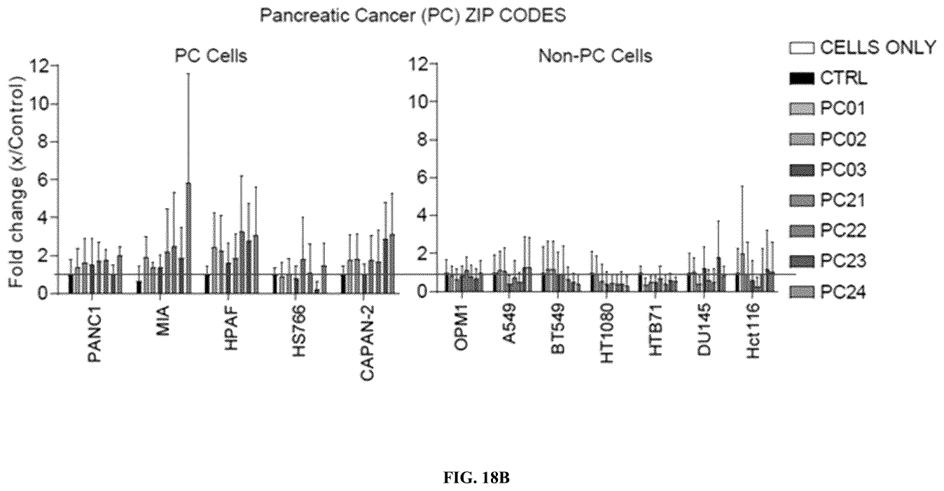

Synthesized, CY5-labeled retrotransposon sequences derived from multiple myeloma (MM) ctDNA were cultured with MM or non-MM cell lines, and synthesized, CY5-labeled retrotransposon sequences derived from pancreatic ductal carcinoma (PDAC) ctDNA were cultured with pancreatic cancer (PC) or non-pancreatic cancer cell lines, for four hours. Positive cells were identified by high throughput image express microscopy. Preferential uptake was demonstrated for the synthesized DNA sequences for the same cell type that the ctDNA originated from (FIGS. 18A-MM01-MM04 and “ZIP2” are MM-derived; FIG. 18B-PC01-03 & PC21-24 are PDAC-derived).

### Example 7: Analysis of MM ctDNA-Derived Sequence to Identify Recognition Sequence

This example demonstrates analysis of a DNA sequence derived from MM ctDNA to identify a region that contributes to uptake by MM cells (e.g., a recognition sequence).

A DNA sequence derived from MM ctDNA with a demonstrated ability to elicit uptake by MM cells (SEQ ID NO: 68) was amplified with suitable primer pairs to generate fragments for testing uptake. Fluorescein 12 dCTP from Perkin Elmer was used to label the PCR products, which were purified after the reaction.

In an initial study, fragments were generated as follows:

TABLE 5

| SEQ | | |
|---|---|---|
| ID | Descrip- | |
| NO | tion | Sequence |
| 68 | MM2 | ACCCGGCCTTGGACACGCCATTTTCAACTCCG |
| | (1-290) | TGGTGCGTTTTTTTTTTTTTTTTTTTTTTTTT |
| | | GTAATGGAGTTTCGCTCTTGTTGCCCAGGATG |
| | | GAGTGCAAGGGATCTTGGCTCACCACAGCCTC |
| | | CGCCTCTTGGGTTCAAGTGATTCTTCTGCCTC |
| | | AGCCTCCCAAGTAGCTGGGATTATAAGCACCC |
| | | ACCACCACGCCCAGCTAATTTTGTATTTTTTA |
| | | GAAGAGATGGAGTTTCTCCAGTTGGCCAGGAT |
| | | GGTCTGTATATCCTGACCTCATGATCTGCCCA |
| | | CC |
| 69 | MM2 | CCAGGATGGAGTGCAAGGGATCTTGGCTCACC |
| | (89-290) | ACAGCCTCCGCCTCTTGGGTTCAAGTGATTCT |
| | | TCTGCCTCAGCCTCCCAAGTAGCTGGGATTAT |
| | | AAGCACCCACCACCACGCCCAGCTAATTTTGT |
| | | ATTTTTTAGAAGAGATGGAGTTTCTCCAGTTG |
| | | GCCAGGATGGTCTGTATATCCTGACCTCATGA |
| | | TCTGCCCACC |
| 70 | MM2 | GGGATTATAAGCACCCACCACCACGCCCAGCT |
| | (177-290) | AATTTTGTATTTTTTAGAAGAGATGGAGTTTC |
| | | TCCAGTTGGCCAGGATGGTCTGTATATCCTGA |
| | | CCTCATGATCTGCCCACC |
| 71 | MM2 | ACCCGGCCTTGGACACGCCATTTTCAACTCCG |
| | (1-113) | TGGTGCGTTTTTTTTTTTTTTTTTTTTTTTTT |
| | | GTAATGGAGTTTCGCTCTTGTTGCCCAGGATG |
| | | GAGTGCAAGGGATCTTG |
| 72 | MM2 | ACCCGGCCTTGGACACGCCATTTTCAACTCCG |
| | (1-201) | TGGTGCGTTTTTTTTTTTTTTTTTTTTTTTTT |
| | | GTAATGGAGTTTCGCTCTTGTTGCCCAGGATG |
| | | GAGTGCAAGGGATCTTGGCTCACCACAGCCTC |
| | | CGCCTCTTGGGTTCAAGTGATTCTTCTGCCTC |
| | | AGCCTCCCAAGTAGCTGGGATTATAAGCACCC |
| | | ACCACCACG |

The fluorescent DNA sequences were incubated with multiple a myeloma cell line and uptake was evaluated by fluorescent microscopy.

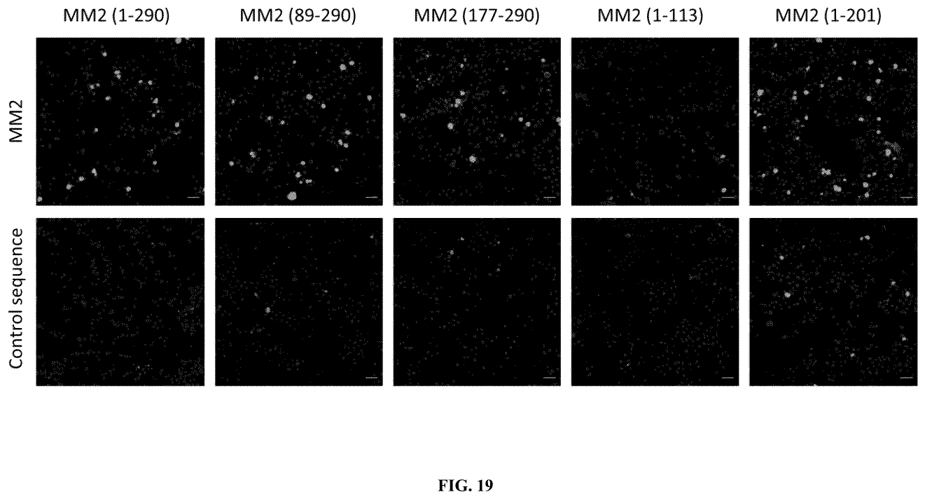

Uptake was observed for the MM2 (1-290), MM2 (89-290), MM2 (177-290), and MM2 (1-201) sequences, but not MM2 (1-113), suggesting that a sequence sufficient for uptake of the ctDNA by MM cells (e.g., a recognition sequence) is present between nucleotides 113-201 of MM2 (1-290) (FIG. 19). The study was repeated with 5′FAM-labelled primers, with similar results.

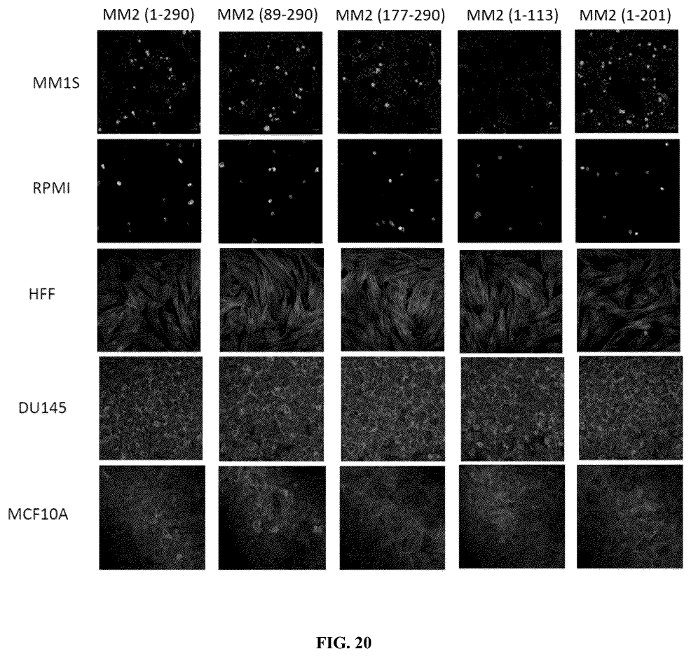

A follow-up experiment evaluated uptake of the fragments in different cell types, with counterstaining to identify nuclei (DAPI) and cytoplasm (Texas red-phalloidin). Uptake for the MM2 (1-290), MM2 (89-290), MM2 (177-290), and MM2 (1-201) sequences, but not MM2 (1-113) was observed for MM/B cell lines (MM1S and RPMI) but not other cell types HFF (fibroblast), DU145 (prostate cancer), and MCF10A (breast) (FIG. 20).

Additional fragments were generated to analyze further the sequence to identify a region that contributes to uptake by MM cells (e.g., a recognition sequence). The following sequences were generated with Fluorescein 12 dCTP or 5′FAM labelling:

TABLE 6

| SEQ | | |
|---|---|---|
| ID | Descrip- | |
| NO | tion | Sequence |
| 73 | MM2 | CCAGGATGGAGTGCAAGGGATCTTGGCTCACC |
| | (88-168) | ACAGCCTCCGCCTCTTGGGTTCAAGTGATTCC |
| | | TTCTGCCTCAGCCTCC |
| 74 | MM2 | CCAGGATGGAGTGCAAGGGATCTTGGCTCACC |
| | (88-201) | ACAGCCTCCGCCTCTTGGGTTCAAGTGATTCT |
| | | TCTGCCTCAGCCTCCCAAGTAGCTGGGATTAT |
| | | AAGCACCCACCACCACG |
| 75 | MM2 | CCAGGATGGAGTGCAAGGGATCTTGGCTCACC |
| | (88-246) | ACAGCCTCCGCCTCTTGGGTTCAAGTGATTCT |
| | | TCTGCCTCAGCCTCCCAAGTAGCTGGGATTAT |
| | | AAGCACCCACCACCACGCCCAGCTAATTTTGT |
| | | ATTTTTTAGAAGAGATGGAGTTTCTCCAGT |
| 76 | MM2 | GTGATTCTTCTGCCTCAGCCTCCCAAGTAGCT |
| | (145-246) | GGGATTATAAGCACCCACCACCACGCCCAGCT |
| | | AATTTTGTATTTTTTAGAAGAGATGGAGTTTC |
| | | TCCAGT |
| 77 | MM2 | GTGATTCTTCTGCCTCAGCCTCCCAAGTAGCT |
| | (145-290) | GGGATTATAAGCACCCACCACCACGCCCAGCT |
| | | AATTTTGTATTTTTTAGAAGAGATGGAGTTTC |
| | | TCCAGTTGGCCAGGATGGTCTGTATATCCTGA |
| | | CCTCATGATCTGCCCACC |

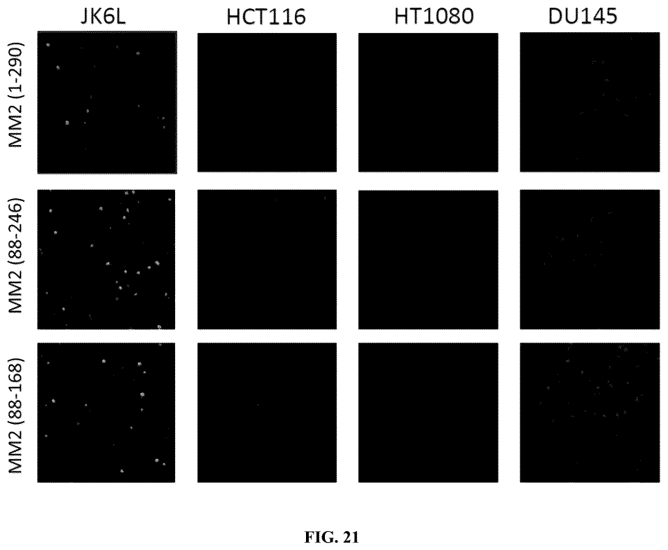

The fluorescent DNA sequences were incubated with multiple cell lines (e.g., MM cell lines MM1S, JK6L, and RPMI, and non-MM cell lines HT1080, HCT116, and DU145) and uptake was evaluated by fluorescent microscopy. The results indicated that the ˜80 base pair 88-168 region is sufficient for uptake of the DNA by MM cells. The longer 157 base pair 88-246 fragment appeared to exhibit the highest uptake of the fragments. Specificity of uptake by MM cells was preserved as shown by a lack of uptake by HT1080 and HCT116 cells (FIG. 21).

TABLE 7 summarizes details of transposons and insertion sites identified in multiple myeloma co-culture in examples of the disclosure. Location relative to insertion can be the location of the transposon relative to the insertion on the contig. Insertion position can be the position of the insertion on the contig. Transposon position can be the position of the transposon on the contig. Transposon orientation can be compared with the human reference genome. Distance from insertion can be distance between insertion and the transposon (e.g., 0 if they overlap).blast chr can be the chromosome with the longest result for BLAST analysis of the full contig. Blast position can be the position on the blast hit. Blast orientation can be BLAST alignment orientation.

TABLE 7

| | Location relative | Contig | Insertion | Transposon | Transposon | Distance from | blast | blast | blast |
|---|---|---|---|---|---|---|---|---|---|
| Transposon | to insertion | position | position | position | orientation | insertion | chr | position | orientation |
| AluSx | intersect | 1- | 1616- | 1570- | reverse | 0 | chr2 | 86528424- | Plus/Plus |
| | | 2229 | 1669 | 1871 | complement | | | 86530643 | |
| L1MB3 | right | 1- | 1616- | 1737- | reverse | −68 | chr2 | 86528424- | Plus/Plus |
| | | 2677 | 1669 | 2677 | complement | | | 86530643 | |
| L2a | intersect | 1- | 1372- | 941- | direct | 0 | chr14 | 32672352 | Plus/Minus |
| | | 2331 | 2331 | 1454 | | | | 32670273 | |
| THE1C | intersect | 1- | 1344- | 2050- | direct | 0 | chr14 | 32672352- | Plus/Minus |
| | | 2519 | 2990 | 2519 | | | | 32670273 | |
| AluY | intersect | 1- | 2355- | 2346- | reverse | 0 | chr13 | 22562455- | Plus/Minus |
| | | 2656 | 2461 | 2656 | complement | | | 22559956 | |
| LIME4b | intersect | 1- | 2390- | 1767- | reverse | 0 | chr13 | 22562455- | Plus/Minus |
| | | 2686 | 2477 | 2686 | complement | | | 22559956 | |
| AluSx | intersect | 1- | 2547- | 1573- | direct | 0 | chr17 | | |
| | | 3772 | 2494 | 1880 | | | | | |
| AluY | intersect | 1- | 419- | 153- | reverse | 0 | chr12 LIM | 58715- | Plus/Minus |
| | | 3123 | 446 | 469 | complement | | domain only 3 | 55601 | |
| AluY | intersect | 1- | 1327- | 1327- | reverse | 0 | chr8 | 15650931- | Plus/Minus |
| | | 1628 | 1433 | 1628 | complement | | | 15649499 | |
| AluY | intersect | 1- | 2445- | 2505- | direct | 0 | chr3 | 156308018- | Plus/Plus |
| | | 2816 | 2709 | 2816 | | | | 156310801 | |
| AluJb | intersect | 1- | 277- | 1- | direct | 0 | chr10 | 103114349- | Plus/Plus |
| | | 1096 | 318 | 317 | | | | 103115218 | |
| AluSx | intersect | 1- | 227- | 1- | direct | 0 | chr10 | 103114349- | Plus/Plus |
| | | 1096 | 318 | 312 | | | | 103115218 | |
| MER11C | intersect | 1- | 1584- | 2188- | reverse | 0 | chr14 | 46404852- | Plus/Plus |
| | | 3272 | 3024 | 3272 | complement | | | 46401883 | |
| AluY | intersect | 1- | 213- | 1- | reverse | 0 | chr2 | 178310609- | Plus/Plus |
| | | 1632 | 261 | 311 | complement | | | 178312037 | |
| MER11B | left | 1- | 2789- | 1- | reverse | 1554 | chr1 | 145617544- | Plus/Plus |
| | | 2915 | 2911 | 1235 | complement | | | 145619382 | |
| AluSx | intersect | 1- | 1031- | 883- | reverse | 0 | chr3 | 180870641- | Plus/Plus |
| | | 1235 | 1111 | 1190 | complement | | | 180871874 | |
| AluY | intersect | 1- | 133- | 1- | direct | 0 | chr6 | 143000057- | Plus/Plus |
| | | 1786 | 284 | 312 | | | | 143001731 | |
| AluSx | intersect | 1- | 381- | 261- | direct | 0 | chr5 | 71047678- | Plus/Minus |
| | | 4743 | 552 | 574 | | | | 71042940 | |
| AluY | intersect | 1- | 381- | 261- | direct | 0 | chr5 | 71047678- | Plus/Minus |
| | | 4743 | 552 | 574 | | | | 71042940 | |
| Tigger3a | right | 1- | 40- | 2599- | direct | 2389 | chr5 | 71047678- | Plus/Minus |
| | | 4764 | 210 | 2941 | | | | 71042940 | |

TABLE 8 summarizes details of transposons and insertion sites identified in pancreatic cancer co-culture in examples of the disclosure. Location relative to insertion can be the location of the transposon relative to the insertion on the contig. Insertion position can be the position of the insertion on the contig. Transposon position can be the position of the transposon on the contig. Transposon orientation can be compared with the human reference genome. Distance from insertion can be distance between insertion and the transposon (e.g., 0 if they overlap).blast chr can be the chromosome with the longest result for BLAST analysis of the full contig. Blast position can be the position on the blast hit. Blast orientation can be BLAST alignment orientation.

TABLE 8

| | Location relative | Contig | Insertion | Transposon | Transposon | Distance from | blast | blast | blast |
|---|---|---|---|---|---|---|---|---|---|
| Transposon | to insertion | position | position | position | orientation | insertion | chr | position | orientation |
| AluJb | right | 1- | 51- | 3394- | direct | +3174 | chr18 | 23191079- | Plus/Minus |
| | | 3705 | 220 | 3705 | | | | 23187483 | |
| AluSx | right | 1- | 145- | 3394- | direct | +3174 | chr18 | 23191079- | Plus/Minus |
| | | 3705 | 220 | 3705 | | | | 23187483 | |
| L1PA15 | right | 1- | 84- | 6793- | reverse | −6514 | chr4 | 74809282- | Plus/Minus |
| | | 7716 | 279 | 7704 | complement | | | 74804853 | |
| L1PB4 | right | 1- | 84- | 6801- | reverse | −6522 | chr4 | 74809282- | Plus/Minus |
| | | 7716 | 279 | 7704 | complement | | | 74804853 | |
| AluSx | intersect | 1- | 198- | 169- | direct | 0 | chr20 | 60039432- | Plus/Minus |
| | | 3368 | 336 | 483 | | | | 60036075 | |
| AluSx | intersect | 1- | 4456- | 4409- | direct | 0 | chr16 | 30075661- | Plus/Plus |
| | | 4730 | 4591 | 4720 | | | | 30080375 | |
| MIRc | left | 1- | 4465- | 2056- | reverse | +2206 | chr16 | 30075661- | Plus/Plus |
| | | 4739 | 4600 | 2259 | complement | | | 30080375 | |
| AluJb | intersect | 1- | 2619- | 2450- | reverse | 0 | chr7 | 78202685- | Plus/Plus |
| | | 3676 | 2651 | 2747 | complement | | | 78206359 | |
| AluSx | intersect | 1- | 2619- | 2450- | reverse | 0 | chr7 | 78202685- | Plus/Plus |
| | | 3676 | 2651 | 2747 | complement | | | 78206359 | |
| AluSx | intersect | 1- | 1963- | 1677- | reverse | 0 | chr8 | 17791254- | Plus/Plus |
| | | 7197 | 1980 | 1989 | complement | | | 17789008 | |
| AluY | intersect | 1- | 6052- | 5828- | reverse | 0 | chr8 | 17791254- | Plus/Plus |
| | | 7197 | 6112 | 6138 | complement | | | 17789008 | |
| MIRb | left | 1- | 5476- | 4109- | direct | −1099 | chr8 | 17791254- | Plus/Plus |
| | | 7240 | 5518 | 4377 | | | | 17789008 | |
| MIRc | left | 1- | 5441- | 4106- | direct | −1117 | chr8 | 17791254- | Plus/Plus |
| | | 7205 | 5483 | 4324 | | | | 17789008 | |
| AluSx | intersect | 1- | 3257- | 3270- | direct | 0 | chr3 | 128761943- | Plus/Plus |
| | | 3669 | 3375 | 3567 | | | | 128759611 | |
| MLT2B1 | intersect | 1- | 3352- | 2852- | direct | 0 | chr3 | 128761943- | Plus/Plus |
| | | 3763 | 3470 | 3356 | | | | 128759611 | |
| AluSx | intersect | 1- | 1421- | 2841- | reverse | 0 | chr21 | 39057278- | Plus/Plus |
| | | 7277 | 3369 | 3166 | complement | | | 39054368 | |
| L2a | intersect | 1- | 1421- | 2474- | direct | 0 | chr21 | 39057278- | Plus/Plus |
| | | 7340 | 3654 | 2881 | | | | 39054368 | |
| MLT1J2 | intersect | 1- | 1473- | 1167- | direct | 0 | chr21 | 39057278- | Plus/Plus |
| | | 7311 | 3625 | 1529 | | | | 39054368 | |
| AluSx | intersect | 1- | 989- | 960- | direct | 0 | chr7 | 22688622- | Plus/Plus |
| | | 4351 | 1127 | 1264 | | | | 22692178 | |
| AluY | intersect | 1- | 989- | 960- | direct | 0 | chr7 | 22688622- | Plus/Plus |
| | | 4251 | 1127 | 1264 | | | | 22692178 | |
| L1ME4b | right | 1- | 989- | 3057- | direct | +1930 | chr7 | 22688622- | Plus/Plus |
| | | 4422 | 1127 | 3720 | | | | 22692178 | |
| MIRb | intersect | 1- | 3869- | 3790- | direct | 0 | chr14 | 101625831- | Plus/Minus |
| | | 9468 | 3929 | 3904 | | | | 101623094 | |
| MER41E | right | 1- | 2493- | 5071- | direct | +2512 | chr2 | 240694078- | Plus/Plus |
| | | 5966 | 2559 | 5660 | | | | 240699987 | |
| AluSx | intersect | 1- | 2220- | 2191- | direct | 0 | chr9 | 77999115- | Plus/Plus |
| | | 7125 | 2359 | 2502 | | | | 78001422 | |
| Mam_R4 | intersect | 1- | 2269- | 1015- | direct | 0 | chr9 | 77999115- | Plus/Plus |
| | | 7466 | 2410 | 4872 | | | | 78001422 | |
| MIRb | left | 1- | 2236- | 281- | reverse | +1686 | chr9 | 77999115- | Plus/Plus |
| | | 7141 | 2375 | 550 | complement | | | 78001422 | |
| MIRb | right | 1- | 2751- | 3949- | reverse | −1166 | chr4 | 155243887- | Plus/Minus |
| | | 10415 | 2783 | 4170 | complement | | | 155240822 | |

1-158. (canceled)

159. A pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence comprises the nucleotide sequence of SEQ ID NO: 73.

160. The pharmaceutical composition of claim 159, wherein the recognition sequence consists essentially of the nucleotide sequence of SEQ ID NO: 73.

161. The pharmaceutical composition of claim 159, wherein the recognition sequence does not contain a nucleotide sequence with at least 90% sequence identity to nucleotides 1-87 of SEQ ID NO: 68.

162. The pharmaceutical composition of claim 159, wherein the recognition sequence comprises a nucleotide sequence with at least 90% sequence identity to SEQ ID NO: 74 or SEQ ID NO: 75.

163. The pharmaceutical composition of claim 159, wherein the recognition sequence facilitates binding of the synthetic delivery system to the target cell.

164. The pharmaceutical composition of claim 159, wherein the recognition sequence facilitates uptake of the synthetic delivery system or component thereof by the target cell.

165. The pharmaceutical composition of claim 159, wherein the recognition sequence facilitates at least 50% higher uptake of the synthetic delivery system by the target cell as compared to a control synthetic delivery system that lacks the recognition sequence.

166. The pharmaceutical composition of claim 159, wherein the recognition sequence facilitates at least 50% higher uptake of the synthetic delivery system by the target cell as compared to a control cell of a different cell type.

167. The pharmaceutical composition of claim 159, wherein the polynucleotide comprises double stranded DNA.

168. The pharmaceutical composition of claim 159, wherein the synthetic delivery system further comprises a cargo.

169. The pharmaceutical composition of claim 168, wherein the cargo comprises an anti-cancer therapeutic agent.

170. The pharmaceutical composition of claim 168, wherein the cargo comprises a cytotoxic cargo.

171. The pharmaceutical composition of claim 168, wherein the cargo comprises a small molecule.

172. The pharmaceutical composition of claim 159, wherein the target cell is a cancer cell.

173. The pharmaceutical composition of claim 159, wherein the target cell is a multiple myeloma cell.

174. The pharmaceutical composition of claim 159, wherein the target cell is a pancreatic cancer cell.

175. A method of delivering a cargo to a target cell, the method comprising contacting the target cell with the pharmaceutical composition or the synthetic delivery system of claim 159.

176. A method of treating a condition in a subject in need thereof, the method comprising administering to the subject the pharmaceutical composition of claim 159.

177. A pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence consists essentially of a nucleotide sequence with at least 80% sequence identity to any one of SEQ ID NOs: 73-75.

178. A pharmaceutical composition comprising a synthetic delivery system and a pharmaceutically-acceptable excipient, wherein the synthetic delivery system comprises a polynucleotide comprising a recognition sequence that facilitates binding of the synthetic delivery system to a target cell or uptake of the synthetic delivery system or a component thereof by the target cell, wherein the recognition sequence comprises:
(a) a nucleotide sequence with at least 80% sequence identity to at least 10 consecutive nucleotides of SEQ ID NO: 13 or 14;
(b) a nucleotide sequence with at least 90% sequence identity to at least 60 consecutive nucleotides of SEQ ID NO: 33 or a nucleotide sequence with at least 91% sequence identity to at least 50 consecutive nucleotides of SEQ ID NO: 33;
(c) a nucleotide sequence with at least 86% sequence identity to any one of SEQ ID NOs: 7, 16, 25, 29, and 31; or
(d) an AluSp, AluSx, AluSg2, ERV2 superfamily, or ERV3 superfamily transposable element, or a functional fragment thereof.
